SEQUENCE LISTING (258 sequences)

SEQ ID NO: 1 - protein, 10 aa, synthetic construct
GYTFTDYAMN

SEQ ID NO: 2 - protein, 10 aa, synthetic construct
WINTQTGKPT

SEQ ID NO: 3 - protein, 11 aa, synthetic construct
LGTGNTKGFAY

SEQ ID NO: 4 - protein, 120 aa, synthetic construct
QIQLVQSGSELKKPGASVKVSCKASGYTFTDYAMNWVRQAPGQGLEWMGWINTQTGKPTYAQGFTGRFVFSLDTSVSTAYLQISSLKAEDTAVYYCTRLGTGNTKGFAYWGQGTLVTVSS

SEQ ID NO: 5 - protein, 120 aa, synthetic construct
QIQLVQSGAEVKKPGSSVKVSCKASGYTFTDYAMNWVRQAPGQGLEWMGWINTQTGKPTYAQKFQGRVTFTLDESTSTAYMELSSLRSEDTAVYYCTRLGTGNTKGFAYWGQGTLVTVSS

SEQ ID NO: 6 - protein, 120 aa, synthetic construct
QIQLVQSGAEVKKPGSSVKVSCKASGYTFTDYAMNWVRQAPGQGLEWMGWINTQTGKPTYAQKFQGRVTFTLDESTSTTYMELSSLRSEDTAVYYCARLGTGNTKGFAYWGQGTLVTVSS

SEQ ID NO: 7 - protein, 120 aa, synthetic construct
QIQLVQSGAEVKKPGSSVKVSCKASGYTFTDYAMNWVRQAPGQGLEWMGWINTQTGKPTYAQKFQGRVTFTLDESTSTTYMELSSLRSEDTAVYYCTRLGTGNTKGFAYWGQGTLVTVSS

SEQ ID NO: 8 - protein, 120 aa, synthetic construct
QIQLVQSGPEVKKPGESVKISCKASGYTFTDYAMNWVKQAPGNGLKWMGWINTQTGKPTYADDFKQRFVFSLETSARTTYLQINNLNIEDTATYFCTRLGTGNTKGFAYWGQGTLVTVSS

SEQ ID NO: 9 - protein, 120 aa, synthetic construct
QIQLVQSGSELKKPGASVKISCKASGYTFTDYAMNWVKQAPGQGLEWMGWINTQTGKPTYAQGFTGRFVFSLDTSVSTAYLQISSLKAEDTAVYFCTRLGTGNTKGFAYWGQGTLVTVSS

SEQ ID NO: 10 - protein, 120 aa, synthetic construct; identical to SEQ ID NO: 9

SEQ ID NO: 11 - protein, 120 aa, synthetic construct
QIQLVQSGSELKKPGASVKISCKASGYTFTDYAMNWVRQAPGQGLEWMGWINTQTGKPTYAQGFTGRFVFSLDTSVRTAYLQISSLKAEDTAVYFCTRLGTGNTKGFAYWGQGTLVTVSS

SEQ ID NO: 12 - protein, 120 aa, synthetic construct
QIQLVQSGSELKKPGASVKISCKASGYTFTDYAMNWVRQAPGQGLEWMGWINTQTGKPTYAQGFTGRFVFSLDTSVSTAYLQISSLKAEDTAVYFCTRLGTGNTKGFAYWGQGTLVTVSS

SEQ ID NO: 13 - protein, 120 aa, synthetic construct
QIQLVQSGSELKKPGASVKISCKASGYTFTDYAMNWVRQAPGQGLEWMGWINTQTGKPTYAQGFTGRFVFSLDTSVSTAYLQISSLKAEDTAVYYCTRLGTGNTKGFAYWGQGTLVTVSS

SEQ ID NO: 14 - protein, 120 aa, synthetic construct
QIQLVQSGSELKKPGASVKVSCKASGYTFTDYAMNWVRQAPGQGLEWMGWINTQTGKPTYAQGFTGRFVFSLDTSVSTAYLQISSLKAEDTAVYFCTRLGTGNTKGFAYWGQGTLVTVSS

SEQ ID NO: 15 - protein, 120 aa, synthetic construct
QVQLVQSGAEVKKPGSSVKVSCKASGYTFTDYAMNWVRQAPGQGLEWMGWINTQTGKPTYAQKFQGRVTFTLDESTSTAYMELSSLRSEDTAVYYCTRLGTGNTKGFAYWGQGTLVTVSS

SEQ ID NO: 16 - protein, 17 aa, synthetic construct
KSSQSLLYSENNQDYLA

SEQ ID NO: 17 - protein, 7 aa, synthetic construct
GASNRHT

SEQ ID NO: 18 - protein, 9 aa, synthetic construct
EQTYRYPFT

SEQ ID NO: 19 - protein, 113 aa, synthetic construct
DIQITQSPSSLSASVGDRVTITCKSSQSLLYSENNQDYLAWYQQKPGKVPKLLIYGASNRHTGVPSRFSGSGSGTDFTLTISSLQPEDVATYYCEQTYRYPFTFGQGTKLEIK

SEQ ID NO: 20 - protein, 113 aa, synthetic construct
DILINQSPASLTVSAGERVTMSCKSSQSLLYSENNQDYLAWYQQKPGQFPKLLIYGASNRHTGVPDRFTGSGSGTDFTLTISSVQAEDLADYYCEQTYRYPFTFGSGTKLEIK

SEQ ID NO: 21 - protein, 113 aa, synthetic construct; identical to SEQ ID NO: 19

SEQ ID NO: 22 - protein, 113 aa, synthetic construct
DIQMTQSPSSLSASVGDRVTITCKSSQSLLYSENNQDYLAWYQQKPGKVPKLLIYGASNRHTGVPSRFSGSGSGTDFTLTISSLQPEDVATYYCEQTYRYPFTFGQGTKLEIK

SEQ ID NO: 23 - protein, 10 aa, synthetic construct
GYTFTSYGIS

SEQ ID NO: 24 - protein, 10 aa, synthetic construct
WISAYNANTN

SEQ ID NO: 25 - protein, 10 aa, synthetic construct
WISAYNANVN

SEQ ID NO: 26 - protein, 10 aa, synthetic construct
WISAYNGNTN

SEQ ID NO: 27 - protein, 10 aa, synthetic construct
WISAYNSNTN

SEQ ID NO: 28 - protein, 11 aa, synthetic construct
VTGITGTTIGP

SEQ ID NO: 29 - protein, 11 aa, synthetic construct
VTGITGTTIDP

SEQ ID NO: 30 - protein, 120 aa, synthetic construct
QVQLVQSGAEVKKPGASVKVSCKASGYTFTSYGISWVRQAPGQGLEWMGWISAYNANTNYAQKLQGRVTMTTDTSTSTAYMELRSLRSDDTAAYYCARVTGITGTTIGPWGQGTMVTVSS

SEQ ID NO: 31 - protein, 120 aa, synthetic construct
QVQLVQSGAEVKKPGASVKVSCKASGYTFTSYGISWVRQAPGQGLEWMGWISAYNANVNYAQKLQGRVTMTTDTSTSTAYMELRSLRSDDTAAYYCARVTGITGTTIGPWGQGTMVTVSS

SEQ ID NO: 32 - protein, 120 aa, synthetic construct
QVQLVQSGAEVKKPGASVKVSCKASGYTFTSYGISWVRQAPGQGLEWMGWISAYNGNTNYAQKLQGRVTMTTDTSTSTAYMELRSLRSDDTAAYYCARVTGITGTTIDPWGQGTMVTVSS

SEQ ID NO: 33 - protein, 120 aa, synthetic construct
QVQLVQSGAEVKKPGASVKVSCKASGYTFTSYGISWVRQAPGQGLEWMGWISAYNSNTNYAQKLQGRVTMTTDTSTSTAYMELRSLRSDDTAAYYCARVTGITGTTIGPWGQGTMVTVSS

SEQ ID NO: 34 - protein, 11 aa, synthetic construct
RASQSISSYLN

SEQ ID NO: 35 - protein, 7 aa, synthetic construct
DASSLES

SEQ ID NO: 36 - protein, 9 aa, synthetic construct
QQYNNWPQT

SEQ ID NO: 37 - protein, 107 aa, synthetic construct
DIQMTQSPSSLSASVGDRVTITCRASQSISSYLNWYQQKPGKAPKLLIYDASSLESGVPSRFSGSGSGTEFTLTISSLQPDDFAVYYCQQYNNWPQTFGQGTKVEIK

SEQ ID NO: 38 - protein, 11 aa, synthetic construct
GFSFSSSYYMA

SEQ ID NO: 39 - protein, 11 aa, synthetic construct
GFSFSSSYYMC

SEQ ID NO: 40 - protein, 11 aa, synthetic construct
GFSFSSSYYMS

SEQ ID NO: 41 - protein, 10 aa, synthetic construct
AIGTTRGSTY

SEQ ID NO: 42 - protein, 10 aa, synthetic construct
CIGTTRGSTY

SEQ ID NO: 43 - protein, 16 aa, synthetic construct
DATGYRINTIGLYFNL

SEQ ID NO: 44 - protein, 126 aa, synthetic construct
EVQLLESGGGLVQPGGSLRLSCAASGFSFSSSYYMCWVRQAPGKGLEWVSAIGTTRGSTYYADSVKGRFTISRDSSKNTVYLQMNSLRAEDTAVYYCARDATGYRINTIGLYFNLWGQGTLVTVSS

SEQ ID NO: 45 - protein, 126 aa, synthetic construct
EVQLLESGGGLVQPGGSLRLSCAASGFSFSSSYYMAWVRQAPGKGLEWVSAIGTTRGSTYYADSVKGRFTISRDNSKNTLYLQMNSLRAEDTAVYYCARDATGYRINTIGLYFNLWGQGTLVTVSS

SEQ ID NO: 46 - protein, 126 aa, synthetic construct
EVQLLESGGGLVQPGGSLRLSCAASGFSFSSSYYMAWVRQAPGKGLEWVSAIGTTRGSTYYADSVKGRFTISRDNSKNTVYLQMNSLRAEDTAVYYCARDATGYRINTIGLYFNLWGQGTLVTVSS

SEQ ID NO: 47 - protein, 126 aa, synthetic construct
EVQLLESGGGLVQPGGSLRLSCAASGFSFSSSYYMAWVRQAPGKGLEWVSAIGTTRGSTYYADSVKGRFTISRDSSKNTLYLQMNSLRAEDTAVYYCARDATGYRINTIGLYFNLWGQGTLVTVSS

SEQ ID NO: 48 - protein, 126 aa, synthetic construct
EVQLLESGGGLVQPGGSLRLSCAASGFSFSSSYYMAWVRQAPGKGLEWVSAIGTTRGSTYYADSVKGRFTISRDSSKNTVYLQMNSLRAEDTAVYFCARDATGYRINTIGLYFNLWGQGTLVTVSS

SEQ ID NO: 49 - protein, 126 aa, synthetic construct
EVQLLESGGGLVQPGGSLRLSCAASGFSFSSSYYMCWVRQAPGKGLEWVSCIGTTRGSTYYADSVKGRFTISRDNSKNTLYLQMNSLRAEDTAVYYCARDATGYRINTIGLYFNLWGQGTLVTVSS

SEQ ID NO: 50 - protein, 126 aa, synthetic construct
EVQLLESGGGLVQPGGSLRLSCAASGFSFSSSYYMCWVRQAPGKGLEWVSCIGTTRGSTYYADSVKGRFTISRDNSKNTVYLQMNSLRAEDTAVYYCARDATGYRINTIGLYFNLWGQGTLVTVSS

SEQ ID NO: 51 - protein, 126 aa, synthetic construct
EVQLLESGGGLVQPGGSLRLSCAASGFSFSSSYYMCWVRQAPGKGLEWVSCIGTTRGSTYYADSVKGRFTISRDSSKNTLYLQMNSLRAEDTAVYYCARDATGYRINTIGLYFNLWGQGTLVTVSS

SEQ ID NO: 52 - protein, 126 aa, synthetic construct
EVQLLESGGGLVQPGGSLRLSCAASGFSFSSSYYMCWVRQAPGKGLEWVSCIGTTRGSTYYADSVKGRFTISRDSSKNTVYLQMNSLRAEDTAVYFCARDATGYRINTIGLYFNLWGQGTLVTVSS

SEQ ID NO: 53 - protein, 126 aa, synthetic construct
EVQLLESGGGLVQPGGSLRLSCAASGFSFSSSYYMSWVRQAPGKGLEWVSAIGTTRGSTYYADSVKGRFTISRDNSKNTLYLQMNSLRAEDTAVYYCARDATGYRINTIGLYFNLWGQGTLVTVSS

SEQ ID NO: 54 - protein, 126 aa, synthetic construct
EVQLLESGGGLVQPGGSLRLSCAASGFSFSSSYYMSWVRQAPGKGLEWVSAIGTTRGSTYYADSVKGRFTISRDNSKNTVYLQMNSLRAEDTAVYYCARDATGYRINTIGLYFNLWGQGTLVTVSS

SEQ ID NO: 55 - protein, 126 aa, synthetic construct
EVQLLESGGGLVQPGGSLRLSCAASGFSFSSSYYMSWVRQAPGKGLEWVSAIGTTRGSTYYADSVKGRFTISRDSSKNTLYLQMNSLRAEDTAVYYCARDATGYRINTIGLYFNLWGQGTLVTVSS

SEQ ID NO: 56 - protein, 126 aa, synthetic construct
EVQLLESGGGLVQPGGSLRLSCAASGFSFSSSYYMSWVRQAPGKGLEWVSAIGTTRGSTYYADSVKGRFTISRDSSKNTVYLQMNSLRAEDTAVYFCARDATGYRINTIGLYFNLWGQGTLVTVSS

SEQ ID NO: 57 - protein, 124 aa, synthetic construct
QSLEESGGDLVKPGASLTLTCRVSGFSFSSSYYMCWVRQAPGKGLEWIACIGTTRGSTYYATWAKGRFTISKISSTTVTLQMTSLTDADTATYFCARDATGYRINTIGLYFNLWGPGTLVTVSS

SEQ ID NO: 58 - protein, 11 aa, synthetic construct
QASQTIYSYLS

SEQ ID NO: 59 - protein, 7 aa, synthetic construct
EASKLAS

SEQ ID NO: 60 - protein, 13 aa, synthetic construct
QSYHGTASTEYNT

SEQ ID NO: 61 - protein, 111 aa, synthetic construct
DIVMTQSPDSLAVSLGERATINCQASQTIYSYLSWYQQKPGQPPKLLIYEASKLASGVPDRFSGSGSGTDYTLTISSLQAEDVAVYYCQSYHGTASTEYNTFGGGTKVEIK

SEQ ID NO: 62 - protein, 109 aa, synthetic construct
ELTQTPSSVEAAVGGTPTIKCQASQTIYSYLSWYQQKPGQPPKLLIYEASKLASGVPSRFSGSGSGTDYTLTISDLECADAATYYCQSYHGTASTEYNTFGGGTEVVVK

SEQ ID NO: 63 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 64 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGK

SEQ ID NO: 65 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 66 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 67 - protein, 326 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 68 - protein, 326 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 69 - protein, 107 aa, synthetic construct [sequence omitted]

SEQ ID NO: 70 - protein, 25 aa, synthetic construct
  VARIANT 1..25: note = This sequence may encompass 1-5 GGGGS repeating units
GGGGSGGGGSGGGGSGGGGSGGGGS

SEQ ID NO: 71 - protein, 25 aa, synthetic construct
  VARIANT 1..25: note = This sequence may encompass 1-5 GGGGA repeating units
GGGGAGGGGAGGGGAGGGGAGGGGA

SEQ ID NO: 72 - protein, 450 aa, synthetic construct
QIQLVQSGSELKKPGASVKVSCKASGYTFTDYAMNWVRQAPGQGLEWMGWINTQTGKPTYAQGFTGRFVFSLDTSVSTAYLQISSLKAEDTAVYYCTRLGTGNTKGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 73 - protein, 450 aa, synthetic construct
QIQLVQSGAEVKKPGSSVKVSCKASGYTFTDYAMNWVRQAPGQGLEWMGWINTQTGKPTYAQKFQGRVTFTLDESTSTAYMELSSLRSEDTAVYYCTRLGTGNTKGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 74 - protein, 450 aa, synthetic construct
QIQLVQSGAEVKKPGSSVKVSCKASGYTFTDYAMNWVRQAPGQGLEWMGWINTQTGKPTYAQKFQGRVTFTLDESTSTTYMELSSLRSEDTAVYYCARLGTGNTKGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 75 - protein, 450 aa, synthetic construct
QIQLVQSGAEVKKPGSSVKVSCKASGYTFTDYAMNWVRQAPGQGLEWMGWINTQTGKPTYAQKFQGRVTFTLDESTSTTYMELSSLRSEDTAVYYCTRLGTGNTKGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 76 - protein, 450 aa, synthetic construct
QIQLVQSGPEVKKPGESVKISCKASGYTFTDYAMNWVKQAPGNGLKWMGWINTQTGKPTYADDFKQRFVFSLETSARTTYLQINNLNIEDTATYFCTRLGTGNTKGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 77 - protein, 450 aa, synthetic construct
QIQLVQSGSELKKPGASVKISCKASGYTFTDYAMNWVKQAPGQGLEWMGWINTQTGKPTYAQGFTGRFVFSLDTSVSTAYLQISSLKAEDTAVYFCTRLGTGNTKGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 78 - protein, 450 aa, synthetic construct; identical to SEQ ID NO: 77

SEQ ID NO: 79 - protein, 450 aa, synthetic construct
QIQLVQSGSELKKPGASVKISCKASGYTFTDYAMNWVRQAPGQGLEWMGWINTQTGKPTYAQGFTGRFVFSLDTSVRTAYLQISSLKAEDTAVYFCTRLGTGNTKGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 80 - protein, 450 aa, synthetic construct
QIQLVQSGSELKKPGASVKISCKASGYTFTDYAMNWVRQAPGQGLEWMGWINTQTGKPTYAQGFTGRFVFSLDTSVSTAYLQISSLKAEDTAVYFCTRLGTGNTKGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 81 - protein, 450 aa, synthetic construct
QIQLVQSGSELKKPGASVKISCKASGYTFTDYAMNWVRQAPGQGLEWMGWINTQTGKPTYAQGFTGRFVFSLDTSVSTAYLQISSLKAEDTAVYYCTRLGTGNTKGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 82 - protein, 450 aa, synthetic construct
QIQLVQSGSELKKPGASVKVSCKASGYTFTDYAMNWVRQAPGQGLEWMGWINTQTGKPTYAQGFTGRFVFSLDTSVSTAYLQISSLKAEDTAVYFCTRLGTGNTKGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 83 - protein, 450 aa, synthetic construct
QVQLVQSGAEVKKPGSSVKVSCKASGYTFTDYAMNWVRQAPGQGLEWMGWINTQTGKPTYAQKFQGRVTFTLDESTSTAYMELSSLRSEDTAVYYCTRLGTGNTKGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 84 - protein, 447 aa, synthetic construct
QIQLVQSGSELKKPGASVKVSCKASGYTFTDYAMNWVRQAPGQGLEWMGWINTQTGKPTYAQGFTGRFVFSLDTSVSTAYLQISSLKAEDTAVYYCTRLGTGNTKGFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGK

SEQ ID NO: 85 - protein, 447 aa, synthetic construct
QIQLVQSGAEVKKPGSSVKVSCKASGYTFTDYAMNWVRQAPGQGLEWMGWINTQTGKPTYAQKFQGRVTFTLDESTSTAYMELSSLRSEDTAVYYCTRLGTGNTKGFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGK

SEQ ID NO: 86 - protein, 447 aa, synthetic construct
QIQLVQSGAEVKKPGSSVKVSCKASGYTFTDYAMNWVRQAPGQGLEWMGWINTQTGKPTYAQKFQGRVTFTLDESTSTTYMELSSLRSEDTAVYYCARLGTGNTKGFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGK

SEQ ID NO: 87 - protein, 447 aa, synthetic construct
QIQLVQSGAEVKKPGSSVKVSCKASGYTFTDYAMNWVRQAPGQGLEWMGWINTQTGKPTYAQKFQGRVTFTLDESTSTTYMELSSLRSEDTAVYYCTRLGTGNTKGFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGK

SEQ ID NO: 88 - protein, 447 aa, synthetic construct
QIQLVQSGPEVKKPGESVKISCKASGYTFTDYAMNWVKQAPGNGLKWMGWINTQTGKPTYADDFKQRFVFSLETSARTTYLQINNLNIEDTATYFCTRLGTGNTKGFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGK

SEQ ID NO: 89 - protein, 447 aa, synthetic construct
QIQLVQSGSELKKPGASVKISCKASGYTFTDYAMNWVKQAPGQGLEWMGWINTQTGKPTYAQGFTGRFVFSLDTSVSTAYLQISSLKAEDTAVYFCTRLGTGNTKGFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGK

SEQ ID NO: 90 - protein, 447 aa, synthetic construct; identical to SEQ ID NO: 89

SEQ ID NO: 91 - protein, 447 aa, synthetic construct
QIQLVQSGSELKKPGASVKISCKASGYTFTDYAMNWVRQAPGQGLEWMGWINTQTGKPTYAQGFTGRFVFSLDTSVRTAYLQISSLKAEDTAVYFCTRLGTGNTKGFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGK

SEQ ID NO: 92 - protein, 447 aa, synthetic construct
QIQLVQSGSELKKPGASVKISCKASGYTFTDYAMNWVRQAPGQGLEWMGWINTQTGKPTYAQGFTGRFVFSLDTSVSTAYLQISSLKAEDTAVYFCTRLGTGNTKGFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGK

SEQ ID NO: 93 - protein, 447 aa, synthetic construct
QIQLVQSGSELKKPGASVKISCKASGYTFTDYAMNWVRQAPGQGLEWMGWINTQTGKPTYAQGFTGRFVFSLDTSVSTAYLQISSLKAEDTAVYYCTRLGTGNTKGFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGK

SEQ ID NO: 94 - protein, 447 aa, synthetic construct
QIQLVQSGSELKKPGASVKVSCKASGYTFTDYAMNWVRQAPGQGLEWMGWINTQTGKPTYAQGFTGRFVFSLDTSVSTAYLQISSLKAEDTAVYFCTRLGTGNTKGFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGK

SEQ ID NO: 95 - protein, 447 aa, synthetic construct
QVQLVQSGAEVKKPGSSVKVSCKASGYTFTDYAMNWVRQAPGQGLEWMGWINTQTGKPTYAQKFQGRVTFTLDESTSTAYMELSSLRSEDTAVYYCTRLGTGNTKGFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGK

SEQ ID NO: 96 - protein, 447 aa, synthetic construct
QIQLVQSGSELKKPGASVKVSCKASGYTFTDYAMNWVRQAPGQGLEWMGWINTQTGKPTYAQGFTGRFVFSLDTSVSTAYLQISSLKAEDTAVYYCTRLGTGNTKGFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 97 - protein, 447 aa, synthetic construct
QIQLVQSGAEVKKPGSSVKVSCKASGYTFTDYAMNWVRQAPGQGLEWMGWINTQTGKPTYAQKFQGRVTFTLDESTSTAYMELSSLRSEDTAVYYCTRLGTGNTKGFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 98 - protein, 447 aa, synthetic construct
QIQLVQSGAEVKKPGSSVKVSCKASGYTFTDYAMNWVRQAPGQGLEWMGWINTQTGKPTYAQKFQGRVTFTLDESTSTTYMELSSLRSEDTAVYYCARLGTGNTKGFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 99 - protein, 447 aa, synthetic construct
QIQLVQSGAEVKKPGSSVKVSCKASGYTFTDYAMNWVRQAPGQGLEWMGWINTQTGKPTYAQKFQGRVTFTLDESTSTTYMELSSLRSEDTAVYYCTRLGTGNTKGFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 100 - protein, 447 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKQIQLVQSGPEVKKPGESVKISCKASGYTFTDYAMNWVKQAPGNGLKWMGWINTQTGKPTYADDFKQRFVFSLETSARTTYLQINNLNIEDTATYFCTRLGTGNTKGFAYWGQGTLVTVSS

SEQ ID NO: 101 - protein, 447 aa, synthetic construct
QIQLVQSGSELKKPGASVKISCKASGYTFTDYAMNWVKQAPGQGLEWMGWINTQTGKPTYAQGFTGRFVFSLDTSVSTAYLQISSLKAEDTAVYFCTRLGTGNTKGFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 102 - protein, 447 aa, synthetic construct; identical to SEQ ID NO: 101

SEQ ID NO: 103 - protein, 447 aa, synthetic construct
QIQLVQSGSELKKPGASVKISCKASGYTFTDYAMNWVRQAPGQGLEWMGWINTQTGKPTYAQGFTGRFVFSLDTSVRTAYLQISSLKAEDTAVYFCTRLGTGNTKGFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 104 - protein, 447 aa, synthetic construct
QIQLVQSGSELKKPGASVKISCKASGYTFTDYAMNWVRQAPGQGLEWMGWINTQTGKPTYAQGFTGRFVFSLDTSVSTAYLQISSLKAEDTAVYFCTRLGTGNTKGFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 105 - protein, 447 aa, synthetic construct
QIQLVQSGSELKKPGASVKISCKASGYTFTDYAMNWVRQAPGQGLEWMGWINTQTGKPTYAQGFTGRFVFSLDTSVSTAYLQISSLKAEDTAVYYCTRLGTGNTKGFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 106 - protein, 447 aa, synthetic construct
QIQLVQSGSELKKPGASVKVSCKASGYTFTDYAMNWVRQAPGQGLEWMGWINTQTGKPTYAQGFTGRFVFSLDTSVSTAYLQISSLKAEDTAVYFCTRLGTGNTKGFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 107 - protein, 447 aa, synthetic construct
QVQLVQSGAEVKKPGSSVKVSCKASGYTFTDYAMNWVRQAPGQGLEWMGWINTQTGKPTYAQKFQGRVTFTLDESTSTAYMELSSLRSEDTAVYYCTRLGTGNTKGFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 108 - protein, 449 aa, synthetic construct
QIQLVQSGSELKKPGASVKVSCKASGYTFTDYAMNWVRQAPGQGLEWMGWINTQTGKPTYAQGFTGRFVFSLDTSVSTAYLQISSLKAEDTAVYYCTRLGTGNTKGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 109 - protein, 449 aa, synthetic construct
QIQLVQSGAEVKKPGSSVKVSCKASGYTFTDYAMNWVRQAPGQGLEWMGWINTQTGKPTYAQKFQGRVTFTLDESTSTAYMELSSLRSEDTAVYYCTRLGTGNTKGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 110 - protein, 449 aa, synthetic construct
QIQLVQSGAEVKKPGSSVKVSCKASGYTFTDYAMNWVRQAPGQGLEWMGWINTQTGKPTYAQKFQGRVTFTLDESTSTTYMELSSLRSEDTAVYYCARLGTGNTKGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 111 - protein, 449 aa, synthetic construct
QIQLVQSGAEVKKPGSSVKVSCKASGYTFTDYAMNWVRQAPGQGLEWMGWINTQTGKPTYAQKFQGRVTFTLDESTSTTYMELSSLRSEDTAVYYCTRLGTGNTKGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 112 - protein, 449 aa, synthetic construct
QIQLVQSGPEVKKPGESVKISCKASGYTFTDYAMNWVKQAPGNGLKWMGWINTQTGKPTYADDFKQRFVFSLETSARTTYLQINNLNIEDTATYFCTRLGTGNTKGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 113 - protein, 449 aa, synthetic construct
QIQLVQSGSELKKPGASVKISCKASGYTFTDYAMNWVKQAPGQGLEWMGWINTQTGKPTYAQGFTGRFVFSLDTSVSTAYLQISSLKAEDTAVYFCTRLGTGNTKGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 114 - protein, 449 aa, synthetic construct; identical to SEQ ID NO: 113

SEQ ID NO: 115 - protein, 449 aa, synthetic construct
QIQLVQSGSELKKPGASVKISCKASGYTFTDYAMNWVRQAPGQGLEWMGWINTQTGKPTYAQGFTGRFVFSLDTSVRTAYLQISSLKAEDTAVYFCTRLGTGNTKGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 116 - protein, 449 aa, synthetic construct
QIQLVQSGSELKKPGASVKISCKASGYTFTDYAMNWVRQAPGQGLEWMGWINTQTGKPTYAQGFTGRFVFSLDTSVSTAYLQISSLKAEDTAVYFCTRLGTGNTKGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 117 - protein, 449 aa, synthetic construct
QIQLVQSGSELKKPGASVKISCKASGYTFTDYAMNWVRQAPGQGLEWMGWINTQTGKPTYAQGFTGRFVFSLDTSVSTAYLQISSLKAEDTAVYYCTRLGTGNTKGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 118 - protein, 449 aa, synthetic construct
QIQLVQSGSELKKPGASVKVSCKASGYTFTDYAMNWVRQAPGQGLEWMGWINTQTGKPTYAQGFTGRFVFSLDTSVSTAYLQISSLKAEDTAVYFCTRLGTGNTKGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 119 - protein, 449 aa, synthetic construct
QVQLVQSGAEVKKPGSSVKVSCKASGYTFTDYAMNWVRQAPGQGLEWMGWINTQTGKPTYAQKFQGRVTFTLDESTSTAYMELSSLRSEDTAVYYCTRLGTGNTKGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 120 - protein, 446 aa, synthetic construct
QIQLVQSGSELKKPGASVKVSCKASGYTFTDYAMNWVRQAPGQGLEWMGWINTQTGKPTYAQGFTGRFVFSLDTSVSTAYLQISSLKAEDTAVYYCTRLGTGNTKGFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 121 - protein, 446 aa, synthetic construct
QIQLVQSGAEVKKPGSSVKVSCKASGYTFTDYAMNWVRQAPGQGLEWMGWINTQTGKPTYAQKFQGRVTFTLDESTSTAYMELSSLRSEDTAVYYCTRLGTGNTKGFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 122 - protein, 446 aa, synthetic construct
QIQLVQSGAEVKKPGSSVKVSCKASGYTFTDYAMNWVRQAPGQGLEWMGWINTQTGKPTYAQKFQGRVTFTLDESTSTTYMELSSLRSEDTAVYYCARLGTGNTKGFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 123 - protein, 446 aa, synthetic construct
QIQLVQSGAEVKKPGSSVKVSCKASGYTFTDYAMNWVRQAPGQGLEWMGWINTQTGKPTYAQKFQGRVTFTLDESTSTTYMELSSLRSEDTAVYYCTRLGTGNTKGFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 124 - protein, 446 aa, synthetic construct
QIQLVQSGPEVKKPGESVKISCKASGYTFTDYAMNWVKQAPGNGLKWMGWINTQTGKPTYADDFKQRFVFSLETSARTTYLQINNLNIEDTATYFCTRLGTGNTKGFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 125 - protein, 446 aa, synthetic construct
QIQLVQSGSELKKPGASVKISCKASGYTFTDYAMNWVKQAPGQGLEWMGWINTQTGKPTYAQGFTGRFVFSLDTSVSTAYLQISSLKAEDTAVYFCTRLGTGNTKGFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 126 - protein, 446 aa, synthetic construct; identical to SEQ ID NO: 125

SEQ ID NO: 127 - protein, 446 aa, synthetic construct
QIQLVQSGSELKKPGASVKISCKASGYTFTDYAMNWVRQAPGQGLEWMGWINTQTGKPTYAQGFTGRFVFSLDTSVRTAYLQISSLKAEDTAVYFCTRLGTGNTKGFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 128 - protein, 446 aa, synthetic construct
QIQLVQSGSELKKPGASVKISCKASGYTFTDYAMNWVRQAPGQGLEWMGWINTQTGKPTYAQGFTGRFVFSLDTSVSTAYLQISSLKAEDTAVYFCTRLGTGNTKGFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 129 - protein, 446 aa, synthetic construct
QIQLVQSGSELKKPGASVKISCKASGYTFTDYAMNWVRQAPGQGLEWMGWINTQTGKPTYAQGFTGRFVFSLDTSVSTAYLQISSLKAEDTAVYYCTRLGTGNTKGFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 130 - protein, 446 aa, synthetic construct
QIQLVQSGSELKKPGASVKVSCKASGYTFTDYAMNWVRQAPGQGLEWMGWINTQTGKPTYAQGFTGRFVFSLDTSVSTAYLQISSLKAEDTAVYFCTRLGTGNTKGFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 131 - protein, 446 aa, synthetic construct
QVQLVQSGAEVKKPGSSVKVSCKASGYTFTDYAMNWVRQAPGQGLEWMGWINTQTGKPTYAQKFQGRVTFTLDESTSTAYMELSSLRSEDTAVYYCTRLGTGNTKGFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 132 - protein, 446 aa, synthetic construct
QIQLVQSGSELKKPGASVKVSCKASGYTFTDYAMNWVRQAPGQGLEWMGWINTQTGKPTYAQGFTGRFVFSLDTSVSTAYLQISSLKAEDTAVYYCTRLGTGNTKGFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 133 - protein, 446 aa, synthetic construct
QIQLVQSGAEVKKPGSSVKVSCKASGYTFTDYAMNWVRQAPGQGLEWMGWINTQTGKPTYAQKFQGRVTFTLDESTSTAYMELSSLRSEDTAVYYCTRLGTGNTKGFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 134 - protein, 446 aa, synthetic construct
QIQLVQSGAEVKKPGSSVKVSCKASGYTFTDYAMNWVRQAPGQGLEWMGWINTQTGKPTYAQKFQGRVTFTLDESTSTTYMELSSLRSEDTAVYYCARLGTGNTKGFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 135 - protein, 446 aa, synthetic construct
QIQLVQSGAEVKKPGSSVKVSCKASGYTFTDYAMNWVRQAPGQGLEWMGWINTQTGKPTYAQKFQGRVTFTLDESTSTTYMELSSLRSEDTAVYYCTRLGTGNTKGFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 136 - protein, 446 aa, synthetic construct
QIQLVQSGPEVKKPGESVKISCKASGYTFTDYAMNWVKQAPGNGLKWMGWINTQTGKPTYADDFKQRFVFSLETSARTTYLQINNLNIEDTATYFCTRLGTGNTKGFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 137 - protein, 446 aa, synthetic construct
QIQLVQSGSELKKPGASVKISCKASGYTFTDYAMNWVKQAPGQGLEWMGWINTQTGKPTYAQGFTGRFVFSLDTSVSTAYLQISSLKAEDTAVYFCTRLGTGNTKGFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 138 - protein, 446 aa, synthetic construct; identical to SEQ ID NO: 137

SEQ ID NO: 139 - protein, 446 aa, synthetic construct
QIQLVQSGSELKKPGASVKISCKASGYTFTDYAMNWVRQAPGQGLEWMGWINTQTGKPTYAQGFTGRFVFSLDTSVRTAYLQISSLKAEDTAVYFCTRLGTGNTKGFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 140 - protein, 446 aa, synthetic construct
QIQLVQSGSELKKPGASVKISCKASGYTFTDYAMNWVRQAPGQGLEWMGWINTQTGKPTYAQGFTGRFVFSLDTSVSTAYLQISSLKAEDTAVYFCTRLGTGNTKGFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 141 - protein, 446 aa, synthetic construct
QIQLVQSGSELKKPGASVKISCKASGYTFTDYAMNWVRQAPGQGLEWMGWINTQTGKPTYAQGFTGRFVFSLDTSVSTAYLQISSLKAEDTAVYYCTRLGTGNTKGFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 142 - protein, 446 aa, synthetic construct
QIQLVQSGSELKKPGASVKVSCKASGYTFTDYAMNWVRQAPGQGLEWMGWINTQTGKPTYAQGFTGRFVFSLDTSVSTAYLQISSLKAEDTAVYFCTRLGTGNTKGFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 143 - protein, 446 aa, synthetic construct
QVQLVQSGAEVKKPGSSVKVSCKASGYTFTDYAMNWVRQAPGQGLEWMGWINTQTGKPTYAQKFQGRVTFTLDESTSTAYMELSSLRSEDTAVYYCTRLGTGNTKGFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 144 - protein, 220 aa, synthetic construct
DIQITQSPSSLSASVGDRVTITCKSSQSLLYSENNQDYLAWYQQKPGKVPKLLIYGASNRHTGVPSRFSGSGSGTDFTLTISSLQPEDVATYYCEQTYRYPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 145 - protein, 220 aa, synthetic construct
DILINQSPASLTVSAGERVTMSCKSSQSLLYSENNQDYLAWYQQKPGQFPKLLIYGASNRHTGVPDRFTGSGSGTDFTLTISSVQAEDLADYYCEQTYRYPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 146 - protein, 220 aa, synthetic construct; identical to SEQ ID NO: 144

SEQ ID NO: 147 - protein, 220 aa, synthetic construct
DIQMTQSPSSLSASVGDRVTITCKSSQSLLYSENNQDYLAWYQQKPGKVPKLLIYGASNRHTGVPSRFSGSGSGTDFTLTISSLQPEDVATYYCEQTYRYPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 148 - protein, 450 aa, synthetic construct
QVQLVQSGAEVKKPGASVKVSCKASGYTFTSYGISWVRQAPGQGLEWMGWISAYNANTNYAQKLQGRVTMTTDTSTSTAYMELRSLRSDDTAAYYCARVTGITGTTIGPWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 149 - protein, 450 aa, synthetic construct
QVQLVQSGAEVKKPGASVKVSCKASGYTFTSYGISWVRQAPGQGLEWMGWISAYNANVNYAQKLQGRVTMTTDTSTSTAYMELRSLRSDDTAAYYCARVTGITGTTIGPWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 150 - protein, 450 aa, synthetic construct
QVQLVQSGAEVKKPGASVKVSCKASGYTFTSYGISWVRQAPGQGLEWMGWISAYNGNTNYAQKLQGRVTMTTDTSTSTAYMELRSLRSDDTAAYYCARVTGITGTTIDPWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 151 - protein, 450 aa, synthetic construct
QVQLVQSGAEVKKPGASVKVSCKASGYTFTSYGISWVRQAPGQGLEWMGWISAYNSNTNYAQKLQGRVTMTTDTSTSTAYMELRSLRSDDTAAYYCARVTGITGTTIGPWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 152 - protein, 447 aa, synthetic construct
QVQLVQSGAEVKKPGASVKVSCKASGYTFTSYGISWVRQAPGQGLEWMGWISAYNANTNYAQKLQGRVTMTTDTSTSTAYMELRSLRSDDTAAYYCARVTGITGTTIGPWGQGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGK

SEQ ID NO: 153 - protein, 447 aa, synthetic construct
QVQLVQSGAEVKKPGASVKVSCKASGYTFTSYGISWVRQAPGQGLEWMGWISAYNANVNYAQKLQGRVTMTTDTSTSTAYMELRSLRSDDTAAYYCARVTGITGTTIGPWGQGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGK

SEQ ID NO: 154 - protein, 447 aa, synthetic construct
QVQLVQSGAEVKKPGASVKVSCKASGYTFTSYGISWVRQAPGQGLEWMGWISAYNGNTNYAQKLQGRVTMTTDTSTSTAYMELRSLRSDDTAAYYCARVTGITGTTIDPWGQGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGK

SEQ ID NO: 155 - protein, 447 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGKQVQLVQSGAEVKKPGASVKVSCKASGYTFTSYGISWVRQAPGQGLEWMGWISAYNSNTNYAQKLQGRVTMTTDTSTSTAYMELRSLRSDDTAAYYCARVTGITGTTIGPWGQGTMVTVSS

SEQ ID NO: 156 - protein, 447 aa, synthetic construct
QVQLVQSGAEVKKPGASVKVSCKASGYTFTSYGISWVRQAPGQGLEWMGWISAYNANTNYAQKLQGRVTMTTDTSTSTAYMELRSLRSDDTAAYYCARVTGITGTTIGPWGQGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 157 - protein, 447 aa, synthetic construct
QVQLVQSGAEVKKPGASVKVSCKASGYTFTSYGISWVRQAPGQGLEWMGWISAYNANVNYAQKLQGRVTMTTDTSTSTAYMELRSLRSDDTAAYYCARVTGITGTTIGPWGQGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 158 - protein, 447 aa, synthetic construct
QVQLVQSGAEVKKPGASVKVSCKASGYTFTSYGISWVRQAPGQGLEWMGWISAYNGNTNYAQKLQGRVTMTTDTSTSTAYMELRSLRSDDTAAYYCARVTGITGTTIDPWGQGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 159 - protein, 447 aa, synthetic construct
QVQLVQSGAEVKKPGASVKVSCKASGYTFTSYGISWVRQAPGQGLEWMGWISAYNSNTNYAQKLQGRVTMTTDTSTSTAYMELRSLRSDDTAAYYCARVTGITGTTIGPWGQGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 160 - protein, 449 aa, synthetic construct
QVQLVQSGAEVKKPGASVKVSCKASGYTFTSYGISWVRQAPGQGLEWMGWISAYNANTNYAQKLQGRVTMTTDTSTSTAYMELRSLRSDDTAAYYCARVTGITGTTIGPWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 161 - protein, 449 aa, synthetic construct
QVQLVQSGAEVKKPGASVKVSCKASGYTFTSYGISWVRQAPGQGLEWMGWISAYNANVNYAQKLQGRVTMTTDTSTSTAYMELRSLRSDDTAAYYCARVTGITGTTIGPWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 162 - protein, 449 aa, synthetic construct
QVQLVQSGAEVKKPGASVKVSCKASGYTFTSYGISWVRQAPGQGLEWMGWISAYNGNTNYAQKLQGRVTMTTDTSTSTAYMELRSLRSDDTAAYYCARVTGITGTTIDPWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 163 - protein, 449 aa, synthetic construct
QVQLVQSGAEVKKPGASVKVSCKASGYTFTSYGISWVRQAPGQGLEWMGWISAYNSNTNYAQKLQGRVTMTTDTSTSTAYMELRSLRSDDTAAYYCARVTGITGTTIGPWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 164 - protein, 446 aa, synthetic construct
QVQLVQSGAEVKKPGASVKVSCKASGYTFTSYGISWVRQAPGQGLEWMGWISAYNANTNYAQKLQGRVTMTTDTSTSTAYMELRSLRSDDTAAYYCARVTGITGTTIGPWGQGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 165 - protein, 446 aa, synthetic construct
QVQLVQSGAEVKKPGASVKVSCKASGYTFTSYGISWVRQAPGQGLEWMGWISAYNANVNYAQKLQGRVTMTTDTSTSTAYMELRSLRSDDTAAYYCARVTGITGTTIGPWGQGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 166 - protein, 446 aa, synthetic construct
QVQLVQSGAEVKKPGASVKVSCKASGYTFTSYGISWVRQAPGQGLEWMGWISAYNGNTNYAQKLQGRVTMTTDTSTSTAYMELRSLRSDDTAAYYCARVTGITGTTIDPWGQGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 167 - protein, 446 aa, synthetic construct
QVQLVQSGAEVKKPGASVKVSCKASGYTFTSYGISWVRQAPGQGLEWMGWISAYNSNTNYAQKLQGRVTMTTDTSTSTAYMELRSLRSDDTAAYYCARVTGITGTTIGPWGQGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 168 - protein, 446 aa, synthetic construct
QVQLVQSGAEVKKPGASVKVSCKASGYTFTSYGISWVRQAPGQGLEWMGWISAYNANTNYAQKLQGRVTMTTDTSTSTAYMELRSLRSDDTAAYYCARVTGITGTTIGPWGQGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 169 - protein, 446 aa, synthetic construct
QVQLVQSGAEVKKPGASVKVSCKASGYTFTSYGISWVRQAPGQGLEWMGWISAYNANVNYAQKLQGRVTMTTDTSTSTAYMELRSLRSDDTAAYYCARVTGITGTTIGPWGQGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 170 - protein, 446 aa, synthetic construct
QVQLVQSGAEVKKPGASVKVSCKASGYTFTSYGISWVRQAPGQGLEWMGWISAYNGNTNYAQKLQGRVTMTTDTSTSTAYMELRSLRSDDTAAYYCARVTGITGTTIDPWGQGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 171 - protein, 446 aa, synthetic construct
QVQLVQSGAEVKKPGASVKVSCKASGYTFTSYGISWVRQAPGQGLEWMGWISAYNSNTNYAQKLQGRVTMTTDTSTSTAYMELRSLRSDDTAAYYCARVTGITGTTIGPWGQGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 172 - protein, 214 aa, synthetic construct
DIQMTQSPSSLSASVGDRVTITCRASQSISSYLNWYQQKPGKAPKLLIYDASSLESGVPSRFSGSGSGTEFTLTISSLQPDDFAVYYCQQYNNWPQTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 173 - protein, 456 aa, synthetic construct
EVQLLESGGGLVQPGGSLRLSCAASGFSFSSSYYMCWVRQAPGKGLEWVSAIGTTRGSTYYADSVKGRFTISRDSSKNTVYLQMNSLRAEDTAVYYCARDATGYRINTIGLYFNLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 174 - protein, 456 aa, synthetic construct
EVQLLESGGGLVQPGGSLRLSCAASGFSFSSSYYMAWVRQAPGKGLEWVSAIGTTRGSTYYADSVKGRFTISRDNSKNTLYLQMNSLRAEDTAVYYCARDATGYRINTIGLYFNLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 175 - protein, 456 aa, synthetic construct
EVQLLESGGGLVQPGGSLRLSCAASGFSFSSSYYMAWVRQAPGKGLEWVSAIGTTRGSTYYADSVKGRFTISRDNSKNTVYLQMNSLRAEDTAVYYCARDATGYRINTIGLYFNLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 176 - protein, 456 aa, synthetic construct
EVQLLESGGGLVQPGGSLRLSCAASGFSFSSSYYMAWVRQAPGKGLEWVSAIGTTRGSTYYADSVKGRFTISRDSSKNTLYLQMNSLRAEDTAVYYCARDATGYRINTIGLYFNLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 177 - protein, 456 aa, synthetic construct
EVQLLESGGGLVQPGGSLRLSCAASGFSFSSSYYMAWVRQAPGKGLEWVSAIGTTRGSTYYADSVKGRFTISRDSSKNTVYLQMNSLRAEDTAVYFCARDATGYRINTIGLYFNLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 178 - protein, 456 aa, synthetic construct
EVQLLESGGGLVQPGGSLRLSCAASGFSFSSSYYMCWVRQAPGKGLEWVSCIGTTRGSTYYADSVKGRFTISRDNSKNTLYLQMNSLRAEDTAVYYCARDATGYRINTIGLYFNLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 179 - protein, 456 aa, synthetic construct
EVQLLESGGGLVQPGGSLRLSCAASGFSFSSSYYMCWVRQAPGKGLEWVSCIGTTRGSTYYADSVKGRFTISRDNSKNTVYLQMNSLRAEDTAVYYCARDATGYRINTIGLYFNLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 180 - protein, 456 aa, synthetic construct
EVQLLESGGGLVQPGGSLRLSCAASGFSFSSSYYMCWVRQAPGKGLEWVSCIGTTRGSTYYADSVKGRFTISRDSSKNTLYLQMNSLRAEDTAVYYCARDATGYRINTIGLYFNLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 181 - protein, 456 aa, synthetic construct
EVQLLESGGGLVQPGGSLRLSCAASGFSFSSSYYMCWVRQAPGKGLEWVSCIGTTRGSTYYADSVKGRFTISRDSSKNTVYLQMNSLRAEDTAVYFCARDATGYRINTIGLYFNLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 182 - protein, 456 aa, synthetic construct
EVQLLESGGGLVQPGGSLRLSCAASGFSFSSSYYMSWVRQAPGKGLEWVSAIGTTRGSTYYADSVKGRFTISRDNSKNTLYLQMNSLRAEDTAVYYCARDATGYRINTIGLYFNLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 183 - protein, 456 aa, synthetic construct
EVQLLESGGGLVQPGGSLRLSCAASGFSFSSSYYMSWVRQAPGKGLEWVSAIGTTRGSTYYADSVKGRFTISRDNSKNTVYLQMNSLRAEDTAVYYCARDATGYRINTIGLYFNLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 184 - protein, 456 aa, synthetic construct
EVQLLESGGGLVQPGGSLRLSCAASGFSFSSSYYMSWVRQAPGKGLEWVSAIGTTRGSTYYADSVKGRFTISRDSSKNTLYLQMNSLRAEDTAVYYCARDATGYRINTIGLYFNLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 185 - protein, 456 aa, synthetic construct
EVQLLESGGGLVQPGGSLRLSCAASGFSFSSSYYMSWVRQAPGKGLEWVSAIGTTRGSTYYADSVKGRFTISRDSSKNTVYLQMNSLRAEDTAVYFCARDATGYRINTIGLYFNLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 186 - protein, 454 aa, synthetic construct
QSLEESGGDLVKPGASLTLTCRVSGFSFSSSYYMCWVRQAPGKGLEWIACIGTTRGSTYYATWAKGRFTISKISSTTVTLQMTSLTDADTATYFCARDATGYRINTIGLYFNLWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 187 - protein, 453 aa, synthetic construct
EVQLLESGGGLVQPGGSLRLSCAASGFSFSSSYYMCWVRQAPGKGLEWVSAIGTTRGSTYYADSVKGRFTISRDSSKNTVYLQMNSLRAEDTAVYYCARDATGYRINTIGLYFNLWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGK

SEQ ID NO: 188 - protein, 453 aa, synthetic construct
EVQLLESGGGLVQPGGSLRLSCAASGFSFSSSYYMAWVRQAPGKGLEWVSAIGTTRGSTYYADSVKGRFTISRDNSKNTLYLQMNSLRAEDTAVYYCARDATGYRINTIGLYFNLWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGK

SEQ ID NO: 189 - protein, 453 aa, synthetic construct
EVQLLESGGGLVQPGGSLRLSCAASGFSFSSSYYMAWVRQAPGKGLEWVSAIGTTRGSTYYADSVKGRFTISRDNSKNTVYLQMNSLRAEDTAVYYCARDATGYRINTIGLYFNLWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGK

SEQ ID NO: 190 - protein, 453 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGKEVQLLESGGGLVQPGGSLRLSCAASGFSFSSSYYMAWVRQAPGKGLEWVSAIGTTRGSTYYADSVKGRFTISRDSSKNTLYLQMNSLRAEDTAVYYCARDATGYRINTIGLYFNLWGQGTLVTVSS

SEQ ID NO: 191 - protein, 453 aa, synthetic construct
EVQLLESGGGLVQPGGSLRLSCAASGFSFSSSYYMAWVRQAPGKGLEWVSAIGTTRGSTYYADSVKGRFTISRDSSKNTVYLQMNSLRAEDTAVYFCARDATGYRINTIGLYFNLWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGK

SEQ ID NO: 192 - protein, 453 aa, synthetic construct
EVQLLESGGGLVQPGGSLRLSCAASGFSFSSSYYMCWVRQAPGKGLEWVSCIGTTRGSTYYADSVKGRFTISRDNSKNTLYLQMNSLRAEDTAVYYCARDATGYRINTIGLYFNLWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGK

SEQ ID NO: 193 - protein, 453 aa, synthetic construct
EVQLLESGGGLVQPGGSLRLSCAASGFSFSSSYYMCWVRQAPGKGLEWVSCIGTTRGSTYYADSVKGRFTISRDNSKNTVYLQMNSLRAEDTAVYYCARDATGYRINTIGLYFNLWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGK

SEQ ID NO: 194 - protein, 453 aa, synthetic construct
EVQLLESGGGLVQPGGSLRLSCAASGFSFSSSYYMCWVRQAPGKGLEWVSCIGTTRGSTYYADSVKGRFTISRDSSKNTLYLQMNSLRAEDTAVYYCARDATGYRINTIGLYFNLWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGK

SEQ ID NO: 195 - protein, 453 aa, synthetic construct
EVQLLESGGGLVQPGGSLRLSCAASGFSFSSSYYMCWVRQAPGKGLEWVSCIGTTRGSTYYADSVKGRFTISRDSSKNTVYLQMNSLRAEDTAVYFCARDATGYRINTIGLYFNLWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGK

SEQ ID NO: 196 - protein, 453 aa, synthetic construct
EVQLLESGGGLVQPGGSLRLSCAASGFSFSSSYYMSWVRQAPGKGLEWVSAIGTTRGSTYYADSVKGRFTISRDNSKNTLYLQMNSLRAEDTAVYYCARDATGYRINTIGLYFNLWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGK

SEQ ID NO: 197 - protein, 453 aa, synthetic construct
EVQLLESGGGLVQPGGSLRLSCAASGFSFSSSYYMSWVRQAPGKGLEWVSAIGTTRGSTYYADSVKGRFTISRDNSKNTVYLQMNSLRAEDTAVYYCARDATGYRINTIGLYFNLWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGK

SEQ ID NO: 198 - protein, 453 aa, synthetic construct
EVQLLESGGGLVQPGGSLRLSCAASGFSFSSSYYMSWVRQAPGKGLEWVSAIGTTRGSTYYADSVKGRFTISRDSSKNTLYLQMNSLRAEDTAVYYCARDATGYRINTIGLYFNLWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGK

SEQ ID NO: 199 - protein, 453 aa, synthetic construct
EVQLLESGGGLVQPGGSLRLSCAASGFSFSSSYYMSWVRQAPGKGLEWVSAIGTTRGSTYYADSVKGRFTISRDSSKNTVYLQMNSLRAEDTAVYFCARDATGYRINTIGLYFNLWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGK

SEQ ID NO: 200 - protein, 451 aa, synthetic construct
QSLEESGGDLVKPGASLTLTCRVSGFSFSSSYYMCWVRQAPGKGLEWIACIGTTRGSTYYATWAKGRFTISKISSTTVTLQMTSLTDADTATYFCARDATGYRINTIGLYFNLWGPGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGK

SEQ ID NO: 201 - protein, 453 aa, synthetic construct
EVQLLESGGGLVQPGGSLRLSCAASGFSFSSSYYMCWVRQAPGKGLEWVSAIGTTRGSTYYADSVKGRFTISRDSSKNTVYLQMNSLRAEDTAVYYCARDATGYRINTIGLYFNLWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 202 - protein, 453 aa, synthetic construct
EVQLLESGGGLVQPGGSLRLSCAASGFSFSSSYYMAWVRQAPGKGLEWVSAIGTTRGSTYYADSVKGRFTISRDNSKNTLYLQMNSLRAEDTAVYYCARDATGYRINTIGLYFNLWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 203 - protein, 453 aa, synthetic construct
EVQLLESGGGLVQPGGSLRLSCAASGFSFSSSYYMAWVRQAPGKGLEWVSAIGTTRGSTYYADSVKGRFTISRDNSKNTVYLQMNSLRAEDTAVYYCARDATGYRINTIGLYFNLWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 204 - protein, 453 aa, synthetic construct
EVQLLESGGGLVQPGGSLRLSCAASGFSFSSSYYMAWVRQAPGKGLEWVSAIGTTRGSTYYADSVKGRFTISRDSSKNTLYLQMNSLRAEDTAVYYCARDATGYRINTIGLYFNLWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 205 - protein, 453 aa, synthetic construct
EVQLLESGGGLVQPGGSLRLSCAASGFSFSSSYYMAWVRQAPGKGLEWVSAIGTTRGSTYYADSVKGRFTISRDSSKNTVYLQMNSLRAEDTAVYFCARDATGYRINTIGLYFNLWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 206 - protein, 453 aa, synthetic construct
EVQLLESGGGLVQPGGSLRLSCAASGFSFSSSYYMCWVRQAPGKGLEWVSCIGTTRGSTYYADSVKGRFTISRDNSKNTLYLQMNSLRAEDTAVYYCARDATGYRINTIGLYFNLWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 207 - protein, 453 aa, synthetic construct
EVQLLESGGGLVQPGGSLRLSCAASGFSFSSSYYMCWVRQAPGKGLEWVSCIGTTRGSTYYADSVKGRFTISRDNSKNTVYLQMNSLRAEDTAVYYCARDATGYRINTIGLYFNLWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 208 - protein, 453 aa, synthetic construct
EVQLLESGGGLVQPGGSLRLSCAASGFSFSSSYYMCWVRQAPGKGLEWVSCIGTTRGSTYYADSVKGRFTISRDSSKNTLYLQMNSLRAEDTAVYYCARDATGYRINTIGLYFNLWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 209 - protein, 453 aa, synthetic construct
EVQLLESGGGLVQPGGSLRLSCAASGFSFSSSYYMCWVRQAPGKGLEWVSCIGTTRGSTYYADSVKGRFTISRDSSKNTVYLQMNSLRAEDTAVYFCARDATGYRINTIGLYFNLWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 210 - protein, 453 aa, synthetic construct
EVQLLESGGGLVQPGGSLRLSCAASGFSFSSSYYMSWVRQAPGKGLEWVSAIGTTRGSTYYADSVKGRFTISRDNSKNTLYLQMNSLRAEDTAVYYCARDATGYRINTIGLYFNLWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 211 - protein, 453 aa, synthetic construct
EVQLLESGGGLVQPGGSLRLSCAASGFSFSSSYYMSWVRQAPGKGLEWVSAIGTTRGSTYYADSVKGRFTISRDNSKNTVYLQMNSLRAEDTAVYYCARDATGYRINTIGLYFNLWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 212 - protein, 453 aa, synthetic construct
EVQLLESGGGLVQPGGSLRLSCAASGFSFSSSYYMSWVRQAPGKGLEWVSAIGTTRGSTYYADSVKGRFTISRDSSKNTLYLQMNSLRAEDTAVYYCARDATGYRINTIGLYFNLWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 213 - protein, 453 aa, synthetic construct
EVQLLESGGGLVQPGGSLRLSCAASGFSFSSSYYMSWVRQAPGKGLEWVSAIGTTRGSTYYADSVKGRFTISRDSSKNTVYLQMNSLRAEDTAVYFCARDATGYRINTIGLYFNLWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 214 - protein, 451 aa, synthetic construct
QSLEESGGDLVKPGASLTLTCRVSGFSFSSSYYMCWVRQAPGKGLEWIACIGTTRGSTYYATWAKGRFTISKISSTTVTLQMTSLTDADTATYFCARDATGYRINTIGLYFNLWGPGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 215 - protein, 455 aa, synthetic construct
EVQLLESGGGLVQPGGSLRLSCAASGFSFSSSYYMCWVRQAPGKGLEWVSAIGTTRGSTYYADSVKGRFTISRDSSKNTVYLQMNSLRAEDTAVYYCARDATGYRINTIGLYFNLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 216 - protein, 455 aa, synthetic construct
EVQLLESGGGLVQPGGSLRLSCAASGFSFSSSYYMAWVRQAPGKGLEWVSAIGTTRGSTYYADSVKGRFTISRDNSKNTLYLQMNSLRAEDTAVYYCARDATGYRINTIGLYFNLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 217 - protein, 455 aa, synthetic construct
EVQLLESGGGLVQPGGSLRLSCAASGFSFSSSYYMAWVRQAPGKGLEWVSAIGTTRGSTYYADSVKGRFTISRDNSKNTVYLQMNSLRAEDTAVYYCARDATGYRINTIGLYFNLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 218 - protein, 455 aa, synthetic construct
EVQLLESGGGLVQPGGSLRLSCAASGFSFSSSYYMAWVRQAPGKGLEWVSAIGTTRGSTYYADSVKGRFTISRDSSKNTLYLQMNSLRAEDTAVYYCARDATGYRINTIGLYFNLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 219 - protein, 455 aa, synthetic construct
EVQLLESGGGLVQPGGSLRLSCAASGFSFSSSYYMAWVRQAPGKGLEWVSAIGTTRGSTYYADSVKGRFTISRDSSKNTVYLQMNSLRAEDTAVYFCARDATGYRINTIGLYFNLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 220 - protein, 455 aa, synthetic construct
EVQLLESGGGLVQPGGSLRLSCAASGFSFSSSYYMCWVRQAPGKGLEWVSCIGTTRGSTYYADSVKGRFTISRDNSKNTLYLQMNSLRAEDTAVYYCARDATGYRINTIGLYFNLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 221 - protein, 455 aa, synthetic construct
EVQLLESGGGLVQPGGSLRLSCAASGFSFSSSYYMCWVRQAPGKGLEWVSCIGTTRGSTYYADSVKGRFTISRDNSKNTVYLQMNSLRAEDTAVYYCARDATGYRINTIGLYFNLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 222 - protein, 455 aa, synthetic construct
EVQLLESGGGLVQPGGSLRLSCAASGFSFSSSYYMCWVRQAPGKGLEWVSCIGTTRGSTYYADSVKGRFTISRDSSKNTLYLQMNSLRAEDTAVYYCARDATGYRINTIGLYFNLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 223 - protein, 455 aa, synthetic construct
EVQLLESGGGLVQPGGSLRLSCAASGFSFSSSYYMCWVRQAPGKGLEWVSCIGTTRGSTYYADSVKGRFTISRDSSKNTVYLQMNSLRAEDTAVYFCARDATGYRINTIGLYFNLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 224 - protein, 455 aa, synthetic construct
EVQLLESGGGLVQPGGSLRLSCAASGFSFSSSYYMSWVRQAPGKGLEWVSAIGTTRGSTYYADSVKGRFTISRDNSKNTLYLQMNSLRAEDTAVYYCARDATGYRINTIGLYFNLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 225 - protein, 455 aa, synthetic construct
EVQLLESGGGLVQPGGSLRLSCAASGFSFSSSYYMSWVRQAPGKGLEWVSAIGTTRGSTYYADSVKGRFTISRDNSKNTVYLQMNSLRAEDTAVYYCARDATGYRINTIGLYFNLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 226 - protein, 455 aa, synthetic construct
EVQLLESGGGLVQPGGSLRLSCAASGFSFSSSYYMSWVRQAPGKGLEWVSAIGTTRGSTYYADSVKGRFTISRDSSKNTLYLQMNSLRAEDTAVYYCARDATGYRINTIGLYFNLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 227 - protein, 455 aa, synthetic construct
EVQLLESGGGLVQPGGSLRLSCAASGFSFSSSYYMSWVRQAPGKGLEWVSAIGTTRGSTYYADSVKGRFTISRDSSKNTVYLQMNSLRAEDTAVYFCARDATGYRINTIGLYFNLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 228 - protein, 453 aa, synthetic construct
QSLEESGGDLVKPGASLTLTCRVSGFSFSSSYYMCWVRQAPGKGLEWIACIGTTRGSTYYATWAKGRFTISKISSTTVTLQMTSLTDADTATYFCARDATGYRINTIGLYFNLWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 229 - protein, 452 aa, synthetic construct
EVQLLESGGGLVQPGGSLRLSCAASGFSFSSSYYMCWVRQAPGKGLEWVSAIGTTRGSTYYADSVKGRFTISRDSSKNTVYLQMNSLRAEDTAVYYCARDATGYRINTIGLYFNLWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 230 - protein, 452 aa, synthetic construct
EVQLLESGGGLVQPGGSLRLSCAASGFSFSSSYYMAWVRQAPGKGLEWVSAIGTTRGSTYYADSVKGRFTISRDNSKNTLYLQMNSLRAEDTAVYYCARDATGYRINTIGLYFNLWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 231 - protein, 452 aa, synthetic construct
EVQLLESGGGLVQPGGSLRLSCAASGFSFSSSYYMAWVRQAPGKGLEWVSAIGTTRGSTYYADSVKGRFTISRDNSKNTVYLQMNSLRAEDTAVYYCARDATGYRINTIGLYFNLWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 232 - protein, 452 aa, synthetic construct
EVQLLESGGGLVQPGGSLRLSCAASGFSFSSSYYMAWVRQAPGKGLEWVSAIGTTRGSTYYADSVKGRFTISRDSSKNTLYLQMNSLRAEDTAVYYCARDATGYRINTIGLYFNLWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 233 - protein, 452 aa, synthetic construct
EVQLLESGGGLVQPGGSLRLSCAASGFSFSSSYYMAWVRQAPGKGLEWVSAIGTTRGSTYYADSVKGRFTISRDSSKNTVYLQMNSLRAEDTAVYFCARDATGYRINTIGLYFNLWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 234 - protein, 452 aa, synthetic construct
EVQLLESGGGLVQPGGSLRLSCAASGFSFSSSYYMCWVRQAPGKGLEWVSCIGTTRGSTYYADSVKGRFTISRDNSKNTLYLQMNSLRAEDTAVYYCARDATGYRINTIGLYFNLWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 235 - protein, 452 aa, synthetic construct
EVQLLESGGGLVQPGGSLRLSCAASGFSFSSSYYMCWVRQAPGKGLEWVSCIGTTRGSTYYADSVKGRFTISRDNSKNTVYLQMNSLRAEDTAVYYCARDATGYRINTIGLYFNLWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 236 - protein, 452 aa, synthetic construct
EVQLLESGGGLVQPGGSLRLSCAASGFSFSSSYYMCWVRQAPGKGLEWVSCIGTTRGSTYYADSVKGRFTISRDSSKNTLYLQMNSLRAEDTAVYYCARDATGYRINTIGLYFNLWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 237 - protein, 452 aa, synthetic construct
EVQLLESGGGLVQPGGSLRLSCAASGFSFSSSYYMCWVRQAPGKGLEWVSCIGTTRGSTYYADSVKGRFTISRDSSKNTVYLQMNSLRAEDTAVYFCARDATGYRINTIGLYFNLWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 238 - protein, 452 aa, synthetic construct
EVQLLESGGGLVQPGGSLRLSCAASGFSFSSSYYMSWVRQAPGKGLEWVSAIGTTRGSTYYADSVKGRFTISRDNSKNTLYLQMNSLRAEDTAVYYCARDATGYRINTIGLYFNLWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 239 - protein, 452 aa, synthetic construct
EVQLLESGGGLVQPGGSLRLSCAASGFSFSSSYYMSWVRQAPGKGLEWVSAIGTTRGSTYYADSVKGRFTISRDNSKNTVYLQMNSLRAEDTAVYYCARDATGYRINTIGLYFNLWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 240 - protein, 452 aa, synthetic construct
EVQLLESGGGLVQPGGSLRLSCAASGFSFSSSYYMSWVRQAPGKGLEWVSAIGTTRGSTYYADSVKGRFTISRDSSKNTLYLQMNSLRAEDTAVYYCARDATGYRINTIGLYFNLWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 241 - protein, 452 aa, synthetic construct
EVQLLESGGGLVQPGGSLRLSCAASGFSFSSSYYMSWVRQAPGKGLEWVSAIGTTRGSTYYADSVKGRFTISRDSSKNTVYLQMNSLRAEDTAVYFCARDATGYRINTIGLYFNLWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 242 - protein, 450 aa, synthetic construct
QSLEESGGDLVKPGASLTLTCRVSGFSFSSSYYMCWVRQAPGKGLEWIACIGTTRGSTYYATWAKGRFTISKISSTTVTLQMTSLTDADTATYFCARDATGYRINTIGLYFNLWGPGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 243 - protein, 452 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGEVQLLESGGGLVQPGGSLRLSCAASGFSFSSSYYMCWVRQAPGKGLEWVSAIGTTRGSTYYADSVKGRFTISRDSSKNTVYLQMNSLRAEDTAVYYCARDATGYRINTIGLYFNLWGQGTLVTVSS

SEQ ID NO: 244 - protein, 452 aa, synthetic construct
EVQLLESGGGLVQPGGSLRLSCAASGFSFSSSYYMAWVRQAPGKGLEWVSAIGTTRGSTYYADSVKGRFTISRDNSKNTLYLQMNSLRAEDTAVYYCARDATGYRINTIGLYFNLWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 245 - protein, 452 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGEVQLLESGGGLVQPGGSLRLSCAASGFSFSSSYYMAWVRQAPGKGLEWVSAIGTTRGSTYYADSVKGRFTISRDNSKNTVYLQMNSLRAEDTAVYYCARDATGYRINTIGLYFNLWGQGTLVTVSS

SEQ ID NO: 246 - protein, 452 aa, synthetic construct
EVQLLESGGGLVQPGGSLRLSCAASGFSFSSSYYMAWVRQAPGKGLEWVSAIGTTRGSTYYADSVKGRFTISRDSSKNTLYLQMNSLRAEDTAVYYCARDATGYRINTIGLYFNLWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 247 - protein, 452 aa, synthetic construct
EVQLLESGGGLVQPGGSLRLSCAASGFSFSSSYYMAWVRQAPGKGLEWVSAIGTTRGSTYYADSVKGRFTISRDSSKNTVYLQMNSLRAEDTAVYFCARDATGYRINTIGLYFNLWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 248 - protein, 452 aa, synthetic construct
EVQLLESGGGLVQPGGSLRLSCAASGFSFSSSYYMCWVRQAPGKGLEWVSCIGTTRGSTYYADSVKGRFTISRDNSKNTLYLQMNSLRAEDTAVYYCARDATGYRINTIGLYFNLWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 249 - protein, 452 aa, synthetic construct
EVQLLESGGGLVQPGGSLRLSCAASGFSFSSSYYMCWVRQAPGKGLEWVSCIGTTRGSTYYADSVKGRFTISRDNSKNTVYLQMNSLRAEDTAVYYCARDATGYRINTIGLYFNLWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 250 - protein, 452 aa, synthetic construct
EVQLLESGGGLVQPGGSLRLSCAASGFSFSSSYYMCWVRQAPGKGLEWVSCIGTTRGSTYYADSVKGRFTISRDSSKNTLYLQMNSLRAEDTAVYYCARDATGYRINTIGLYFNLWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 251 - protein, 452 aa, synthetic construct
EVQLLESGGGLVQPGGSLRLSCAASGFSFSSSYYMCWVRQAPGKGLEWVSCIGTTRGSTYYADSVKGRFTISRDSSKNTVYLQMNSLRAEDTAVYFCARDATGYRINTIGLYFNLWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 252 - protein, 452 aa, synthetic construct
EVQLLESGGGLVQPGGSLRLSCAASGFSFSSSYYMSWVRQAPGKGLEWVSAIGTTRGSTYYADSVKGRFTISRDNSKNTLYLQMNSLRAEDTAVYYCARDATGYRINTIGLYFNLWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 253 - protein, 452 aa, synthetic construct
EVQLLESGGGLVQPGGSLRLSCAASGFSFSSSYYMSWVRQAPGKGLEWVSAIGTTRGSTYYADSVKGRFTISRDNSKNTVYLQMNSLRAEDTAVYYCARDATGYRINTIGLYFNLWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 254 - protein, 452 aa, synthetic construct
EVQLLESGGGLVQPGGSLRLSCAASGFSFSSSYYMSWVRQAPGKGLEWVSAIGTTRGSTYYADSVKGRFTISRDSSKNTLYLQMNSLRAEDTAVYYCARDATGYRINTIGLYFNLWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 255 - protein, 452 aa, synthetic construct
EVQLLESGGGLVQPGGSLRLSCAASGFSFSSSYYMSWVRQAPGKGLEWVSAIGTTRGSTYYADSVKGRFTISRDSSKNTVYLQMNSLRAEDTAVYFCARDATGYRINTIGLYFNLWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 256 - protein, 450 aa, synthetic construct
QSLEESGGDLVKPGASLTLTCRVSGFSFSSSYYMCWVRQAPGKGLEWIACIGTTRGSTYYATWAKGRFTISKISSTTVTLQMTSLTDADTATYFCARDATGYRINTIGLYFNLWGPGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 257 - protein, 218 aa, synthetic construct
DIVMTQSPDSLAVSLGERATINCQASQTIYSYLSWYQQKPGQPPKLLIYEASKLASGVPDRFSGSGSGTDYTLTISSLQAEDVAVYYCQSYHGTASTEYNTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 258 - protein, 216 aa, synthetic construct
ELTQTPSSVEAAVGGTPTIKCQASQTIYSYLSWYQQKPGQPPKLLIYEASKLASGVPSRFSGSGSGTDYTLTISDLECADAATYYCQSYHGTASTEYNTFGGGTEVVVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2026/0184794 A1
(43) Pub. Date: Jul. 2, 2026

(54) ANTIBODIES AGAINST INTEGRIN ALPHA 11 BETA 1 AND USES THEREOF

(71) Applicant: Momenta Pharmaceuticals, Inc., Titusville, NJ (US)

(72) Inventors: Elma Kurtagic, Milton, MA (US); Cholpon Tilegenova, Dresher, PA (US)

(21) Appl. No.: 18/877,643

(22) PCT Filed: Jun. 22, 2023
(86) PCT No.: PCT/US2023/068875

§ 371 Date: Dec. 20, 2024

Related U.S. Application Data
(60) Provisional application No. 63/366,811, filed on Jun. 22, 2022.

(51) Int. Cl. C07K 16/28; A61K 39/00; A61P 19/02

(52) U.S. Cl. CPC C07K 16/2842; A61P 19/02; A61K 2039/54; C07K 2317/24; C07K 2317/565

(57) ABSTRACT

Embodiments that are provided for herein relate to antibodies and compositions that bind to α11β1. Also provided are methods of producing the antibodies of the present disclosure, as well as uses of the provided antibodies and compositions for the treatment of α11β1 mediated diseases and disorders.

### CROSS REFERENCE TO RELATED APPLICATIONS

This application claims priority to U.S. Provisional Application No. 63/366,881, filed Jun. 22, 2022, which is hereby incorporated by reference in its entirety.

### SEQUENCE LISTING

This application contains a Sequence Listing which has been submitted electronically in XML file format and is hereby incorporated by reference in its entirety. The XML file is titled 258199_010202_SL.xml, was created on Jun. 22, 2023, and is 312,715 bytes in size.

### FIELD

The field of the invention is immunology, in particular therapeutic antibodies, and treatment of disease with those antibodies.

### BACKGROUND

Fibrosis is a process of scarring that manifests itself in many tissues in the body, typically as a result of inflammation or tissue damage. One such disease that involves inflammation and fibrosis is rheumatoid arthritis (RA). RA is a chronic autoimmune disease that typically involves inflammation of joints, especially the small joints in the hands, wrists, and feet. As the disease progresses, the continual inflammation leads to a loss of joint mobility and eventually deformity. Additionally, RA can also affect other organs of the body. This can include the lungs, leading to pulmonary fibrosis, where the lung tissue becomes scarred. Disclosed herein are compositions for the treatment of RA and other fibrosis, inflammation, and autoimmune diseases listed herein.

### SUMMARY

In some embodiments, an antibody, or an antigen-binding fragment thereof, is provided, wherein the antibody or antigen-binding fragment thereof, comprises: (i) a heavy chain variable region comprising heavy chain HCDR1, HCDR2, and HCDR3 sequences, wherein the heavy chain HCDR1 sequence has the amino acid sequence of SEQ ID NO: 1, the heavy chain HCDR2 has the amino acid sequence of SEQ ID NO: 2 and the heavy chain HCDR3 sequence has the amino acid sequence of SEQ ID NO: 3; or variants of any of the foregoing; and (ii) a light chain variable region comprising light chain LCDR1, LCDR2, and LCDR3 sequences, wherein the light chain LCDR1 sequence has the amino acid sequence SEQ ID NO: 16; the light chain LCDR2 sequence has the amino acid sequence of SEQ ID NO: 17; and the light chain LCDR3 sequence has the amino acid sequence of SEQ ID NO: 18; or variants of any of the foregoing. In some embodiments, an antibody, or an antigen-binding fragment thereof, wherein the antibody, or the antigen-binding fragment thereof, comprises: (i) a heavy chain variable region comprising heavy chain HCDR1, HCDR2, and HCDR3 sequences, wherein the heavy chain HCDR1 sequence has the amino acid sequence of SEQ ID NO: 23; the heavy chain HCDR2 has the amino acid sequence of SEQ ID NO: 24, SEQ ID NO: 25, SEQ ID NO: 26, or SEQ ID NO: 27, and the heavy chain HCDR3 sequence has the amino acid sequence of SEQ ID NO: 28, or SEQ ID NO: 29, or variants of any of the foregoing; and (ii) a light chain variable region comprising light chain LCDR1, LCDR2, and LCDR3 sequences, wherein the light chain LCDR1 sequence has the amino acid sequence SEQ ID NO: 34; the light chain LCDR2 sequence has the amino acid sequence of SEQ ID NO: 35; and the light chain LCDR3 sequence has the amino acid sequence of SEQ ID NO: 36; or variants of any of the foregoing.

In some embodiments, an antibody, or an antigen-binding fragment thereof, wherein the antibody or antigen-binding fragment thereof, comprises: (i) a heavy chain variable region comprising heavy chain HCDR1, HCDR2, and HCDR3 sequences, wherein the heavy chain HCDR1 sequence has the amino acid sequence of SEQ ID NO: 38, SEQ ID NO: 39, or SEQ ID NO: 40; the heavy chain HCDR2 has the amino acid sequence of SEQ ID NO: 41 or SEQ ID NO: 42; and the heavy chain HCDR3 sequence has the amino acid sequence of SEQ ID NO: 43; or variants of any of the foregoing; and (ii) a light chain variable region comprising light chain LCDR1, LCDR2, and LCDR3 sequences, wherein the light chain LCDR1 sequence has the amino acid sequence SEQ ID NO: 58; the light chain LCDR2 sequence has the amino acid sequence of SEQ ID NO: 59; and the light chain LCDR3 sequence has the amino acid sequence of SEQ ID NO: 60; or variants of any of the foregoing. In some embodiments, the antibody, or antigen-binding fragment thereof provided for herein, is or comprises a scFv antibody, a Fab fragment, a Fab′ fragment, or an F(ab′)₂ fragment. In some embodiments, the antibody is a monoclonal antibody. In some embodiments, the antibody is a humanized antibody.

In some embodiments, the antibody, or antigen-binding fragment thereof, comprises a constant region selected from SEQ ID NO: 63, SEQ ID NO: 64, SEQ ID NO: 65, SEQ ID NO: 66, SEQ ID NO: 67, SEQ ID NO: 68, or SEQ ID NO: 69.

In some embodiments, a method of producing a polypeptide comprising a heavy chain variable region and/or light chain variable region is provided, the method comprising: (a) growing a host cell comprising a polypeptide encoding for an antibody provided for herein under conditions so that the host cell expresses the polypeptide comprising the heavy chain variable region or the light chain variable region; and (b) purifying the polypeptide comprising the heavy chain variable region and/or the light chain variable region.

In some embodiments, a method of producing an antibody that binds human α11β1 integrin, or an antigen-binding fragment thereof, is provided, the method comprising: (a) growing a host cell comprising a polypeptide encoding for an antibody provided for herein under conditions so that the host cell expresses a polypeptide or polypeptides comprising the immunoglobulin heavy chain variable region and/or the immunoglobulin light chain variable region, thereby producing the antibody or the antigen-binding fragment of the antibody; and (b) purifying the antibody or the antigen-binding fragment thereof.

In some embodiments, a method of treating a subject with α11β1 mediated disorder is provided, the method comprising administering to the subject an antibody, or antigen-binding fragment thereof provided for herein. In some embodiments, the disorder is a fibrotic disorder, an autoimmune disorder, an inflammation disorder, or cancer.

### BRIEF DESCRIPTION OF THE DRAWINGS

FIG. 1 shows representations of an integrin structure. The panels illustrate the structure of collagen-binding integrins and three different conformations integrins can exist in on the surface of a cell.

FIGS. 2A, 2B, and 2C show that anti-α11β1 antibodies provide an inhibition of secreted pro-fibrotic and pro-inflammatory mediator Col1a1. FIG. 2A is antibody 1994-01-C07; FIG. 2B is antibody 79E3E3; and FIG. 2C is antibody 16E10.

FIGS. 3A, 3B, and 3C show that anti-α11β1 antibodies provide an inhibition of secreted pro-fibrotic and pro-inflammatory mediator IL-11. FIG. 3A is antibody 1994-01-C07; FIG. 3B is antibody 79E3E3; and FIG. 3C is antibody 16E10.

FIGS. 4A-4C depict assays for 1994-01-C7 antibody variants. FIG. 4A depicts a CHO-hu α11 binding assay; FIG. 4B depicts a myofibroblast binding assay; and FIG. 4C depicts an inhibition of cell adhesion assay.

FIGS. 5A and 5B depict assays for 16E10 antibody variants. FIG. 5A depicts a CHO-hu all binding assay; and FIG. 5B depicts a CHO-α11 adhesion inhibition assay.

FIGS. 6A and 6B show that α11β1 is strongly expressed in stromal cells of subjects with rheumatoid arthritis (FIG. 6A), compared to other cells or cells from subjects with osteoarthritis (FIG. 6B). FIGS. 7A and 7B show that α11β1 is highly expressed in cells of subjects that are TNF inadequate responders (TNF-IR; FIG. 7A) and α11β1 is expressed in low, moderate, and severe rheumatoid arthritis.

FIG. 8 shows that α11β1 is selectively expressed in subliming fibroblast cells.

### DETAILED DESCRIPTION

Provided herein are binding proteins, e.g., antibodies, or fragments, thereof, that selectively bind to type I collagen receptor integrin alpha 11 beta 1 (α11β1). Also provided herein are nucleic acids that encode for antibodies and antibody fragments that bind to α11β1 and methods of using the antibodies and antibody fragments to treat fibrosis and other diseases disclosed herein.

Before the present compositions and methods are described, it is to be understood that the scope of the invention is not limited to the particular processes, compositions, or methodologies described herein, as these may vary. It is also to be understood that the terminology used in the description is for the purpose of describing the particular versions or embodiments only and is not intended to limit the scope of the present invention. Unless defined otherwise, all technical and scientific terms used herein have the same meanings as commonly understood by one of ordinary skill in the art. Although any methods and materials similar or equivalent to those described herein can be used in the practice or testing of embodiments of the methods and systems disclosed herein, the preferred methods, devices, and materials are now described.

The following explanations of terms and methods are provided to better describe the present disclosure and to guide those of ordinary skill in the art in the practice of the present disclosure.

As used herein, “comprising” means “including” and the singular forms “a” or “an” or “the” include plural references unless the context clearly dictates otherwise. For example, reference to “comprising a therapeutic agent” includes one or a plurality of such therapeutic agents. The term “or” refers to a single element of stated alternative elements unless the context clearly indicates otherwise. For example, the phrase “A or B” refers to A alone or B alone. The phrase “A, B, or a combination thereof” refers to A alone, B alone, or a combination of A and B. Similarly, “one or more of A and B” refers to A, B, or a combination of both A and B. The phrase “A and B” refers to a combination of A and B. Furthermore, the various elements, features and steps discussed herein, as well as other known equivalents for each such element, feature, or step, can be mixed and matched by one of ordinary skill in this art to perform methods in accordance with principles described herein. Among the various elements, features, and steps some will be specifically included and others specifically excluded in particular examples.

Unless otherwise defined, all technical and scientific terms used herein have the same meaning as commonly understood to one of ordinary skill in the art. Although methods and materials similar or equivalent to those described herein can be used in the practice or testing of the present disclosure, suitable methods and materials are described below. The materials, methods, and examples are illustrative only and not intended to be limiting. All references cited herein are incorporated by reference in their entirety.

As used herein and unless otherwise indicated, the term “about” is intended to mean ±5% of the value it modifies. Thus, about 100 means 95 to 105.

As used herein, the term “antibody” refers to any form of antibody that exhibits the desired biological activity. Thus, it is used in the broadest sense and specifically covers, but is not limited to, monoclonal antibodies (including full length monoclonal antibodies), polyclonal antibodies, multispecific antibodies (e.g., bispecific antibodies), humanized, fully human antibodies, chimeric antibodies and camelized single domain antibodies. “Parental antibodies” are antibodies obtained by exposure of an immune system to an antigen prior to modification of the antibodies for an intended use, such as humanization of an antibody for use as a human therapeutic antibody.

As used herein, unless otherwise indicated, “antibody fragment” or “antigen-binding fragment” refers to antigen-binding fragments of antibodies, i.e., antibody fragments that retain the ability to bind specifically to the antigen bound by the full-length antibody, e.g., fragments that retain one or more CDR regions. Examples of antibody binding fragments include, but are not limited to, Fab, Fab′, F(ab′)₂, and Fv fragments; diabodies; linear antibodies; single-chain antibody molecules, e.g., sc-Fv; nanobodies (single domain antibody) and multispecific antibodies formed from antibody fragments.

A “Fab fragment” is comprised of one light chain and the C_{H}1 and variable regions of one heavy chain. The heavy chain of a Fab molecule cannot form a disulfide bond with another heavy chain molecule.

An “Fc” region contains two heavy chain fragments comprising the C_{H}2 and C_{H}3 domains of an antibody. The two heavy chain fragments are held together by two or more disulfide bonds and by hydrophobic interactions of the C_{H}3 domains.

A “Fab′ fragment” contains one light chain and a portion or fragment of one heavy chain that contains the V_{H} domain and the C_{H}1 domain and also the region between the C_{H}1 and C_{H}2 domains, such that an interchain disulfide bond can be formed between the two heavy chains of two Fab′ fragments to form a F(ab′)₂ molecule.

A “F(ab′)₂ fragment” contains two light chains and two heavy chains containing a portion of the constant region between the C_{H}1 and C_{H}2 domains, such that an interchain disulfide bond is formed between the two heavy chains. A F(ab′)₂ fragment thus is composed of two Fab′ fragments that are held together by a disulfide bond between the two heavy chains.

The “Fv region” comprises the variable regions from both the heavy and light chains but lacks the constant regions.

The term “single-chain Fv” or “scFv” antibody refers to antibody fragments comprising the V_{H} and V_{L} domains of an antibody, wherein these domains are present in a single polypeptide chain. Generally, the Fv polypeptide further comprises a polypeptide linker between the V_{H} and V_{L} domains which enables the scFv to form the desired structure for antigen-binding. For a review of scFv, see Pluckthun (1994) THE PHARMACOLOGY OF MONOCLONAL ANTIBODIES, vol. 113, Rosenburg and Moore eds. Springer-Verlag, New York, pp. 269-315. See also, International Patent Application Publication No. WO 88/01649 and U.S. Pat. Nos. 4,946,778 and 5,260,203.

A “single-domain antibody” is an immunologically functional immunoglobulin fragment containing only the variable region of a heavy chain or the variable region of a light chain. In some instances, two or more V_{H} regions are covalently joined with a peptide linker to create a bivalent domain antibody. The two V_{H} regions of a bivalent domain antibody may target the same or different antigens.

A “bivalent antibody” comprises two antigen-binding sites. In some instances, the two binding sites have the same antigen specificities. However, bivalent antibodies may be bispecific (see below).

In certain embodiments, monoclonal antibodies herein also include camelized single domain antibodies. See, e.g., Muyldermans et al. (2001) Trends Biochem. Sci. 26:230; Reichmann et al. (1999) J. Immunol. Methods 231:25; WO 94/04678; WO 94/25591; U.S. Pat. No. 6,005,079). In one embodiment, the present invention provides single domain antibodies comprising two V_{H} domains with modifications such that single domain antibodies are formed.

As used herein, the term “diabodies” refers to small antibody fragments with two antigen-binding sites, which fragments comprise a heavy chain variable domain (V_{H}) connected to a light chain variable domain (V_{L}) in the same polypeptide chain (V_{H}-V_{L} or V_{L}-V_{H}). By using a linker that is too short to allow pairing between the two domains on the same chain, the domains are forced to pair with the complementary domains of another chain and create two antigen-binding sites. Diabodies are described more fully in, e.g., EP 404,097; WO 93/11161; and Holliger et al. (1993) Proc. Natl. Acad. Sci. USA 90:6444-6448. For a review of engineered antibody variants generally see Holliger and Hudson (2005) Nat. Biotechnol. 23:1126-1136.

Typically, a variant antibody or antigen-binding fragment of the antibodies provided herein retain at least 10% of its A11B1s binding activity (when compared to a parental antibody that is modified) when that activity is expressed on a molar basis. In some embodiments, a variant antibody (or antigen fragment thereof), or antigen-binding fragment of an antibody provided herein, retains at least 20%, 50%, 70%, 80%, 90%, 95% or 100% or more of the A11B1s binding affinity as the parental antibody. As described herein, it is also intended that an antibody or antigen-binding fragment of the invention can include conservative or non-conservative amino acid substitutions, which can also be referred to as “conservative variants” or “function conserved variants” of the antibody, that do not substantially alter its biologic activity.

“Isolated antibody” refers to the purification status of a binding compound and in such context means the molecule is substantially free of other biological molecules such as nucleic acids, proteins, lipids, carbohydrates, or other material such as cellular debris and growth media. Generally, the term “isolated” is not intended to refer to a complete absence of such material or to an absence of water, buffers, or salts, unless they are present in amounts that substantially interfere with experimental or therapeutic use of the binding compound as described herein.

The term “monoclonal antibody”, as used herein, refers to population of substantially homogeneous antibodies, i.e., the antibody molecules comprising the population are identical in amino acid sequence except for possible naturally occurring mutations and/or post-translational modifications that may be present in minor amounts. In contrast, conventional (polyclonal) antibody preparations typically include a multitude of different antibodies having different amino acid sequences in their variable domains, particularly their CDRs, that are often specific for different epitopes. The modifier “monoclonal” indicates the character of the antibody as being obtained from a substantially homogeneous population of antibodies and is not to be construed as requiring production of the antibody by any particular method. For example, the monoclonal antibodies to be used in accordance with the present invention may be made by the hybridoma method first described by Kohler et al. (1975) Nature 256:495, or may be made by recombinant DNA methods (see, e.g., U.S. Pat. No. 4,816,567). The “monoclonal antibodies” may also be isolated from phage antibody libraries using the techniques described in Clackson et al. (1991) Nature 352:624-628 and Marks et al. (1991) J. Mol. Biol. 222:581-597, for example. See also Presta (2005) J. Allergy Clin. Immunol. 116:731.

As used herein, a “chimeric antibody” is an antibody having the variable domain from a first antibody and constant domain from a second antibody, where the first and second antibodies are from different species. (U.S. Pat. No. 4,816,567; and Morrison et al., (1984) Proc. Natl. Acad. Sci. USA 81:6851-6855). Typically, the variable domains are obtained from an antibody from an experimental animal (the “parental antibody”), such as a rodent, and the constant domain sequences are obtained from human antibodies, so that the resulting chimeric antibody will be less likely to elicit an adverse immune response in a human subject than the parental (e.g., rodent) antibody.

As used herein, the term “humanized antibody” refers to forms of antibodies that contain sequences from both human and non-human (e.g., murine, rat) antibodies. In general, the humanized antibody will comprise substantially all of at least one, and typically two, variable domains, in which all or substantially all of the hypervariable loops correspond to those of a non-human immunoglobulin, and all or substantially all of the framework (FR) regions are those of a human immunoglobulin sequence. The humanized antibody may optionally comprise at least a portion of a human immunoglobulin constant region (Fc).

The term “fully human antibody” refers to an antibody that comprises human immunoglobulin protein sequences only. A fully human antibody may contain murine carbohydrate chains if produced in a mouse, in a mouse cell, or in a hybridoma derived from a mouse cell. Similarly, “mouse antibody” refers to an antibody that comprises mouse immunoglobulin sequences only. Alternatively, a fully human antibody may contain rat carbohydrate chains if produced in a rat, in a rat cell, or in a hybridoma derived from a rat cell. Similarly, “rat antibody” refers to an antibody that comprises rat immunoglobulin sequences only.

In some embodiments, the basic antibody structural unit comprises a tetramer. Each tetramer includes two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of the heavy chain may define a constant region primarily responsible for effector function. Typically, human light chains are classified as kappa and lambda light chains. Furthermore, human heavy chains are typically classified as mu, delta, gamma, alpha, or epsilon, and define the antibody's isotype as IgM, IgD, IgG, IgA, and IgE, respectively. Within light and heavy chains, the variable and constant regions are joined by a “J” region of about 12 or more amino acids, with the heavy chain also including a “D” region of about 10 more amino acids. See generally, Fundamental Immunology Ch. 7 (Paul, W., ed., 2nd ed. Raven Press, N.Y. (1989).

The variable regions of each light/heavy chain pair form the antibody binding site. Thus, in general, an intact antibody has two binding sites. However, in bifunctional or bispecific antibodies, the two binding sites are, in general, not the same.

Typically, the variable domains of both the heavy and light chains comprise three hypervariable regions, also called complementarity determining regions (CDRs), located within relatively conserved framework regions (FR). The CDRs are usually aligned by the framework regions, enabling binding to a specific epitope. In general, from N-terminal to C-terminal, both light and heavy chains variable domains comprise FR1, CDR1, FR2, CDR2, FR3, CDR3 and FR4. The assignment of amino acids to each domain is, generally, in accordance with the definitions of Sequences of Proteins of Immunological Interest, Kabat, et al.; National Institutes of Health, Bethesda, Md.; 5^{th} ed.; NIH Publ. No. 91-3242 (1991); Kabat (1978) Adv. Prot. Chem. 32:1-75; Kabat, et al., (1977) J. Biol. Chem. 252:6609-6616; Chothia, et al., (1987) J Mol. Biol. 196:901-917 or Chothia, et al., (1989) Nature 342:878-883.

As used herein, the term “hypervariable region” refers to the amino acid residues of an antibody that are responsible for antigen-binding. The hypervariable region comprises amino acid residues from a “complementarity determining region” or “CDR” (i.e. residues 24-34 (CDRL1), 50-56 (CDRL2) and 89-97 (CDRL3) in the light chain variable domain and residues 31-35 (CDRH1), 50-65 (CDRH2) and 95-102 (CDRH3) in the heavy chain variable domain; Kabat et al. (1991) Sequences of Proteins of Immunological Interest, 5th Ed. Public Health Service, National Institutes of Health, Bethesda, Md.) and/or those residues from a “hypervariable loop” (i.e. residues 26-32 (CDRL1), 50-52 (CDRL2) and 91-96 (CDRL3) in the light chain variable domain and 26-32 (CDRH1), 53-55 (CDRH2) and 96-101 (CDRH3) in the heavy chain variable domain; Chothia and Lesk (1987) J. Mol. Biol. 196:901-917). The CDRs can also be referenced according to the IMGT system for the identification of CDRs, which is described in Lefranc M P. Unique database numbering system for immunogenetic analysis. Immunol Today (1997) 18:509. As used herein, the term “framework” or “FR” residues refers to those variable domain residues other than the hypervariable region residues defined herein as CDR residues. CDRs provide the majority of contact residues for the binding of the antibody to the antigen or epitope. CDRs of interest can be derived from donor antibody variable heavy and light chain sequences, and include analogs of the naturally occurring CDRs, which analogs also share or retain the same antigen-binding specificity and/or neutralizing ability as the donor antibody from which they were derived.

As used herein, “specific binding” or “immunospecific binding” or “binds immunospecifically” refer to antibody binding to a predetermined antigen at a much higher affinity than for another antigen(s) (e.g., selectively binds the active form of complement component A11B1s as compared to inactive A11B1s, which can also be referred to as proA11B1s zymogen). In some embodiments, the antibody binds the predetermined antigen with a dissociation constant (K_{D}) of 10⁻⁷ M or less, and such K_{D} is at least two-fold less than its K_{D} for binding to a non-specific antigen (e.g., BSA, casein, or another non-specific polypeptide).

The phrases “an antibody recognizing A11B1s” and “an antibody specific for A11B1s” are used interchangeably herein with the term “an antibody which binds immunospecifically to A11B1s.” In some embodiments, the antibody binds specifically or preferentially to A11B1s, such as the active form of A11B1s over other proteins, such as, but not limited to, the inactive form of A11B1s (proA11B1s). The degree of specificity necessary for an anti-A11B1s antibody may depend on the intended use of the antibody, and at any rate is defined by its suitability for use for an intended purpose. In some embodiments, the antibody, or binding compound derived from the antigen-binding site of an antibody, of the contemplated method binds to its antigen (active form of A11B1s), with an affinity that is at least two-fold greater, at least ten times greater, at least 20-times greater, or at least 100-times greater than the affinity with any other antigen, including, but not limited to inactive A11B1s.

Methods for determining mAb specificity and affinity by competitive inhibition can be found in Harlow, et al., Antibodies: A Laboratory Manual, Cold Spring Harbor Laboratory Press, Cold Spring Harbor, N.Y., 1988), Colligan et al., eds., Current Protocols in Immunology, Greene Publishing Assoc, and Wiley Interscience, N.Y., (1992, 1993), and Muller, Meth. Enzymol. 92:589 601 (1983), which references are entirely incorporated herein by reference.

The term “homolog” means protein sequences having between 40% and 100% sequence identity to a reference sequence. Percent identity between two peptide chains can be determined by pair wise alignment using the default settings of the AlignX module of Vector NTI v.9.0.0 (Invitrogen Corp., Carlsbad, Calif.) or other suitable alignment software, such as BLAST. In some embodiments, the antibody, or antigen-binding fragment thereof has, at least 50, 60, 70, 80, 90, 91, 92, 93, 94, 95, 96, 97, 98, or 99% homology or identity to a sequence described herein. In some embodiments, the antibody has conservative substitutions as compared to a sequence described herein. Exemplary conservative substitutions are illustrated in Table 1 and are encompassed within the scope of the disclosed subject matter. The conservative substitution may reside in the framework regions, or in antigen-binding sites, as long they do not adversely affect the properties of the antibody. Substitutions may be made to improve antibody properties, for example stability or affinity. Conservative substitutions will produce molecules having functional and chemical characteristics similar to those molecules into which such modifications are made. Exemplary amino acid substitutions are shown in the table below.

TABLE 1
Exemplary Conservative Substitutions

| | Exemplary |
|---|---|
| Original | Conservative |
| Residue | Substitutions |
| Ala | Val, Leu, Ile |
| Arg | Lys, Gln, Asn |
| Asn | Gln |
| Asp | Glu |
| Cys | Ser, Ala |
| Gln | Asn |
| Gly | Pro, Ala |
| His | Asn, Gln, Lys, Arg |
| Ile | Leu, Val, Met, Ala, Phe |
| Leu | Ile, Val, Met, Ala, Phe |
| Lys | Arg, Gln, Asn |
| Met | Leu, Phe, Ile |
| Phe | Leu, Val, Ile, Ala, Tyr |
| Pro | Ala |
| Ser | Thr, Ala, Cys |
| Thr | Ser |
| Trp | Tyr, Phe |
| Tyr | Trp, Phe, Thr, Ser |
| Val | Ile, Met, Leu, Phe, Ala |

In some embodiments, variants of the proteins and peptides provided herein are provided. In some embodiments, a variant comprises a substitution, deletions, or insertion. In some embodiments, the variant comprises 1, 2, 3, 4, 5, 6, 7, 8, 9, or 10 (e.g., 1-10) substitutions. As described herein, the substitutions can be conservative substitutions. In some embodiments, the substitution is non-conservative. In some embodiments, the variant comprises 1, 2, 3, 4, 5, 6, 7, 8, 9, or 10 (e.g., 1-10) deletions. In some embodiments, the variant comprises 1, 2, 3, 4, 5, 6, 7, 8, 9, or 10 (e.g., 1-10) insertions. In some embodiments, the substitutions, deletions, or insertions are present in the CDRs provided for herein. In some embodiments, the substitutions, deletions, or insertions are not present in the CDRs provided for herein.

The term “in combination with” as used herein means that the described agents can be administered to an animal or subject together in a mixture, concurrently as single agents or sequentially as single agents in any order.

The term “epitope” is meant to refer to that portion of any molecule capable of being recognized by and bound by an antibody at one or more of the Ab's antigen-binding regions. Epitopes usually consist of chemically active surface groupings of molecules such as amino acids or sugar side chains and have specific three-dimensional structural characteristics as well as specific charge characteristics

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., Sendai viruses, lentiviruses, retroviruses, adenoviruses, and adeno-associated viruses) that incorporate the recombinant polynucleotide.

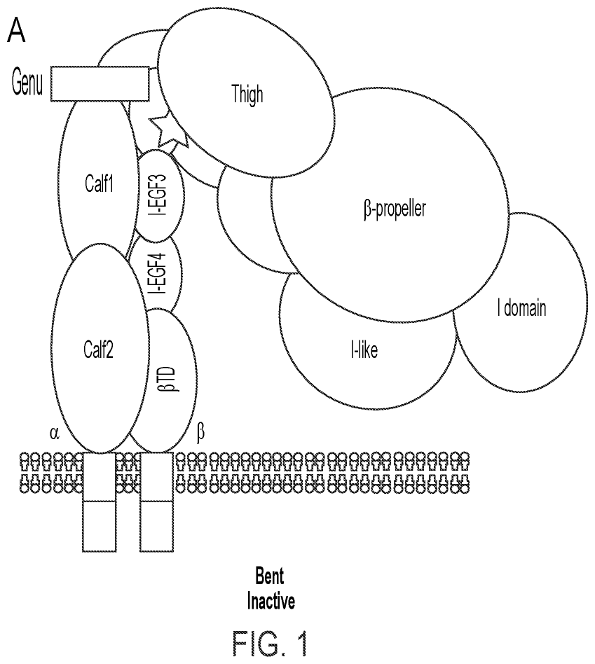

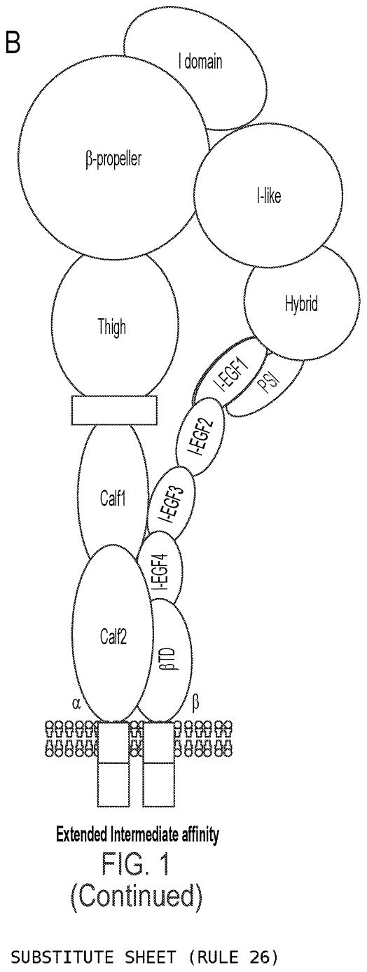

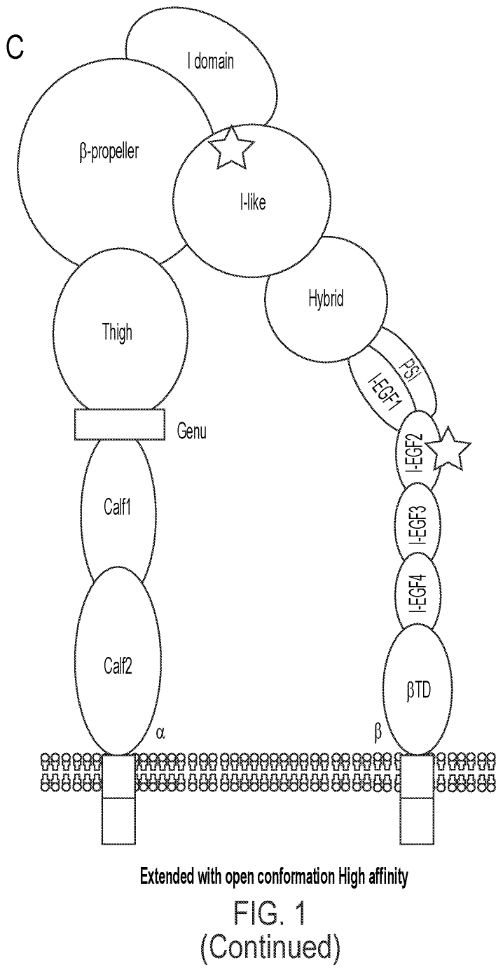

“Integrins” are a large family of type I transmembrane heterodimeric glycoprotein receptors and act as major receptors for cell adhesion. The integrin family of receptors plays key roles in modulating signal transduction pathways that control cell adhesion, migration, proliferation, differentiation and apoptosis. There are 18 α and 8 β subunits, which combine to form 24 integrin heterodimers. Each integrin receptor comprises two non-covalently bound subunits, α and β. Integrins α1β1, α2β1, α10β1, and α11β1 are the primary collagen receptors. α and β subunits are transmembrane proteins with large, modular, extracellular domains, single transmembrane helices, and short cytoplasmic regions, which mediate cytoskeletal interactions. Extracellular domains of integrins are generally large, approximately 80-150 kDa structures. The extracellular domains can be seen as comprising a headpiece connected to two legs (see FIG. 1 for structure of collagen-binding integrins). Collagen binding integrins contain an I domain, which serves as the ligand-binding site. The aï-domain contains a conserved “metal-ion-dependent adhesion site” (MIDAS) that binds divalent metal cations (Mg2+) and plays important role in ligand binding.

Integrins can exist in three different conformations: 1) a resting, low affinity state (bent conformation, FIG. 1, panel A) where the head piece containing ligand binding site is turned towards the membrane; 2) an extended, intermediate affinity state, where the integrin is extended but the head piece remains ‘closed’ (FIG. 1, panel B) and 3) an extended, high affinity state where the integrin is fully activated and readily binds the ligand. The complexity of the different integrin states allows for both allosteric and ligand-blocking ways of inhibiting integrin function. As marked with a star in FIG. 1, one of the allosteric ways to block the function of an integrin is to generate a monoclonal antibody that prevents the integrin from reaching the fully extended conformation from the extended intermediate conformation. Another allosteric option is to bind an integrin in its bent/inactive conformation and to keep it from extending to either of the two other states. A non-allosteric way of inhibiting integrin function is to bind to the I domain a prevent the integrin from attaching to collagen. Binding to the ligand binding site directly runs the risk of generating a recombinant activator of integrin function.

As cell surface receptors, integrins sense the stiffness of the surrounding matrix, triggering the cells to further produce and remodel connective tissue, which can perpetuate a fibrotic phenotype. Many integrins are overexpressed in fibrosis, but it is not clear which alpha subunit is sufficient for fibrosis to occur. α11β1 integrin is specifically expressed on a subset of fibroblasts and myofibroblasts (i.e., terminal scar producing cells). Recent literature has provided strong evidence that α11β1 is one of the main drivers of a fibrotic phenotype in cardiac tissue, liver, lungs and kidney (Romaine, A. et. al. Overexpression of integrin alpha 11 induces cardiac fibrosis in mice. Acta Physiol February 2018, 222 (2); Bansal, R. et. al. Integrin alpha 11 in the regulation of the myofibroblast phenotype: implications for fibrotic diseases. Exp Mol Med. 2017 Nov. 17: 49 (11)). Blocking α11β1 function may inhibit myofibroblast differentiation and extracellular matrix deposition (i.e., the major event in scar formation) and blocking al Ibï function may provide a mechanism for local, injury-specific attenuation of fibrosis which could fundamentally change fibrotic microenvironment and modify disease progression in all diseases that have a fibrotic component.

In some embodiments, an anti-α11β1 antibody, or antigen-binding fragment thereof, of the present disclosure reduces interaction of α11β1 with collagen in human α11β1-expressing cells. In some embodiments, reducing interaction of α11β1 with collagen in human α11β1-expressing cells comprises an anti-α11β1 antibody, or antigen-binding fragment thereof, interacting with α11β1 that is in a resting, low affinity state (bent conformation). In some embodiments, reducing interaction of α11β1 with collagen in human α11β1-expressing cells comprises an anti-α11β1 antibody, or antigen-binding fragment thereof, interacting with α11β1 that is in an extended, intermediate affinity state. In some embodiments, reducing interaction of α11β1 with collagen in human α11β1-expressing cells comprises an anti-α11β1 antibody, or antigen-binding fragment thereof, interacting with α11β1 that is in an extended, high affinity state.

### Compositions

The present disclosure provides an anti-α11β1 antibody or antigen-binding fragment thereof. In some embodiments, the antibody is a monoclonal antibody or fragment thereof which binds to α11β1. In some embodiments, the antibody is a homodimeric monoclonal antibody or fragment thereof which binds to α11β1. In some embodiments, the antibody is a heterodimeric monoclonal antibody or fragment thereof which binds to α11β1. In some embodiments, an antibody is, e.g., a typical antibody or a diabody, triabody, tetrabody, minibody, maxibody, tandab, DVD, BiTe, scFv, TandAb scFv, Fab, Fab2, Fab3, F(ab′)₂, or the like, or any combination thereof.

The term “purified” with reference to an antibody refers to an antibody that is substantially free of other material that associates with the molecule in its natural environment. For instance, a purified protein is substantially free of the cellular material or other proteins from the cell or tissue from which it is derived. The term refers to preparations where the isolated protein is sufficiently pure to be analyzed, or at least 70% to 80% (w/w) pure, at least 80%-90% (w/w) pure, 90-95% pure; and at least 95%, 96%, 97%, 98%, 99%, or 100% (w/w) pure. In some embodiments, the antibody is purified.

The antibodies provided for herein may also be conjugated to a chemical moiety. The chemical moiety may be, inter alia, a polymer, a radionuclide or a cytotoxic factor. In some embodiments, this can be referred to as an antibody drug conjugate. In some embodiments, the chemical moiety is a polymer which increases the half-life of the antibody molecule in the body of a subject. Suitable polymers include, but are not limited to, polyethylene glycol (PEG) (e.g., PEG with a molecular weight of 2 kDa, 5 kDa, 10 kDa, 12 kDa, 20 kDa, 30 kDa or 40 kDa), dextran and monomethoxypolyethylene glycol (mPEG). Lee, et al., (1999) (Bioconj. Chem. 10:973-981) discloses PEG conjugated single-chain antibodies. Wen, et al., (2001) (Bioconj. Chem. 12:545-553) disclose conjugating antibodies with PEG which is attached to a radiometal chelator (diethylenetriaminpentaacetic acid (DTPA)). Examples of chemical moieties include, but are not limited to, anti-mitoties, such as calicheamicins (e.g., ozogamicin), monometbyl auristatin E, mertansine, and the like. Other examples include, but are not limited to, biologically active anti-microtubule agents, alkylating agents and DNA minor groove binding agents. Other examples of are provided herein and below. The chemical moiety can be linked to the antibody through a linking group (maleimide), a cleavable linker, such as a cathepsin cleavable linkers (valine-citrulline), and in some embodiments, one or more spacers (e.g., para-aminobenzylcarbamate).

The antibodies and antibody fragments of the invention may also be conjugated with labels such as ⁹⁹Tc, ⁹⁰Y, ¹¹¹In, ³²P, ¹⁴C, ¹²⁵I, ³H, ¹³¹I, ¹¹C, ¹⁵O, ¹³N, ¹⁸F, ³⁵S, ⁵¹Cr, ⁵⁷To, ²²⁶Ra, ⁶⁰Co, ⁵⁹Fe, ⁵⁷Se, ¹⁵²Eu, ⁶⁷CU, ²¹⁷Ci, ²¹¹At, ²¹²Pb, ⁴⁷Sc, ¹⁰⁹Pd, ²³⁴Th, and ⁴⁰K, ¹⁵⁷Gd, ⁵⁵Mn, ⁵²Tr and ⁵⁶Fe.

The antibodies and antibody fragments may also be conjugated with fluorescent or chemiluminescent labels, including fluorophores such as rare earth chelates, fluorescein and its derivatives, rhodamine and its derivatives, isothiocyanate, phycoerythrin, phycocyanin, allophycocyanin, o-phthaladehyde, fluorescamine, 152Eu, dansyl, umbelliferone, luciferin, luminal label, isoluminal label, an aromatic acridinium ester label, an imidazole label, an acridimium salt label, an oxalate ester label, an aequorin label, 2,3-dihydrophthalazinediones, biotin/avidin, spin labels and stable free radicals.

The antibody molecules may also be conjugated to a cytotoxic factor such as diptheria toxin, Pseudomonas aeruginosa exotoxin A chain, ricin A chain, abrin A chain, modeccin A chain, alpha-sarcin, Aleurites fordii proteins and compounds (e.g., fatty acids), dianthin proteins, Phytoiacca americana proteins PAPI, PAPII, and PAP-S, Momordica charantia inhibitor, curcin, crotin, Saponaria officinalis inhibitor, mitogellin, restrictocin, phenomycin, and enomycin.

Any method known in the art for conjugating the antibody molecules of the invention to the various moieties may be employed, including those methods described by Hunter, et al., (1962) Nature 144:945; David, et al., (1974) Biochemistry 13:1014; Pain, et al., (1981) J. Immunol. Meth. 40:219; and Nygren, J., (1982) Histochem, and Cytochem. 30:407. Methods for conjugating antibodies are conventional and very well known in the art.

In some embodiments, the antibody comprises a heavy chain CDR peptide having one of the following sequences, or a variant thereof (Table 2):

TABLE 2
heavy chain CDRs

| | |
|---|---|
| SEQ ID NO: 1 | GYTFTDYAMN |
| SEQ ID NO: 2 | WINTQTGKPT |
| SEQ ID NO: 3 | LGTGNTKGFAY |
| SEQ ID NO: 23 | GYTFTSYGIS |
| SEQ ID NO: 24 | WISAYNANTN |
| SEQ ID NO: 25 | WISAYNANVN |
| SEQ ID NO: 26 | WISAYNGNTN |
| SEQ ID NO: 27 | WISAYNSNTN |
| SEQ ID NO: 28 | VTGITGTTIGP |
| SEQ ID NO: 29 | VTGITGTTIDP |
| SEQ ID NO: 38 | GFSFSSSYYMA |
| SEQ ID NO: 39 | GFSFSSSYYMC |
| SEQ ID NO: 40 | GFSFSSSYYMS |
| SEQ ID NO: 41 | AIGTTRGSTY |
| SEQ ID NO: 42 | CIGTTRGSTY |
| SEQ ID NO: 43 | DATGYRINTIGLYFNL |

In some embodiments, the antibody comprises a light chain CDR peptide having one of the following sequences, or a variant thereof (Table 3):

TABLE 3
light chain CDRs

| | |
|---|---|
| SEQ ID NO: 16 | KSSQSLLYSENNQDYLA |
| SEQ ID NO: 17 | GASNRHT |
| SEQ ID NO: 18 | EQTYRYPFT |
| SEQ ID NO: 34 | RASQSISSYLN |
| SEQ ID NO: 35 | DASSLES |
| SEQ ID NO: 36 | QQYNNWPQT |
| SEQ ID NO: 58 | QASQTIYSYLS |
| SEQ ID NO: 59 | EASKLAS |
| SEQ ID NO: 60 | QSYHGTASTEYNT |

In some embodiments, an antibody, or antibody binding fragment thereof, comprises a heavy chain CDR having a sequence of SEQ ID NO: 1, SEQ ID NO: 2, SEQ ID NO: 3, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 29, SEQ ID NO: 39, SEQ ID NO: 40, SEQ ID NO: 41, SEQ ID NO: 42, SEQ ID NO: 43, or a combination thereof. In some embodiments, the antibody, or antibody binding fragment thereof, comprises a heavy chain CDR having a sequence of SEQ ID NO: 1. In some embodiments, the antibody, or antibody binding fragment thereof, comprises a heavy chain CDR having a sequence of SEQ ID NO: 2. In some embodiments, the antibody, or antibody binding fragment thereof, comprises a heavy chain CDR having a sequence of SEQ ID NO: 3. In some embodiments, the antibody, or antibody binding fragment thereof, comprises a heavy chain CDR having a sequence of SEQ ID NO: 23. In some embodiments, the antibody, or antibody binding fragment thereof, comprises a heavy chain CDR having a sequence of SEQ ID NO: 24. In some embodiments, the antibody, or antibody binding fragment thereof, comprises a heavy chain CDR having a sequence of SEQ ID NO: 25. In some embodiments, the antibody, or antibody binding fragment thereof, comprises a heavy chain CDR having a sequence of SEQ ID NO: 26. In some embodiments, the antibody, or antibody binding fragment thereof, comprises a heavy chain CDR having a sequence of SEQ ID NO: 27. In some embodiments, the antibody, or antibody binding fragment thereof, comprises a heavy chain CDR having a sequence of SEQ ID NO: 28. In some embodiments, the antibody, or antibody binding fragment thereof, comprises a heavy chain CDR having a sequence of SEQ ID NO: 29. In some embodiments, the antibody, or antibody binding fragment thereof, comprises a heavy chain CDR having a sequence of SEQ ID NO: 38. In some embodiments, the antibody, or antibody binding fragment thereof, comprises a heavy chain CDR having a sequence of SEQ ID NO: 39. In some embodiments, the antibody, or antibody binding fragment thereof, comprises a heavy chain CDR having a sequence of SEQ ID NO: 40. In some embodiments, the antibody, or antibody binding fragment thereof, comprises a heavy chain CDR having a sequence of SEQ ID NO: 41. In some embodiments, the antibody, or antibody binding fragment thereof, comprises a heavy chain CDR having a sequence of SEQ ID NO: 42. In some embodiments, the antibody, or antibody binding fragment thereof, comprises a heavy chain CDR having a sequence of SEQ ID NO: 43.

In some embodiments, an antibody, or antibody binding fragment thereof, comprises a light chain CDR having a sequence of SEQ ID NO: 16, SEQ ID NO: 17, SEQ ID NO: 18, SEQ ID NO: 34, SEQ ID NO: 35, SEQ ID NO: 36, SEQ ID NO: 58, SEQ ID NO: 59, SEQ ID NO: 60, or a combination thereof. In some embodiments, the antibody, or antibody binding fragment thereof, comprises a light chain CDR having a sequence of SEQ ID NO: 16. In some embodiments, the antibody, or antibody binding fragment thereof, comprises a light chain CDR having a sequence of SEQ ID NO: 17. In some embodiments, the antibody, or antibody binding fragment thereof, comprises a light chain CDR having a sequence of SEQ ID NO: 18. In some embodiments, the antibody, or antibody binding fragment thereof, comprises a light chain CDR having a sequence of SEQ ID NO: 34. In some embodiments, the antibody, or antibody binding fragment thereof, comprises a light chain CDR having a sequence of SEQ ID NO: 35. In some embodiments, the antibody, or antibody binding fragment thereof, comprises a light chain CDR having a sequence of SEQ ID NO: 36. In some embodiments, the antibody, or antibody binding fragment thereof, comprises a light chain CDR having a sequence of SEQ ID NO: 58. In some embodiments, the antibody, or antibody binding fragment thereof, comprises a light chain CDR having a sequence of SEQ ID NO: 59. In some embodiments, the antibody, or antibody binding fragment thereof, comprises a light chain CDR having a sequence of SEQ ID NO: 60.

In some embodiments, an antibody, or antibody binding fragment thereof, comprises a heavy chain variable region having a HCDR1, a HCDR2, and a HCDR3, wherein the HCDR1 has a sequence of SEQ ID NO: 1, the HCDR2 has a sequence of SEQ ID NO: 2, and the HCDR3 has a sequence of SEQ ID NO: 3.

In some embodiments, an antibody, or antibody binding fragment thereof, comprises a heavy chain variable region having a HCDR1, a HCDR2, and a HCDR3, wherein the HCDR1 has a sequence of SEQ ID NO: 23, the HCDR2 has a sequence of SEQ ID NO: 24, and the HCDR3 has a sequence of SEQ ID NO: 28.

In some embodiments, an antibody, or antibody binding fragment thereof, comprises a heavy chain variable region having a HCDR1, a HCDR2, and a HCDR3, wherein the HCDR1 has a sequence of SEQ ID NO: 23, the HCDR2 has a sequence of SEQ ID NO: 24, and the HCDR3 has a sequence of SEQ ID NO: 29.

In some embodiments, an antibody, or antibody binding fragment thereof, comprises a heavy chain variable region having a HCDR1, a HCDR2, and a HCDR3, wherein the HCDR1 has a sequence of SEQ ID NO: 23, the HCDR2 has a sequence of SEQ ID NO: 25, and the HCDR3 has a sequence of SEQ ID NO: 28.

In some embodiments, an antibody, or antibody binding fragment thereof, comprises a heavy chain variable region having a HCDR1, a HCDR2, and a HCDR3, wherein the HCDR1 has a sequence of SEQ ID NO: 23, the HCDR2 has a sequence of SEQ ID NO: 25, and the HCDR3 has a sequence of SEQ ID NO: 29.

In some embodiments, an antibody, or antibody binding fragment thereof, comprises a heavy chain variable region having a HCDR1, a HCDR2, and a HCDR3, wherein the HCDR1 has a sequence of SEQ ID NO: 23, the HCDR2 has a sequence of SEQ ID NO: 26, and the HCDR3 has a sequence of SEQ ID NO: 28.

In some embodiments, an antibody, or antibody binding fragment thereof, comprises a heavy chain variable region having a HCDR1, a HCDR2, and a HCDR3, wherein the HCDR1 has a sequence of SEQ ID NO: 23, the HCDR2 has a sequence of SEQ ID NO: 26, and the HCDR3 has a sequence of SEQ ID NO: 29.

In some embodiments, an antibody, or antibody binding fragment thereof, comprises a heavy chain variable region having a HCDR1, a HCDR2, and a HCDR3, wherein the HCDR1 has a sequence of SEQ ID NO: 23, the HCDR2 has a sequence of SEQ ID NO: 27, and the HCDR3 has a sequence of SEQ ID NO: 28.

In some embodiments, an antibody, or antibody binding fragment thereof, comprises a heavy chain variable region having a HCDR1, a HCDR2, and a HCDR3, wherein the HCDR1 has a sequence of SEQ ID NO: 23, the HCDR2 has a sequence of SEQ ID NO: 27, and the HCDR3 has a sequence of SEQ ID NO: 29.

In some embodiments, an antibody, or antibody binding fragment thereof, comprises a heavy chain variable region having a HCDR1, a HCDR2, and a HCDR3, wherein the HCDR1 has a sequence of SEQ ID NO: 38, the HCDR2 has a sequence of SEQ ID NO: 41, and the HCDR3 has a sequence of SEQ ID NO: 43.

In some embodiments, an antibody, or antibody binding fragment thereof, comprises a heavy chain variable region having a HCDR1, a HCDR2, and a HCDR3, wherein the HCDR1 has a sequence of SEQ ID NO: 38, the HCDR2 has a sequence of SEQ ID NO: 42, and the HCDR3 has a sequence of SEQ ID NO: 43.

In some embodiments, an antibody, or antibody binding fragment thereof, comprises a heavy chain variable region having a HCDR1, a HCDR2, and a HCDR3, wherein the HCDR1 has a sequence of SEQ ID NO: 39, the HCDR2 has a sequence of SEQ ID NO: 41, and the HCDR3 has a sequence of SEQ ID NO: 43.

In some embodiments, an antibody, or antibody binding fragment thereof, comprises a heavy chain variable region having a HCDR1, a HCDR2, and a HCDR3, wherein the HCDR1 has a sequence of SEQ ID NO: 39, the HCDR2 has a sequence of SEQ ID NO: 42, and the HCDR3 has a sequence of SEQ ID NO: 43.

In some embodiments, an antibody, or antibody binding fragment thereof, comprises a heavy chain variable region having a HCDR1, a HCDR2, and a HCDR3, wherein the HCDR1 has a sequence of SEQ ID NO: 40, the HCDR2 has a sequence of SEQ ID NO: 41, and the HCDR3 has a sequence of SEQ ID NO: 43.

In some embodiments, an antibody, or antibody binding fragment thereof, comprises a heavy chain variable region having a HCDR1, a HCDR2, and a HCDR3, wherein the HCDR1 has a sequence of SEQ ID NO: 40, the HCDR2 has a sequence of SEQ ID NO: 42, and the HCDR3 has a sequence of SEQ ID NO: 43.

In some embodiments, an antibody, or antibody binding fragment thereof, comprises a light chain variable region having a LCDR1, a LCDR2, and a LCDR3, wherein the LCDR1 has a sequence of SEQ ID NO: 16, the LCDR2 has a sequence of SEQ ID NO: 17, and the LCDR3 has a sequence of SEQ ID NO: 18.

In some embodiments, an antibody, or antibody binding fragment thereof, comprises a light chain variable region having a LCDR1, a LCDR2, and a LCDR3, wherein the LCDR1 has a sequence of SEQ ID NO: 34, the LCDR2 has a sequence of SEQ ID NO: 35, and the LCDR3 has a sequence of SEQ ID NO: 36.

In some embodiments, an antibody, or antibody binding fragment thereof, comprises a light chain variable region having a LCDR1, a LCDR2, and a LCDR3, wherein the LCDR1 has a sequence of SEQ ID NO: 58, the LCDR2 has a sequence of SEQ ID NO: 59, and the LCDR3 has a sequence of SEQ ID NO: 60.

For clarity, the following tables are provided depicting non-limiting exemplary combinations of heavy chain (Table 4) and light chain (Table 5) CDRs.

TABLE 4
heavy chain CDR combinations

| | HCDR1 | HCDR2 | HCDR3 |
|---|---|---|---|
| V_{H}A | SEQ ID NO: 1 | SEQ ID NO: 2 | SEQ ID NO: 3 |
| V_{H}B | SEQ ID NO: 23 | SEQ ID NO: 24 | SEQ ID NO: 28 |
| V_{H}C | SEQ ID NO: 23 | SEQ ID NO: 24 | SEQ ID NO: 29 |
| V_{H}D | SEQ ID NO: 23 | SEQ ID NO: 25 | SEQ ID NO: 28 |
| V_{H}E | SEQ ID NO: 23 | SEQ ID NO: 25 | SEQ ID NO: 29 |
| V_{H}F | SEQ ID NO: 23 | SEQ ID NO: 26 | SEQ ID NO: 28 |
| V_{H}G | SEQ ID NO: 23 | SEQ ID NO: 26 | SEQ ID NO: 29 |
| V_{H}H | SEQ ID NO: 23 | SEQ ID NO: 27 | SEQ ID NO: 28 |
| V_{H}I | SEQ ID NO: 23 | SEQ ID NO: 27 | SEQ ID NO: 29 |
| V_{H}J | SEQ ID NO: 38 | SEQ ID NO: 41 | SEQ ID NO: 43 |
| V_{H}K | SEQ ID NO: 38 | SEQ ID NO: 42 | SEQ ID NO: 43 |
| V_{H}L | SEQ ID NO: 39 | SEQ ID NO: 41 | SEQ ID NO: 43 |
| V_{H}M | SEQ ID NO: 39 | SEQ ID NO: 42 | SEQ ID NO: 43 |
| V_{H}N | SEQ ID NO: 40 | SEQ ID NO: 41 | SEQ ID NO: 43 |
| V_{H}O | SEQ ID NO: 40 | SEQ ID NO: 42 | SEQ ID NO: 43 |

TABLE 5
light chain CDR combinations

| | LCDR1 | LCDR2 | LCDR3 |
|---|---|---|---|
| V_{L}A | SEQ ID NO: 16 | SEQ ID NO: 17 | SEQ ID NO: 18 |
| V_{L}B | SEQ ID NO: 34 | SEQ ID NO: 35 | SEQ ID NO: 36 |
| V_{L}C | SEQ ID NO: 58 | SEQ ID NO: 59 | SEQ ID NO: 60 |

In some embodiments, an antibody or antibody binding fragment thereof, comprises: (i) a heavy chain having any one of the foregoing recited combinations of HCDR1, HCDR2, and HCDR3 sequences; and (ii) a light chain having any one of the foregoing recited combinations of LCDR1, LCDR2, and LCDR3 sequences. The following combinations are provided with reference to Table 4 and 5 above.

Thus, for example, an antibody or antibody binding fragment thereof comprising V_{H}A and V_{L}A is understood to comprise a heavy chain having a HCDR1 of SEQ ID NO: 1, a HCDR2 of SEQ ID NO: 2, a HCDR3 of SEQ ID NO: 3, a LCDR1 of SEQ ID NO: 16, a LCDR2 of SEQ ID NO: 17, and a LCDR3 of SEQ ID NO: 18.

Accordingly, in some embodiments, an antibody or antibody binding fragment thereof comprises V_{H}A and V_{L}A. In some embodiments, an antibody or antibody binding fragment thereof comprises V_{H}A and V_{L}B. In some embodiments, an antibody or antibody binding fragment thereof comprises V_{H}A and V_{L}C.

In some embodiments, an antibody or antibody binding fragment thereof comprises V_{H}B and V_{L}A. In some embodiments, an antibody or antibody binding fragment thereof comprises V_{H}B and V_{L}B. In some embodiments, an antibody or antibody binding fragment thereof comprises V_{H}B and V_{L}C.

In some embodiments, an antibody or antibody binding fragment thereof comprises V_{H}C and V_{L}A. In some embodiments, an antibody or antibody binding fragment thereof comprises V_{H}C and V_{L}B. In some embodiments, an antibody or antibody binding fragment thereof comprises V_{H}C and V_{L}C.

In some embodiments, an antibody or antibody binding fragment thereof comprises V_{A}D and V_{L}A. In some embodiments, an antibody or antibody binding fragment thereof comprises V_{H}D and V_{L}B. In some embodiments, an antibody or antibody binding fragment thereof comprises V_{H}D and V_{L}C.

In some embodiments, an antibody or antibody binding fragment thereof comprises V_{H}E and V_{L}A. In some embodiments, an antibody or antibody binding fragment thereof comprises V_{H}E and V_{L}B. In some embodiments, an antibody or antibody binding fragment thereof comprises V_{H}E and V_{L}C.

In some embodiments, an antibody or antibody binding fragment thereof comprises V_{H}F and V_{L}A. In some embodiments, an antibody or antibody binding fragment thereof comprises V_{H}F and V_{L}B. In some embodiments, an antibody or antibody binding fragment thereof comprises V_{H}F and V_{L}C.

In some embodiments, an antibody or antibody binding fragment thereof comprises V_{H}G and V_{L}A. In some embodiments, an antibody or antibody binding fragment thereof comprises V_{H}G and V_{L}B. In some embodiments, an antibody or antibody binding fragment thereof comprises V_{H}G and V_{L}C.

In some embodiments, an antibody or antibody binding fragment thereof comprises V_{H}H and V_{L}A. In some embodiments, an antibody or antibody binding fragment thereof comprises V_{H}H and V_{L}B. In some embodiments, an antibody or antibody binding fragment thereof comprises V_{H}H and V_{L}C.

In some embodiments, an antibody or antibody binding fragment thereof comprises V_{H}I and V_{L}A. In some embodiments, an antibody or antibody binding fragment thereof comprises V_{H}I and V_{L}B. In some embodiments, an antibody or antibody binding fragment thereof comprises V_{H}I and V_{L}C.

In some embodiments, an antibody or antibody binding fragment thereof comprises V_{H}J and V_{L}A. In some embodiments, an antibody or antibody binding fragment thereof comprises V_{H}J and V_{L}B. In some embodiments, an antibody or antibody binding fragment thereof comprises V_{H}J and V_{L}C.

In some embodiments, an antibody or antibody binding fragment thereof comprises V_{H}K and V_{L}A. In some embodiments, an antibody or antibody binding fragment thereof comprises V_{H}K and V_{L}B. In some embodiments, an antibody or antibody binding fragment thereof comprises V_{H}K and V_{L}C.

In some embodiments, an antibody or antibody binding fragment thereof comprises V_{H}M and V_{L}A. In some embodiments, an antibody or antibody binding fragment thereof comprises V_{H}M and V_{L}B. In some embodiments, an antibody or antibody binding fragment thereof comprises V_{H}M and V_{L}C.

In some embodiments, an antibody or antibody binding fragment thereof comprises V_{H}N and V_{L}A. In some embodiments, an antibody or antibody binding fragment thereof comprises V_{H}N and V_{L}B. In some embodiments, an antibody or antibody binding fragment thereof comprises V_{H}N and V_{L}C.

In some embodiments, an antibody or antibody binding fragment thereof comprises V_{H}O and V_{L}A. In some embodiments, an antibody or antibody binding fragment thereof comprises V_{H}O and V_{L}B. In some embodiments, an antibody or antibody binding fragment thereof comprises V_{H}O and V_{L}C.

In some embodiments, the light chain variable region LCDR1 is replaced with any of the other light chain LCDR1 sequences. In some embodiments, the light chain variable region LCDR2 is replaced with any of the other light chain LCDR2 sequences. In some embodiments, the light chain variable region LCDR3 is replaced with any of the other light chain LCDR3 sequences. In some embodiments, the heavy chain variable region HCDR1 is replaced with any of the other heavy chain HCDR1 sequences. In some embodiments, the heavy chain variable region HCDR2 is replaced with any of the other heavy chain HCDR2 sequences. In some embodiments, the heavy chain variable region HCDR3 is replaced with any of the other heavy chain HCDR3 sequences.

In some embodiments, the antibody comprises a heavy chain variable region peptide having one of the following sequences, or a variant thereof (Table 6):

TABLE 6
heavy chain variable regions

| | |
|---|---|
| SEQ ID NO: 4 | QIQLVQSGSELKKPGASVKVSCKASGYTFTDYAMNWVRQAPGQGL |
| | EWMGWINTQTGKPTYAQGFTGRFVFSLDTSVSTAYLQISSLKAEDT |
| | AVYYCTRLGTGNTKGFAYWGQGTLVTVSS |
| SEQ ID NO: 5 | QIQLVQSGAEVKKPGSSVKVSCKASGYTFTDYAMNWVRQAPGQGL |
| | EWMGWINTQTGKPTYAQKFQGRVTFTLDESTSTAYMELSSLRSEDT |
| | AVYYCTRLGTGNTKGFAYWGQGTLVTVSS |
| SEQ ID NO: 6 | QIQLVQSGAEVKKPGSSVKVSCKASGYTFTDYAMNWVRQAPGQGL |
| | EWMGWINTQTGKPTYAQKFQGRVTFTLDESTSTTYMELSSLRSEDT |
| | AVYYCARLGTGNTKGFAYWGQGTLVTVSS |
| SEQ ID NO: 7 | QIQLVQSGAEVKKPGSSVKVSCKASGYTFTDYAMNWVRQAPGQGL |
| | EWMGWINTQTGKPTYAQKFQGRVTFTLDESTSTTYMELSSLRSEDT |
| | AVYYCTRLGTGNTKGFAYWGQGTLVTVSS |
| SEQ ID NO: 8 | QIQLVQSGPEVKKPGESVKISCKASGYTFTDYAMNWVKQAPGNGL |
| | KWMGWINTQTGKPTYADDFKQRFVFSLETSARTTYLQINNLNIEDT |
| | ATYFCTRLGTGNTKGFAYWGQGTLVTVSS |
| SEQ ID NO: 9 | QIQLVQSGSELKKPGASVKISCKASGYTFTDYAMNWVKQAPGQGL |
| | EWMGWINTQTGKPTYAQGFTGRFVFSLDTSVSTAYLQISSLKAEDT |
| | AVYFCTRLGTGNTKGFAYWGQGTLVTVSS |
| SEQ ID NO: 10 | QIQLVQSGSELKKPGASVKISCKASGYTFTDYAMNWVKQAPGQGL |
| | EWMGWINTQTGKPTYAQGFTGRFVFSLDTSVSTAYLQISSLKAEDT |
| | AVYFCTRLGTGNTKGFAYWGQGTLVTVSS |
| SEQ ID NO: 11 | QIQLVQSGSELKKPGASVKISCKASGYTFTDYAMNWVRQAPGQGL |
| | EWMGWINTQTGKPTYAQGFTGRFVFSLDTSVRTAYLQISSLKAEDT |
| | AVYFCTRLGTGNTKGFAYWGQGTLVTVSS |
| SEQ ID NO: 12 | QIQLVQSGSELKKPGASVKISCKASGYTFTDYAMNWVRQAPGQGL |
| | EWMGWINTQTGKPTYAQGFTGRFVFSLDTSVSTAYLQISSLKAEDT |
| | AVYFCTRLGTGNTKGFAYWGQGTLVTVSS |
| SEQ ID NO: 13 | QIQLVQSGSELKKPGASVKISCKASGYTFTDYAMNWVRQAPGQGL |
| | EWMGWINTQTGKPTYAQGFTGRFVFSLDTSVSTAYLQISSLKAEDT |
| | AVYYCTRLGTGNTKGFAYWGQGTLVTVSS |
| SEQ ID NO: 14 | QIQLVQSGSELKKPGASVKVSCKASGYTFTDYAMNWVRQAPGQGL |
| | EWMGWINTQTGKPTYAQGFTGRFVFSLDTSVSTAYLQISSLKAEDT |
| | AVYFCTRLGTGNTKGFAYWGQGTLVTVSS |
| SEQ ID NO: 15 | QVQLVQSGAEVKKPGSSVKVSCKASGYTFTDYAMNWVRQAPGQG |
| | LEWMGWINTQTGKPTYAQKFQGRVTFTLDESTSTAYMELSSLRSED |
| | TAVYYCTRLGTGNTKGFAYWGQGTLVTVSS |
| SEQ ID NO: 30 | QVQLVQSGAEVKKPGASVKVSCKASGYTFTSYGISWVRQAPGQGL |
| | EWMGWISAYNANTNYAQKLQGRVTMTTDTSTSTAYMELRSLRSD |
| | DTAAYYCARVTGITGTTIGPWGQGTMVTVSS |
| SEQ ID NO: 31 | QVQLVQSGAEVKKPGASVKVSCKASGYTFTSYGISWVRQAPGQGL |
| | EWMGWISAYNANVNYAQKLQGRVTMTTDTSTSTAYMELRSLRSD |
| | DTAAYYCARVTGITGTTIGPWGQGTMVTVSS |
| SEQ ID NO: 32 | QVQLVQSGAEVKKPGASVKVSCKASGYTFTSYGISWVRQAPGQGL |
| | EWMGWISAYNGNTNYAQKLQGRVTMTTDTSTSTAYMELRSLRSD |
| | DTAAYYCARVTGITGTTIDPWGQGTMVTVSS |
| SEQ ID NO: 33 | QVQLVQSGAEVKKPGASVKVSCKASGYTFTSYGISWVRQAPGQGL |
| | EWMGWISAYNSNTNYAQKLQGRVTMTTDTSTSTAYMELRSLRSDD |
| | TAAYYCARVTGITGTTIGPWGQGTMVTVSS |
| SEQ ID NO: 44 | EVQLLESGGGLVQPGGSLRLSCAASGFSFSSSYYMCWVRQAPGKGL |
| | EWVSAIGTTRGSTYYADSVKGRFTISRDSSKNTVYLQMNSLRAEDT |
| | AVYYCARDATGYRINTIGLYFNLWGQGTLVTVSS |
| SEQ ID NO: 45 | EVQLLESGGGLVQPGGSLRLSCAASGFSFSSSYYMAWVRQAPGKGL |
| | EWVSAIGTTRGSTYYADSVKGRFTISRDNSKNTLYLQMNSLRAEDT |
| | AVYYCARDATGYRINTIGLYFNLWGQGTLVTVSS |
| SEQ ID NO: 46 | EVQLLESGGGLVQPGGSLRLSCAASGFSFSSSYYMAWVRQAPGKGL |
| | EWVSAIGTTRGSTYYADSVKGRFTISRDNSKNTVYLQMNSLRAEDT |
| | AVYYCARDATGYRINTIGLYFNLWGQGTLVTVSS |
| SEQ ID NO: 47 | EVQLLESGGGLVQPGGSLRLSCAASGFSFSSSYYMAWVRQAPGKGL |
| | EWVSAIGTTRGSTYYADSVKGRFTISRDSSKNTLYLQMNSLRAEDT |
| | AVYYCARDATGYRINTIGLYFNLWGQGTLVTVSS |
| SEQ ID NO: 48 | EVQLLESGGGLVQPGGSLRLSCAASGFSFSSSYYMAWVRQAPGKGL |
| | EWVSAIGTTRGSTYYADSVKGRFTISRDSSKNTVYLQMNSLRAEDT |
| | AVYFCARDATGYRINTIGLYFNLWGQGTLVTVSS |
| SEQ ID NO: 49 | EVQLLESGGGLVQPGGSLRLSCAASGFSFSSSYYMCWVRQAPGKGL |
| | EWVSCIGTTRGSTYYADSVKGRFTISRDNSKNTLYLQMNSLRAEDT |
| | AVYYCARDATGYRINTIGLYFNLWGQGTLVTVSS |
| SEQ ID NO: 50 | EVQLLESGGGLVQPGGSLRLSCAASGFSFSSSYYMCWVRQAPGKGL |
| | EWVSCIGTTRGSTYYADSVKGRFTISRDNSKNTVYLQMNSLRAEDT |
| | AVYYCARDATGYRINTIGLYFNLWGQGTLVTVSS |
| SEQ ID NO: 51 | EVQLLESGGGLVQPGGSLRLSCAASGFSFSSSYYMCWVRQAPGKGL |
| | EWVSCIGTTRGSTYYADSVKGRFTISRDSSKNTLYLQMNSLRAEDT |
| | AVYYCARDATGYRINTIGLYFNLWGQGTLVTVSS |
| SEQ ID NO: 52 | EVQLLESGGGLVQPGGSLRLSCAASGFSFSSSYYMCWVRQAPGKGL |
| | EWVSCIGTTRGSTYYADSVKGRFTISRDSSKNTVYLQMNSLRAEDT |
| | AVYFCARDATGYRINTIGLYFNLWGQGTLVTVSS |
| SEQ ID NO: 53 | EVQLLESGGGLVQPGGSLRLSCAASGFSFSSSYYMSWVRQAPGKGL |
| | EWVSAIGTTRGSTYYADSVKGRFTISRDNSKNTLYLQMNSLRAEDT |
| | AVYYCARDATGYRINTIGLYFNLWGQGTLVTVSS |
| SEQ ID NO: 54 | EVQLLESGGGLVQPGGSLRLSCAASGFSFSSSYYMSWVRQAPGKGL |
| | EWVSAIGTTRGSTYYADSVKGRFTISRDNSKNTVYLQMNSLRAEDT |
| | AVYYCARDATGYRINTIGLYFNLWGQGTLVTVSS |
| SEQ ID NO: 55 | EVQLLESGGGLVQPGGSLRLSCAASGFSFSSSYYMSWVRQAPGKGL |
| | EWVSAIGTTRGSTYYADSVKGRFTISRDSSKNTLYLQMNSLRAEDT |
| | AVYYCARDATGYRINTIGLYFNLWGQGTLVTVSS |
| SEQ ID NO: 56 | EVQLLESGGGLVQPGGSLRLSCAASGFSFSSSYYMSWVRQAPGKGL |
| | EWVSAIGTTRGSTYYADSVKGRFTISRDSSKNTVYLQMNSLRAEDT |
| | AVYFCARDATGYRINTIGLYFNLWGQGTLVTVSS |
| SEQ ID NO: 57 | QSLEESGGDLVKPGASLTLTCRVSGFSFSSSYYMCWVRQAPGKGLE |
| | WIACIGTTRGSTYYATWAKGRFTISKISSTTVTLQMTSLTDADTATY |
| | FCARDATGYRINTIGLYFNLWGPGTLVTVSS |

In some embodiments, the antibody comprises a light chain variable region peptide having one of the following sequences, or a variant thereof (Table 7):

TABLE 7
light chain variable regions

| | |
|---|---|
| SEQ ID NO: 19 | DIQITQSPSSLSASVGDRVTITCKSSQSLLYSENNQDYLAWYQQKPG |
| | KVPKLLIYGASNRHTGVPSRFSGSGSGTDFTLTISSLQPEDVATYYCE |
| | QTYRYPFTFGQGTKLEIK |
| SEQ ID NO: 20 | DILINQSPASLTVSAGERVTMSCKSSQSLLYSENNQDYLAWYQQKP |
| | GQFPKLLIYGASNRHTGVPDRFTGSGSGTDFTLTISSVQAEDLADYY |
| | CEQTYRYPFTFGSGTKLEIK |
| SEQ ID NO: 21 | DIQITQSPSSLSASVGDRVTITCKSSQSLLYSENNQDYLAWYQQKPG |
| | KVPKLLIYGASNRHTGVPSRFSGSGSGTDFTLTISSLQPEDVATYYCE |
| | QTYRYPFTFGQGTKLEIK |
| SEQ ID NO: 22 | DIQMTQSPSSLSASVGDRVTITCKSSQSLLYSENNQDYLAWYQQKP |
| | GKVPKLLIYGASNRHTGVPSRFSGSGSGTDFTLTISSLQPEDVATYY |
| | CEQTYRYPFTFGQGTKLEIK |
| SEQ ID NO: 37 | DIQMTQSPSSLSASVGDRVTITCRASQSISSYLNWYQQKPGKAPKLLI |
| | YDASSLESGVPSRFSGSGSGTEFTLTISSLQPDDFAVYYCQQYNNWP |
| | QTFGQGTKVEIK |
| SEQ ID NO: 61 | DIVMTQSPDSLAVSLGERATINCQASQTIYSYLSWYQQKPGQPPKLL |
| | IYEASKLASGVPDRFSGSGSGTDYTLTISSLQAEDVAVYYCQSYHGT |
| | ASTEYNTFGGGTKVEIK |
| SEQ ID NO: 62 | ELTQTPSSVEAAVGGTPTIKCQASQTIYSYLSWYQQKPGQPPKLLIY |
| | EASKLASGVPSRFSGSGSGTDYTLTISDLECADAATYYCQSYHGTAS |
| | TEYNTFGGGTEVVVK |

In some embodiments, a heavy chain variable region and/or a light chain variable region is described as comprising an amino acid sequence having at least 90%, 91%, 92%, 93%, 94%, 95%, 96%, 97%, 98%, or 99% identity, or is identical, to a recited sequence identifier, provided that heavy chain variable region and/or a light chain variable region, as context dictates, comprises a specific set of HCDRs and/or LCDRs. As used herein, the clause “provided that heavy chain variable region and/or a light chain variable region comprises a specific set of HCDRs and/or LCDRs” or similar language used herein, means that the recited HCDRs or the LCDRs, as context dictates, are identical to the sequences referenced by the sequence identifiers, while the overall percent identity of the heavy chain variable region and/or the light chain variable region is at least 90%, 91%, 92%, 93%, 94%, 95%, 96%, 97%, 98%, or 99% identical to the referenced sequence.

In some embodiments, an antibody, or antigen-binding fragment thereof, comprises a V_{H} peptide selected from one or more of SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 30, SEQ ID NO: 31, SEQ ID NO: 32, SEQ ID NO: 33, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, or any variants thereof.

In some embodiments, an antibody, or antigen-binding fragment thereof, comprises a V_{H} peptide having at least 85% identity to SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 30, SEQ ID NO: 31, SEQ ID NO: 32, SEQ ID NO: 33, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, or any variants thereof.

In some embodiments, an antibody, or antigen-binding fragment thereof, comprises a V_{H} peptide having at least 85%, 86%, 87%, 88%, 89%, 90%, 91%, 92%, 93%, 94%, 95%, 96%, 97%, 98%, or 99% identity to SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 30, SEQ ID NO: 31, SEQ ID NO: 32, SEQ ID NO: 33, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, or any variants thereof.

In some embodiments, an antibody, or antigen-binding fragment thereof, comprises a V_{H} peptide having at least 85%, 86%, 87%, 88%, 89%, 90%, 91%, 92%, 93%, 94%, 95%, 96%, 97%, 98%, or 99% identity, or is identical, to SEQ ID NO: 4. In some embodiments, an antibody, or antigen-binding fragment thereof, comprises a V_{H} peptide having at least 85%, 86%, 87%, 88%, 89%, 90%, 91%, 92%, 93%, 94%, 95%, 96%, 97%, 98%, or 99% identity, or is identical, to SEQ ID NO: 4, provided that the antibody or antigen-binding fragment thereof comprises a HCDR1 sequence of SEQ ID NO: 1, a HCDR2 sequence of SEQ ID NO: 2, and a HCDR3 sequence of SEQ ID NO: 3.

In some embodiments, an antibody, or antigen-binding fragment thereof, comprises a V_{H} peptide having at least 85%, 86%, 87%, 88%, 89%, 90%, 91%, 92%, 93%, 94%, 95%, 96%, 97%, 98%, or 99% identity, or is identical, to SEQ ID NO: 5. In some embodiments, an antibody, or antigen-binding fragment thereof, comprises a V_{H} peptide having at least 85%, 86%, 87%, 88%, 89%, 90%, 91%, 92%, 93%, 94%, 95%, 96%, 97%, 98%, or 99% identity, or is identical, to SEQ ID NO: 5, provided that the antibody or antigen-binding fragment thereof comprises a HCDR1 sequence of SEQ ID NO: 1, a HCDR2 sequence of SEQ ID NO: 2, and a HCDR3 sequence of SEQ ID NO: 3.

In some embodiments, an antibody, or antigen-binding fragment thereof, comprises a V_{H} peptide having at least 85%, 86%, 87%, 88%, 89%, 90%, 91%, 92%, 93%, 94%, 95%, 96%, 97%, 98%, or 99% identity, or is identical, to SEQ ID NO: 6. In some embodiments, an antibody, or antigen-binding fragment thereof, comprises a V_{H} peptide having at least 85%, 86%, 87%, 88%, 89%, 90%, 91%, 92%, 93%, 94%, 95%, 96%, 97%, 98%, or 99% identity, or is identical, to SEQ ID NO: 6, provided that the antibody or antigen-binding fragment thereof comprises a HCDR1 sequence of SEQ ID NO: 1, a HCDR2 sequence of SEQ ID NO: 2, and a HCDR3 sequence of SEQ ID NO: 3.

In some embodiments, an antibody, or antigen-binding fragment thereof, comprises a V_{H} peptide having at least 85%, 86%, 87%, 88%, 89%, 90%, 91%, 92%, 93%, 94%, 95%, 96%, 97%, 98%, or 99% identity, or is identical, to SEQ ID NO: 7. In some embodiments, an antibody, or antigen-binding fragment thereof, comprises a V_{H} peptide having at least 85%, 86%, 87%, 88%, 89%, 90%, 91%, 92%, 93%, 94%, 95%, 96%, 97%, 98%, or 99% identity, or is identical, to SEQ ID NO: 7, provided that the antibody or antigen-binding fragment thereof comprises a HCDR1 sequence of SEQ ID NO: 1, a HCDR2 sequence of SEQ ID NO: 2, and a HCDR3 sequence of SEQ ID NO: 3.

In some embodiments, an antibody, or antigen-binding fragment thereof, comprises a V_{H} peptide having at least 85%, 86%, 87%, 88%, 89%, 90%, 91%, 92%, 93%, 94%, 95%, 96%, 97%, 98%, or 99% identity, or is identical, to SEQ ID NO: 8. In some embodiments, an antibody, or antigen-binding fragment thereof, comprises a V_{H} peptide having at least 85%, 86%, 87%, 88%, 89%, 90%, 91%, 92%, 93%, 94%, 95%, 96%, 97%, 98%, or 99% identity, or is identical, to SEQ ID NO: 8, provided that the antibody or antigen-binding fragment thereof comprises a HCDR1 sequence of SEQ ID NO: 1, a HCDR2 sequence of SEQ ID NO: 2, and a HCDR3 sequence of SEQ ID NO: 3.

In some embodiments, an antibody, or antigen-binding fragment thereof, comprises a V_{H} peptide having at least 85%, 86%, 87%, 88%, 89%, 90%, 91%, 92%, 93%, 94%, 95%, 96%, 97%, 98%, or 99% identity, or is identical, to SEQ ID NO: 9. In some embodiments, an antibody, or antigen-binding fragment thereof, comprises a V_{H} peptide having at least 85%, 86%, 87%, 88%, 89%, 90%, 91%, 92%, 93%, 94%, 95%, 96%, 97%, 98%, or 99% identity, or is identical, to SEQ ID NO: 9, provided that the antibody or antigen-binding fragment thereof comprises a HCDR1 sequence of SEQ ID NO: 1, a HCDR2 sequence of SEQ ID NO: 2, and a HCDR3 sequence of SEQ ID NO: 3.

In some embodiments, an antibody, or antigen-binding fragment thereof, comprises a V_{H} peptide having at least 85%, 86%, 87%, 88%, 89%, 90%, 91%, 92%, 93%, 94%, 95%, 96%, 97%, 98%, or 99% identity, or is identical, to SEQ ID NO: 10. In some embodiments, an antibody, or antigen-binding fragment thereof, comprises a V_{H} peptide having at least 85%, 86%, 87%, 88%, 89%, 90%, 91%, 92%, 93%, 94%, 95%, 96%, 97%, 98%, or 99% identity, or is identical, to SEQ ID NO: 10, provided that the antibody or antigen-binding fragment thereof comprises a HCDR1 sequence of SEQ ID NO: 1, a HCDR2 sequence of SEQ ID NO: 2, and a HCDR3 sequence of SEQ ID NO: 3.

In some embodiments, an antibody, or antigen-binding fragment thereof, comprises a V_{H} peptide having at least 85%, 86%, 87%, 88%, 89%, 90%, 91%, 92%, 93%, 94%, 95%, 96%, 97%, 98%, or 99% identity, or is identical, to SEQ ID NO: 11. In some embodiments, an antibody, or antigen-binding fragment thereof, comprises a V_{H} peptide having at least 85%, 86%, 87%, 88%, 89%, 90%, 91%, 92%, 93%, 94%, 95%, 96%, 97%, 98%, or 99% identity, or is identical, to SEQ ID NO: 11, provided that the antibody or antigen-binding fragment thereof comprises a HCDR1 sequence of SEQ ID NO: 1, a HCDR2 sequence of SEQ ID NO: 2, and a HCDR3 sequence of SEQ ID NO: 3.

In some embodiments, an antibody, or antigen-binding fragment thereof, comprises a V_{H} peptide having at least 85%, 86%, 87%, 88%, 89%, 90%, 91%, 92%, 93%, 94%, 95%, 96%, 97%, 98%, or 99% identity, or is identical, to SEQ ID NO: 12. In some embodiments, an antibody, or antigen-binding fragment thereof, comprises a V_{H} peptide having at least 85%, 86%, 87%, 88%, 89%, 90%, 91%, 92%, 93%, 94%, 95%, 96%, 97%, 98%, or 99% identity, or is identical, to SEQ ID NO: 12, provided that the antibody or antigen-binding fragment thereof comprises a HCDR1 sequence of SEQ ID NO: 1, a HCDR2 sequence of SEQ ID NO: 2, and a HCDR3 sequence of SEQ ID NO: 3.

In some embodiments, an antibody, or antigen-binding fragment thereof, comprises a V_{H} peptide having at least 85%, 86%, 87%, 88%, 89%, 90%, 91%, 92%, 93%, 94%, 95%, 96%, 97%, 98%, or 99% identity, or is identical, to SEQ ID NO: 13. In some embodiments, an antibody, or antigen-binding fragment thereof, comprises a V_{H} peptide having at least 85%, 86%, 87%, 88%, 89%, 90%, 91%, 92%, 93%, 94%, 95%, 96%, 97%, 98%, or 99% identity, or is identical, to SEQ ID NO: 13, provided that the antibody or antigen-binding fragment thereof comprises a HCDR1 sequence of SEQ ID NO: 1, a HCDR2 sequence of SEQ ID NO: 2, and a HCDR3 sequence of SEQ ID NO: 3.

In some embodiments, an antibody, or antigen-binding fragment thereof, comprises a V_{H} peptide having at least 85%, 86%, 87%, 88%, 89%, 90%, 91%, 92%, 93%, 94%, 95%, 96%, 97%, 98%, or 99% identity, or is identical, to SEQ ID NO: 14. In some embodiments, an antibody, or antigen-binding fragment thereof, comprises a V_{H} peptide having at least 85%, 86%, 87%, 88%, 89%, 90%, 91%, 92%, 93%, 94%, 95%, 96%, 97%, 98%, or 99% identity, or is identical, to SEQ ID NO: 14, provided that the antibody or antigen-binding fragment thereof comprises a HCDR1 sequence of SEQ ID NO: 1, a HCDR2 sequence of SEQ ID NO: 2, and a HCDR3 sequence of SEQ ID NO: 3.

In some embodiments, an antibody, or antigen-binding fragment thereof, comprises a V_{H} peptide having at least 85%, 86%, 87%, 88%, 89%, 90%, 91%, 92%, 93%, 94%, 95%, 96%, 97%, 98%, or 99% identity, or is identical, to SEQ ID NO: 15. In some embodiments, an antibody, or antigen-binding fragment thereof, comprises a V_{H} peptide having at least 85%, 86%, 87%, 88%, 89%, 90%, 91%, 92%, 93%, 94%, 95%, 96%, 97%, 98%, or 99% identity, or is identical, to SEQ ID NO: 15, provided that the antibody or antigen-binding fragment thereof comprises a HCDR1 sequence of SEQ ID NO: 1, a HCDR2 sequence of SEQ ID NO: 2, and a HCDR3 sequence of SEQ ID NO: 3.

In some embodiments, an antibody, or antigen-binding fragment thereof, comprises a V_{H} peptide having at least 85%, 86%, 87%, 88%, 89%, 90%, 91%, 92%, 93%, 94%, 95%, 96%, 97%, 98%, or 99% identity, or is identical, to SEQ ID NO: 30. In some embodiments, an antibody, or antigen-binding fragment thereof, comprises a V_{H} peptide having at least 85%, 86%, 87%, 88%, 89%, 90%, 91%, 92%, 93%, 94%, 95%, 96%, 97%, 98%, or 99% identity, or is identical, to SEQ ID NO: 30, provided that the antibody or antigen-binding fragment thereof comprises a HCDR1 sequence of SEQ ID NO: 23, a HCDR2 sequence of SEQ ID NO: 24, SEQ ID NO: 25, SEQ ID NO: 26, or SEQ ID NO: 27, and a HCDR3 sequence of SEQ ID NO: 28 or SEQ ID NO: 29.

In some embodiments, an antibody, or antigen-binding fragment thereof, comprises a V_{H} peptide having at least 85%, 86%, 87%, 88%, 89%, 90%, 91%, 92%, 93%, 94%, 95%, 96%, 97%, 98%, or 99% identity, or is identical, to SEQ ID NO: 31. In some embodiments, an antibody, or antigen-binding fragment thereof, comprises a V_{H} peptide having at least 85%, 86%, 87%, 88%, 89%, 90%, 91%, 92%, 93%, 94%, 95%, 96%, 97%, 98%, or 99% identity, or is identical, to SEQ ID NO: 31, provided that the antibody or antigen-binding fragment thereof comprises a HCDR1 sequence of SEQ ID NO: 23, a HCDR2 sequence of SEQ ID NO: 24, SEQ ID NO: 25, SEQ ID NO: 26, or SEQ ID NO: 27, and a HCDR3 sequence of SEQ ID NO: 28 or SEQ ID NO: 29.

In some embodiments, an antibody, or antigen-binding fragment thereof, comprises a V_{H} peptide having at least 85%, 86%, 87%, 88%, 89%, 90%, 91%, 92%, 93%, 94%, 95%, 96%, 97%, 98%, or 99% identity, or is identical, to SEQ ID NO: 32. In some embodiments, an antibody, or antigen-binding fragment thereof, comprises a V_{H} peptide having at least 85%, 86%, 87%, 88%, 89%, 90%, 91%, 92%, 93%, 94%, 95%, 96%, 97%, 98%, or 99% identity, or is identical, to SEQ ID NO: 32, provided that the antibody or antigen-binding fragment thereof comprises a HCDR1 sequence of SEQ ID NO: 23, a HCDR2 sequence of SEQ ID NO: 24, SEQ ID NO: 25, SEQ ID NO: 26, or SEQ ID NO: 27, and a HCDR3 sequence of SEQ ID NO: 28 or SEQ ID NO: 29.

In some embodiments, an antibody, or antigen-binding fragment thereof, comprises a V_{H} peptide having at least 85%, 86%, 87%, 88%, 89%, 90%, 91%, 92%, 93%, 94%, 95%, 96%, 97%, 98%, or 99% identity, or is identical, to SEQ ID NO: 33. In some embodiments, an antibody, or antigen-binding fragment thereof, comprises a V_{H} peptide having at least 85%, 86%, 87%, 88%, 89%, 90%, 91%, 92%, 93%, 94%, 95%, 96%, 97%, 98%, or 99% identity, or is identical, to SEQ ID NO: 33, provided that the antibody or antigen-binding fragment thereof comprises a HCDR1 sequence of SEQ ID NO: 23, a HCDR2 sequence of SEQ ID NO: 24, SEQ ID NO: 25, SEQ ID NO: 26, or SEQ ID NO: 27, and a HCDR3 sequence of SEQ ID NO: 28 or SEQ ID NO: 29.

In some embodiments, an antibody, or antigen-binding fragment thereof, comprises a V_{H} peptide having at least 85%, 86%, 87%, 88%, 89%, 90%, 91%, 92%, 93%, 94%, 95%, 96%, 97%, 98%, or 99% identity, or is identical, to SEQ ID NO: 44. In some embodiments, an antibody, or antigen-binding fragment thereof, comprises a V_{H} peptide having at least 85%, 86%, 87%, 88%, 89%, 90%, 91%, 92%, 93%, 94%, 95%, 96%, 97%, 98%, or 99% identity, or is identical, to SEQ ID NO: 44, provided that the antibody or antigen-binding fragment thereof comprises a HCDR1 sequence of SEQ ID NO: 38, SEQ ID NO: 39, or SEQ ID NO: 40, a HCDR2 sequence of SEQ ID NO: 41, or SEQ ID NO: 42, and a HCDR3 sequence of SEQ ID NO: 43.

In some embodiments, an antibody, or antigen-binding fragment thereof, comprises a V_{H} peptide having at least 85%, 86%, 87%, 88%, 89%, 90%, 91%, 92%, 93%, 94%, 95%, 96%, 97%, 98%, or 99% identity, or is identical, to SEQ ID NO: 45. In some embodiments, an antibody, or antigen-binding fragment thereof, comprises a V_{H} peptide having at least 85%, 86%, 87%, 88%, 89%, 90%, 91%, 92%, 93%, 94%, 95%, 96%, 97%, 98%, or 99% identity, or is identical, to SEQ ID NO: 45, provided that the antibody or antigen-binding fragment thereof comprises a HCDR1 sequence of SEQ ID NO: 38, SEQ ID NO: 39, or SEQ ID NO: 40, a HCDR2 sequence of SEQ ID NO: 41, or SEQ ID NO: 42, and a HCDR3 sequence of SEQ ID NO: 43.

In some embodiments, an antibody, or antigen-binding fragment thereof, comprises a V_{H} peptide having at least 85%, 86%, 87%, 88%, 89%, 90%, 91%, 92%, 93%, 94%, 95%, 96%, 97%, 98%, or 99% identity, or is identical, to SEQ ID NO: 46. In some embodiments, an antibody, or antigen-binding fragment thereof, comprises a V_{H} peptide having at least 85%, 86%, 87%, 88%, 89%, 90%, 91%, 92%, 93%, 94%, 95%, 96%, 97%, 98%, or 99% identity, or is identical, to SEQ ID NO: 46, provided that the antibody or antigen-binding fragment thereof comprises a HCDR1 sequence of SEQ ID NO: 38, SEQ ID NO: 39, or SEQ ID NO: 40, a HCDR2 sequence of SEQ ID NO: 41, or SEQ ID NO: 42, and a HCDR3 sequence of SEQ ID NO: 43.

In some embodiments, an antibody, or antigen-binding fragment thereof, comprises a V_{H} peptide having at least 85%, 86%, 87%, 88%, 89%, 90%, 91%, 92%, 93%, 94%, 95%, 96%, 97%, 98%, or 99% identity, or is identical, to SEQ ID NO: 47. In some embodiments, an antibody, or antigen-binding fragment thereof, comprises a V_{H} peptide having at least 85%, 86%, 87%, 88%, 89%, 90%, 91%, 92%, 93%, 94%, 95%, 96%, 97%, 98%, or 99% identity, or is identical, to SEQ ID NO: 47, provided that the antibody or antigen-binding fragment thereof comprises a HCDR1 sequence of SEQ ID NO: 38, SEQ ID NO: 39, or SEQ ID NO: 40, a HCDR2 sequence of SEQ ID NO: 41, or SEQ ID NO: 42, and a HCDR3 sequence of SEQ ID NO: 43.

In some embodiments, an antibody, or antigen-binding fragment thereof, comprises a V_{H} peptide having at least 85%, 86%, 87%, 88%, 89%, 90%, 91%, 92%, 93%, 94%, 95%, 96%, 97%, 98%, or 99% identity, or is identical, to SEQ ID NO: 48. In some embodiments, an antibody, or antigen-binding fragment thereof, comprises a V_{H} peptide having at least 85%, 86%, 87%, 88%, 89%, 90%, 91%, 92%, 93%, 94%, 95%, 96%, 97%, 98%, or 99% identity, or is identical, to SEQ ID NO: 48, provided that the antibody or antigen-binding fragment thereof comprises a HCDR1 sequence of SEQ ID NO: 38, SEQ ID NO: 39, or SEQ ID NO: 40, a HCDR2 sequence of SEQ ID NO: 41, or SEQ ID NO: 42, and a HCDR3 sequence of SEQ ID NO: 43.

In some embodiments, an antibody, or antigen-binding fragment thereof, comprises a V_{H} peptide having at least 85%, 86%, 87%, 88%, 89%, 90%, 91%, 92%, 93%, 94%, 95%, 96%, 97%, 98%, or 99% identity, or is identical, to SEQ ID NO: 49. In some embodiments, an antibody, or antigen-binding fragment thereof, comprises a V_{H} peptide having at least 85%, 86%, 87%, 88%, 89%, 90%, 91%, 92%, 93%, 94%, 95%, 96%, 97%, 98%, or 99% identity, or is identical, to SEQ ID NO: 49, provided that the antibody or antigen-binding fragment thereof comprises a HCDR1 sequence of SEQ ID NO: 38, SEQ ID NO: 39, or SEQ ID NO: 40, a HCDR2 sequence of SEQ ID NO: 41, or SEQ ID NO: 42, and a HCDR3 sequence of SEQ ID NO: 43.

In some embodiments, an antibody, or antigen-binding fragment thereof, comprises a V_{H} peptide having at least 85%, 86%, 87%, 88%, 89%, 90%, 91%, 92%, 93%, 94%, 95%, 96%, 97%, 98%, or 99% identity, or is identical, to SEQ ID NO: 50. In some embodiments, an antibody, or antigen-binding fragment thereof, comprises a V_{H} peptide having at least 85%, 86%, 87%, 88%, 89%, 90%, 91%, 92%, 93%, 94%, 95%, 96%, 97%, 98%, or 99% identity, or is identical, to SEQ ID NO: 50, provided that the antibody or antigen-binding fragment thereof comprises a HCDR1 sequence of SEQ ID NO: 38, SEQ ID NO: 39, or SEQ ID NO: 40, a HCDR2 sequence of SEQ ID NO: 41, or SEQ ID NO: 42, and a HCDR3 sequence of SEQ ID NO: 43.

In some embodiments, an antibody, or antigen-binding fragment thereof, comprises a V_{H} peptide having at least 85%, 86%, 87%, 88%, 89%, 90%, 91%, 92%, 93%, 94%, 95%, 96%, 97%, 98%, or 99% identity, or is identical, to SEQ ID NO: 51. In some embodiments, an antibody, or antigen-binding fragment thereof, comprises a V_{H} peptide having at least 85%, 86%, 87%, 88%, 89%, 90%, 91%, 92%, 93%, 94%, 95%, 96%, 97%, 98%, or 99% identity, or is identical, to SEQ ID NO: 51, provided that the antibody or antigen-binding fragment thereof comprises a HCDR1 sequence of SEQ ID NO: 38, SEQ ID NO: 39, or SEQ ID NO: 40, a HCDR2 sequence of SEQ ID NO: 41, or SEQ ID NO: 42, and a HCDR3 sequence of SEQ ID NO: 43.

In some embodiments, an antibody, or antigen-binding fragment thereof, comprises a V_{H} peptide having at least 85%, 86%, 87%, 88%, 89%, 90%, 91%, 92%, 93%, 94%, 95%, 96%, 97%, 98%, or 99% identity, or is identical, to SEQ ID NO: 52. In some embodiments, an antibody, or antigen-binding fragment thereof, comprises a V_{H} peptide having at least 85%, 86%, 87%, 88%, 89%, 90%, 91%, 92%, 93%, 94%, 95%, 96%, 97%, 98%, or 99% identity, or is identical, to SEQ ID NO: 52, provided that the antibody or antigen-binding fragment thereof comprises a HCDR1 sequence of SEQ ID NO: 38, SEQ ID NO: 39, or SEQ ID NO: 40, a HCDR2 sequence of SEQ ID NO: 41, or SEQ ID NO: 42, and a HCDR3 sequence of SEQ ID NO: 43.

In some embodiments, an antibody, or antigen-binding fragment thereof, comprises a V_{H} peptide having at least 85%, 86%, 87%, 88%, 89%, 90%, 91%, 92%, 93%, 94%, 95%, 96%, 97%, 98%, or 99% identity, or is identical, to SEQ ID NO: 53. In some embodiments, an antibody, or antigen-binding fragment thereof, comprises a V_{H} peptide having at least 85%, 86%, 87%, 88%, 89%, 90%, 91%, 92%, 93%, 94%, 95%, 96%, 97%, 98%, or 99% identity, or is identical, to SEQ ID NO: 53, provided that the antibody or antigen-binding fragment thereof comprises a HCDR1 sequence of SEQ ID NO: 38, SEQ ID NO: 39, or SEQ ID NO: 40, a HCDR2 sequence of SEQ ID NO: 41, or SEQ ID NO: 42, and a HCDR3 sequence of SEQ ID NO: 43.

In some embodiments, an antibody, or antigen-binding fragment thereof, comprises a V_{H} peptide having at least 85%, 86%, 87%, 88%, 89%, 90%, 91%, 92%, 93%, 94%, 95%, 96%, 97%, 98%, or 99% identity, or is identical, to SEQ ID NO: 54. In some embodiments, an antibody, or antigen-binding fragment thereof, comprises a V_{H} peptide having at least 85%, 86%, 87%, 88%, 89%, 90%, 91%, 92%, 93%, 94%, 95%, 96%, 97%, 98%, or 99% identity, or is identical, to SEQ ID NO: 54, provided that the antibody or antigen-binding fragment thereof comprises a HCDR1 sequence of SEQ ID NO: 38, SEQ ID NO: 39, or SEQ ID NO: 40, a HCDR2 sequence of SEQ ID NO: 41, or SEQ ID NO: 42, and a HCDR3 sequence of SEQ ID NO: 43.

In some embodiments, an antibody, or antigen-binding fragment thereof, comprises a V_{H} peptide having at least 85%, 86%, 87%, 88%, 89%, 90%, 91%, 92%, 93%, 94%, 95%, 96%, 97%, 98%, or 99% identity, or is identical, to SEQ ID NO: 55. In some embodiments, an antibody, or antigen-binding fragment thereof, comprises a V_{H} peptide having at least 85%, 86%, 87%, 88%, 89%, 90%, 91%, 92%, 93%, 94%, 95%, 96%, 97%, 98%, or 99% identity, or is identical, to SEQ ID NO: 55, provided that the antibody or antigen-binding fragment thereof comprises a HCDR1 sequence of SEQ ID NO: 38, SEQ ID NO: 39, or SEQ ID NO: 40, a HCDR2 sequence of SEQ ID NO: 41, or SEQ ID NO: 42, and a HCDR3 sequence of SEQ ID NO: 43.

In some embodiments, an antibody, or antigen-binding fragment thereof, comprises a V_{H} peptide having at least 85%, 86%, 87%, 88%, 89%, 90%, 91%, 92%, 93%, 94%, 95%, 96%, 97%, 98%, or 99% identity, or is identical, to SEQ ID NO: 56. In some embodiments, an antibody, or antigen-binding fragment thereof, comprises a V_{H} peptide having at least 85%, 86%, 87%, 88%, 89%, 90%, 91%, 92%, 93%, 94%, 95%, 96%, 97%, 98%, or 99% identity, or is identical, to SEQ ID NO: 56, provided that the antibody or antigen-binding fragment thereof comprises a HCDR1 sequence of SEQ ID NO: 38, SEQ ID NO: 39, or SEQ ID NO: 40, a HCDR2 sequence of SEQ ID NO: 41, or SEQ ID NO: 42, and a HCDR3 sequence of SEQ ID NO: 43.

In some embodiments, an antibody, or antigen-binding fragment thereof, comprises a V_{H} peptide having at least 85%, 86%, 87%, 88%, 89%, 90%, 91%, 92%, 93%, 94%, 95%, 96%, 97%, 98%, or 99% identity, or is identical, to SEQ ID NO: 57. In some embodiments, an antibody, or antigen-binding fragment thereof, comprises a V_{H} peptide having at least 85%, 86%, 87%, 88%, 89%, 90%, 91%, 92%, 93%, 94%, 95%, 96%, 97%, 98%, or 99% identity, or is identical, to SEQ ID NO: 57, provided that the antibody or antigen-binding fragment thereof comprises a HCDR1 sequence of SEQ ID NO: 38, SEQ ID NO: 39, or SEQ ID NO: 40, a HCDR2 sequence of SEQ ID NO: 41, or SEQ ID NO: 42, and a HCDR3 sequence of SEQ ID NO: 43.

In some embodiments, an antibody, or antigen-binding fragment thereof, comprises a V_{L} peptide selected from one or more of SEQ ID NO: 19, SEQ ID NO: 20, SEQ ID NO: 21, SEQ ID NO: 22, SEQ ID NO: 37, SEQ ID NO: 61, SEQ ID NO: 62, or any variants thereof.

In some embodiments, an antibody, or antigen-binding fragment thereof, comprises a V_{L} peptide having at least 85% identity to SEQ ID NO: 19, SEQ ID NO: 20, SEQ ID NO: 21, SEQ ID NO: 22, SEQ ID NO: 37, SEQ ID NO: 61, SEQ ID NO: 62, or any variants thereof.

In some embodiments, an antibody, or antigen-binding fragment thereof, comprises a V_{L} peptide having at least 85%, 86%, 87%, 88%, 89%, 90%, 91%, 92%, 93%, 94%, 95%, 96%, 97%, 98%, or 99% identity, or is identical, to SEQ ID NO: 19, SEQ ID NO: 20, SEQ ID NO: 21, SEQ ID NO: 22, SEQ ID NO: 37, SEQ ID NO: 61, SEQ ID NO: 62, or any variants thereof

In some embodiments, an antibody, or antigen-binding fragment thereof, comprises a V_{L} peptide having at least 85%, 86%, 87%, 88%, 89%, 90%, 91%, 92%, 93%, 94%, 95%, 96%, 97%, 98%, or 99% identity, or is identical, to SEQ ID NO: 19. In some embodiments, an antibody, or antigen-binding fragment thereof, comprises a V_{L} peptide having at least 85%, 86%, 87%, 88%, 89%, 90%, 91%, 92%, 93%, 94%, 95%, 96%, 97%, 98%, or 99% identity, or is identical, to SEQ ID NO: 19, provided that the antibody or antigen-binding fragment thereof comprises a LCDR1 sequence of SEQ ID NO: 16, a LCDR2 sequence of SEQ ID NO: 17, and a LCDR3 sequence of SEQ ID NO: 18.

In some embodiments, an antibody, or antigen-binding fragment thereof, comprises a V_{L} peptide having at least 85%, 86%, 87%, 88%, 89%, 90%, 91%, 92%, 93%, 94%, 95%, 96%, 97%, 98%, or 99% identity, or is identical, to SEQ ID NO: 20. In some embodiments, an antibody, or antigen-binding fragment thereof, comprises a V_{L} peptide having at least 85%, 86%, 87%, 88%, 89%, 90%, 91%, 92%, 93%, 94%, 95%, 96%, 97%, 98%, or 99% identity, or is identical, to SEQ ID NO: 20, provided that the antibody or antigen-binding fragment thereof comprises a LCDR1 sequence of SEQ ID NO: 16, a LCDR2 sequence of SEQ ID NO: 17, and a LCDR3 sequence of SEQ ID NO: 18.

In some embodiments, an antibody, or antigen-binding fragment thereof, comprises a V_{L} peptide having at least 85%, 86%, 87%, 88%, 89%, 90%, 91%, 92%, 93%, 94%, 95%, 96%, 97%, 98%, or 99% identity, or is identical, to SEQ ID NO: 21. In some embodiments, an antibody, or antigen-binding fragment thereof, comprises a V_{L} peptide having at least 85%, 86%, 87%, 88%, 89%, 90%, 91%, 92%, 93%, 94%, 95%, 96%, 97%, 98%, or 99% identity, or is identical, to SEQ ID NO: 21, provided that the antibody or antigen-binding fragment thereof comprises a LCDR1 sequence of SEQ ID NO: 16, a LCDR2 sequence of SEQ ID NO: 17, and a LCDR3 sequence of SEQ ID NO: 18.

In some embodiments, an antibody, or antigen-binding fragment thereof, comprises a V_{L} peptide having at least 85%, 86%, 87%, 88%, 89%, 90%, 91%, 92%, 93%, 94%, 95%, 96%, 97%, 98%, or 99% identity, or is identical, to SEQ ID NO: 22. In some embodiments, an antibody, or antigen-binding fragment thereof, comprises a V_{L} peptide having at least 85%, 86%, 87%, 88%, 89%, 90%, 91%, 92%, 93%, 94%, 95%, 96%, 97%, 98%, or 99% identity, or is identical, to SEQ ID NO: 22, provided that the antibody or antigen-binding fragment thereof comprises a LCDR1 sequence of SEQ ID NO: 16, a LCDR2 sequence of SEQ ID NO: 17, and a LCDR3 sequence of SEQ ID NO: 18.

In some embodiments, an antibody, or antigen-binding fragment thereof, comprises a V_{L} peptide having at least 85%, 86%, 87%, 88%, 89%, 90%, 91%, 92%, 93%, 94%, 95%, 96%, 97%, 98%, or 99% identity, or is identical, to SEQ ID NO: 37. In some embodiments, an antibody, or antigen-binding fragment thereof, comprises a V_{L} peptide having at least 85%, 86%, 87%, 88%, 89%, 90%, 91%, 92%, 93%, 94%, 95%, 96%, 97%, 98%, or 99% identity, or is identical, to SEQ ID NO: 37, provided that the antibody or antigen-binding fragment thereof comprises a LCDR1 sequence of SEQ ID NO: 34, a LCDR2 sequence of SEQ ID NO: 35, and a LCDR3 sequence of SEQ ID NO: 36.

In some embodiments, an antibody, or antigen-binding fragment thereof, comprises a V_{L} peptide having at least 85%, 86%, 87%, 88%, 89%, 90%, 91%, 92%, 93%, 94%, 95%, 96%, 97%, 98%, or 99% identity, or is identical, to SEQ ID NO: 61. In some embodiments, an antibody, or antigen-binding fragment thereof, comprises a V_{L} peptide having at least 85%, 86%, 87%, 88%, 89%, 90%, 91%, 92%, 93%, 94%, 95%, 96%, 97%, 98%, or 99% identity, or is identical, to SEQ ID NO: 61, provided that the antibody or antigen-binding fragment thereof comprises a LCDR1 sequence of SEQ ID NO: 58, a LCDR2 sequence of SEQ ID NO: 59, and a LCDR3 sequence of SEQ ID NO: 60.

In some embodiments, an antibody, or antigen-binding fragment thereof, comprises a V_{L} peptide having at least 85%, 86%, 87%, 88%, 89%, 90%, 91%, 92%, 93%, 94%, 95%, 96%, 97%, 98%, or 99% identity, or is identical, to SEQ ID NO: 62. In some embodiments, an antibody, or antigen-binding fragment thereof, comprises a V_{L} peptide having at least 85%, 86%, 87%, 88%, 89%, 90%, 91%, 92%, 93%, 94%, 95%, 96%, 97%, 98%, or 99% identity, or is identical, to SEQ ID NO: 62, provided that the antibody or antigen-binding fragment thereof comprises a LCDR1 sequence of SEQ ID NO: 58, a LCDR2 sequence of SEQ ID NO: 59, and a LCDR3 sequence of SEQ ID NO: 60.

The VH and the VL sequences can be in any format, including, but not limited to an scFv format where the VH and VL regions are linked with a peptide linker. Examples of peptide linkers that can be used to link various peptides provided for herein include but are not limited to: (GGGGS)ₙ (SEQ ID NO: 70); (GGGGA)ₙ (SEQ ID NO: 71), or any combination thereof, wherein each n is independently 1-5. In some embodiments, each n is, independently, 1. In some embodiments, each n is, independently, 2. In some embodiments, each n is, independently, 3. In some embodiments, each n is, independently, 4. In some embodiments, each n is, independently, 5. In some embodiments, the variable regions are not linked with a peptide linker.

As provided for herein, the different peptides (V_{H} or V_{L}) described herein can be linked with a peptide linker or not linked with a peptide linker and instead form a contiguous sequence. In some embodiments, the heavy chain variable region and the light chain variable region are not linked by a linker. In some embodiments, the heavy chain variable region and the light chain variable region are linked via a peptide linker. In some embodiments, the peptide linker comprises a sequence of (GGGGS)ₙ (SEQ ID NO: 70); (GGGGA)ₙ (SEQ ID NO: 71), or any combination thereof, wherein each n is independently 1-5. In some embodiments, each n is, independently, 1. In some embodiments, each n is, independently, 2. In some embodiments, each nis, independently, 3. In some embodiments, each n is, independently, 4. In some embodiments, each n is, independently, 5. The linked peptide format can be represented by a formula of V_{H}—Z-V_{L} or V_{L}-Z-V_{H}, wherein Z is the peptide linker. In some embodiments, Z is (GGGGS)ₙ (SEQ ID NO: 70); (GGGGA)ₙ (SEQ ID NO: 71), or any combination thereof, wherein each n is independently 1-5. In some embodiments, each nis, independently, 1. In some embodiments, each n is, independently, 2. In some embodiments, each n is, independently, 3. In some embodiments, each n is, independently, 4. In some embodiments, each n is, independently, 5.

In some embodiments, the V_{H} and V_{L} polypeptides are linked to a Fc region, such as “IgG1-AAS constant region”, “IgG4 S228P L235E LS” or “IgG4 S228P L235E YTE”. In some embodiments, the Fc region is as provided for herein.

In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, or, SEQ ID NO: 15; and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 19, SEQ ID NO: 20, SEQ ID NO: 21, or SEQ ID NO: 22.

In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, or, SEQ ID NO: 15; and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 19, SEQ ID NO: 20, SEQ ID NO: 21, or SEQ ID NO: 22; provided that the V_{H} peptide and a V_{L} peptide comprises at least one HCDR having a sequence of SEQ ID NO: 1, SEQ ID NO: 2, or SEQ ID NO: 3; and/or at least one LCDR having a sequence of SEQ ID NO: 16, SEQ ID NO: 17, or SEQ ID NO: 18. In some embodiments, the CDRs in the V_{H} or V_{L} chain are as set forth in the combinations provided for herein.

In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, or, SEQ ID NO: 15; and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 19, SEQ ID NO: 20, SEQ ID NO: 21, or SEQ ID NO: 22; provided that the V_{H} peptide comprises a HCDR1 having a sequence of SEQ ID NO: 1, a HCDR2 having a sequence of SEQ ID NO: 2, and a HCDR3 having a sequence of SEQ ID NO: 3; and/or the V_{L} peptide comprises a LCDR1 having a sequence of SEQ ID NO: 16, a LCDR2 having a sequence of SEQ ID NO: 17, and a LCDR3 having a sequence of SEQ ID NO: 18.

In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 4, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 19. In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 4, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 19, provided that the V_{H} peptide comprises a HCDR1 having a sequence of SEQ ID NO: 1, a HCDR2 having a sequence of SEQ ID NO: 2, and a HCDR3 having a sequence of SEQ ID NO: 3; and/or the V_{L} peptide comprises a LCDR1 having a sequence of SEQ ID NO: 16, a LCDR2 having a sequence of SEQ ID NO: 17, and a LCDR3 having a sequence of SEQ ID NO: 28. In some embodiments, an antibody, or antigen-binding fragment thereof, comprises a V_{H} peptide of SEQ ID NO: 4 and a V_{L} peptide of SEQ ID NO: 19.

In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 4, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 20. In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 4, and the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 20, provided that the V_{H} peptide comprises a HCDR1 having a sequence of SEQ ID NO: 1, a HCDR2 having a sequence of SEQ ID NO: 2, and a HCDR3 having a sequence of SEQ ID NO: 3; and/or the V_{L} peptide comprises a LCDR1 having a sequence of SEQ ID NO: 16, a LCDR2 having a sequence of SEQ ID NO: 17, and a LCDR3 having a sequence of SEQ ID NO: 28. In some embodiments, an antibody, or antigen-binding fragment thereof, comprises a V_{H} peptide of SEQ ID NO: 4 and a V_{L} peptide of SEQ ID NO: 20.

In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 4, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 21. In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 4, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 21, provided that the V_{H} peptide comprises a HCDR1 having a sequence of SEQ ID NO: 1, a HCDR2 having a sequence of SEQ ID NO: 2, and a HCDR3 having a sequence of SEQ ID NO: 3; and/or the V_{L} peptide comprises a LCDR1 having a sequence of SEQ ID NO: 16, a LCDR2 having a sequence of SEQ ID NO: 17, and a LCDR3 having a sequence of SEQ ID NO: 28. In some embodiments, an antibody, or antigen-binding fragment thereof, comprises a V_{H} peptide of SEQ ID NO: 4 and a V_{L} peptide of SEQ ID NO: 21.

In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 4, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 22. In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 4, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 22, provided that the V_{H} peptide comprises a HCDR1 having a sequence of SEQ ID NO: 1, a HCDR2 having a sequence of SEQ ID NO: 2, and a HCDR3 having a sequence of SEQ ID NO: 3; and/or the V_{L} peptide comprises a LCDR1 having a sequence of SEQ ID NO: 16, a LCDR2 having a sequence of SEQ ID NO: 17, and a LCDR3 having a sequence of SEQ ID NO: 28. In some embodiments, an antibody, or antigen-binding fragment thereof, comprises a V_{H} peptide of SEQ ID NO: 4 and a V_{L} peptide of SEQ ID NO: 22.

In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 5, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 19. In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 5, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 19, provided that the V_{H} peptide comprises a HCDR1 having a sequence of SEQ ID NO: 1, a HCDR2 having a sequence of SEQ ID NO: 2, and a HCDR3 having a sequence of SEQ ID NO: 3; and/or the V_{L} peptide comprises a LCDR1 having a sequence of SEQ ID NO: 16, a LCDR2 having a sequence of SEQ ID NO: 17, and a LCDR3 having a sequence of SEQ ID NO: 28. In some embodiments, an antibody, or antigen-binding fragment thereof, comprises a V_{H} peptide of SEQ ID NO: 5 and a V_{L} peptide of SEQ ID NO: 19.

In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 5, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 20. In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 5, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 20, provided that the V_{H} peptide comprises a HCDR1 having a sequence of SEQ ID NO: 1, a HCDR2 having a sequence of SEQ ID NO: 2, and a HCDR3 having a sequence of SEQ ID NO: 3; and/or the V_{L} peptide comprises a LCDR1 having a sequence of SEQ ID NO: 16, a LCDR2 having a sequence of SEQ ID NO: 17, and a LCDR3 having a sequence of SEQ ID NO: 28. In some embodiments, an antibody, or antigen-binding fragment thereof, comprises a V_{H} peptide of SEQ ID NO: 5 and a V_{L} peptide of SEQ ID NO: 20.

In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 5, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 21. In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 5, and the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 21, provided that the V_{H} peptide comprises a HCDR1 having a sequence of SEQ ID NO: 1, a HCDR2 having a sequence of SEQ ID NO: 2, and a HCDR3 having a sequence of SEQ ID NO: 3; and/or the V_{L} peptide comprises a LCDR1 having a sequence of SEQ ID NO: 16, a LCDR2 having a sequence of SEQ ID NO: 17, and a LCDR3 having a sequence of SEQ ID NO: 28. In some embodiments, an antibody, or antigen-binding fragment thereof, comprises a V_{H} peptide of SEQ ID NO: 5 and a V_{L} peptide of SEQ ID NO: 21.

In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 5, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 22. In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 5, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 22, provided that the V_{H} peptide comprises a HCDR1 having a sequence of SEQ ID NO: 1, a HCDR2 having a sequence of SEQ ID NO: 2, and a HCDR3 having a sequence of SEQ ID NO: 3; and/or the V_{L} peptide comprises a LCDR1 having a sequence of SEQ ID NO: 16, a LCDR2 having a sequence of SEQ ID NO: 17, and a LCDR3 having a sequence of SEQ ID NO: 28. In some embodiments, an antibody, or antigen-binding fragment thereof, comprises a V_{H} peptide of SEQ ID NO: 5 and a V_{L} peptide of SEQ ID NO: 22.

In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 6, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 19. In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 6, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 19, provided that the V_{H} peptide comprises a HCDR1 having a sequence of SEQ ID NO: 1, a HCDR2 having a sequence of SEQ ID NO: 2, and a HCDR3 having a sequence of SEQ ID NO: 3; and/or the V_{L} peptide comprises a LCDR1 having a sequence of SEQ ID NO: 16, a LCDR2 having a sequence of SEQ ID NO: 17, and a LCDR3 having a sequence of SEQ ID NO: 28. In some embodiments, an antibody, or antigen-binding fragment thereof, comprises a V_{H} peptide of SEQ ID NO: 6 and a V_{L} peptide of SEQ ID NO: 19.

In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 6, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 20. In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 6, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 20, provided that the V_{H} peptide comprises a HCDR1 having a sequence of SEQ ID NO: 1, a HCDR2 having a sequence of SEQ ID NO: 2, and a HCDR3 having a sequence of SEQ ID NO: 3; and/or the V_{L} peptide comprises a LCDR1 having a sequence of SEQ ID NO: 16, a LCDR2 having a sequence of SEQ ID NO: 17, and a LCDR3 having a sequence of SEQ ID NO: 28. In some embodiments, an antibody, or antigen-binding fragment thereof, comprises a V_{H} peptide of SEQ ID NO: 6 and a V_{L} peptide of SEQ ID NO: 20.

In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 6, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 21. In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 6, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 21, provided that the V_{H} peptide comprises a HCDR1 having a sequence of SEQ ID NO: 1, a HCDR2 having a sequence of SEQ ID NO: 2, and a HCDR3 having a sequence of SEQ ID NO: 3; and/or the V_{L} peptide comprises a LCDR1 having a sequence of SEQ ID NO: 16, a LCDR2 having a sequence of SEQ ID NO: 17, and a LCDR3 having a sequence of SEQ ID NO: 28. In some embodiments, an antibody, or antigen-binding fragment thereof, comprises a V_{H} peptide of SEQ ID NO: 6 and a V_{L} peptide of SEQ ID NO: 21.

In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 6, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 22. In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 6, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 22, provided that the V_{H} peptide comprises a HCDR1 having a sequence of SEQ ID NO: 1, a HCDR2 having a sequence of SEQ ID NO: 2, and a HCDR3 having a sequence of SEQ ID NO: 3; and/or the V_{L} peptide comprises a LCDR1 having a sequence of SEQ ID NO: 16, a LCDR2 having a sequence of SEQ ID NO: 17, and a LCDR3 having a sequence of SEQ ID NO: 28. In some embodiments, an antibody, or antigen-binding fragment thereof, comprises a V_{H} peptide of SEQ ID NO: 6 and a V_{L} peptide of SEQ ID NO: 22.

In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 7, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 19. In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 7, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 19, provided that the V_{H} peptide comprises a HCDR1 having a sequence of SEQ ID NO: 1, a HCDR2 having a sequence of SEQ ID NO: 2, and a HCDR3 having a sequence of SEQ ID NO: 3; and/or the V_{L} peptide comprises a LCDR1 having a sequence of SEQ ID NO: 16, a LCDR2 having a sequence of SEQ ID NO: 17, and a LCDR3 having a sequence of SEQ ID NO: 28. In some embodiments, an antibody, or antigen-binding fragment thereof, comprises a V_{H} peptide of SEQ ID NO: 7 and a V_{L} peptide of SEQ ID NO: 19.

In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 7, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 20. In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 7, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 20, provided that the V_{H} peptide comprises a HCDR1 having a sequence of SEQ ID NO: 1, a HCDR2 having a sequence of SEQ ID NO: 2, and a HCDR3 having a sequence of SEQ ID NO: 3; and/or the V_{L} peptide comprises a LCDR1 having a sequence of SEQ ID NO: 16, a LCDR2 having a sequence of SEQ ID NO: 17, and a LCDR3 having a sequence of SEQ ID NO: 28. In some embodiments, an antibody, or antigen-binding fragment thereof, comprises a V_{H} peptide of SEQ ID NO: 7 and a V_{L} peptide of SEQ ID NO: 20.

In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 7, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 21. In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 7, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 21, provided that the V_{H} peptide comprises a HCDR1 having a sequence of SEQ ID NO: 1, a HCDR2 having a sequence of SEQ ID NO: 2, and a HCDR3 having a sequence of SEQ ID NO: 3; and/or the V_{L} peptide comprises a LCDR1 having a sequence of SEQ ID NO: 16, a LCDR2 having a sequence of SEQ ID NO: 17, and a LCDR3 having a sequence of SEQ ID NO: 28. In some embodiments, an antibody, or antigen-binding fragment thereof, comprises a V_{H} peptide of SEQ ID NO: 7 and a V_{L} peptide of SEQ ID NO: 21.

In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 7, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 22. In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 7, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 22, provided that the V_{H} peptide comprises a HCDR1 having a sequence of SEQ ID NO: 1, a HCDR2 having a sequence of SEQ ID NO: 2, and a HCDR3 having a sequence of SEQ ID NO: 3; and/or the V_{L} peptide comprises a LCDR1 having a sequence of SEQ ID NO: 16, a LCDR2 having a sequence of SEQ ID NO: 17, and a LCDR3 having a sequence of SEQ ID NO: 28. In some embodiments, an antibody, or antigen-binding fragment thereof, comprises a V_{H} peptide of SEQ ID NO: 7 and a V_{L} peptide of SEQ ID NO: 22.

In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 8, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 19. In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 8, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 19, provided that the V_{H} peptide comprises a HCDR1 having a sequence of SEQ ID NO: 1, a HCDR2 having a sequence of SEQ ID NO: 2, and a HCDR3 having a sequence of SEQ ID NO: 3; and/or the V_{L} peptide comprises a LCDR1 having a sequence of SEQ ID NO: 16, a LCDR2 having a sequence of SEQ ID NO: 17, and a LCDR3 having a sequence of SEQ ID NO: 28. In some embodiments, an antibody, or antigen-binding fragment thereof, comprises a V_{H} peptide of SEQ ID NO: 8 and a V_{L} peptide of SEQ ID NO: 19.

In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 8, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 20. In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 8, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 20, provided that the V_{H} peptide comprises a HCDR1 having a sequence of SEQ ID NO: 1, a HCDR2 having a sequence of SEQ ID NO: 2, and a HCDR3 having a sequence of SEQ ID NO: 3; and/or the V_{L} peptide comprises a LCDR1 having a sequence of SEQ ID NO: 16, a LCDR2 having a sequence of SEQ ID NO: 17, and a LCDR3 having a sequence of SEQ ID NO: 28. In some embodiments, an antibody, or antigen-binding fragment thereof, comprises a V_{H} peptide of SEQ ID NO: 8 and a V_{L} peptide of SEQ ID NO: 20.

In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 8, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 21. In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 8, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 21, provided that the V_{H} peptide comprises a HCDR1 having a sequence of SEQ ID NO: 1, a HCDR2 having a sequence of SEQ ID NO: 2, and a HCDR3 having a sequence of SEQ ID NO: 3; and/or the V_{L} peptide comprises a LCDR1 having a sequence of SEQ ID NO: 16, a LCDR2 having a sequence of SEQ ID NO: 17, and a LCDR3 having a sequence of SEQ ID NO: 28. In some embodiments, an antibody, or antigen-binding fragment thereof, comprises a V_{H} peptide of SEQ ID NO: 8 and a V_{L} peptide of SEQ ID NO: 21.

In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 8, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 22. In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 8, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 22, provided that the V_{H} peptide comprises a HCDR1 having a sequence of SEQ ID NO: 1, a HCDR2 having a sequence of SEQ ID NO: 2, and a HCDR3 having a sequence of SEQ ID NO: 3, and/or the V_{L} peptide comprises a LCDR1 having a sequence of SEQ ID NO: 16, a LCDR2 having a sequence of SEQ ID NO: 17, and a LCDR3 having a sequence of SEQ ID NO: 28. In some embodiments, an antibody, or antigen-binding fragment thereof, comprises a V_{H} peptide of SEQ ID NO: 8 and a V_{L} peptide of SEQ ID NO: 22.

In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 9, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 19. In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 9, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 19, provided that the V_{H} peptide comprises a HCDR1 having a sequence of SEQ ID NO: 1, a HCDR2 having a sequence of SEQ ID NO: 2, and a HCDR3 having a sequence of SEQ ID NO: 3; and/or the V_{L} peptide comprises a LCDR1 having a sequence of SEQ ID NO: 16, a LCDR2 having a sequence of SEQ ID NO: 17, and a LCDR3 having a sequence of SEQ ID NO: 28. In some embodiments, an antibody, or antigen-binding fragment thereof, comprises a V_{H} peptide of SEQ ID NO: 9 and a V_{L} peptide of SEQ ID NO: 19.

In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 9, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 20. In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 9, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 20, provided that the V_{H} peptide comprises a HCDR1 having a sequence of SEQ ID NO: 1, a HCDR2 having a sequence of SEQ ID NO: 2, and a HCDR3 having a sequence of SEQ ID NO: 3; and/or the V_{L} peptide comprises a LCDR1 having a sequence of SEQ ID NO: 16, a LCDR2 having a sequence of SEQ ID NO: 17, and a LCDR3 having a sequence of SEQ ID NO: 28. In some embodiments, an antibody, or antigen-binding fragment thereof, comprises a V_{H} peptide of SEQ ID NO: 9 and a V_{L} peptide of SEQ ID NO: 20.

In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 9, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 21. In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 9, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 21, provided that the V_{H} peptide comprises a HCDR1 having a sequence of SEQ ID NO: 1, a HCDR2 having a sequence of SEQ ID NO: 2, and a HCDR3 having a sequence of SEQ ID NO: 3; and/or the V_{L} peptide comprises a LCDR1 having a sequence of SEQ ID NO: 16, a LCDR2 having a sequence of SEQ ID NO: 17, and a LCDR3 having a sequence of SEQ ID NO: 28. In some embodiments, an antibody, or antigen-binding fragment thereof, comprises a V_{H} peptide of SEQ ID NO: 9 and a V_{L} peptide of SEQ ID NO: 21.

In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 9, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 22. In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 9, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 22, provided that the V_{H} peptide comprises a HCDR1 having a sequence of SEQ ID NO: 1, a HCDR2 having a sequence of SEQ ID NO: 2, and a HCDR3 having a sequence of SEQ ID NO: 3; and/or the V_{L} peptide comprises a LCDR1 having a sequence of SEQ ID NO: 16, a LCDR2 having a sequence of SEQ ID NO: 17, and a LCDR3 having a sequence of SEQ ID NO: 28. In some embodiments, an antibody, or antigen-binding fragment thereof, comprises a V_{H} peptide of SEQ ID NO: 9 and a V_{L} peptide of SEQ ID NO: 22.

In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 10, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 19. In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 10, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 19, provided that the V_{H} peptide comprises a HCDR1 having a sequence of SEQ ID NO: 1, a HCDR2 having a sequence of SEQ ID NO: 2, and a HCDR3 having a sequence of SEQ ID NO: 3; and/or the V_{L} peptide comprises a LCDR1 having a sequence of SEQ ID NO: 16, a LCDR2 having a sequence of SEQ ID NO: 17, and a LCDR3 having a sequence of SEQ ID NO: 28. In some embodiments, an antibody, or antigen-binding fragment thereof, comprises a V_{H} peptide of SEQ ID NO: 10 and a V_{L} peptide of SEQ ID NO: 19.

In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 10, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 20. In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 10, and the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 20, provided that the V_{H} peptide comprises a HCDR1 having a sequence of SEQ ID NO: 1, a HCDR2 having a sequence of SEQ ID NO: 2, and a HCDR3 having a sequence of SEQ ID NO: 3; and/or the V_{L} peptide comprises a LCDR1 having a sequence of SEQ ID NO: 16, a LCDR2 having a sequence of SEQ ID NO: 17, and a LCDR3 having a sequence of SEQ ID NO: 28. In some embodiments, an antibody, or antigen-binding fragment thereof, comprises a V_{H} peptide of SEQ ID NO: 10 and a V_{L} peptide of SEQ ID NO: 20.

In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 10, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 21. In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 10, and the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 21, provided that the V_{H} peptide comprises a HCDR1 having a sequence of SEQ ID NO: 1, a HCDR2 having a sequence of SEQ ID NO: 2, and a HCDR3 having a sequence of SEQ ID NO: 3; and/or the V_{L} peptide comprises a LCDR1 having a sequence of SEQ ID NO: 16, a LCDR2 having a sequence of SEQ ID NO: 17, and a LCDR3 having a sequence of SEQ ID NO: 28. In some embodiments, an antibody, or antigen-binding fragment thereof, comprises a V peptide of SEQ ID NO: 10 and a V_{L} peptide of SEQ ID NO: 21.

In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 10, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 22. In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 10, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 22, provided that the V_{H} peptide comprises a HCDR1 having a sequence of SEQ ID NO: 1, a HCDR2 having a sequence of SEQ ID NO: 2, and a HCDR3 having a sequence of SEQ ID NO: 3; and/or the V_{L} peptide comprises a LCDR1 having a sequence of SEQ ID NO: 16, a LCDR2 having a sequence of SEQ ID NO: 17, and a LCDR3 having a sequence of SEQ ID NO: 28. In some embodiments, an antibody, or antigen-binding fragment thereof, comprises a V peptide of SEQ ID NO: 10 and a V_{L} peptide of SEQ ID NO: 22.

In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 11, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 19. In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 11, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 19, provided that the V_{H} peptide comprises a HCDR1 having a sequence of SEQ ID NO: 1, a HCDR2 having a sequence of SEQ ID NO: 2, and a HCDR3 having a sequence of SEQ ID NO: 3; and/or the V_{L} peptide comprises a LCDR1 having a sequence of SEQ ID NO: 16, a LCDR2 having a sequence of SEQ ID NO: 17, and a LCDR3 having a sequence of SEQ ID NO: 28. In some embodiments, an antibody, or antigen-binding fragment thereof, comprises a V_{H} peptide of SEQ ID NO: 11 and a V_{L} peptide of SEQ ID NO: 19.

In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 11, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 20. In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 11, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 20, provided that the V_{H} peptide comprises a HCDR1 having a sequence of SEQ ID NO: 1, a HCDR2 having a sequence of SEQ ID NO: 2, and a HCDR3 having a sequence of SEQ ID NO: 3; and/or the V_{L} peptide comprises a LCDR1 having a sequence of SEQ ID NO: 16, a LCDR2 having a sequence of SEQ ID NO: 17, and a LCDR3 having a sequence of SEQ ID NO: 28. In some embodiments, an antibody, or antigen-binding fragment thereof, comprises a V_{H} peptide of SEQ ID NO: 11 and a V_{L} peptide of SEQ ID NO: 20.

In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 11, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 21. In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 11, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 21, provided that the V_{H} peptide comprises a HCDR1 having a sequence of SEQ ID NO: 1, a HCDR2 having a sequence of SEQ ID NO: 2, and a HCDR3 having a sequence of SEQ ID NO: 3; and/or the V_{L} peptide comprises a LCDR1 having a sequence of SEQ ID NO: 16, a LCDR2 having a sequence of SEQ ID NO: 17, and a LCDR3 having a sequence of SEQ ID NO: 28. In some embodiments, an antibody, or antigen-binding fragment thereof, comprises a V_{H} peptide of SEQ ID NO: 11 and a V_{L} peptide of SEQ ID NO: 21.

In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 11, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 22. In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 11, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 22, provided that the V_{H} peptide comprises a HCDR1 having a sequence of SEQ ID NO: 1, a HCDR2 having a sequence of SEQ ID NO: 2, and a HCDR3 having a sequence of SEQ ID NO: 3; and/or the V_{L} peptide comprises a LCDR1 having a sequence of SEQ ID NO: 16, a LCDR2 having a sequence of SEQ ID NO: 17, and a LCDR3 having a sequence of SEQ ID NO: 28. In some embodiments, an antibody, or antigen-binding fragment thereof, comprises a V_{H} peptide of SEQ ID NO: 11 and a V_{L} peptide of SEQ ID NO: 22.

In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 12, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 19. In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 12, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 19, provided that the V_{H} peptide comprises a HCDR1 having a sequence of SEQ ID NO: 1, a HCDR2 having a sequence of SEQ ID NO: 2, and a HCDR3 having a sequence of SEQ ID NO: 3; and/or the V_{L} peptide comprises a LCDR1 having a sequence of SEQ ID NO: 16, a LCDR2 having a sequence of SEQ ID NO: 17, and a LCDR3 having a sequence of SEQ ID NO: 28. In some embodiments, an antibody, or antigen-binding fragment thereof, comprises a V_{H} peptide of SEQ ID NO: 12 and a V_{L} peptide of SEQ ID NO: 19.

In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 12, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 20. In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 12, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 20, provided that the V_{H} peptide comprises a HCDR1 having a sequence of SEQ ID NO: 1, a HCDR2 having a sequence of SEQ ID NO: 2, and a HCDR3 having a sequence of SEQ ID NO: 3; and/or the V_{L} peptide comprises a LCDR1 having a sequence of SEQ ID NO: 16, a LCDR2 having a sequence of SEQ ID NO: 17, and a LCDR3 having a sequence of SEQ ID NO: 28. In some embodiments, an antibody, or antigen-binding fragment thereof, comprises a V_{H} peptide of SEQ ID NO: 12 and a V_{L} peptide of SEQ ID NO: 20.

In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 12, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 21. In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 12, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 21, provided that the V_{H} peptide comprises a HCDR1 having a sequence of SEQ ID NO: 1, a HCDR2 having a sequence of SEQ ID NO: 2, and a HCDR3 having a sequence of SEQ ID NO: 3; and/or the V_{L} peptide comprises a LCDR1 having a sequence of SEQ ID NO: 16, a LCDR2 having a sequence of SEQ ID NO: 17, and a LCDR3 having a sequence of SEQ ID NO: 28. In some embodiments, an antibody, or antigen-binding fragment thereof, comprises a V_{H} peptide of SEQ ID NO: 12 and a V_{L} peptide of SEQ ID NO: 21.

In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 12, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 22. In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 12, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 22, provided that the V_{H} peptide comprises a HCDR1 having a sequence of SEQ ID NO: 1, a HCDR2 having a sequence of SEQ ID NO: 2, and a HCDR3 having a sequence of SEQ ID NO: 3; and/or the V_{L} peptide comprises a LCDR1 having a sequence of SEQ ID NO: 16, a LCDR2 having a sequence of SEQ ID NO: 17, and a LCDR3 having a sequence of SEQ ID NO: 28. In some embodiments, an antibody, or antigen-binding fragment thereof, comprises a V_{H} peptide of SEQ ID NO: 12 and a V_{L} peptide of SEQ ID NO: 22.

In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 13, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 19. In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 13, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 19, provided that the V_{H} peptide comprises a HCDR1 having a sequence of SEQ ID NO: 1, a HCDR2 having a sequence of SEQ ID NO: 2, and a HCDR3 having a sequence of SEQ ID NO: 3; and/or the V_{L} peptide comprises a LCDR1 having a sequence of SEQ ID NO: 16, a LCDR2 having a sequence of SEQ ID NO: 17, and a LCDR3 having a sequence of SEQ ID NO: 28. In some embodiments, an antibody, or antigen-binding fragment thereof, comprises a V_{H} peptide of SEQ ID NO: 13 and a V_{L} peptide of SEQ ID NO: 19.

In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 13, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 20. In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 13, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 20, provided that the V_{H} peptide comprises a HCDR1 having a sequence of SEQ ID NO: 1, a HCDR2 having a sequence of SEQ ID NO: 2, and a HCDR3 having a sequence of SEQ ID NO: 3; and/or the V_{L} peptide comprises a LCDR1 having a sequence of SEQ ID NO: 16, a LCDR2 having a sequence of SEQ ID NO: 17, and a LCDR3 having a sequence of SEQ ID NO: 28. In some embodiments, an antibody, or antigen-binding fragment thereof, comprises a V_{H} peptide of SEQ ID NO: 13 and a V_{L} peptide of SEQ ID NO: 20.

In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 13, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 21. In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 13, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 21, provided that the V_{H} peptide comprises a HCDR1 having a sequence of SEQ ID NO: 1, a HCDR2 having a sequence of SEQ ID NO: 2, and a HCDR3 having a sequence of SEQ ID NO: 3; and/or the V_{L} peptide comprises a LCDR1 having a sequence of SEQ ID NO: 16, a LCDR2 having a sequence of SEQ ID NO: 17, and a LCDR3 having a sequence of SEQ ID NO: 28. In some embodiments, an antibody, or antigen-binding fragment thereof, comprises a V_{H} peptide of SEQ ID NO: 13 and a V_{L} peptide of SEQ ID NO: 21.

In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 13, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 22. In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 13, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 22, provided that the V_{H} peptide comprises a HCDR1 having a sequence of SEQ ID NO: 1, a HCDR2 having a sequence of SEQ ID NO: 2, and a HCDR3 having a sequence of SEQ ID NO: 3, and/or the V_{L} peptide comprises a LCDR1 having a sequence of SEQ ID NO: 16, a LCDR2 having a sequence of SEQ ID NO: 17, and a LCDR3 having a sequence of SEQ ID NO: 28. In some embodiments, an antibody, or antigen-binding fragment thereof, comprises a V_{H} peptide of SEQ ID NO: 13 and a V_{L} peptide of SEQ ID NO: 22.

In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 14, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 19. In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 14, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 19, provided that the V_{H} peptide comprises a HCDR1 having a sequence of SEQ ID NO: 1, a HCDR2 having a sequence of SEQ ID NO: 2, and a HCDR3 having a sequence of SEQ ID NO: 3; and/or the V_{L} peptide comprises a LCDR1 having a sequence of SEQ ID NO: 16, a LCDR2 having a sequence of SEQ ID NO: 17, and a LCDR3 having a sequence of SEQ ID NO: 28. In some embodiments, an antibody, or antigen-binding fragment thereof, comprises a V_{H} peptide of SEQ ID NO: 14 and a V_{L} peptide of SEQ ID NO: 19.

In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 14, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 20. In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 14, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 20, provided that the V_{H} peptide comprises a HCDR1 having a sequence of SEQ ID NO: 1, a HCDR2 having a sequence of SEQ ID NO: 2, and a HCDR3 having a sequence of SEQ ID NO: 3; and/or the V_{L} peptide comprises a LCDR1 having a sequence of SEQ ID NO: 16, a LCDR2 having a sequence of SEQ ID NO: 17, and a LCDR3 having a sequence of SEQ ID NO: 28. In some embodiments, an antibody, or antigen-binding fragment thereof, comprises a V_{H} peptide of SEQ ID NO: 14 and a V_{L} peptide of SEQ ID NO: 20.

In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 14, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 21. In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 14, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 21, provided that the V_{H} peptide comprises a HCDR1 having a sequence of SEQ ID NO: 1, a HCDR2 having a sequence of SEQ ID NO: 2, and a HCDR3 having a sequence of SEQ ID NO: 3; and/or the V_{L} peptide comprises a LCDR1 having a sequence of SEQ ID NO: 16, a LCDR2 having a sequence of SEQ ID NO: 17, and a LCDR3 having a sequence of SEQ ID NO: 28. In some embodiments, an antibody, or antigen-binding fragment thereof, comprises a V_{H} peptide of SEQ ID NO: 14 and a V_{L} peptide of SEQ ID NO: 21.

In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 14, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 22. In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 14, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 22, provided that the V_{H} peptide comprises a HCDR1 having a sequence of SEQ ID NO: 1, a HCDR2 having a sequence of SEQ ID NO: 2, and a HCDR3 having a sequence of SEQ ID NO: 3; and/or the V_{L} peptide comprises a LCDR1 having a sequence of SEQ ID NO: 16, a LCDR2 having a sequence of SEQ ID NO: 17, and a LCDR3 having a sequence of SEQ ID NO: 28. In some embodiments, an antibody, or antigen-binding fragment thereof, comprises a V_{H} peptide of SEQ ID NO: 14 and a V_{L} peptide of SEQ ID NO: 22.

In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 15, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 19. In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 15, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 19, provided that the V_{H} peptide comprises a HCDR1 having a sequence of SEQ ID NO: 1, a HCDR2 having a sequence of SEQ ID NO: 2, and a HCDR3 having a sequence of SEQ ID NO: 3; and/or the V_{L} peptide comprises a LCDR1 having a sequence of SEQ ID NO: 16, a LCDR2 having a sequence of SEQ ID NO: 17, and a LCDR3 having a sequence of SEQ ID NO: 28. In some embodiments, an antibody, or antigen-binding fragment thereof, comprises a V_{H} peptide of SEQ ID NO: 15 and a V_{L} peptide of SEQ ID NO: 19.

In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 15, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 20. In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 15, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 20, provided that the V_{H} peptide comprises a HCDR1 having a sequence of SEQ ID NO: 1, a HCDR2 having a sequence of SEQ ID NO: 2, and a HCDR3 having a sequence of SEQ ID NO: 3; and/or the V_{L} peptide comprises a LCDR1 having a sequence of SEQ ID NO: 16, a LCDR2 having a sequence of SEQ ID NO: 17, and a LCDR3 having a sequence of SEQ ID NO: 28. In some embodiments, an antibody, or antigen-binding fragment thereof, comprises a V_{H} peptide of SEQ ID NO: 15 and a V_{L} peptide of SEQ ID NO: 20.

In some embodiments, an antibody, or antigen-binding fragment thereof, comprises a V_{H} peptide and a V peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 15, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 21. In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 15, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 21, provided that the V_{H} peptide comprises a HCDR1 having a sequence of SEQ ID NO: 1, a HCDR2 having a sequence of SEQ ID NO: 2, and a HCDR3 having a sequence of SEQ ID NO: 3; and/or the V_{L} peptide comprises a LCDR1 having a sequence of SEQ ID NO: 16, a LCDR2 having a sequence of SEQ ID NO: 17, and a LCDR3 having a sequence of SEQ ID NO: 28. In some embodiments, an antibody, or antigen-binding fragment thereof, comprises a V peptide of SEQ ID NO: 15 and a V_{L} peptide of SEQ ID NO: 21.

In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 15, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 22. In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 15, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 22, provided that the V_{H} peptide comprises a HCDR1 having a sequence of SEQ ID NO: 1, a HCDR2 having a sequence of SEQ ID NO: 2, and a HCDR3 having a sequence of SEQ ID NO: 3; and/or the V_{L} peptide comprises a LCDR1 having a sequence of SEQ ID NO: 16, a LCDR2 having a sequence of SEQ ID NO: 17, and a LCDR3 having a sequence of SEQ ID NO: 28. In some embodiments, an antibody, or antigen-binding fragment thereof, comprises a V peptide of SEQ ID NO: 15 and a V_{L} peptide of SEQ ID NO: 22.

In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 30, SEQ ID NO: 31, SEQ ID NO: 32, or SEQ ID NO: 33; and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 37.

In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 30, SEQ ID NO: 31, SEQ ID NO: 32, or SEQ ID NO: 33; and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 37; provided that the V_{H} peptide and a V_{L} peptide comprises at least one HCDR having a sequence of SEQ ID NO: 23, SEQ ID NO: 24, SEQ ID NO: 25, SEQ ID NO: 26, SEQ ID NO: 27, SEQ ID NO: 28, or SEQ ID NO: 29; and/or at least one LCDR having a sequence of SEQ ID NO: 34, SEQ ID NO: 35, or SEQ ID NO: 36. In some embodiments, the CDRs in the V_{H} or V_{L} chain are as set forth in the combinations provided for herein.

In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 30, SEQ ID NO: 31, SEQ ID NO: 32, or SEQ ID NO: 33; and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 37; provided that the V_{H} peptide comprises a HCDR1 having a sequence of SEQ ID NO: 23, a HCDR2 having a sequence of SEQ ID NO: 24, SEQ ID NO: 25, SEQ ID NO: 26, or SEQ ID NO: 27, and a HCDR3 having a sequence of SEQ ID NO: 28, or SEQ ID NO: 29; and/or the V_{L} peptide comprises a LCDR1 having a sequence of SEQ ID NO: 34, a LCDR2 having a sequence of SEQ ID NO: 35, and a LCDR3 having a sequence of SEQ ID NO: 36.

In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 30, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 37. In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 30, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 37, provided that the V_{H} peptide comprises a HCDR1 having a sequence of SEQ ID NO: 23, a HCDR2 having a sequence of SEQ ID NO: 24, SEQ ID NO: 25, SEQ ID NO: 26, or SEQ ID NO: 27, and a HCDR3 having a sequence of SEQ ID NO: 28, or SEQ ID NO: 29; and/or the V_{L} peptide comprises a LCDR1 having a sequence of SEQ ID NO: 34, a LCDR2 having a sequence of SEQ ID NO: 35, and a LCDR3 having a sequence of SEQ ID NO: 36. In some embodiments, an antibody, or antigen-binding fragment thereof, comprises a V_{H} peptide of SEQ ID NO: 30 and a V_{L} peptide of SEQ ID NO: 37.

In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 31, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 37. In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 31, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 37, provided that the V_{H} peptide comprises a HCDR1 having a sequence of SEQ ID NO: 23, a HCDR2 having a sequence of SEQ ID NO: 24, SEQ ID NO: 25, SEQ ID NO: 26, or SEQ ID NO: 27, and a HCDR3 having a sequence of SEQ ID NO: 28, or SEQ ID NO: 29; and/or the V_{L} peptide comprises a LCDR1 having a sequence of SEQ ID NO: 34, a LCDR2 having a sequence of SEQ ID NO: 35, and a LCDR3 having a sequence of SEQ ID NO: 36. In some embodiments, an antibody, or antigen-binding fragment thereof, comprises a V_{H} peptide of SEQ ID NO: 31 and a V_{L} peptide of SEQ ID NO: 37.

In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 32, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 37. In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 32, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 37, provided that the V_{H} peptide comprises a HCDR1 having a sequence of SEQ ID NO: 23, a HCDR2 having a sequence of SEQ ID NO: 24, SEQ ID NO: 25, SEQ ID NO: 26, or SEQ ID NO: 27, and a HCDR3 having a sequence of SEQ ID NO: 28, or SEQ ID NO: 29; and/or the V_{L} peptide comprises a LCDR1 having a sequence of SEQ ID NO: 34, a LCDR2 having a sequence of SEQ ID NO: 35, and a LCDR3 having a sequence of SEQ ID NO: 36. In some embodiments, an antibody, or antigen-binding fragment thereof, comprises a V_{H} peptide of SEQ ID NO: 32 and a V_{L} peptide of SEQ ID NO: 37.

In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 33, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 37. In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 33, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 37, provided that the V_{H} peptide comprises a HCDR1 having a sequence of SEQ ID NO: 23, a HCDR2 having a sequence of SEQ ID NO: 24, SEQ ID NO: 25, SEQ ID NO: 26, or SEQ ID NO: 27, and a HCDR3 having a sequence of SEQ ID NO: 28, or SEQ ID NO: 29; and/or the V_{L} peptide comprises a LCDR1 having a sequence of SEQ ID NO: 34, a LCDR2 having a sequence of SEQ ID NO: 35, and a LCDR3 having a sequence of SEQ ID NO: 36. In some embodiments, an antibody, or antigen-binding fragment thereof, comprises a V_{H} peptide of SEQ ID NO: 33 and a V_{L} peptide of SEQ ID NO: 37.

In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, or SEQ ID NO: 57; and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 61, or SEQ ID NO: 62.

In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, or SEQ ID NO: 57; and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 61, or SEQ ID NO: 62; provided that the V_{H} peptide and a V_{L} peptide comprises at least one HCDR having a sequence of SEQ ID NO: 38, SEQ ID NO: 39, SEQ ID NO: 40, SEQ ID NO: 41, SEQ ID NO: 42, or SEQ ID NO: 43; and/or at least one LCDR having a sequence of SEQ ID NO: 58, SEQ ID NO: 59, or SEQ ID NO: 60. In some embodiments, the CDRs in the V_{H} or V_{L} chain are as set forth in the combinations provided for herein.

In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, or SEQ ID NO: 57, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 61, or SEQ ID NO: 62; provided that the V_{H} peptide comprises a HCDR1 having a sequence of SEQ ID NO: 38, SEQ ID NO: 39, or SEQ ID NO: 40, a HCDR2 having a sequence of SEQ ID NO: 41, or SEQ ID NO: 42, and a HCDR3 having a sequence of SEQ ID NO: 43; and/or the V_{L} peptide comprises a LCDR1 having a sequence of SEQ ID NO: 58, a LCDR2 having a sequence of SEQ ID NO: 59, and a LCDR3 having a sequence of SEQ ID NO: 60.

In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 44, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 61. In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 44, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 61, provided that the V_{H} peptide comprises a HCDR1 having a sequence of SEQ ID NO: 38, SEQ ID NO: 39, or SEQ ID NO: 40, a HCDR2 having a sequence of SEQ ID NO: 41, or SEQ ID NO: 42, and a HCDR3 having a sequence of SEQ ID NO: 43; and/or the V_{L} peptide comprises a LCDR1 having a sequence of SEQ ID NO: 58, a LCDR2 having a sequence of SEQ ID NO: 59, and a LCDR3 having a sequence of SEQ ID NO: 60. In some embodiments, an antibody, or antigen-binding fragment thereof, comprises a V_{H} peptide of SEQ ID NO: 44 and a V_{L} peptide of SEQ ID NO: 61.

In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 44, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 62. In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 44, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 62, provided that the V_{H} peptide comprises a HCDR1 having a sequence of SEQ ID NO: 38, SEQ ID NO: 39, or SEQ ID NO: 40, a HCDR2 having a sequence of SEQ ID NO: 41, or SEQ ID NO: 42, and a HCDR3 having a sequence of SEQ ID NO: 43; and/or the V_{L} peptide comprises a LCDR1 having a sequence of SEQ ID NO: 58, a LCDR2 having a sequence of SEQ ID NO: 59, and a LCDR3 having a sequence of SEQ ID NO: 60. In some embodiments, an antibody, or antigen-binding fragment thereof, comprises a V_{H} peptide of SEQ ID NO: 44 and a V_{L} peptide of SEQ ID NO: 62.

In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 45, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 61. In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 45, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 61, provided that the V_{H} peptide comprises a HCDR1 having a sequence of SEQ ID NO: 38, SEQ ID NO: 39, or SEQ ID NO: 40, a HCDR2 having a sequence of SEQ ID NO: 41, or SEQ ID NO: 42, and a HCDR3 having a sequence of SEQ ID NO: 43; and/or the V_{L} peptide comprises a LCDR1 having a sequence of SEQ ID NO: 58, a LCDR2 having a sequence of SEQ ID NO: 59, and a LCDR3 having a sequence of SEQ ID NO: 60. In some embodiments, an antibody, or antigen-binding fragment thereof, comprises a V_{H} peptide of SEQ ID NO: 45 and a V_{L} peptide of SEQ ID NO: 61.

In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 45, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 62. In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 45, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 62, provided that the V_{H} peptide comprises a HCDR1 having a sequence of SEQ ID NO: 38, SEQ ID NO: 39, or SEQ ID NO: 40, a HCDR2 having a sequence of SEQ ID NO: 41, or SEQ ID NO: 42, and a HCDR3 having a sequence of SEQ ID NO: 43; and/or the Vt peptide comprises a LCDR1 having a sequence of SEQ ID NO: 58, a LCDR2 having a sequence of SEQ ID NO: 59, and a LCDR3 having a sequence of SEQ ID NO: 60. In some embodiments, an antibody, or antigen-binding fragment thereof, comprises a V_{H} peptide of SEQ ID NO: 45 and a V_{L} peptide of SEQ ID NO: 62.

In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 46, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 61. In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 46, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 61, provided that the V_{H} peptide comprises a HCDR1 having a sequence of SEQ ID NO: 38, SEQ ID NO: 39, or SEQ ID NO: 40, a HCDR2 having a sequence of SEQ ID NO: 41, or SEQ ID NO: 42, and a HCDR3 having a sequence of SEQ ID NO: 43; and/or the V_{L} peptide comprises a LCDR1 having a sequence of SEQ ID NO: 58, a LCDR2 having a sequence of SEQ ID NO: 59, and a LCDR3 having a sequence of SEQ ID NO: 60. In some embodiments, an antibody, or antigen-binding fragment thereof, comprises a V_{H} peptide of SEQ ID NO: 46 and a V_{L} peptide of SEQ ID NO: 61.

In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 46, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 62. In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 46, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 62, provided that the V_{H} peptide comprises a HCDR1 having a sequence of SEQ ID NO: 38, SEQ ID NO: 39, or SEQ ID NO: 40, a HCDR2 having a sequence of SEQ ID NO: 41, or SEQ ID NO: 42, and a HCDR3 having a sequence of SEQ ID NO: 43; and/or the V_{L} peptide comprises a LCDR1 having a sequence of SEQ ID NO: 58, a LCDR2 having a sequence of SEQ ID NO: 59, and a LCDR3 having a sequence of SEQ ID NO: 60. In some embodiments, an antibody, or antigen-binding fragment thereof, comprises a V_{H} peptide of SEQ ID NO: 46 and a V_{L} peptide of SEQ ID NO: 62.

In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 47, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 61. In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 47, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 61, provided that the V_{H} peptide comprises a HCDR1 having a sequence of SEQ ID NO: 38, SEQ ID NO: 39, or SEQ ID NO: 40, a HCDR2 having a sequence of SEQ ID NO: 41, or SEQ ID NO: 42, and a HCDR3 having a sequence of SEQ ID NO: 43; and/or the V_{L} peptide comprises a LCDR1 having a sequence of SEQ ID NO: 58, a LCDR2 having a sequence of SEQ ID NO: 59, and a LCDR3 having a sequence of SEQ ID NO: 60. In some embodiments, an antibody, or antigen-binding fragment thereof, comprises a V_{H} peptide of SEQ ID NO: 47 and a V_{L} peptide of SEQ ID NO: 61.

In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 47, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 62. In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 47, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 62, provided that the V_{H} peptide comprises a HCDR1 having a sequence of SEQ ID NO: 38, SEQ ID NO: 39, or SEQ ID NO: 40, a HCDR2 having a sequence of SEQ ID NO: 41, or SEQ ID NO: 42, and a HCDR3 having a sequence of SEQ ID NO: 43; and/or the V_{L} peptide comprises a LCDR1 having a sequence of SEQ ID NO: 58, a LCDR2 having a sequence of SEQ ID NO: 59, and a LCDR3 having a sequence of SEQ ID NO: 60. In some embodiments, an antibody, or antigen-binding fragment thereof, comprises a V_{H} peptide of SEQ ID NO: 47 and a V_{L} peptide of SEQ ID NO: 62.

In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 48, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 61. In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 48, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 61, provided that the V_{H} peptide comprises a HCDR1 having a sequence of SEQ ID NO: 38, SEQ ID NO: 39, or SEQ ID NO: 40, a HCDR2 having a sequence of SEQ ID NO: 41, or SEQ ID NO: 42, and a HCDR3 having a sequence of SEQ ID NO: 43; and/or the V_{L} peptide comprises a LCDR1 having a sequence of SEQ ID NO: 58, a LCDR2 having a sequence of SEQ ID NO: 59, and a LCDR3 having a sequence of SEQ ID NO: 60. In some embodiments, an antibody, or antigen-binding fragment thereof, comprises a V_{H} peptide of SEQ ID NO: 48 and a V_{L} peptide of SEQ ID NO: 61.

In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 48, and the Vt peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 62. In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 48, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 62, provided that the V_{H} peptide comprises a HCDR1 having a sequence of SEQ ID NO: 38, SEQ ID NO: 39, or SEQ ID NO: 40, a HCDR2 having a sequence of SEQ ID NO: 41, or SEQ ID NO: 42, and a HCDR3 having a sequence of SEQ ID NO: 43; and/or the V_{L} peptide comprises a LCDR1 having a sequence of SEQ ID NO: 58, a LCDR2 having a sequence of SEQ ID NO: 59, and a LCDR3 having a sequence of SEQ ID NO: 60. In some embodiments, an antibody, or antigen-binding fragment thereof, comprises a V_{H} peptide of SEQ ID NO: 48 and a V_{L} peptide of SEQ ID NO: 62.

In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 49, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 61. In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 49, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 61, provided that the V_{H} peptide comprises a HCDR1 having a sequence of SEQ ID NO: 38, SEQ ID NO: 39, or SEQ ID NO: 40, a HCDR2 having a sequence of SEQ ID NO: 41, or SEQ ID NO: 42, and a HCDR3 having a sequence of SEQ ID NO: 43; and/or the V_{L} peptide comprises a LCDR1 having a sequence of SEQ ID NO: 58, a LCDR2 having a sequence of SEQ ID NO: 59, and a LCDR3 having a sequence of SEQ ID NO: 60. In some embodiments, an antibody, or antigen-binding fragment thereof, comprises a V_{H} peptide of SEQ ID NO: 49 and a V_{L} peptide of SEQ ID NO: 61.

In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 49, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 62. In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 49, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 62, provided that the V_{H} peptide comprises a HCDR1 having a sequence of SEQ ID NO: 38, SEQ ID NO: 39, or SEQ ID NO: 40, a HCDR2 having a sequence of SEQ ID NO: 41, or SEQ ID NO: 42, and a HCDR3 having a sequence of SEQ ID NO: 43; and/or the V_{L} peptide comprises a LCDR1 having a sequence of SEQ ID NO: 58, a LCDR2 having a sequence of SEQ ID NO: 59, and a LCDR3 having a sequence of SEQ ID NO: 60. In some embodiments, an antibody, or antigen-binding fragment thereof, comprises a V_{H} peptide of SEQ ID NO: 49 and a V_{L} peptide of SEQ ID NO: 62.

In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 50, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 61. In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 50, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 61, provided that the V_{H} peptide comprises a HCDR1 having a sequence of SEQ ID NO: 38, SEQ ID NO: 39, or SEQ ID NO: 40, a HCDR2 having a sequence of SEQ ID NO: 41, or SEQ ID NO: 42, and a HCDR3 having a sequence of SEQ ID NO: 43; and/or the V_{L} peptide comprises a LCDR1 having a sequence of SEQ ID NO: 58, a LCDR2 having a sequence of SEQ ID NO: 59, and a LCDR3 having a sequence of SEQ ID NO: 60. In some embodiments, an antibody, or antigen-binding fragment thereof, comprises a V_{H} peptide of SEQ ID NO: 50 and a V_{L} peptide of SEQ ID NO: 61.

In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 50, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 62. In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 50, and the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 62, provided that the V_{H} peptide comprises a HCDR1 having a sequence of SEQ ID NO: 38, SEQ ID NO: 39, or SEQ ID NO: 40, a HCDR2 having a sequence of SEQ ID NO: 41, or SEQ ID NO: 42, and a HCDR3 having a sequence of SEQ ID NO: 43; and/or the V_{L} peptide comprises a LCDR1 having a sequence of SEQ ID NO: 58, a LCDR2 having a sequence of SEQ ID NO: 59, and a LCDR3 having a sequence of SEQ ID NO: 60. In some embodiments, an antibody, or antigen-binding fragment thereof, comprises a V_{H} peptide of SEQ ID NO: 50 and a V_{L} peptide of SEQ ID NO: 62.

In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 51, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 61. In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 51, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 61, provided that the V_{H} peptide comprises a HCDR1 having a sequence of SEQ ID NO: 38, SEQ ID NO: 39, or SEQ ID NO: 40, a HCDR2 having a sequence of SEQ ID NO: 41, or SEQ ID NO: 42, and a HCDR3 having a sequence of SEQ ID NO: 43; and/or the V_{L} peptide comprises a LCDR1 having a sequence of SEQ ID NO: 58, a LCDR2 having a sequence of SEQ ID NO: 59, and a LCDR3 having a sequence of SEQ ID NO: 60. In some embodiments, an antibody, or antigen-binding fragment thereof, comprises a V_{H} peptide of SEQ ID NO: 51 and a V_{L} peptide of SEQ ID NO: 61.

In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 51, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 62. In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 51, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 62, provided that the V_{H} peptide comprises a HCDR1 having a sequence of SEQ ID NO: 38, SEQ ID NO: 39, or SEQ ID NO: 40, a HCDR2 having a sequence of SEQ ID NO: 41, or SEQ ID NO: 42, and a HCDR3 having a sequence of SEQ ID NO: 43; and/or the V_{L} peptide comprises a LCDR1 having a sequence of SEQ ID NO: 58, a LCDR2 having a sequence of SEQ ID NO: 59, and a LCDR3 having a sequence of SEQ ID NO: 60. In some embodiments, an antibody, or antigen-binding fragment thereof, comprises a V_{H} peptide of SEQ ID NO: 51 and a V_{L} peptide of SEQ ID NO: 62.

In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 52, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 61. In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 52, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 61, provided that the V_{H} peptide comprises a HCDR1 having a sequence of SEQ ID NO: 38, SEQ ID NO: 39, or SEQ ID NO: 40, a HCDR2 having a sequence of SEQ ID NO: 41, or SEQ ID NO: 42, and a HCDR3 having a sequence of SEQ ID NO: 43; and/or the V_{L} peptide comprises a LCDR1 having a sequence of SEQ ID NO: 58, a LCDR2 having a sequence of SEQ ID NO: 59, and a LCDR3 having a sequence of SEQ ID NO: 60. In some embodiments, an antibody, or antigen-binding fragment thereof, comprises a V_{H} peptide of SEQ ID NO: 52 and a V_{L} peptide of SEQ ID NO: 61.

In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 52, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 62. In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 52, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 62, provided that the V_{H} peptide comprises a HCDR1 having a sequence of SEQ ID NO: 38, SEQ ID NO: 39, or SEQ ID NO: 40, a HCDR2 having a sequence of SEQ ID NO: 41, or SEQ ID NO: 42, and a HCDR3 having a sequence of SEQ ID NO: 43; and/or the V_{L} peptide comprises a LCDR1 having a sequence of SEQ ID NO: 58, a LCDR2 having a sequence of SEQ ID NO: 59, and a LCDR3 having a sequence of SEQ ID NO: 60. In some embodiments, an antibody, or antigen-binding fragment thereof, comprises a V_{H} peptide of SEQ ID NO: 52 and a V_{L} peptide of SEQ ID NO: 62.

In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 53, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 61. In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 53, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 61, provided that the V_{H} peptide comprises a HCDR1 having a sequence of SEQ ID NO: 38, SEQ ID NO: 39, or SEQ ID NO: 40, a HCDR2 having a sequence of SEQ ID NO: 41, or SEQ ID NO: 42, and a HCDR3 having a sequence of SEQ ID NO: 43; and/or the V_{L} peptide comprises a LCDR1 having a sequence of SEQ ID NO: 58, a LCDR2 having a sequence of SEQ ID NO: 59, and a LCDR3 having a sequence of SEQ ID NO: 60. In some embodiments, an antibody, or antigen-binding fragment thereof, comprises a V_{H} peptide of SEQ ID NO: 53 and a V_{L} peptide of SEQ ID NO: 61.

In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 53, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 62. In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 53, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 62, provided that the V_{H} peptide comprises a HCDR1 having a sequence of SEQ ID NO: 38, SEQ ID NO: 39, or SEQ ID NO: 40, a HCDR2 having a sequence of SEQ ID NO: 41, or SEQ ID NO: 42, and a HCDR3 having a sequence of SEQ ID NO: 43; and/or the V_{L} peptide comprises a LCDR1 having a sequence of SEQ ID NO: 58, a LCDR2 having a sequence of SEQ ID NO: 59, and a LCDR3 having a sequence of SEQ ID NO: 60. In some embodiments, an antibody, or antigen-binding fragment thereof, comprises a V_{H} peptide of SEQ ID NO: 53 and a V_{L} peptide of SEQ ID NO: 62.

In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 54, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 61. In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 54, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 61, provided that the V_{H} peptide comprises a HCDR1 having a sequence of SEQ ID NO: 38, SEQ ID NO: 39, or SEQ ID NO: 40, a HCDR2 having a sequence of SEQ ID NO: 41, or SEQ ID NO: 42, and a HCDR3 having a sequence of SEQ ID NO: 43; and/or the V_{L} peptide comprises a LCDR1 having a sequence of SEQ ID NO: 58, a LCDR2 having a sequence of SEQ ID NO: 59, and a LCDR3 having a sequence of SEQ ID NO: 60. In some embodiments, an antibody, or antigen-binding fragment thereof, comprises a V_{H} peptide of SEQ ID NO: 54 and a V_{L} peptide of SEQ ID NO: 61.

In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 54, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 62. In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 54, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 62, provided that the V_{H} peptide comprises a HCDR1 having a sequence of SEQ ID NO: 38, SEQ ID NO: 39, or SEQ ID NO: 40, a HCDR2 having a sequence of SEQ ID NO: 41, or SEQ ID NO: 42, and a HCDR3 having a sequence of SEQ ID NO: 43; and/or the V_{L} peptide comprises a LCDR1 having a sequence of SEQ ID NO: 58, a LCDR2 having a sequence of SEQ ID NO: 59, and a LCDR3 having a sequence of SEQ ID NO: 60. In some embodiments, an antibody, or antigen-binding fragment thereof, comprises a V_{H} peptide of SEQ ID NO: 54 and a V_{L} peptide of SEQ ID NO: 62.

In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 55, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 61. In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 55, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 61, provided that the V_{H} peptide comprises a HCDR1 having a sequence of SEQ ID NO: 38, SEQ ID NO: 39, or SEQ ID NO: 40, a HCDR2 having a sequence of SEQ ID NO: 41, or SEQ ID NO: 42, and a HCDR3 having a sequence of SEQ ID NO: 43; and/or the V_{L} peptide comprises a LCDR1 having a sequence of SEQ ID NO: 58, a LCDR2 having a sequence of SEQ ID NO: 59, and a LCDR3 having a sequence of SEQ ID NO: 60. In some embodiments, an antibody, or antigen-binding fragment thereof, comprises a V_{H} peptide of SEQ ID NO: 55 and a V_{L} peptide of SEQ ID NO: 61.

In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 55, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 62. In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 55, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 62, provided that the V_{H} peptide comprises a HCDR1 having a sequence of SEQ ID NO: 38, SEQ ID NO: 39, or SEQ ID NO: 40, a HCDR2 having a sequence of SEQ ID NO: 41, or SEQ ID NO: 42, and a HCDR3 having a sequence of SEQ ID NO: 43; and/or the V_{L} peptide comprises a LCDR1 having a sequence of SEQ ID NO: 58, a LCDR2 having a sequence of SEQ ID NO: 59, and a LCDR3 having a sequence of SEQ ID NO: 60. In some embodiments, an antibody, or antigen-binding fragment thereof, comprises a V_{H} peptide of SEQ ID NO: 55 and a V_{L} peptide of SEQ ID NO: 62.

In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 56, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 61. In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 56, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 61, provided that the V_{H} peptide comprises a HCDR1 having a sequence of SEQ ID NO: 38, SEQ ID NO: 39, or SEQ ID NO: 40, a HCDR2 having a sequence of SEQ ID NO: 41, or SEQ ID NO: 42, and a HCDR3 having a sequence of SEQ ID NO: 43; and/or the V_{L} peptide comprises a LCDR1 having a sequence of SEQ ID NO: 58, a LCDR2 having a sequence of SEQ ID NO: 59, and a LCDR3 having a sequence of SEQ ID NO: 60. In some embodiments, an antibody, or antigen-binding fragment thereof, comprises a V_{H} peptide of SEQ ID NO: 56 and a V_{L} peptide of SEQ ID NO: 61.

In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 56, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 62. In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 56, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 62, provided that the V_{H} peptide comprises a HCDR1 having a sequence of SEQ ID NO: 38, SEQ ID NO: 39, or SEQ ID NO: 40, a HCDR2 having a sequence of SEQ ID NO: 41, or SEQ ID NO: 42, and a HCDR3 having a sequence of SEQ ID NO: 43; and/or the V_{L} peptide comprises a LCDR1 having a sequence of SEQ ID NO: 58, a LCDR2 having a sequence of SEQ ID NO: 59, and a LCDR3 having a sequence of SEQ ID NO: 60. In some embodiments, an antibody, or antigen-binding fragment thereof, comprises a V_{H} peptide of SEQ ID NO: 56 and a V_{L} peptide of SEQ ID NO: 62.

In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 57, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 61. In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 57, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 61, provided that the V_{H} peptide comprises a HCDR1 having a sequence of SEQ ID NO: 38, SEQ ID NO: 39, or SEQ ID NO: 40, a HCDR2 having a sequence of SEQ ID NO: 41, or SEQ ID NO: 42, and a HCDR3 having a sequence of SEQ ID NO: 43; and/or the V_{L} peptide comprises a LCDR1 having a sequence of SEQ ID NO: 58, a LCDR2 having a sequence of SEQ ID NO: 59, and a LCDR3 having a sequence of SEQ ID NO: 60. In some embodiments, an antibody, or antigen-binding fragment thereof, comprises a V_{H} peptide of SEQ ID NO: 57 and a V_{L} peptide of SEQ ID NO: 61.

In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 57, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 62. In some embodiments, an antibody, or antigen-binding fragment thereof, comprises a V_{H} peptide and a V_{L} peptide, wherein the V_{H} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 57, and the V_{L} peptide comprises a sequence that is at least 85%, 86%, 87%, 88%, 89%, 90%, 91%, 92%, 93%, 94%, 95%, 96%, 97%, 98%, 99%, or 100% identical to a sequence of SEQ ID NO: 62, provided that the V_{H} peptide comprises a HCDR1 having a sequence of SEQ ID NO: 38, SEQ ID NO: 39, or SEQ ID NO: 40, a HCDR2 having a sequence of SEQ ID NO: 41, or SEQ ID NO: 42, and a HCDR3 having a sequence of SEQ ID NO: 43; and/or the V_{L} peptide comprises a LCDR1 having a sequence of SEQ ID NO: 58, a LCDR2 having a sequence of SEQ ID NO: 59, and a LCDR3 having a sequence of SEQ ID NO: 60. In some embodiments, an antibody, or antigen-binding fragment thereof, comprises a V_{H} peptide of SEQ ID NO: 57 and a V_{L} peptide of SEQ ID NO: 62.

In some embodiments, the antibody comprises a Fc domain. The Fc domain can be linked to the heavy or light chain of the antibody. In some embodiments, the Fc domain comprises a mutation to extend the half-life of the antibody. In some embodiments, the Fc domain comprises a mutation such as those described in U.S. Pat. No. 7,670,600, which is hereby incorporated by reference in its entirety. In some embodiment, the constant region comprises a mutation at position at amino acid residue 428 relative to a wild-type human IgG constant domain, numbered according to the EU numbering index of Kabat. Without being bound to any particular theory, an antibody comprising a mutation that corresponds to residue 428 can have an increased half-life compared to the half-life of an IgG having the wild-type human IgG constant domain. In some embodiments, the mutation is a substitution of the native residue with a threonine, leucine, phenylalanine or serine. In some embodiments, the antibody further comprises one or more amino acid substitutions relative to the corresponding wild-type human IgG constant domain at one or more of amino acid residues 251-256, 285-290, 308-314, 385-389, and 429-436, numbered according to the Kabat EU numbering index. The specific mutations or substitutions at these positions are described in U.S. Pat. No. 7,670,600, which is hereby incorporated by reference in its entirety.

Other mutations can be used in the Fc domain, such as those provided for in U.S. Pat. No. 8,394,925, which is hereby incorporated by reference in its entirety. In some embodiments, the Fc region is a variant Fc region comprising amino acid substitutions at positions 428 and 434, wherein the amino acid substitutions are a leucine that is not the wild-type amino acid at position 428 and a serine that is not the wild-type amino acid at position 434, wherein the polypeptide is an antibody and wherein numbering is according to the EU Index in Kabat et al. In some embodiments, the Fc region comprises a S228P, L235E, M428L, or N434S substitution. In some embodiments, the Fc region comprises a M428L substitution. In some embodiments, the Fc region comprises a N434S substitution. In some embodiments, the Fc region comprises a M428L and a N434S substitution. In some embodiments, the Fc region comprises a M252Y, S254T, and/or T256E substitution.

In some embodiments, the antibody comprises a constant region as set forth below with or without the mutations provided for the list below.

IgG1-AAS constant region
(SEQ ID NO: 63)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPEAAGGPSVFLFPPKPKDTLMISRTPEVTCVVVSVS
HEDPEVKFNWYVDGVEVHNAKTKPREEQYNSTYRVVSVLTVLHQDWLNGK
EYKCKVSNKALPAPIEKTISKAKGQPREPQVYTLPPSREEMTKNQVSLTC
LVKGFYPSDIAVEWESNGQPENNYKTTPPVLDSDGSFFLYSKLTVDKSRW
QQGNVFSCSVMHEALHNHYTQKSLSLSPGK
IgG4 S228P L235E LS
(SEQ ID NO: 64)
ASTKGPSVFPLAPCSRSTSESTAALGCLVKDYFPEPVTVSWNSGALTSGV
HTFPAVLQSSGLYSLSSVVTVPSSSLGTKTYTCNVDHKPSNTKVDKRVES
KYGPPCPPCPAPEFEGGPSVFLFPPKPKDTLMISRTPEVTCVVVDVSQED
PEVQFNWYVDGVEVHNAKTKPREEQFNSTYRVVSVLTVLHQDWLNGKEYK
CKVSNKGLPSSIEKTISKAKGQPREPQVYTLPPSQEEMTKNQVSLTCLVK
GFYPSDIAVEWESNGQPENNYKTTPPVLDSDGSFFLYSRLTVDKSRWQEG
NVFSCSVLHEALHSHYTQKSLSLSLGK
IgG4 S228P L235E YTE
(SEQ ID NO: 65)
ASTKGPSVFPLAPCSRSTSESTAALGCLVKDYFPEPVTVSWNSGALTSGV
HTFPAVLQSSGLYSLSSVVTVPSSSLGTKTYTCNVDHKPSNTKVDKRVES
KYGPPCPPCPAPEFEGGPSVFLFPPKPKDTLYITREPEVTCVVVDVSQED
PEVQFNWYVDGVEVHNAKTKPREEQFNSTYRVVSVLTVLHQDWLNGKEYK
CKVSNKGLPSSIEKTISKAKGQPREPQVYTLPPSQEEMTKNQVSLTCLVK
GFYPSDIAVEWESNGQPENNYKTTPPVLDSDGSFFLYSRLTVDKSRWQEG
NVFSCSVMHEALHNHYTQKSLSLSLGK

In some embodiments, the antibody comprises a constant region as provided herein, wherein the C-terminal lysine (K) amino acid has been deleted. In some embodiments, the antibody comprises a constant region as set follows:

IgG1-AAS constant trunc
(SEQ ID NO: 66)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPEAAGGPSVFLFPPKPKDTLMISRTPEVTCVVVSVS
HEDPEVKFNWYVDGVEVHNAKTKPREEQYNSTYRVVSVLTVLHQDWLNGK
EYKCKVSNKALPAPIEKTISKAKGQPREPQVYTLPPSREEMTKNQVSLTC
LVKGFYPSDIAVEWESNGQPENNYKTTPPVLDSDGSFFLYSKLTVDKSRW
QQGNVFSCSVMHEALHNHYTQKSLSLSPG
IgG4 S228P L235E LS-trunc
(SEQ ID NO: 67)
ASTKGPSVFPLAPCSRSTSESTAALGCLVKDYFPEPVTVSWNSGALTSGV
HTFPAVLQSSGLYSLSSVVTVPSSSLGTKTYTCNVDHKPSNTKVDKRVES
KYGPPCPPCPAPEFEGGPSVFLFPPKPKDTLMISRTPEVTCVVVDVSQED
PEVQFNWYVDGVEVHNAKTKPREEQFNSTYRVVSVLTVLHQDWLNGKEYK
CKVSNKGLPSSIEKTISKAKGQPREPQVYTLPPSQEEMTKNQVSLTCLVK
GFYPSDIAVEWESNGQPENNYKTTPPVLDSDGSFFLYSRLTVDKSRWQEG
NVFSCSVLHEALHSHYTQKSLSLSLG
IgG4 S228P L235E YTE-trunc
(SEQ ID NO: 68)
ASTKGPSVFPLAPCSRSTSESTAALGCLVKDYFPEPVTVSWNSGALTSGV
HTFPAVLQSSGLYSLSSVVTVPSSSLGTKTYTCNVDHKPSNTKVDKRVES
KYGPPCPPCPAPEFEGGPSVFLFPPKPKDTLYITREPEVTCVVVDVSQED
PEVQFNWYVDGVEVHNAKTKPREEQFNSTYRVVSVLTVLHQDWLNGKEYK
CKVSNKGLPSSIEKTISKAKGQPREPQVYTLPPSQEEMTKNQVSLTCLVK
GFYPSDIAVEWESNGQPENNYKTTPPVLDSDGSFFLYSRLTVDKSRWQEG
NVFSCSVMHEALHNHYTQKSLSLSLG

In some embodiments, the antibody, such as the light chain, comprises a kappa constant region, such as the human constant domain, which can comprise a sequence of:

Human kappa constant domain
(SEQ ID NO: 69)
RTVAAPSVFIFPPSDEQLKSGTASVVCLLNNFYPREAKVQWKVDNALQSG
NSQESVTEQDSKDSTYSLSSTLTLSKADYEKHKVYACEVTHQGLSSPVTK
SFNRGEC

In some embodiments, the antibody or antigen-binding fragment thereof of any of the embodiments as provided for herein may comprise any constant domain known, such as but not limited to an IgG constant domain. In some embodiments, the constant domain is as provided for herein. In some embodiments, the constant domain is selected from the group including, but not limited to, SEQ ID NO: 63, SEQ ID NO: 64, SEQ ID NO: 65, SEQ ID NO: 66, SEQ ID NO: 67, SEQ ID NO: 68, SEQ ID NO: 69, or a variant thereof.

In some embodiments, the antibody comprises a heavy chain comprising the variable region of SEQ ID NO: 4 and the constant domain of SEQ ID NO: 63. In some embodiments, the antibody comprises a heavy chain comprising the variable region of SEQ ID NO: 4 and the constant domain of SEQ ID NO: 64. In some embodiments, the antibody comprises a heavy chain comprising the variable region of SEQ ID NO: 4 and the constant domain of SEQ ID NO: 65. In some embodiments, the antibody comprises a heavy chain comprising the variable region of SEQ ID NO: 4 and the constant domain of SEQ ID NO: 66. In some embodiments, the antibody comprises a heavy chain comprising the variable region of SEQ ID NO: 4 and the constant domain of SEQ ID NO: 67. In some embodiments, the antibody comprises a heavy chain comprising the variable region of SEQ ID NO: 4 and the constant domain of SEQ ID NO: 68.

In some embodiments, the antibody comprises a heavy chain comprising the variable region of SEQ ID NO: 5 and the constant domain of SEQ ID NO: 63. In some embodiments, the antibody comprises a heavy chain comprising the variable region of SEQ ID NO: 5 and the constant domain of SEQ ID NO: 64. In some embodiments, the antibody comprises a heavy chain comprising the variable region of SEQ ID NO: 5 and the constant domain of SEQ ID NO: 65. In some embodiments, the antibody comprises a heavy chain comprising the variable region of SEQ ID NO: 5 and the constant domain of SEQ ID NO: 66. In some embodiments, the antibody comprises a heavy chain comprising the variable region of SEQ ID NO: 5 and the constant domain of SEQ ID NO: 67. In some embodiments, the antibody comprises a heavy chain comprising the variable region of SEQ ID NO: 5 and the constant domain of SEQ ID NO: 68.

In some embodiments, the antibody comprises a heavy chain comprising the variable region of SEQ ID NO: 6 and the constant domain of SEQ ID NO: 63. In some embodiments, the antibody comprises a heavy chain comprising the variable region of SEQ ID NO: 6 and the constant domain of SEQ ID NO: 64. In some embodiments, the antibody comprises a heavy chain comprising the variable region of SEQ ID NO: 6 and the constant domain of SEQ ID NO: 65. In some embodiments, the antibody comprises a heavy chain comprising the variable region of SEQ ID NO: 6 and the constant domain of SEQ ID NO: 66. In some embodiments, the antibody comprises a heavy chain comprising the variable region of SEQ ID NO: 6 and the constant domain of SEQ ID NO: 67. In some embodiments, the antibody comprises a heavy chain comprising the variable region of SEQ ID NO: 6 and the constant domain of SEQ ID NO: 68.

In some embodiments, the antibody comprises a heavy chain comprising the variable region of SEQ ID NO: 7 and the constant domain of SEQ ID NO: 63. In some embodiments, the antibody comprises a heavy chain comprising the variable region of SEQ ID NO: 7 and the constant domain of SEQ ID NO: 64. In some embodiments, the antibody comprises a heavy chain comprising the variable region of SEQ ID NO: 7 and the constant domain of SEQ ID NO: 65. In some embodiments, the antibody comprises a heavy chain comprising the variable region of SEQ ID NO: 7 and the constant domain of SEQ ID NO: 66. In some embodiments, the antibody comprises a heavy chain comprising the variable region of SEQ ID NO: 7 and the constant domain of SEQ ID NO: 67. In some embodiments, the antibody comprises a heavy chain comprising the variable region of SEQ ID NO: 7 and the constant domain of SEQ ID NO: 68.

In some embodiments, the antibody comprises a heavy chain comprising the variable region of SEQ ID NO: 8 and the constant domain of SEQ ID NO: 63. In some embodiments, the antibody comprises a heavy chain comprising the variable region of SEQ ID NO: 8 and the constant domain of SEQ ID NO: 64. In some embodiments, the antibody comprises a heavy chain comprising the variable region of SEQ ID NO: 8 and the constant domain of SEQ ID NO: 65. In some embodiments, the antibody comprises a heavy chain comprising the variable region of SEQ ID NO: 8 and the constant domain of SEQ ID NO: 66. In some embodiments, the antibody comprises a heavy chain comprising the variable region of SEQ ID NO: 8 and the constant domain of SEQ ID NO: 67. In some embodiments, the antibody comprises a heavy chain comprising the variable region of SEQ ID NO: 8 and the constant domain of SEQ ID NO: 68.

In some embodiments, the antibody comprises a heavy chain comprising the variable region of SEQ ID NO: 9 and the constant domain of SEQ ID NO: 63. In some embodiments, the antibody comprises a heavy chain comprising the variable region of SEQ ID NO: 9 and the constant domain of SEQ ID NO: 64. In some embodiments, the antibody comprises a heavy chain comprising the variable region of SEQ ID NO: 9 and the constant domain of SEQ ID NO: 65. In some embodiments, the antibody comprises a heavy chain comprising the variable region of SEQ ID NO: 9 and the constant domain of SEQ ID NO: 66. In some embodiments, the antibody comprises a heavy chain comprising the variable region of SEQ ID NO: 9 and the constant domain of SEQ ID NO: 67. In some embodiments, the antibody comprises a heavy chain comprising the variable region of SEQ ID NO: 9 and the constant domain of SEQ ID NO: 68.

In some embodiments, the antibody comprises a heavy chain comprising the variable region of SEQ ID NO: 10 and the constant domain of SEQ ID NO: 63. In some embodiments, the antibody comprises a heavy chain comprising the variable region of SEQ ID NO: 10 and the constant domain of SEQ ID NO: 64. In some embodiments, the antibody comprises a heavy chain comprising the variable region of SEQ ID NO: 10 and the constant domain of SEQ ID NO: 65. In some embodiments, the antibody comprises a heavy chain comprising the variable region of SEQ ID NO: 10 and the constant domain of SEQ ID NO: 66. In some embodiments, the antibody comprises a heavy chain comprising the variable region of SEQ ID NO: 10 and the constant domain of SEQ ID NO: 67. In some embodiments, the antibody comprises a heavy chain comprising the variable region of SEQ ID NO: 10 and the constant domain of SEQ ID NO: 68.

In some embodiments, the antibody comprises a heavy chain comprising the variable region of SEQ ID NO: 11 and the constant domain of SEQ ID NO: 63. In some embodiments, the antibody comprises a heavy chain comprising the variable region of SEQ ID NO: 11 and the constant domain of SEQ ID NO: 64. In some embodiments, the antibody comprises a heavy chain comprising the variable region of SEQ ID NO: 11 and the constant domain of SEQ ID NO: 65. In some embodiments, the antibody comprises a heavy chain comprising the variable region of SEQ ID NO: 11 and the constant domain of SEQ ID NO: 66. In some embodiments, the antibody comprises a heavy chain comprising the variable region of SEQ ID NO: 11 and the constant domain of SEQ ID NO: 67. In some embodiments, the antibody comprises a heavy chain comprising the variable region of SEQ ID NO: 11 and the constant domain of SEQ ID NO: 68.

In some embodiments, the antibody comprises a heavy chain comprising the variable region of SEQ ID NO: 12 and the constant domain of SEQ ID NO: 63. In some embodiments, the antibody comprises a heavy chain comprising the variable region of SEQ ID NO: 12 and the constant domain of SEQ ID NO: 64. In some embodiments, the antibody comprises a heavy chain comprising the variable region of SEQ ID NO: 12 and the constant domain of SEQ ID NO: 65. In some embodiments, the antibody comprises a heavy chain comprising the variable region of SEQ ID NO: 12 and the constant domain of SEQ ID NO: 66. In some embodiments, the antibody comprises a heavy chain comprising the variable region of SEQ ID NO: 12 and the constant domain of SEQ ID NO: 67. In some embodiments, the antibody comprises a heavy chain comprising the variable region of SEQ ID NO: 12 and the constant domain of SEQ ID NO: 68.

In some embodiments, the antibody comprises a heavy chain comprising the variable region of SEQ ID NO: 13 and the constant domain of SEQ ID NO: 63. In some embodiments, the antibody comprises a heavy chain comprising the variable region of SEQ ID NO: 13 and the constant domain of SEQ ID NO: 64. In some embodiments, the antibody comprises a heavy chain comprising the variable region of SEQ ID NO: 13 and the constant domain of SEQ ID NO: 65. In some embodiments, the antibody comprises a heavy chain comprising the variable region of SEQ ID NO: 13 and the constant domain of SEQ ID NO: 66. In some embodiments, the antibody comprises a heavy chain comprising the variable region of SEQ ID NO: 13 and the constant domain of SEQ ID NO: 67. In some embodiments, the antibody comprises a heavy chain comprising the variable region of SEQ ID NO: 13 and the constant domain of SEQ ID NO: 68.

In some embodiments, the antibody comprises a heavy chain comprising the variable region of SEQ ID NO: 14 and the constant domain of SEQ ID NO: 63. In some embodiments, the antibody comprises a heavy chain comprising the variable region of SEQ ID NO: 14 and the constant domain of SEQ ID NO: 64. In some embodiments, the antibody comprises a heavy chain comprising the variable region of SEQ ID NO: 14 and the constant domain of SEQ ID NO: 65. In some embodiments, the antibody comprises a heavy chain comprising the variable region of SEQ ID NO: 14 and the constant domain of SEQ ID NO: 66. In some embodiments, the antibody comprises a heavy chain comprising the variable region of SEQ ID NO: 14 and the constant domain of SEQ ID NO: 67. In some embodiments, the antibody comprises a heavy chain comprising the variable region of SEQ ID NO: 14 and the constant domain of SEQ ID NO: 68.

In some embodiments, the antibody comprises a heavy chain comprising the variable region of SEQ ID NO: 15 and the constant domain of SEQ ID NO: 63. In some embodiments, the antibody comprises a heavy chain comprising the variable region of SEQ ID NO: 15 and the constant domain of SEQ ID NO: 64. In some embodiments, the antibody comprises a heavy chain comprising the variable region of SEQ ID NO: 15 and the constant domain of SEQ ID NO: 65. In some embodiments, the antibody comprises a heavy chain comprising the variable region of SEQ ID NO: 15 and the constant domain of SEQ ID NO: 66. In some embodiments, the antibody comprises a heavy chain comprising the variable region of SEQ ID NO: 15 and the constant domain of SEQ ID NO: 67. In some embodiments, the antibody comprises a heavy chain comprising the variable region of SEQ ID NO: 15 and the constant domain of SEQ ID NO: 68.

In some embodiments, the antibody comprises a heavy chain comprising the variable region of SEQ ID NO: 30 and the constant domain of SEQ ID NO: 63. In some embodiments, the antibody comprises a heavy chain comprising the variable region of SEQ ID NO: 30 and the constant domain of SEQ ID NO: 64. In some embodiments, the antibody comprises a heavy chain comprising the variable region of SEQ ID NO: 30 and the constant domain of SEQ ID NO: 65. In some embodiments, the antibody comprises a heavy chain comprising the variable region of SEQ ID NO: 30 and the constant domain of SEQ ID NO: 66. In some embodiments, the antibody comprises a heavy chain comprising the variable region of SEQ ID NO: 30 and the constant domain of SEQ ID NO: 67. In some embodiments, the antibody comprises a heavy chain comprising the variable region of SEQ ID NO: 30 and the constant domain of SEQ ID NO: 68.

In some embodiments, the antibody comprises a heavy chain comprising the variable region of SEQ ID NO: 31 and the constant domain of SEQ ID NO: 63. In some embodiments, the antibody comprises a heavy chain comprising the variable region of SEQ ID NO: 31 and the constant domain of SEQ ID NO: 64. In some embodiments, the antibody comprises a heavy chain comprising the variable region of SEQ ID NO: 31 and the constant domain of SEQ ID NO: 65. In some embodiments, the antibody comprises a heavy chain comprising the variable region of SEQ ID NO: 31 and the constant domain of SEQ ID NO: 66. In some embodiments, the antibody comprises a heavy chain comprising the variable region of SEQ ID NO: 31 and the constant domain of SEQ ID NO: 67. In some embodiments, the antibody comprises a heavy chain comprising the variable region of SEQ ID NO: 31 and the constant domain of SEQ ID NO: 68.

In some embodiments, the antibody comprises a heavy chain comprising the variable region of SEQ ID NO: 32 and the constant domain of SEQ ID NO: 63. In some embodiments, the antibody comprises a heavy chain comprising the variable region of SEQ ID NO: 32 and the constant domain of SEQ ID NO: 64. In some embodiments, the antibody comprises a heavy chain comprising the variable region of SEQ ID NO: 32 and the constant domain of SEQ ID NO: 65. In some embodiments, the antibody comprises a heavy chain comprising the variable region of SEQ ID NO: 32 and the constant domain of SEQ ID NO: 66. In some embodiments, the antibody comprises a heavy chain comprising the variable region of SEQ ID NO: 32 and the constant domain of SEQ ID NO: 67. In some embodiments, the antibody comprises a heavy chain comprising the variable region of SEQ ID NO: 32 and the constant domain of SEQ ID NO: 68.

In some embodiments, the antibody comprises a heavy chain comprising the variable region of SEQ ID NO: 33 and the constant domain of SEQ ID NO: 63. In some embodiments, the antibody comprises a heavy chain comprising the variable region of SEQ ID NO: 33 and the constant domain of SEQ ID NO: 64. In some embodiments, the antibody comprises a heavy chain comprising the variable region of SEQ ID NO: 33 and the constant domain of SEQ ID NO: 65. In some embodiments, the antibody comprises a heavy chain comprising the variable region of SEQ ID NO: 33 and the constant domain of SEQ ID NO: 66. In some embodiments, the antibody comprises a heavy chain comprising the variable region of SEQ ID NO: 33 and the constant domain of SEQ ID NO: 67. In some embodiments, the antibody comprises a heavy chain comprising the variable region of SEQ ID NO: 33 and the constant domain of SEQ ID NO: 68.

In some embodiments, the antibody comprises a heavy chain comprising the variable region of SEQ ID NO: 44 and the constant domain of SEQ ID NO: 63. In some embodiments, the antibody comprises a heavy chain comprising the variable region of SEQ ID NO: 44 and the constant domain of SEQ ID NO: 64. In some embodiments, the antibody comprises a heavy chain comprising the variable region of SEQ ID NO: 44 and the constant domain of SEQ ID NO: 65. In some embodiments, the antibody comprises a heavy chain comprising the variable region of SEQ ID NO: 44 and the constant domain of SEQ ID NO: 66. In some embodiments, the antibody comprises a heavy chain comprising the variable region of SEQ ID NO: 44 and the constant domain of SEQ ID NO: 67. In some embodiments, the antibody comprises a heavy chain comprising the variable region of SEQ ID NO: 44 and the constant domain of SEQ ID NO: 68.

In some embodiments, the antibody comprises a heavy chain comprising the variable region of SEQ ID NO: 45 and the constant domain of SEQ ID NO: 63. In some embodiments, the antibody comprises a heavy chain comprising the variable region of SEQ ID NO: 45 and the constant domain of SEQ ID NO: 64. In some embodiments, the antibody comprises a heavy chain comprising the variable region of SEQ ID NO: 45 and the constant domain of SEQ ID NO: 65. In some embodiments, the antibody comprises a heavy chain comprising the variable region of SEQ ID NO: 45 and the constant domain of SEQ ID NO: 66. In some embodiments, the antibody comprises a heavy chain comprising the variable region of SEQ ID NO: 45 and the constant domain of SEQ ID NO: 67. In some embodiments, the antibody comprises a heavy chain comprising the variable region of SEQ ID NO: 45 and the constant domain of SEQ ID NO: 68.

In some embodiments, the antibody comprises a heavy chain comprising the variable region of SEQ ID NO: 46 and the constant domain of SEQ ID NO: 63. In some embodiments, the antibody comprises a heavy chain comprising the variable region of SEQ ID NO: 46 and the constant domain of SEQ ID NO: 64. In some embodiments, the antibody comprises a heavy chain comprising the variable region of SEQ ID NO: 46 and the constant domain of SEQ ID NO: 65. In some embodiments, the antibody comprises a heavy chain comprising the variable region of SEQ ID NO: 46 and the constant domain of SEQ ID NO: 66. In some embodiments, the antibody comprises a heavy chain comprising the variable region of SEQ ID NO: 46 and the constant domain of SEQ ID NO: 67. In some embodiments, the antibody comprises a heavy chain comprising the variable region of SEQ ID NO: 46 and the constant domain of SEQ ID NO: 68.

In some embodiments, the antibody comprises a heavy chain comprising the variable region of SEQ ID NO: 47 and the constant domain of SEQ ID NO: 63. In some embodiments, the antibody comprises a heavy chain comprising the variable region of SEQ ID NO: 47 and the constant domain of SEQ ID NO: 64. In some embodiments, the antibody comprises a heavy chain comprising the variable region of SEQ ID NO: 47 and the constant domain of SEQ ID NO: 65. In some embodiments, the antibody comprises a heavy chain comprising the variable region of SEQ ID NO: 47 and the constant domain of SEQ ID NO: 66. In some embodiments, the antibody comprises a heavy chain comprising the variable region of SEQ ID NO: 47 and the constant domain of SEQ ID NO: 67. In some embodiments, the antibody comprises a heavy chain comprising the variable region of SEQ ID NO: 47 and the constant domain of SEQ ID NO: 68.

In some embodiments, the antibody comprises a heavy chain comprising the variable region of SEQ ID NO: 48 and the constant domain of SEQ ID NO: 63. In some embodiments, the antibody comprises a heavy chain comprising the variable region of SEQ ID NO: 48 and the constant domain of SEQ ID NO: 64. In some embodiments, the antibody comprises a heavy chain comprising the variable region of SEQ ID NO: 48 and the constant domain of SEQ ID NO: 65. In some embodiments, the antibody comprises a heavy chain comprising the variable region of SEQ ID NO: 48 and the constant domain of SEQ ID NO: 66. In some embodiments, the antibody comprises a heavy chain comprising the variable region of SEQ ID NO: 48 and the constant domain of SEQ ID NO: 67. In some embodiments, the antibody comprises a heavy chain comprising the variable region of SEQ ID NO: 48 and the constant domain of SEQ ID NO: 68.

In some embodiments, the antibody comprises a heavy chain comprising the variable region of SEQ ID NO: 49 and the constant domain of SEQ ID NO: 63. In some embodiments, the antibody comprises a heavy chain comprising the variable region of SEQ ID NO: 49 and the constant domain of SEQ ID NO: 64. In some embodiments, the antibody comprises a heavy chain comprising the variable region of SEQ ID NO: 64 and the constant domain of SEQ ID NO: 65. In some embodiments, the antibody comprises a heavy chain comprising the variable region of SEQ ID NO: 49 and the constant domain of SEQ ID NO: 66. In some embodiments, the antibody comprises a heavy chain comprising the variable region of SEQ ID NO: 49 and the constant domain of SEQ ID NO: 67. In some embodiments, the antibody comprises a heavy chain comprising the variable region of SEQ ID NO: 49 and the constant domain of SEQ ID NO: 68.

In some embodiments, the antibody comprises a heavy chain comprising the variable region of SEQ ID NO: 50 and the constant domain of SEQ ID NO: 63. In some embodiments, the antibody comprises a heavy chain comprising the variable region of SEQ ID NO: 50 and the constant domain of SEQ ID NO: 64. In some embodiments, the antibody comprises a heavy chain comprising the variable region of SEQ ID NO: 50 and the constant domain of SEQ ID NO: 65. In some embodiments, the antibody comprises a heavy chain comprising the variable region of SEQ ID NO: 50 and the constant domain of SEQ ID NO: 66. In some embodiments, the antibody comprises a heavy chain comprising the variable region of SEQ ID NO: 50 and the constant domain of SEQ ID NO: 67. In some embodiments, the antibody comprises a heavy chain comprising the variable region of SEQ ID NO: 50 and the constant domain of SEQ ID NO: 68.

In some embodiments, the antibody comprises a heavy chain comprising the variable region of SEQ ID NO: 51 and the constant domain of SEQ ID NO: 63. In some embodiments, the antibody comprises a heavy chain comprising the variable region of SEQ ID NO: 51 and the constant domain of SEQ ID NO: 64. In some embodiments, the antibody comprises a heavy chain comprising the variable region of SEQ ID NO: 51 and the constant domain of SEQ ID NO: 65. In some embodiments, the antibody comprises a heavy chain comprising the variable region of SEQ ID NO: 51 and the constant domain of SEQ ID NO: 66. In some embodiments, the antibody comprises a heavy chain comprising the variable region of SEQ ID NO: 51 and the constant domain of SEQ ID NO: 67. In some embodiments, the antibody comprises a heavy chain comprising the variable region of SEQ ID NO: 51 and the constant domain of SEQ ID NO: 68.

In some embodiments, the antibody comprises a heavy chain comprising the variable region of SEQ ID NO: 52 and the constant domain of SEQ ID NO: 63. In some embodiments, the antibody comprises a heavy chain comprising the variable region of SEQ ID NO: 52 and the constant domain of SEQ ID NO: 64. In some embodiments, the antibody comprises a heavy chain comprising the variable region of SEQ ID NO: 52 and the constant domain of SEQ ID NO: 65. In some embodiments, the antibody comprises a heavy chain comprising the variable region of SEQ ID NO: 52 and the constant domain of SEQ ID NO: 66. In some embodiments, the antibody comprises a heavy chain comprising the variable region of SEQ ID NO: 52 and the constant domain of SEQ ID NO: 67. In some embodiments, the antibody comprises a heavy chain comprising the variable region of SEQ ID NO: 52 and the constant domain of SEQ ID NO: 68.

In some embodiments, the antibody comprises a heavy chain comprising the variable region of SEQ ID NO: 53 and the constant domain of SEQ ID NO: 63. In some embodiments, the antibody comprises a heavy chain comprising the variable region of SEQ ID NO: 53 and the constant domain of SEQ ID NO: 64. In some embodiments, the antibody comprises a heavy chain comprising the variable region of SEQ ID NO: 53 and the constant domain of SEQ ID NO: 65. In some embodiments, the antibody comprises a heavy chain comprising the variable region of SEQ ID NO: 53 and the constant domain of SEQ ID NO: 66. In some embodiments, the antibody comprises a heavy chain comprising the variable region of SEQ ID NO: 53 and the constant domain of SEQ ID NO: 67. In some embodiments, the antibody comprises a heavy chain comprising the variable region of SEQ ID NO: 53 and the constant domain of SEQ ID NO: 68.

In some embodiments, the antibody comprises a heavy chain comprising the variable region of SEQ ID NO: 54 and the constant domain of SEQ ID NO: 63. In some embodiments, the antibody comprises a heavy chain comprising the variable region of SEQ ID NO: 54 and the constant domain of SEQ ID NO: 64. In some embodiments, the antibody comprises a heavy chain comprising the variable region of SEQ ID NO: 54 and the constant domain of SEQ ID NO: 65. In some embodiments, the antibody comprises a heavy chain comprising the variable region of SEQ ID NO: 54 and the constant domain of SEQ ID NO: 66. In some embodiments, the antibody comprises a heavy chain comprising the variable region of SEQ ID NO: 54 and the constant domain of SEQ ID NO: 67. In some embodiments, the antibody comprises a heavy chain comprising the variable region of SEQ ID NO: 54 and the constant domain of SEQ ID NO: 68.

In some embodiments, the antibody comprises a heavy chain comprising the variable region of SEQ ID NO: 55 and the constant domain of SEQ ID NO: 63. In some embodiments, the antibody comprises a heavy chain comprising the variable region of SEQ ID NO: 55 and the constant domain of SEQ ID NO: 64. In some embodiments, the antibody comprises a heavy chain comprising the variable region of SEQ ID NO: 55 and the constant domain of SEQ ID NO: 65. In some embodiments, the antibody comprises a heavy chain comprising the variable region of SEQ ID NO: 55 and the constant domain of SEQ ID NO: 66. In some embodiments, the antibody comprises a heavy chain comprising the variable region of SEQ ID NO: 55 and the constant domain of SEQ ID NO: 67. In some embodiments, the antibody comprises a heavy chain comprising the variable region of SEQ ID NO: 55 and the constant domain of SEQ ID NO: 68.

In some embodiments, the antibody comprises a heavy chain comprising the variable region of SEQ ID NO: 56 and the constant domain of SEQ ID NO: 63. In some embodiments, the antibody comprises a heavy chain comprising the variable region of SEQ ID NO: 56 and the constant domain of SEQ ID NO: 64. In some embodiments, the antibody comprises a heavy chain comprising the variable region of SEQ ID NO: 56 and the constant domain of SEQ ID NO: 65. In some embodiments, the antibody comprises a heavy chain comprising the variable region of SEQ ID NO: 56 and the constant domain of SEQ ID NO: 66. In some embodiments, the antibody comprises a heavy chain comprising the variable region of SEQ ID NO: 56 and the constant domain of SEQ ID NO: 67. In some embodiments, the antibody comprises a heavy chain comprising the variable region of SEQ ID NO: 56 and the constant domain of SEQ ID NO: 68.

In some embodiments, the antibody comprises a heavy chain comprising the variable region of SEQ ID NO: 57 and the constant domain of SEQ ID NO: 63. In some embodiments, the antibody comprises a heavy chain comprising the variable region of SEQ ID NO: 57 and the constant domain of SEQ ID NO: 64. In some embodiments, the antibody comprises a heavy chain comprising the variable region of SEQ ID NO: 57 and the constant domain of SEQ ID NO: 65. In some embodiments, the antibody comprises a heavy chain comprising the variable region of SEQ ID NO: 57 and the constant domain of SEQ ID NO: 66. In some embodiments, the antibody comprises a heavy chain comprising the variable region of SEQ ID NO: 57 and the constant domain of SEQ ID NO: 67. In some embodiments, the antibody comprises a heavy chain comprising the variable region of SEQ ID NO: 57 and the constant domain of SEQ ID NO: 68.

In some embodiments, the antibody comprises a light chain comprising the variable region of SEQ ID NO: 19 and the constant domain of SEQ ID NO: 69. In some embodiments, the antibody comprises a light chain comprising the variable region of SEQ ID NO: 20 and the constant domain of SEQ ID NO: 69. In some embodiments, the antibody comprises a light chain comprising the variable region of SEQ ID NO: 21 and the constant domain of SEQ ID NO: 69. In some embodiments, the antibody comprises a light chain comprising the variable region of SEQ ID NO: 22 and the constant domain of SEQ ID NO: 69. In some embodiments, the antibody comprises a light chain comprising the variable region of SEQ ID NO: 37 and the constant domain of SEQ ID NO: 69. In some embodiments, the antibody comprises a light chain comprising the variable region of SEQ ID NO: 61 and the constant domain of SEQ ID NO: 69. In some embodiments, the antibody comprises a light chain comprising the variable region of SEQ ID NO: 62 and the constant domain of SEQ ID NO: 69.

In some embodiments, the antibody, or any antibody fragment thereof, comprises a heavy chain selected from the Table 8 below.

TABLE 8
antibody heavy chains

| | |
|---|---|
| SEQ | QIQLVQSGSELKKPGASVKVSCKASGYTFTDYAMNWVRQAPGQGLEWMGW |
| ID | INTQTGKPTYAQGFTGRFVFSLDTSVSTAYLQISSLKAEDTAVYYCTRLGTGN |
| NO: | TKGFAYWGQGTLVTVSSASTKGPSVFPLAPSSKSTSGGTAALGCLVKDYFPEP |
| 72 | VTVSWNSGALTSGVHTFPAVLQSSGLYSLSSVVTVPSSSLGTQTYICNVNHKP |
| | SNTKVDKKVEPKSCDKTHTCPPCPAPEAAGGPSVFLFPPKPKDTLMISRTPEVT |
| | CVVVSVSHEDPEVKFNWYVDGVEVHNAKTKPREEQYNSTYRVVSVLTVLHQ |
| | DWLNGKEYKCKVSNKALPAPIEKTISKAKGQPREPQVYTLPPSREEMTKNQV |
| | SLTCLVKGFYPSDIAVEWESNGQPENNYKTTPPVLDSDGSFFLYSKLTVDKSR |
| | WQQGNVFSCSVMHEALHNHYTQKSLSLSPGK |
| SEQ | QIQLVQSGAEVKKPGSSVKVSCKASGYTFTDYAMNWVRQAPGQGLEWMGW |
| ID | INTQTGKPTYAQKFQGRVTFTLDESTSTAYMELSSLRSEDTAVYYCTRLGTGN |
| NO: | TKGFAYWGQGTLVTVSSASTKGPSVFPLAPSSKSTSGGTAALGCLVKDYFPEP |
| 73 | VTVSWNSGALTSGVHTFPAVLQSSGLYSLSSVVTVPSSSLGTQTYICNVNHKP |
| | SNTKVDKKVEPKSCDKTHTCPPCPAPEAAGGPSVFLFPPKPKDTLMISRTPEVT |
| | CVVVSVSHEDPEVKFNWYVDGVEVHNAKTKPREEQYNSTYRVVSVLTVLHQ |
| | DWLNGKEYKCKVSNKALPAPIEKTISKAKGQPREPQVYTLPPSREEMTKNQV |
| | SLTCLVKGFYPSDIAVEWESNGQPENNYKTTPPVLDSDGSFFLYSKLTVDKSR |
| | WQQGNVFSCSVMHEALHNHYTQKSLSLSPGK |
| SEQ | QIQLVQSGAEVKKPGSSVKVSCKASGYTFTDYAMNWVRQAPGQGLEWMGW |
| ID | INTQTGKPTYAQKFQGRVTFTLDESTSTTYMELSSLRSEDTAVYYCARLGTGN |
| NO: | TKGFAYWGQGTLVTVSSASTKGPSVFPLAPSSKSTSGGTAALGCLVKDYFPEP |
| 74 | VTVSWNSGALTSGVHTFPAVLQSSGLYSLSSVVTVPSSSLGTQTYICNVNHKP |
| | SNTKVDKKVEPKSCDKTHTCPPCPAPEAAGGPSVFLFPPKPKDTLMISRTPEVT |
| | CVVVSVSHEDPEVKFNWYVDGVEVHNAKTKPREEQYNSTYRVVSVLTVLHQ |
| | DWLNGKEYKCKVSNKALPAPIEKTISKAKGQPREPQVYTLPPSREEMTKNQV |
| | SLTCLVKGFYPSDIAVEWESNGQPENNYKTTPPVLDSDGSFFLYSKLTVDKSR |
| | WQQGNVFSCSVMHEALHNHYTQKSLSLSPGK |
| SEQ | QIQLVQSGAEVKKPGSSVKVSCKASGYTFTDYAMNWVRQAPGQGLEWMGW |
| ID | INTQTGKPTYAQKFQGRVTFTLDESTSTTYMELSSLRSEDTAVYYCTRLGTGN |
| NO: | TKGFAYWGQGTLVTVSSASTKGPSVFPLAPSSKSTSGGTAALGCLVKDYFPEP |
| 75 | VTVSWNSGALTSGVHTFPAVLQSSGLYSLSSVVTVPSSSLGTQTYICNVNHKP |
| | SNTKVDKKVEPKSCDKTHTCPPCPAPEAAGGPSVFLFPPKPKDTLMISRTPEVT |
| | CVVVSVSHEDPEVKFNWYVDGVEVHNAKTKPREEQYNSTYRVVSVLTVLHQ |
| | DWLNGKEYKCKVSNKALPAPIEKTISKAKGQPREPQVYTLPPSREEMTKNQV |
| | SLTCLVKGFYPSDIAVEWESNGQPENNYKTTPPVLDSDGSFFLYSKLTVDKSR |
| | WQQGNVFSCSVMHEALHNHYTQKSLSLSPGK |
| SEQ | QIQLVQSGPEVKKPGESVKISCKASGYTFTDYAMNWVKQAPGNGLKWMGWI |
| ID | NTQTGKPTYADDFKQRFVFSLETSARTTYLQINNLNIEDTATYFCTRLGTGNT |
| NO: | KGFAYWGQGTLVTVSSASTKGPSVFPLAPSSKSTSGGTAALGCLVKDYFPEPV |
| 76 | TVSWNSGALTSGVHTFPAVLQSSGLYSLSSVVTVPSSSLGTQTYICNVNHKPS |
| | NTKVDKKVEPKSCDKTHTCPPCPAPEAAGGPSVFLFPPKPKDTLMISRTPEVT |
| | CVVVSVSHEDPEVKFNWYVDGVEVHNAKTKPREEQYNSTYRVVSVLTVLHQ |
| | DWLNGKEYKCKVSNKALPAPIEKTISKAKGQPREPQVYTLPPSREEMTKNQV |
| | SLTCLVKGFYPSDIAVEWESNGQPENNYKTTPPVLDSDGSFFLYSKLTVDKSR |
| | WQQGNVFSCSVMHEALHNHYTQKSLSLSPGK |
| SEQ | QIQLVQSGSELKKPGASVKISCKASGYTFTDYAMNWVKQAPGQGLEWMGWI |
| ID | NTQTGKPTYAQGFTGRFVFSLDTSVSTAYLQISSLKAEDTAVYFCTRLGTGNT |
| NO: | KGFAYWGQGTLVTVSSASTKGPSVFPLAPSSKSTSGGTAALGCLVKDYFPEPV |
| 77 | TVSWNSGALTSGVHTFPAVLQSSGLYSLSSVVTVPSSSLGTQTYICNVNHKPS |
| | NTKVDKKVEPKSCDKTHTCPPCPAPEAAGGPSVFLFPPKPKDTLMISRTPEVT |
| | CVVVSVSHEDPEVKFNWYVDGVEVHNAKTKPREEQYNSTYRVVSVLTVLHQ |
| | DWLNGKEYKCKVSNKALPAPIEKTISKAKGQPREPQVYTLPPSREEMTKNQV |
| | SLTCLVKGFYPSDIAVEWESNGQPENNYKTTPPVLDSDGSFFLYSKLTVDKSR |
| | WQQGNVFSCSVMHEALHNHYTQKSLSLSPGK |
| SEQ | QIQLVQSGSELKKPGASVKISCKASGYTFTDYAMNWVKQAPGQGLEWMGWI |
| ID | NTQTGKPTYAQGFTGRFVFSLDTSVSTAYLQISSLKAEDTAVYFCTRLGTGNT |
| NO: | KGFAYWGQGTLVTVSSASTKGPSVFPLAPSSKSTSGGTAALGCLVKDYFPEPV |
| 78 | TVSWNSGALTSGVHTFPAVLQSSGLYSLSSVVTVPSSSLGTQTYICNVNHKPS |
| | NTKVDKKVEPKSCDKTHTCPPCPAPEAAGGPSVFLFPPKPKDTLMISRTPEVT |
| | CVVVSVSHEDPEVKFNWYVDGVEVHNAKTKPREEQYNSTYRVVSVLTVLHQ |
| | DWLNGKEYKCKVSNKALPAPIEKTISKAKGQPREPQVYTLPPSREEMTKNQV |
| | SLTCLVKGFYPSDIAVEWESNGQPENNYKTTPPVLDSDGSFFLYSKLTVDKSR |
| | WQQGNVFSCSVMHEALHNHYTQKSLSLSPGK |
| SEQ | QIQLVQSGSELKKPGASVKISCKASGYTFTDYAMNWVRQAPGQGLEWMGWI |
| ID | NTQTGKPTYAQGFTGRFVFSLDTSVRTAYLQISSLKAEDTAVYFCTRLGTGNT |
| NO: | KGFAYWGQGTLVTVSSASTKGPSVFPLAPSSKSTSGGTAALGCLVKDYFPEPV |
| 79 | TVSWNSGALTSGVHTFPAVLQSSGLYSLSSVVTVPSSSLGTQTYICNVNHKPS |
| | NTKVDKKVEPKSCDKTHTCPPCPAPEAAGGPSVFLFPPKPKDTLMISRTPEVT |
| | CVVVSVSHEDPEVKFNWYVDGVEVHNAKTKPREEQYNSTYRVVSVLTVLHQ |
| | DWLNGKEYKCKVSNKALPAPIEKTISKAKGQPREPQVYTLPPSREEMTKNQV |
| | SLTCLVKGFYPSDIAVEWESNGQPENNYKTTPPVLDSDGSFFLYSKLTVDKSR |
| | WQQGNVFSCSVMHEALHNHYTQKSLSLSPGK |
| SEQ | QIQLVQSGSELKKPGASVKISCKASGYTFTDYAMNWVRQAPGQGLEWMGWI |
| ID | NTQTGKPTYAQGFTGRFVFSLDTSVSTAYLQISSLKAEDTAVYFCTRLGTGNT |
| NO: | KGFAYWGQGTLVTVSSASTKGPSVFPLAPSSKSTSGGTAALGCLVKDYFPEPV |
| 80 | TVSWNSGALTSGVHTFPAVLQSSGLYSLSSVVTVPSSSLGTQTYICNVNHKPS |
| | NTKVDKKVEPKSCDKTHTCPPCPAPEAAGGPSVFLFPPKPKDTLMISRTPEVT |
| | CVVVSVSHEDPEVKFNWYVDGVEVHNAKTKPREEQYNSTYRVVSVLTVLHQ |
| | DWLNGKEYKCKVSNKALPAPIEKTISKAKGQPREPQVYTLPPSREEMTKNQV |
| | SLTCLVKGFYPSDIAVEWESNGQPENNYKTTPPVLDSDGSFFLYSKLTVDKSR |
| | WQQGNVFSCSVMHEALHNHYTQKSLSLSPGK |
| SEQ | QIQLVQSGSELKKPGASVKISCKASGYTFTDYAMNWVRQAPGQGLEWMGWI |
| ID | NTQTGKPTYAQGFTGRFVFSLDTSVSTAYLQISSLKAEDTAVYYCTRLGTGNT |
| NO: | KGFAYWGQGTLVTVSSASTKGPSVFPLAPSSKSTSGGTAALGCLVKDYFPEPV |
| 81 | TVSWNSGALTSGVHTFPAVLQSSGLYSLSSVVTVPSSSLGTQTYICNVNHKPS |
| | NTKVDKKVEPKSCDKTHTCPPCPAPEAAGGPSVFLFPPKPKDTLMISRTPEVT |
| | CVVVSVSHEDPEVKFNWYVDGVEVHNAKTKPREEQYNSTYRVVSVLTVLHQ |
| | DWLNGKEYKCKVSNKALPAPIEKTISKAKGQPREPQVYTLPPSREEMTKNQV |
| | SLTCLVKGFYPSDIAVEWESNGQPENNYKTTPPVLDSDGSFFLYSKLTVDKSR |
| | WQQGNVFSCSVMHEALHNHYTQKSLSLSPGK |
| SEQ | QIQLVQSGSELKKPGASVKVSCKASGYTFTDYAMNWVRQAPGQGLEWMGW |
| ID | INTQTGKPTYAQGFTGRFVFSLDTSVSTAYLQISSLKAEDTAVYFCTRLGTGN |
| NO: | TKGFAYWGQGTLVTVSSASTKGPSVFPLAPSSKSTSGGTAALGCLVKDYFPEP |
| 82 | VTVSWNSGALTSGVHTFPAVLQSSGLYSLSSVVTVPSSSLGTQTYICNVNHKP |
| | SNTKVDKKVEPKSCDKTHTCPPCPAPEAAGGPSVFLFPPKPKDTLMISRTPEVT |
| | CVVVSVSHEDPEVKFNWYVDGVEVHNAKTKPREEQYNSTYRVVSVLTVLHQ |
| | DWLNGKEYKCKVSNKALPAPIEKTISKAKGQPREPQVYTLPPSREEMTKNQV |
| | SLTCLVKGFYPSDIAVEWESNGQPENNYKTTPPVLDSDGSFFLYSKLTVDKSR |
| | WQQGNVFSCSVMHEALHNHYTQKSLSLSPGK |
| SEQ | QVQLVQSGAEVKKPGSSVKVSCKASGYTFTDYAMNWVRQAPGQGLEWMG |
| ID | WINTQTGKPTYAQKFQGRVTFTLDESTSTAYMELSSLRSEDTAVYYCTRLGT |
| NO: | GNTKGFAYWGQGTLVTVSSASTKGPSVFPLAPSSKSTSGGTAALGCLVKDYF |
| 83 | PEPVTVSWNSGALTSGVHTFPAVLQSSGLYSLSSVVTVPSSSLGTQTYICNVN |
| | HKPSNTKVDKKVEPKSCDKTHTCPPCPAPEAAGGPSVFLFPPKPKDTLMISRT |
| | PEVTCVVVSVSHEDPEVKFNWYVDGVEVHNAKTKPREEQYNSTYRVVSVLT |
| | VLHQDWLNGKEYKCKVSNKALPAPIEKTISKAKGQPREPQVYTLPPSREEMT |
| | KNQVSLTCLVKGFYPSDIAVEWESNGQPENNYKTTPPVLDSDGSFFLYSKLTV |
| | DKSRWQQGNVFSCSVMHEALHNHYTQKSLSLSPGK |
| SEQ | QIQLVQSGSELKKPGASVKVSCKASGYTFTDYAMNWVRQAPGQGLEWMGW |
| ID | INTQTGKPTYAQGFTGRFVFSLDTSVSTAYLQISSLKAEDTAVYYCTRLGTGN |
| NO: | TKGFAYWGQGTLVTVSSASTKGPSVFPLAPCSRSTSESTAALGCLVKDYFPEP |
| 84 | VTVSWNSGALTSGVHTFPAVLQSSGLYSLSSVVTVPSSSLGTKTYTCNVDHKP |
| | SNTKVDKRVESKYGPPCPPCPAPEFEGGPSVFLFPPKPKDTLMISRTPEVTCVV |
| | VDVSQEDPEVQFNWYVDGVEVHNAKTKPREEQFNSTYRVVSVLTVLHQDW |
| | LNGKEYKCKVSNKGLPSSIEKTISKAKGQPREPQVYTLPPSQEEMTKNQVSLT |
| | CLVKGFYPSDIAVEWESNGQPENNYKTTPPVLDSDGSFFLYSRLTVDKSRWQ |
| | EGNVFSCSVLHEALHSHYTQKSLSLSLGK |
| SEQ | QIQLVQSGAEVKKPGSSVKVSCKASGYTFTDYAMNWVRQAPGQGLEWMGW |
| ID | INTQTGKPTYAQKFQGRVTFTLDESTSTAYMELSSLRSEDTAVYYCTRLGTGN |
| NO: | TKGFAYWGQGTLVTVSSASTKGPSVFPLAPCSRSTSESTAALGCLVKDYFPEP |
| 85 | VTVSWNSGALTSGVHTFPAVLQSSGLYSLSSVVTVPSSSLGTKTYTCNVDHKP |
| | SNTKVDKRVESKYGPPCPPCPAPEFEGGPSVFLFPPKPKDTLMISRTPEVTCVV |
| | VDVSQEDPEVQFNWYVDGVEVHNAKTKPREEQFNSTYRVVSVLTVLHQDW |
| | LNGKEYKCKVSNKGLPSSIEKTISKAKGQPREPQVYTLPPSQEEMTKNQVSLT |
| | CLVKGFYPSDIAVEWESNGQPENNYKTTPPVLDSDGSFFLYSRLTVDKSRWQ |
| | EGNVFSCSVLHEALHSHYTQKSLSLSLGK |
| SEQ | QIQLVQSGAEVKKPGSSVKVSCKASGYTFTDYAMNWVRQAPGQGLEWMGW |
| ID | INTQTGKPTYAQKFQGRVTFTLDESTSTTYMELSSLRSEDTAVYYCARLGTGN |
| NO: | TKGFAYWGQGTLVTVSSASTKGPSVFPLAPCSRSTSESTAALGCLVKDYFPEP |
| 86 | VTVSWNSGALTSGVHTFPAVLQSSGLYSLSSVVTVPSSSLGTKTYTCNVDHKP |
| | SNTKVDKRVESKYGPPCPPCPAPEFEGGPSVFLFPPKPKDTLMISRTPEVTCVV |
| | VDVSQEDPEVQFNWYVDGVEVHNAKTKPREEQFNSTYRVVSVLTVLHQDW |
| | LNGKEYKCKVSNKGLPSSIEKTISKAKGQPREPQVYTLPPSQEEMTKNQVSLT |
| | CLVKGFYPSDIAVEWESNGQPENNYKTTPPVLDSDGSFFLYSRLTVDKSRWQ |
| | EGNVFSCSVLHEALHSHYTQKSLSLSLGK |
| SEQ | QIQLVQSGAEVKKPGSSVKVSCKASGYTFTDYAMNWVRQAPGQGLEWMGW |
| ID | INTQTGKPTYAQKFQGRVTFTLDESTSTTYMELSSLRSEDTAVYYCTRLGTGN |
| NO: | TKGFAYWGQGTLVTVSSASTKGPSVFPLAPCSRSTSESTAALGCLVKDYFPEP |
| 87 | VTVSWNSGALTSGVHTFPAVLQSSGLYSLSSVVTVPSSSLGTKTYTCNVDHKP |
| | SNTKVDKRVESKYGPPCPPCPAPEFEGGPSVFLFPPKPKDTLMISRTPEVTCVV |
| | VDVSQEDPEVQFNWYVDGVEVHNAKTKPREEQFNSTYRVVSVLTVLHQDW |
| | LNGKEYKCKVSNKGLPSSIEKTISKAKGQPREPQVYTLPPSQEEMTKNQVSLT |
| | CLVKGFYPSDIAVEWESNGQPENNYKTTPPVLDSDGSFFLYSRLTVDKSRWQ |
| | EGNVFSCSVLHEALHSHYTQKSLSLSLGK |
| SEQ | QIQLVQSGPEVKKPGESVKISCKASGYTFTDYAMNWVKQAPGNGLKWMGWI |
| ID | NTQTGKPTYADDFKQRFVFSLETSARTTYLQINNLNIEDTATYFCTRLGTGNT |
| NO: | KGFAYWGQGTLVTVSSASTKGPSVFPLAPCSRSTSESTAALGCLVKDYFPEPV |
| 88 | TVSWNSGALTSGVHTFPAVLQSSGLYSLSSVVTVPSSSLGTKTYTCNVDHKPS |
| | NTKVDKRVESKYGPPCPPCPAPEFEGGPSVFLFPPKPKDTLMISRTPEVTCVVV |
| | DVSQEDPEVQFNWYVDGVEVHNAKTKPREEQFNSTYRVVSVLTVLHQDWL |
| | NGKEYKCKVSNKGLPSSIEKTISKAKGQPREPQVYTLPPSQEEMTKNQVSLTC |
| | LVKGFYPSDIAVEWESNGQPENNYKTTPPVLDSDGSFFLYSRLTVDKSRWQE |
| | GNVFSCSVLHEALHSHYTQKSLSLSLGK |
| SEQ | QIQLVQSGSELKKPGASVKISCKASGYTFTDYAMNWVKQAPGQGLEWMGWI |
| ID | NTQTGKPTYAQGFTGRFVFSLDTSVSTAYLQISSLKAEDTAVYFCTRLGTGNT |
| NO: | KGFAYWGQGTLVTVSSASTKGPSVFPLAPCSRSTSESTAALGCLVKDYFPEPV |
| 89 | TVSWNSGALTSGVHTFPAVLQSSGLYSLSSVVTVPSSSLGTKTYTCNVDHKPS |
| | NTKVDKRVESKYGPPCPPCPAPEFEGGPSVFLFPPKPKDTLMISRTPEVTCVVV |
| | DVSQEDPEVQFNWYVDGVEVHNAKTKPREEQFNSTYRVVSVLTVLHQDWL |
| | NGKEYKCKVSNKGLPSSIEKTISKAKGQPREPQVYTLPPSQEEMTKNQVSLTC |
| | LVKGFYPSDIAVEWESNGQPENNYKTTPPVLDSDGSFFLYSRLTVDKSRWQE |
| | GNVFSCSVLHEALHSHYTQKSLSLSLGK |
| SEQ | QIQLVQSGSELKKPGASVKISCKASGYTFTDYAMNWVKQAPGQGLEWMGWI |
| ID | NTQTGKPTYAQGFTGRFVFSLDTSVSTAYLQISSLKAEDTAVYFCTRLGTGNT |
| NO: | KGFAYWGQGTLVTVSSASTKGPSVFPLAPCSRSTSESTAALGCLVKDYFPEPV |
| 90 | TVSWNSGALTSGVHTFPAVLQSSGLYSLSSVVTVPSSSLGTKTYTCNVDHKPS |
| | NTKVDKRVESKYGPPCPPCPAPEFEGGPSVFLFPPKPKDTLMISRTPEVTCVVV |
| | DVSQEDPEVQFNWYVDGVEVHNAKTKPREEQFNSTYRVVSVLTVLHQDWL |
| | NGKEYKCKVSNKGLPSSIEKTISKAKGQPREPQVYTLPPSQEEMTKNQVSLTC |
| | LVKGFYPSDIAVEWESNGQPENNYKTTPPVLDSDGSFFLYSRLTVDKSRWQE |
| | GNVFSCSVLHEALHSHYTQKSLSLSLGK |
| SEQ | QIQLVQSGSELKKPGASVKISCKASGYTFTDYAMNWVRQAPGQGLEWMGWI |
| ID | NTQTGKPTYAQGFTGRFVFSLDTSVRTAYLQISSLKAEDTAVYFCTRLGTGNT |
| NO: | KGFAYWGQGTLVTVSSASTKGPSVFPLAPCSRSTSESTAALGCLVKDYFPEPV |
| 91 | TVSWNSGALTSGVHTFPAVLQSSGLYSLSSVVTVPSSSLGTKTYTCNVDHKPS |
| | NTKVDKRVESKYGPPCPPCPAPEFEGGPSVFLFPPKPKDTLMISRTPEVTCVVV |
| | DVSQEDPEVQFNWYVDGVEVHNAKTKPREEQFNSTYRVVSVLTVLHQDWL |
| | NGKEYKCKVSNKGLPSSIEKTISKAKGQPREPQVYTLPPSQEEMTKNQVSLTC |
| | LVKGFYPSDIAVEWESNGQPENNYKTTPPVLDSDGSFFLYSRLTVDKSRWQE |
| | GNVFSCSVLHEALHSHYTQKSLSLSLGK |
| SEQ | QIQLVQSGSELKKPGASVKISCKASGYTFTDYAMNWVRQAPGQGLEWMGWI |
| ID | NTQTGKPTYAQGFTGRFVFSLDTSVSTAYLQISSLKAEDTAVYFCTRLGTGNT |
| NO: | KGFAYWGQGTLVTVSSASTKGPSVFPLAPCSRSTSESTAALGCLVKDYFPEPV |
| 92 | TVSWNSGALTSGVHTFPAVLQSSGLYSLSSVVTVPSSSLGTKTYTCNVDHKPS |
| | NTKVDKRVESKYGPPCPPCPAPEFEGGPSVFLFPPKPKDTLMISRTPEVTCVVV |
| | DVSQEDPEVQFNWYVDGVEVHNAKTKPREEQFNSTYRVVSVLTVLHQDWL |
| | NGKEYKCKVSNKGLPSSIEKTISKAKGQPREPQVYTLPPSQEEMTKNQVSLTC |
| | LVKGFYPSDIAVEWESNGQPENNYKTTPPVLDSDGSFFLYSRLTVDKSRWQE |
| | GNVFSCSVLHEALHSHYTQKSLSLSLGK |
| SEQ | QIQLVQSGSELKKPGASVKISCKASGYTFTDYAMNWVRQAPGQGLEWMGWI |
| ID | NTQTGKPTYAQGFTGRFVFSLDTSVSTAYLQISSLKAEDTAVYYCTRLGTGNT |
| NO: | KGFAYWGQGTLVTVSSASTKGPSVFPLAPCSRSTSESTAALGCLVKDYFPEPV |
| 93 | TVSWNSGALTSGVHTFPAVLQSSGLYSLSSVVTVPSSSLGTKTYTCNVDHKPS |
| | NTKVDKRVESKYGPPCPPCPAPEFEGGPSVFLFPPKPKDTLMISRTPEVTCVVV |
| | DVSQEDPEVQFNWYVDGVEVHNAKTKPREEQFNSTYRVVSVLTVLHQDWL |
| | NGKEYKCKVSNKGLPSSIEKTISKAKGQPREPQVYTLPPSQEEMTKNQVSLTC |
| | LVKGFYPSDIAVEWESNGQPENNYKTTPPVLDSDGSFFLYSRLTVDKSRWQE |
| | GNVFSCSVLHEALHSHYTQKSLSLSLGK |
| SEQ | QIQLVQSGSELKKPGASVKVSCKASGYTFTDYAMNWVRQAPGQGLEWMGW |
| ID | INTQTGKPTYAQGFTGRFVFSLDTSVSTAYLQISSLKAEDTAVYFCTRLGTGN |
| NO: | TKGFAYWGQGTLVTVSSASTKGPSVFPLAPCSRSTSESTAALGCLVKDYFPEP |
| 94 | VTVSWNSGALTSGVHTFPAVLQSSGLYSLSSVVTVPSSSLGTKTYTCNVDHKP |
| | SNTKVDKRVESKYGPPCPPCPAPEFEGGPSVFLFPPKPKDTLMISRTPEVTCVV |
| | VDVSQEDPEVQFNWYVDGVEVHNAKTKPREEQFNSTYRVVSVLTVLHQDW |
| | LNGKEYKCKVSNKGLPSSIEKTISKAKGQPREPQVYTLPPSQEEMTKNQVSLT |
| | CLVKGFYPSDIAVEWESNGQPENNYKTTPPVLDSDGSFFLYSRLTVDKSRWQ |
| | EGNVFSCSVLHEALHSHYTQKSLSLSLGK |
| SEQ | QVQLVQSGAEVKKPGSSVKVSCKASGYTFTDYAMNWVRQAPGQGLEWMG |
| ID | WINTQTGKPTYAQKFQGRVTFTLDESTSTAYMELSSLRSEDTAVYYCTRLGT |
| NO: | GNTKGFAYWGQGTLVTVSSASTKGPSVFPLAPCSRSTSESTAALGCLVKDYFP |
| 95 | EPVTVSWNSGALTSGVHTFPAVLQSSGLYSLSSVVTVPSSSLGTKTYTCNVDH |
| | KPSNTKVDKRVESKYGPPCPPCPAPEFEGGPSVFLFPPKPKDTLMISRTPEVTC |
| | VVVDVSQEDPEVQFNWYVDGVEVHNAKTKPREEQFNSTYRVVSVLTVLHQD |
| | WLNGKEYKCKVSNKGLPSSIEKTISKAKGQPREPQVYTLPPSQEEMTKNQVSL |
| | TCLVKGFYPSDIAVEWESNGQPENNYKTTPPVLDSDGSFFLYSRLTVDKSRW |
| | QEGNVFSCSVLHEALHSHYTQKSLSLSLGK |
| SEQ | QIQLVQSGSELKKPGASVKVSCKASGYTFTDYAMNWVRQAPGQGLEWMGW |
| ID | INTQTGKPTYAQGFTGRFVFSLDTSVSTAYLQISSLKAEDTAVYYCTRLGTGN |
| NO: | TKGFAYWGQGTLVTVSSASTKGPSVFPLAPCSRSTSESTAALGCLVKDYFPEP |
| 96 | VTVSWNSGALTSGVHTFPAVLQSSGLYSLSSVVTVPSSSLGTKTYTCNVDHKP |
| | SNTKVDKRVESKYGPPCPPCPAPEFEGGPSVFLFPPKPKDTLYITREPEVTCVV |
| | VDVSQEDPEVQFNWYVDGVEVHNAKTKPREEQFNSTYRVVSVLTVLHQDW |
| | LNGKEYKCKVSNKGLPSSIEKTISKAKGQPREPQVYTLPPSQEEMTKNQVSLT |
| | CLVKGFYPSDIAVEWESNGQPENNYKTTPPVLDSDGSFFLYSRLTVDKSRWQ |
| | EGNVFSCSVMHEALHNHYTQKSLSLSLGK |
| SEQ | QIQLVQSGAEVKKPGSSVKVSCKASGYTFTDYAMNWVRQAPGQGLEWMGW |
| ID | INTQTGKPTYAQKFQGRVTFTLDESTSTAYMELSSLRSEDTAVYYCTRLGTGN |
| NO: | TKGFAYWGQGTLVTVSSASTKGPSVFPLAPCSRSTSESTAALGCLVKDYFPEP |
| 97 | VTVSWNSGALTSGVHTFPAVLQSSGLYSLSSVVTVPSSSLGTKTYTCNVDHKP |
| | SNTKVDKRVESKYGPPCPPCPAPEFEGGPSVFLFPPKPKDTLYITREPEVTCVV |
| | VDVSQEDPEVQFNWYVDGVEVHNAKTKPREEQFNSTYRVVSVLTVLHQDW |
| | LNGKEYKCKVSNKGLPSSIEKTISKAKGQPREPQVYTLPPSQEEMTKNQVSLT |
| | CLVKGFYPSDIAVEWESNGQPENNYKTTPPVLDSDGSFFLYSRLTVDKSRWQ |
| | EGNVFSCSVMHEALHNHYTQKSLSLSLGK |
| SEQ | QIQLVQSGAEVKKPGSSVKVSCKASGYTFTDYAMNWVRQAPGQGLEWMGW |
| ID | INTQTGKPTYAQKFQGRVTFTLDESTSTTYMELSSLRSEDTAVYYCARLGTGN |
| NO: | TKGFAYWGQGTLVTVSSASTKGPSVFPLAPCSRSTSESTAALGCLVKDYFPEP |
| 98 | VTVSWNSGALTSGVHTFPAVLQSSGLYSLSSVVTVPSSSLGTKTYTCNVDHKP |
| | SNTKVDKRVESKYGPPCPPCPAPEFEGGPSVFLFPPKPKDTLYITREPEVTCVV |
| | VDVSQEDPEVQFNWYVDGVEVHNAKTKPREEQFNSTYRVVSVLTVLHQDW |
| | LNGKEYKCKVSNKGLPSSIEKTISKAKGQPREPQVYTLPPSQEEMTKNQVSLT |
| | CLVKGFYPSDIAVEWESNGQPENNYKTTPPVLDSDGSFFLYSRLTVDKSRWQ |
| | EGNVFSCSVMHEALHNHYTQKSLSLSLGK |
| SEQ | QIQLVQSGAEVKKPGSSVKVSCKASGYTFTDYAMNWVRQAPGQGLEWMGW |
| ID | INTQTGKPTYAQKFQGRVTFTLDESTSTTYMELSSLRSEDTAVYYCTRLGTGN |
| NO: | TKGFAYWGQGTLVTVSSASTKGPSVFPLAPCSRSTSESTAALGCLVKDYFPEP |
| 99 | VTVSWNSGALTSGVHTFPAVLQSSGLYSLSSVVTVPSSSLGTKTYTCNVDHKP |
| | SNTKVDKRVESKYGPPCPPCPAPEFEGGPSVFLFPPKPKDTLYITREPEVTCVV |
| | VDVSQEDPEVQFNWYVDGVEVHNAKTKPREEQFNSTYRVVSVLTVLHQDW |
| | LNGKEYKCKVSNKGLPSSIEKTISKAKGQPREPQVYTLPPSQEEMTKNQVSLT |
| | CLVKGFYPSDIAVEWESNGQPENNYKTTPPVLDSDGSFFLYSRLTVDKSRWQ |
| | EGNVFSCSVMHEALHNHYTQKSLSLSLGK |
| SEQ | ASTKGPSVFPLAPCSRSTSESTAALGCLVKDYFPEPVTVSWNSGALTSGVHTF |
| ID | PAVLQSSGLYSLSSVVTVPSSSLGTKTYTCNVDHKPSNTKVDKRVESKYGPPC |
| NO: | PPCPAPEFEGGPSVFLFPPKPKDTLYITREPEVTCVVVDVSQEDPEVQFNWYV |
| 100 | DGVEVHNAKTKPREEQFNSTYRVVSVLIVLHQDWLNGKEYKCKVSNKGLPS |
| | SIEKTISKAKGQPREPQVYTLPPSQEEMTKNQVSLTCLVKGFYPSDIAVEWESN |
| | GQPENNYKTTPPVLDSDGSFFLYSRLTVDKSRWQEGNVFSCSVMHEALHNHY |
| | TQKSLSLSLGKQIQLVQSGPEVKKPGESVKISCKASGYTFTDYAMNWVKQAP |
| | GNGLKWMGWINTQTGKPTYADDFKQRFVFSLETSARTTYLQINNLNIEDTAT |
| | YFCTRLGTGNTKGFAYWGQGTLVTVSS |
| SEQ | QIQLVQSGSELKKPGASVKISCKASGYTFTDYAMNWVKQAPGQGLEWMGWI |
| ID | NTQTGKPTYAQGFTGRFVFSLDTSVSTAYLQISSLKAEDTAVYFCTRLGTGNT |
| NO: | KGFAYWGQGTLVTVSSASTKGPSVFPLAPCSRSTSESTAALGCLVKDYFPEPV |
| 101 | TVSWNSGALTSGVHTFPAVLQSSGLYSLSSVVTVPSSSLGTKTYTCNVDHKPS |
| | NTKVDKRVESKYGPPCPPCPAPEFEGGPSVFLFPPKPKDTLYITREPEVTCVVV |
| | DVSQEDPEVQFNWYVDGVEVHNAKTKPREEQFNSTYRVVSVLTVLHQDWL |
| | NGKEYKCKVSNKGLPSSIEKTISKAKGQPREPQVYTLPPSQEEMTKNQVSLTC |
| | LVKGFYPSDIAVEWESNGQPENNYKTTPPVLDSDGSFFLYSRLTVDKSRWQE |
| | GNVFSCSVMHEALHNHYTQKSLSLSLGK |
| SEQ | QIQLVQSGSELKKPGASVKISCKASGYTFTDYAMNWVKQAPGQGLEWMGWI |
| ID | NTQTGKPTYAQGFTGRFVFSLDTSVSTAYLQISSLKAEDTAVYFCTRLGTGNT |
| NO: | KGFAYWGQGTLVTVSSASTKGPSVFPLAPCSRSTSESTAALGCLVKDYFPEPV |
| 102 | TVSWNSGALTSGVHTFPAVLQSSGLYSLSSVVTVPSSSLGTKTYTCNVDHKPS |
| | NTKVDKRVESKYGPPCPPCPAPEFEGGPSVFLFPPKPKDTLYITREPEVTCVVV |
| | DVSQEDPEVQFNWYVDGVEVHNAKTKPREEQFNSTYRVVSVLTVLHQDWL |
| | NGKEYKCKVSNKGLPSSIEKTISKAKGQPREPQVYTLPPSQEEMTKNQVSLTC |
| | LVKGFYPSDIAVEWESNGQPENNYKTTPPVLDSDGSFFLYSRLTVDKSRWQE |
| | GNVFSCSVMHEALHNHYTQKSLSLSLGK |
| SEQ | QIQLVQSGSELKKPGASVKISCKASGYTFTDYAMNWVRQAPGQGLEWMGWI |
| ID | NTQTGKPTYAQGFTGRFVFSLDTSVRTAYLQISSLKAEDTAVYFCTRLGTGNT |
| NO: | KGFAYWGQGTLVTVSSASTKGPSVFPLAPCSRSTSESTAALGCLVKDYFPEPV |
| 103 | TVSWNSGALTSGVHTFPAVLQSSGLYSLSSVVTVPSSSLGTKTYTCNVDHKPS |
| | NTKVDKRVESKYGPPCPPCPAPEFEGGPSVFLFPPKPKDTLYITREPEVTCVVV |
| | DVSQEDPEVQFNWYVDGVEVHNAKTKPREEQFNSTYRVVSVLTVLHQDWL |
| | NGKEYKCKVSNKGLPSSIEKTISKAKGQPREPQVYTLPPSQEEMTKNQVSLTC |
| | LVKGFYPSDIAVEWESNGQPENNYKTTPPVLDSDGSFFLYSRLTVDKSRWQE |
| | GNVFSCSVMHEALHNHYTQKSLSLSLGK |
| SEQ | QIQLVQSGSELKKPGASVKISCKASGYTFTDYAMNWVRQAPGQGLEWMGWI |
| ID | NTQTGKPTYAQGFTGRFVFSLDTSVSTAYLQISSLKAEDTAVYFCTRLGTGNT |
| NO: | KGFAYWGQGTLVTVSSASTKGPSVFPLAPCSRSTSESTAALGCLVKDYFPEPV |
| 104 | TVSWNSGALTSGVHTFPAVLQSSGLYSLSSVVTVPSSSLGTKTYTCNVDHKPS |
| | NTKVDKRVESKYGPPCPPCPAPEFEGGPSVFLEPPKPKDTLYITREPEVTCVVV |
| | DVSQEDPEVQFNWYVDGVEVHNAKTKPREEQFNSTYRVVSVLTVLHQDWL |
| | NGKEYKCKVSNKGLPSSIEKTISKAKGQPREPQVYTLPPSQEEMTKNQVSLTC |
| | LVKGFYPSDIAVEWESNGQPENNYKTTPPVLDSDGSFFLYSRLTVDKSRWQE |
| | GNVFSCSVMHEALHNHYTQKSLSLSLGK |
| SEQ | QIQLVQSGSELKKPGASVKISCKASGYTFTDYAMNWVRQAPGQGLEWMGWI |
| ID | NTQTGKPTYAQGFTGRFVFSLDTSVSTAYLQISSLKAEDTAVYYCTRLGTGNT |
| NO: | KGFAYWGQGTLVTVSSASTKGPSVFPLAPCSRSTSESTAALGCLVKDYFPEPV |
| 105 | TVSWNSGALTSGVHTFPAVLQSSGLYSLSSVVTVPSSSLGTKTYTCNVDHKPS |
| | NTKVDKRVESKYGPPCPPCPAPEFEGGPSVFLFPPKPKDTLYITREPEVTCVVV |
| | DVSQEDPEVQFNWYVDGVEVHNAKTKPREEQFNSTYRVVSVLIVLHQDWL |
| | NGKEYKCKVSNKGLPSSIEKTISKAKGQPREPQVYTLPPSQEEMTKNQVSLTC |
| | LVKGFYPSDIAVEWESNGQPENNYKTTPPVLDSDGSFFLYSRLTVDKSRWQE |
| | GNVFSCSVMHEALHNHYTQKSLSLSLGK |
| SEQ | QIQLVQSGSELKKPGASVKVSCKASGYTFTDYAMNWVRQAPGQGLEWMGW |
| ID | INTQTGKPTYAQGFTGRFVFSLDTSVSTAYLQISSLKAEDTAVYFCTRLGTGN |
| NO: | TKGFAYWGQGTLVTVSSASTKGPSVFPLAPCSRSTSESTAALGCLVKDYFPEP |
| 106 | VTVSWNSGALTSGVHTFPAVLQSSGLYSLSSVVTVPSSSLGTKTYTCNVDHKP |
| | SNTKVDKRVESKYGPPCPPCPAPEFEGGPSVFLFPPKPKDTLYITREPEVTCVV |
| | VDVSQEDPEVQFNWYVDGVEVHNAKTKPREEQFNSTYRVVSVLTVLHQDW |
| | LNGKEYKCKVSNKGLPSSIEKTISKAKGQPREPQVYTLPPSQEEMTKNQVSLT |
| | CLVKGFYPSDIAVEWESNGQPENNYKTTPPVLDSDGSFFLYSRLTVDKSRWQ |
| | EGNVFSCSVMHEALHNHYTQKSLSLSLGK |
| SEQ | QVQLVQSGAEVKKPGSSVKVSCKASGYTFTDYAMNWVRQAPGQGLEWMG |
| ID | WINTQTGKPTYAQKFQGRVTFTLDESTSTAYMELSSLRSEDTAVYYCTRLGT |
| NO: | GNTKGFAYWGQGTLVTVSSASTKGPSVFPLAPCSRSTSESTAALGCLVKDYFP |
| 107 | EPVTVSWNSGALTSGVHTFPAVLQSSGLYSLSSVVTVPSSSLGTKTYTCNVDH |
| | KPSNTKVDKRVESKYGPPCPPCPAPEFEGGPSVFLFPPKPKDTLYITREPEVTC |
| | VVVDVSQEDPEVQFNWYVDGVEVHNAKTKPREEQFNSTYRVVSVLTVLHQD |
| | WLNGKEYKCKVSNKGLPSSIEKTISKAKGQPREPQVYTLPPSQEEMTKNQVSL |
| | TCLVKGFYPSDIAVEWESNGQPENNYKTTPPVLDSDGSFFLYSRLTVDKSRW |
| | QEGNVFSCSVMHEALHNHYTQKSLSLSLGK |
| SEQ | QIQLVQSGSELKKPGASVKVSCKASGYTFTDYAMNWVRQAPGQGLEWMGW |
| ID | INTQTGKPTYAQGFTGRFVFSLDTSVSTAYLQISSLKAEDTAVYYCTRLGTGN |
| NO: | TKGFAYWGQGTLVTVSSASTKGPSVFPLAPSSKSTSGGTAALGCLVKDYFPEP |
| 108 | VTVSWNSGALTSGVHTFPAVLQSSGLYSLSSVVTVPSSSLGTQTYICNVNHKP |
| | SNTKVDKKVEPKSCDKTHTCPPCPAPEAAGGPSVFLFPPKPKDTLMISRTPEVT |
| | CVVVSVSHEDPEVKFNWYVDGVEVHNAKTKPREEQYNSTYRVVSVLTVLHQ |
| | DWLNGKEYKCKVSNKALPAPIEKTISKAKGQPREPQVYTLPPSREEMTKNQV |
| | SLTCLVKGFYPSDIAVEWESNGQPENNYKTTPPVLDSDGSFFLYSKLTVDKSR |
| | WQQGNVFSCSVMHEALHNHYTQKSLSLSPG |
| SEQ | QIQLVQSGAEVKKPGSSVKVSCKASGYTFTDYAMNWVRQAPGQGLEWMGW |
| ID | INTQTGKPTYAQKFQGRVTFTLDESTSTAYMELSSLRSEDTAVYYCTRLGTGN |
| NO: | TKGFAYWGQGTLVTVSSASTKGPSVFPLAPSSKSTSGGTAALGCLVKDYFPEP |
| 109 | VTVSWNSGALTSGVHTFPAVLQSSGLYSLSSVVTVPSSSLGTQTYICNVNHKP |
| | SNTKVDKKVEPKSCDKTHTCPPCPAPEAAGGPSVFLFPPKPKDTLMISRTPEVT |
| | CVVVSVSHEDPEVKFNWYVDGVEVHNAKTKPREEQYNSTYRVVSVLTVLHQ |
| | DWLNGKEYKCKVSNKALPAPIEKTISKAKGQPREPQVYTLPPSREEMTKNQV |
| | SLTCLVKGFYPSDIAVEWESNGQPENNYKTTPPVLDSDGSFFLYSKLTVDKSR |
| | WQQGNVFSCSVMHEALHNHYTQKSLSLSPG |
| SEQ | QIQLVQSGAEVKKPGSSVKVSCKASGYTFTDYAMNWVRQAPGQGLEWMGW |
| ID | INTQTGKPTYAQKFQGRVTFTLDESTSTTYMELSSLRSEDTAVYYCARLGTGN |
| NO: | TKGFAYWGQGTLVTVSSASTKGPSVFPLAPSSKSTSGGTAALGCLVKDYFPEP |
| 110 | VTVSWNSGALTSGVHTFPAVLQSSGLYSLSSVVTVPSSSLGTQTYICNVNHKP |
| | SNTKVDKKVEPKSCDKTHTCPPCPAPEAAGGPSVFLFPPKPKDTLMISRTPEVT |
| | CVVVSVSHEDPEVKFNWYVDGVEVHNAKTKPREEQYNSTYRVVSVLTVLHQ |
| | DWLNGKEYKCKVSNKALPAPIEKTISKAKGQPREPQVYTLPPSREEMTKNQV |
| | SLTCLVKGFYPSDIAVEWESNGQPENNYKTTPPVLDSDGSFFLYSKLTVDKSR |
| | WQQGNVFSCSVMHEALHNHYTQKSLSLSPG |
| SEQ | QIQLVQSGAEVKKPGSSVKVSCKASGYTFTDYAMNWVRQAPGQGLEWMGW |
| ID | INTQTGKPTYAQKFQGRVTFTLDESTSTTYMELSSLRSEDTAVYYCTRLGTGN |
| NO: | TKGFAYWGQGTLVTVSSASTKGPSVFPLAPSSKSTSGGTAALGCLVKDYFPEP |
| 111 | VTVSWNSGALTSGVHTFPAVLQSSGLYSLSSVVTVPSSSLGTQTYICNVNHKP |
| | SNTKVDKKVEPKSCDKTHTCPPCPAPEAAGGPSVFLFPPKPKDTLMISRTPEVT |
| | CVVVSVSHEDPEVKFNWYVDGVEVHNAKTKPREEQYNSTYRVVSVLTVLHQ |
| | DWLNGKEYKCKVSNKALPAPIEKTISKAKGQPREPQVYTLPPSREEMTKNQV |
| | SLTCLVKGFYPSDIAVEWESNGQPENNYKTTPPVLDSDGSFFLYSKLTVDKSR |
| | WQQGNVFSCSVMHEALHNHYTQKSLSLSPG |
| SEQ | QIQLVQSGPEVKKPGESVKISCKASGYTFTDYAMNWVKQAPGNGLKWMGWI |
| ID | NTQTGKPTYADDFKQRFVFSLETSARTTYLQINNLNIEDTATYFCTRLGTGNT |
| NO: | KGFAYWGQGTLVTVSSASTKGPSVFPLAPSSKSTSGGTAALGCLVKDYFPEPV |
| 112 | TVSWNSGALTSGVHTFPAVLQSSGLYSLSSVVTVPSSSLGTQTYICNVNHKPS |
| | NTKVDKKVEPKSCDKTHTCPPCPAPEAAGGPSVFLFPPKPKDTLMISRTPEVT |
| | CVVVSVSHEDPEVKFNWYVDGVEVHNAKTKPREEQYNSTYRVVSVLTVLHQ |
| | DWLNGKEYKCKVSNKALPAPIEKTISKAKGQPREPQVYTLPPSREEMTKNQV |
| | SLTCLVKGFYPSDIAVEWESNGQPENNYKTTPPVLDSDGSFFLYSKLTVDKSR |
| | WQQGNVFSCSVMHEALHNHYTQKSLSLSPG |
| SEQ | QIQLVQSGSELKKPGASVKISCKASGYTFTDYAMNWVKQAPGQGLEWMGWI |
| ID | NTQTGKPTYAQGFTGRFVFSLDTSVSTAYLQISSLKAEDTAVYFCTRLGTGNT |
| NO: | KGFAYWGQGTLVTVSSASTKGPSVFPLAPSSKSTSGGTAALGCLVKDYFPEPV |
| 113 | TVSWNSGALTSGVHTFPAVLQSSGLYSLSSVVTVPSSSLGTQTYICNVNHKPS |
| | NTKVDKKVEPKSCDKTHTCPPCPAPEAAGGPSVFLFPPKPKDTLMISRTPEVT |
| | CVVVSVSHEDPEVKFNWYVDGVEVHNAKTKPREEQYNSTYRVVSVLTVLHQ |
| | DWLNGKEYKCKVSNKALPAPIEKTISKAKGQPREPQVYTLPPSREEMTKNQV |
| | SLTCLVKGFYPSDIAVEWESNGQPENNYKTTPPVLDSDGSFFLYSKLTVDKSR |
| | WQQGNVFSCSVMHEALHNHYTQKSLSLSPG |
| SEQ | QIQLVQSGSELKKPGASVKISCKASGYTFTDYAMNWVKQAPGQGLEWMGWI |
| ID | NTQTGKPTYAQGFTGRFVFSLDTSVSTAYLQISSLKAEDTAVYFCTRLGTGNT |
| NO: | KGFAYWGQGTLVTVSSASTKGPSVFPLAPSSKSTSGGTAALGCLVKDYFPEPV |
| 114 | TVSWNSGALTSGVHTFPAVLQSSGLYSLSSVVTVPSSSLGTQTYICNVNHKPS |
| | NTKVDKKVEPKSCDKTHTCPPCPAPEAAGGPSVFLFPPKPKDTLMISRTPEVT |
| | CVVVSVSHEDPEVKFNWYVDGVEVHNAKTKPREEQYNSTYRVVSVLTVLHQ |
| | DWLNGKEYKCKVSNKALPAPIEKTISKAKGQPREPQVYTLPPSREEMTKNQV |
| | SLTCLVKGFYPSDIAVEWESNGQPENNYKTTPPVLDSDGSFFLYSKLTVDKSR |
| | WQQGNVFSCSVMHEALHNHYTQKSLSLSPG |
| SEQ | QIQLVQSGSELKKPGASVKISCKASGYTFTDYAMNWVRQAPGQGLEWMGWI |
| ID | NTQTGKPTYAQGFTGRFVFSLDTSVRTAYLQISSLKAEDTAVYFCTRLGTGNT |
| NO: | KGFAYWGQGTLVTVSSASTKGPSVFPLAPSSKSTSGGTAALGCLVKDYFPEPV |
| 115 | TVSWNSGALTSGVHTFPAVLQSSGLYSLSSVVTVPSSSLGTQTYICNVNHKPS |
| | NTKVDKKVEPKSCDKTHTCPPCPAPEAAGGPSVFLFPPKPKDTLMISRTPEVT |
| | CVVVSVSHEDPEVKFNWYVDGVEVHNAKTKPREEQYNSTYRVVSVLTVLHQ |
| | DWLNGKEYKCKVSNKALPAPIEKTISKAKGQPREPQVYTLPPSREEMTKNQV |
| | SLTCLVKGFYPSDIAVEWESNGQPENNYKTTPPVLDSDGSFFLYSKLTVDKSR |
| | WQQGNVFSCSVMHEALHNHYTQKSLSLSPG |
| SEQ | QIQLVQSGSELKKPGASVKISCKASGYTFTDYAMNWVRQAPGQGLEWMGWI |
| ID | NTQTGKPTYAQGFTGRFVFSLDTSVSTAYLQISSLKAEDTAVYFCTRLGTGNT |
| NO: | KGFAYWGQGTLVTVSSASTKGPSVFPLAPSSKSTSGGTAALGCLVKDYFPEPV |
| 116 | TVSWNSGALTSGVHTFPAVLQSSGLYSLSSVVTVPSSSLGTQTYICNVNHKPS |
| | NTKVDKKVEPKSCDKTHTCPPCPAPEAAGGPSVFLFPPKPKDTLMISRTPEVT |
| | CVVVSVSHEDPEVKFNWYVDGVEVHNAKTKPREEQYNSTYRVVSVLTVLHQ |
| | DWLNGKEYKCKVSNKALPAPIEKTISKAKGQPREPQVYTLPPSREEMTKNQV |
| | SLTCLVKGFYPSDIAVEWESNGQPENNYKTTPPVLDSDGSFFLYSKLTVDKSR |
| | WQQGNVFSCSVMHEALHNHYTQKSLSLSPG |
| SEQ | QIQLVQSGSELKKPGASVKISCKASGYTFTDYAMNWVRQAPGQGLEWMGWI |
| ID | NTQTGKPTYAQGFTGRFVFSLDTSVSTAYLQISSLKAEDTAVYYCTRLGTGNT |
| NO: | KGFAYWGQGTLVTVSSASTKGPSVFPLAPSSKSTSGGTAALGCLVKDYFPEPV |
| 117 | TVSWNSGALTSGVHTFPAVLQSSGLYSLSSVVTVPSSSLGTQTYICNVNHKPS |
| | NTKVDKKVEPKSCDKTHTCPPCPAPEAAGGPSVFLFPPKPKDTLMISRTPEVT |
| | CVVVSVSHEDPEVKFNWYVDGVEVHNAKTKPREEQYNSTYRVVSVLTVLHQ |
| | DWLNGKEYKCKVSNKALPAPIEKTISKAKGQPREPQVYTLPPSREEMTKNQV |
| | SLTCLVKGFYPSDIAVEWESNGQPENNYKTTPPVLDSDGSFFLYSKLTVDKSR |
| | WQQGNVFSCSVMHEALHNHYTQKSLSLSPG |
| SEQ | QIQLVQSGSELKKPGASVKVSCKASGYTFTDYAMNWVRQAPGQGLEWMGW |
| ID | INTQTGKPTYAQGFTGRFVFSLDTSVSTAYLQISSLKAEDTAVYFCTRLGTGN |
| NO: | TKGFAYWGQGTLVTVSSASTKGPSVFPLAPSSKSTSGGTAALGCLVKDYFPEP |
| 118 | VTVSWNSGALTSGVHTFPAVLQSSGLYSLSSVVTVPSSSLGTQTYICNVNHKP |
| | SNTKVDKKVEPKSCDKTHTCPPCPAPEAAGGPSVFLFPPKPKDTLMISRTPEVT |
| | CVVVSVSHEDPEVKFNWYVDGVEVHNAKTKPREEQYNSTYRVVSVLTVLHQ |
| | DWLNGKEYKCKVSNKALPAPIEKTISKAKGQPREPQVYTLPPSREEMTKNQV |
| | SLTCLVKGFYPSDIAVEWESNGQPENNYKTTPPVLDSDGSFFLYSKLTVDKSR |
| | WQQGNVFSCSVMHEALHNHYTQKSLSLSPG |
| SEQ | QVQLVQSGAEVKKPGSSVKVSCKASGYTFTDYAMNWVRQAPGQGLEWMG |
| ID | WINTQTGKPTYAQKFQGRVTFTLDESTSTAYMELSSLRSEDTAVYYCTRLGT |
| NO: | GNTKGFAYWGQGTLVTVSSASTKGPSVFPLAPSSKSTSGGTAALGCLVKDYF |
| 119 | PEPVTVSWNSGALTSGVHTFPAVLQSSGLYSLSSVVTVPSSSLGTQTYICNVN |
| | HKPSNTKVDKKVEPKSCDKTHTCPPCPAPEAAGGPSVFLFPPKPKDTLMISRT |
| | PEVTCVVVSVSHEDPEVKFNWYVDGVEVHNAKTKPREEQYNSTYRVVSVLT |
| | VLHQDWLNGKEYKCKVSNKALPAPIEKTISKAKGQPREPQVYTLPPSREEMT |
| | KNQVSLTCLVKGFYPSDIAVEWESNGQPENNYKTTPPVLDSDGSFFLYSKLTV |
| | DKSRWQQGNVFSCSVMHEALHNHYTQKSLSLSPG |
| SEQ | QIQLVQSGSELKKPGASVKVSCKASGYTFTDYAMNWVRQAPGQGLEWMGW |
| ID | INTQTGKPTYAQGFTGRFVFSLDTSVSTAYLQISSLKAEDTAVYYCTRLGTGN |
| NO: | TKGFAYWGQGTLVTVSSASTKGPSVFPLAPCSRSTSESTAALGCLVKDYFPEP |
| 120 | VTVSWNSGALTSGVHTFPAVLQSSGLYSLSSVVTVPSSSLGTKTYTCNVDHKP |
| | SNTKVDKRVESKYGPPCPPCPAPEFEGGPSVFLFPPKPKDTLMISRTPEVTCVV |
| | VDVSQEDPEVQFNWYVDGVEVHNAKTKPREEQFNSTYRVVSVLTVLHQDW |
| | LNGKEYKCKVSNKGLPSSIEKTISKAKGQPREPQVYTLPPSQEEMTKNQVSLT |
| | CLVKGFYPSDIAVEWESNGQPENNYKTTPPVLDSDGSFFLYSRLTVDKSRWQ |
| | EGNVFSCSVLHEALHSHYTQKSLSLSLG |
| SEQ | QIQLVQSGAEVKKPGSSVKVSCKASGYTFTDYAMNWVRQAPGQGLEWMGW |
| ID | INTQTGKPTYAQKFQGRVTFTLDESTSTAYMELSSLRSEDTAVYYCTRLGTGN |
| NO: | TKGFAYWGQGTLVTVSSASTKGPSVFPLAPCSRSTSESTAALGCLVKDYFPEP |
| 121 | VTVSWNSGALTSGVHTFPAVLQSSGLYSLSSVVTVPSSSLGTKTYTCNVDHKP |
| | SNTKVDKRVESKYGPPCPPCPAPEFEGGPSVFLFPPKPKDTLMISRTPEVTCVV |
| | VDVSQEDPEVQFNWYVDGVEVHNAKTKPREEQFNSTYRVVSVLTVLHQDW |
| | LNGKEYKCKVSNKGLPSSIEKTISKAKGQPREPQVYTLPPSQEEMTKNQVSLT |
| | CLVKGFYPSDIAVEWESNGQPENNYKTTPPVLDSDGSFFLYSRLTVDKSRWQ |
| | EGNVFSCSVLHEALHSHYTQKSLSLSLG |
| SEQ | QIQLVQSGAEVKKPGSSVKVSCKASGYTFTDYAMNWVRQAPGQGLEWMGW |
| ID | INTQTGKPTYAQKFQGRVTFTLDESTSTTYMELSSLRSEDTAVYYCARLGTGN |
| NO: | TKGFAYWGQGTLVTVSSASTKGPSVFPLAPCSRSTSESTAALGCLVKDYFPEP |
| 122 | VTVSWNSGALTSGVHTFPAVLQSSGLYSLSSVVTVPSSSLGTKTYTCNVDHKP |
| | SNTKVDKRVESKYGPPCPPCPAPEFEGGPSVFLFPPKPKDTLMISRTPEVTCVV |
| | VDVSQEDPEVQFNWYVDGVEVHNAKTKPREEQFNSTYRVVSVLTVLHQDW |
| | LNGKEYKCKVSNKGLPSSIEKTISKAKGQPREPQVYTLPPSQEEMTKNQVSLT |
| | CLVKGFYPSDIAVEWESNGQPENNYKTTPPVLDSDGSFFLYSRLTVDKSRWQ |
| | EGNVFSCSVLHEALHSHYTQKSLSLSLG |
| SEQ | QIQLVQSGAEVKKPGSSVKVSCKASGYTFTDYAMNWVRQAPGQGLEWMGW |
| ID | INTQTGKPTYAQKFQGRVTFTLDESTSTTYMELSSLRSEDTAVYYCTRLGTGN |
| NO: | TKGFAYWGQGTLVTVSSASTKGPSVFPLAPCSRSTSESTAALGCLVKDYFPEP |
| 123 | VTVSWNSGALTSGVHTFPAVLQSSGLYSLSSVVTVPSSSLGTKTYTCNVDHKP |
| | SNTKVDKRVESKYGPPCPPCPAPEFEGGPSVFLFPPKPKDTLMISRTPEVTCVV |
| | VDVSQEDPEVQFNWYVDGVEVHNAKTKPREEQFNSTYRVVSVLTVLHQDW |
| | LNGKEYKCKVSNKGLPSSIEKTISKAKGQPREPQVYTLPPSQEEMTKNQVSLT |
| | CLVKGFYPSDIAVEWESNGQPENNYKTTPPVLDSDGSFFLYSRLTVDKSRWQ |
| | EGNVFSCSVLHEALHSHYTQKSLSLSLG |
| SEQ | QIQLVQSGPEVKKPGESVKISCKASGYTFTDYAMNWVKQAPGNGLKWMGWI |
| ID | NTQTGKPTYADDFKQRFVFSLETSARTTYLQINNLNIEDTATYFCTRLGTGNT |
| NO: | KGFAYWGQGTLVTVSSASTKGPSVFPLAPCSRSTSESTAALGCLVKDYFPEPV |
| 124 | TVSWNSGALTSGVHTFPAVLQSSGLYSLSSVVTVPSSSLGTKTYTCNVDHKPS |
| | NTKVDKRVESKYGPPCPPCPAPEFEGGPSVFLFPPKPKDTLMISRTPEVTCVVV |
| | DVSQEDPEVQFNWYVDGVEVHNAKTKPREEQFNSTYRVVSVLTVLHQDWL |
| | NGKEYKCKVSNKGLPSSIEKTISKAKGQPREPQVYTLPPSQEEMTKNQVSLTC |
| | LVKGFYPSDIAVEWESNGQPENNYKTTPPVLDSDGSFFLYSRLTVDKSRWQE |
| | GNVFSCSVLHEALHSHYTQKSLSLSLG |
| SEQ | QIQLVQSGSELKKPGASVKISCKASGYTFTDYAMNWVKQAPGQGLEWMGWI |
| ID | NTQTGKPTYAQGFTGRFVFSLDTSVSTAYLQISSLKAEDTAVYFCTRLGTGNT |
| NO: | KGFAYWGQGTLVTVSSASTKGPSVFPLAPCSRSTSESTAALGCLVKDYFPEPV |
| 125 | TVSWNSGALTSGVHTFPAVLQSSGLYSLSSVVTVPSSSLGTKTYTCNVDHKPS |
| | NTKVDKRVESKYGPPCPPCPAPEFEGGPSVFLFPPKPKDTLMISRTPEVTCVVV |
| | DVSQEDPEVQFNWYVDGVEVHNAKTKPREEQFNSTYRVVSVLTVLHQDWL |
| | NGKEYKCKVSNKGLPSSIEKTISKAKGQPREPQVYTLPPSQEEMTKNQVSLTC |
| | LVKGFYPSDIAVEWESNGQPENNYKTTPPVLDSDGSFFLYSRLTVDKSRWQE |
| | GNVFSCSVLHEALHSHYTQKSLSLSLG |
| SEQ | QIQLVQSGSELKKPGASVKISCKASGYTFTDYAMNWVKQAPGQGLEWMGWI |
| ID | NTQTGKPTYAQGFTGRFVFSLDTSVSTAYLQISSLKAEDTAVYFCTRLGTGNT |
| NO: | KGFAYWGQGTLVTVSSASTKGPSVFPLAPCSRSTSESTAALGCLVKDYFPEPV |
| 126 | TVSWNSGALTSGVHTFPAVLQSSGLYSLSSVVTVPSSSLGTKTYTCNVDHKPS |
| | NTKVDKRVESKYGPPCPPCPAPEFEGGPSVFLFPPKPKDTLMISRTPEVTCVVV |
| | DVSQEDPEVQFNWYVDGVEVHNAKTKPREEQFNSTYRVVSVLTVLHQDWL |
| | NGKEYKCKVSNKGLPSSIEKTISKAKGQPREPQVYTLPPSQEEMTKNQVSLTC |
| | LVKGFYPSDIAVEWESNGQPENNYKTTPPVLDSDGSFFLYSRLTVDKSRWQE |
| | GNVFSCSVLHEALHSHYTQKSLSLSLG |
| SEQ | QIQLVQSGSELKKPGASVKISCKASGYTFTDYAMNWVRQAPGQGLEWMGWI |
| ID | NTQTGKPTYAQGFTGRFVFSLDTSVRTAYLQISSLKAEDTAVYFCTRLGTGNT |
| NO: | KGFAYWGQGTLVTVSSASTKGPSVFPLAPCSRSTSESTAALGCLVKDYFPEPV |
| 127 | TVSWNSGALTSGVHTFPAVLQSSGLYSLSSVVTVPSSSLGTKTYTCNVDHKPS |
| | NTKVDKRVESKYGPPCPPCPAPEFEGGPSVFLFPPKPKDTLMISRTPEVTCVVV |
| | DVSQEDPEVQFNWYVDGVEVHNAKTKPREEQFNSTYRVVSVLTVLHQDWL |
| | NGKEYKCKVSNKGLPSSIEKTISKAKGQPREPQVYTLPPSQEEMTKNQVSLTC |
| | LVKGFYPSDIAVEWESNGQPENNYKTTPPVLDSDGSFFLYSRLTVDKSRWQE |
| | GNVFSCSVLHEALHSHYTQKSLSLSLG |
| SEQ | QIQLVQSGSELKKPGASVKISCKASGYTFTDYAMNWVRQAPGQGLEWMGWI |
| ID | NTQTGKPTYAQGFTGRFVFSLDTSVSTAYLQISSLKAEDTAVYFCTRLGTGNT |
| NO: | KGFAYWGQGTLVTVSSASTKGPSVFPLAPCSRSTSESTAALGCLVKDYFPEPV |
| 128 | TVSWNSGALTSGVHTFPAVLQSSGLYSLSSVVTVPSSSLGTKTYTCNVDHKPS |
| | NTKVDKRVESKYGPPCPPCPAPEFEGGPSVFLFPPKPKDTLMISRTPEVTCVVV |
| | DVSQEDPEVQFNWYVDGVEVHNAKTKPREEQFNSTYRVVSVLTVLHQDWL |
| | NGKEYKCKVSNKGLPSSIEKTISKAKGQPREPQVYTLPPSQEEMTKNQVSLTC |
| | LVKGFYPSDIAVEWESNGQPENNYKTTPPVLDSDGSFFLYSRLTVDKSRWQE |
| | GNVFSCSVLHEALHSHYTQKSLSLSLG |
| SEQ | QIQLVQSGSELKKPGASVKISCKASGYTFTDYAMNWVRQAPGQGLEWMGWI |
| ID | NTQTGKPTYAQGFTGRFVFSLDTSVSTAYLQISSLKAEDTAVYYCTRLGTGNT |
| NO: | KGFAYWGQGTLVTVSSASTKGPSVFPLAPCSRSTSESTAALGCLVKDYFPEPV |
| 129 | TVSWNSGALTSGVHTFPAVLQSSGLYSLSSVVTVPSSSLGTKTYTCNVDHKPS |
| | NTKVDKRVESKYGPPCPPCPAPEFEGGPSVFLFPPKPKDTLMISRTPEVTCVVV |
| | DVSQEDPEVQFNWYVDGVEVHNAKTKPREEQFNSTYRVVSVLTVLHQDWL |
| | NGKEYKCKVSNKGLPSSIEKTISKAKGQPREPQVYTLPPSQEEMTKNQVSLTC |
| | LVKGFYPSDIAVEWESNGQPENNYKTTPPVLDSDGSFFLYSRLTVDKSRWQE |
| | GNVFSCSVLHEALHSHYTQKSLSLSLG |
| SEQ | QIQLVQSGSELKKPGASVKVSCKASGYTFTDYAMNWVRQAPGQGLEWMGW |
| ID | INTQTGKPTYAQGFTGRFVFSLDTSVSTAYLQISSLKAEDTAVYFCTRLGTGN |
| NO: | TKGFAYWGQGTLVTVSSASTKGPSVFPLAPCSRSTSESTAALGCLVKDYFPEP |
| 130 | VTVSWNSGALTSGVHTFPAVLQSSGLYSLSSVVTVPSSSLGTKTYTCNVDHKP |
| | SNTKVDKRVESKYGPPCPPCPAPEFEGGPSVFLFPPKPKDTLMISRTPEVTCVV |
| | VDVSQEDPEVQFNWYVDGVEVHNAKTKPREEQFNSTYRVVSVLTVLHQDW |
| | LNGKEYKCKVSNKGLPSSIEKTISKAKGQPREPQVYTLPPSQEEMTKNQVSLT |
| | CLVKGFYPSDIAVEWESNGQPENNYKTTPPVLDSDGSFFLYSRLTVDKSRWQ |
| | EGNVFSCSVLHEALHSHYTQKSLSLSLG |
| SEQ | QVQLVQSGAEVKKPGSSVKVSCKASGYTFTDYAMNWVRQAPGQGLEWMG |
| ID | WINTQTGKPTYAQKFQGRVTFTLDESTSTAYMELSSLRSEDTAVYYCTRLGT |
| NO: | GNTKGFAYWGQGTLVTVSSASTKGPSVFPLAPCSRSTSESTAALGCLVKDYFP |
| 131 | EPVTVSWNSGALTSGVHTFPAVLQSSGLYSLSSVVTVPSSSLGTKTYTCNVDH |
| | KPSNTKVDKRVESKYGPPCPPCPAPEFEGGPSVFLFPPKPKDTLMISRTPEVTC |
| | VVVDVSQEDPEVQFNWYVDGVEVHNAKTKPREEQFNSTYRVVSVLTVLHQD |
| | WLNGKEYKCKVSNKGLPSSIEKTISKAKGQPREPQVYTLPPSQEEMTKNQVSL |
| | TCLVKGFYPSDIAVEWESNGQPENNYKTTPPVLDSDGSFFLYSRLTVDKSRW |
| | QEGNVFSCSVLHEALHSHYTQKSLSLSLG |
| SEQ | QIQLVQSGSELKKPGASVKVSCKASGYTFTDYAMNWVRQAPGQGLEWMGW |
| ID | INTQTGKPTYAQGFTGRFVFSLDTSVSTAYLQISSLKAEDTAVYYCTRLGTGN |
| NO: | TKGFAYWGQGTLVTVSSASTKGPSVFPLAPCSRSTSESTAALGCLVKDYFPEP |
| 132 | VTVSWNSGALTSGVHTFPAVLQSSGLYSLSSVVTVPSSSLGTKTYTCNVDHKP |
| | SNTKVDKRVESKYGPPCPPCPAPEFEGGPSVFLFPPKPKDTLYITREPEVTCVV |
| | VDVSQEDPEVQFNWYVDGVEVHNAKTKPREEQFNSTYRVVSVLTVLHQDW |
| | LNGKEYKCKVSNKGLPSSIEKTISKAKGQPREPQVYTLPPSQEEMTKNQVSLT |
| | CLVKGFYPSDIAVEWESNGQPENNYKTTPPVLDSDGSFFLYSRLTVDKSRWQ |
| | EGNVFSCSVMHEALHNHYTQKSLSLSLG |
| SEQ | QIQLVQSGAEVKKPGSSVKVSCKASGYTFTDYAMNWVRQAPGQGLEWMGW |
| ID | INTQTGKPTYAQKFQGRVTFTLDESTSTAYMELSSLRSEDTAVYYCTRLGTGN |
| NO: | TKGFAYWGQGTLVTVSSASTKGPSVFPLAPCSRSTSESTAALGCLVKDYFPEP |
| 133 | VTVSWNSGALTSGVHTFPAVLQSSGLYSLSSVVTVPSSSLGTKTYTCNVDHKP |
| | SNTKVDKRVESKYGPPCPPCPAPEFEGGPSVFLFPPKPKDTLYITREPEVTCVV |
| | VDVSQEDPEVQFNWYVDGVEVHNAKTKPREEQFNSTYRVVSVLTVLHQDW |
| | LNGKEYKCKVSNKGLPSSIEKTISKAKGQPREPQVYTLPPSQEEMTKNQVSLT |
| | CLVKGFYPSDIAVEWESNGQPENNYKTTPPVLDSDGSFFLYSRLTVDKSRWQ |
| | EGNVFSCSVMHEALHNHYTQKSLSLSLG |
| SEQ | QIQLVQSGAEVKKPGSSVKVSCKASGYTFTDYAMNWVRQAPGQGLEWMGW |
| ID | INTQTGKPTYAQKFQGRVTFTLDESTSTTYMELSSLRSEDTAVYYCARLGTGN |
| NO: | TKGFAYWGQGTLVTVSSASTKGPSVFPLAPCSRSTSESTAALGCLVKDYFPEP |
| 134 | VTVSWNSGALTSGVHTFPAVLQSSGLYSLSSVVTVPSSSLGTKTYTCNVDHKP |
| | SNTKVDKRVESKYGPPCPPCPAPEFEGGPSVFLFPPKPKDTLYITREPEVTCVV |
| | VDVSQEDPEVQFNWYVDGVEVHNAKTKPREEQFNSTYRVVSVLTVLHQDW |
| | LNGKEYKCKVSNKGLPSSIEKTISKAKGQPREPQVYTLPPSQEEMTKNQVSLT |
| | CLVKGFYPSDIAVEWESNGQPENNYKTTPPVLDSDGSFFLYSRLTVDKSRWQ |
| | EGNVFSCSVMHEALHNHYTQKSLSLSLG |
| SEQ | QIQLVQSGAEVKKPGSSVKVSCKASGYTFTDYAMNWVRQAPGQGLEWMGW |
| ID | INTQTGKPTYAQKFQGRVTFTLDESTSTTYMELSSLRSEDTAVYYCTRLGTGN |
| NO: | TKGFAYWGQGTLVTVSSASTKGPSVFPLAPCSRSTSESTAALGCLVKDYFPEP |
| 135 | VTVSWNSGALTSGVHTFPAVLQSSGLYSLSSVVTVPSSSLGTKTYTCNVDHKP |
| | SNTKVDKRVESKYGPPCPPCPAPEFEGGPSVFLFPPKPKDTLYITREPEVTCVV |
| | VDVSQEDPEVQFNWYVDGVEVHNAKTKPREEQFNSTYRVVSVLTVLHQDW |
| | LNGKEYKCKVSNKGLPSSIEKTISKAKGQPREPQVYTLPPSQEEMTKNQVSLT |
| | CLVKGFYPSDIAVEWESNGQPENNYKTTPPVLDSDGSFFLYSRLTVDKSRWQ |
| | EGNVFSCSVMHEALHNHYTQKSLSLSLG |
| SEQ | QIQLVQSGPEVKKPGESVKISCKASGYTFTDYAMNWVKQAPGNGLKWMGWI |
| ID | NTQTGKPTYADDFKQRFVFSLETSARTTYLQINNLNIEDTATYFCTRLGTGNT |
| NO: | KGFAYWGQGTLVTVSSASTKGPSVFPLAPCSRSTSESTAALGCLVKDYFPEPV |
| 136 | TVSWNSGALTSGVHTFPAVLQSSGLYSLSSVVTVPSSSLGTKTYTCNVDHKPS |
| | NTKVDKRVESKYGPPCPPCPAPEFEGGPSVFLFPPKPKDTLYITREPEVTCVVV |
| | DVSQEDPEVQFNWYVDGVEVHNAKTKPREEQFNSTYRVVSVLTVLHQDWL |
| | NGKEYKCKVSNKGLPSSIEKTISKAKGQPREPQVYTLPPSQEEMTKNQVSLTC |
| | LVKGFYPSDIAVEWESNGQPENNYKTTPPVLDSDGSFFLYSRLTVDKSRWQE |
| | GNVFSCSVMHEALHNHYTQKSLSLSLG |
| SEQ | QIQLVQSGSELKKPGASVKISCKASGYTFTDYAMNWVKQAPGQGLEWMGWI |
| ID | NTQTGKPTYAQGFTGRFVFSLDTSVSTAYLQISSLKAEDTAVYFCTRLGTGNT |
| NO: | KGFAYWGQGTLVTVSSASTKGPSVFPLAPCSRSTSESTAALGCLVKDYFPEPV |
| 137 | TVSWNSGALTSGVHTFPAVLQSSGLYSLSSVVTVPSSSLGTKTYTCNVDHKPS |
| | NTKVDKRVESKYGPPCPPCPAPEFEGGPSVFLFPPKPKDTLYITREPEVTCVVV |
| | DVSQEDPEVQFNWYVDGVEVHNAKTKPREEQFNSTYRVVSVLTVLHQDWL |
| | NGKEYKCKVSNKGLPSSIEKTISKAKGQPREPQVYTLPPSQEEMTKNQVSLTC |
| | LVKGFYPSDIAVEWESNGQPENNYKTTPPVLDSDGSFFLYSRLTVDKSRWQE |
| | GNVFSCSVMHEALHNHYTQKSLSLSLG |
| SEQ | QIQLVQSGSELKKPGASVKISCKASGYTFTDYAMNWVKQAPGQGLEWMGWI |
| ID | NTQTGKPTYAQGFTGRFVFSLDTSVSTAYLQISSLKAEDTAVYFCTRLGTGNT |
| NO: | KGFAYWGQGTLVTVSSASTKGPSVFPLAPCSRSTSESTAALGCLVKDYFPEPV |
| 138 | TVSWNSGALTSGVHTFPAVLQSSGLYSLSSVVTVPSSSLGTKTYTCNVDHKPS |
| | NTKVDKRVESKYGPPCPPCPAPEFEGGPSVFLFPPKPKDTLYITREPEVTCVVV |
| | DVSQEDPEVQFNWYVDGVEVHNAKTKPREEQFNSTYRVVSVLTVLHQDWL |
| | NGKEYKCKVSNKGLPSSIEKTISKAKGQPREPQVYTLPPSQEEMTKNQVSLTC |
| | LVKGFYPSDIAVEWESNGQPENNYKTTPPVLDSDGSFFLYSRLTVDKSRWQE |
| | GNVFSCSVMHEALHNHYTQKSLSLSLG |
| SEQ | QIQLVQSGSELKKPGASVKISCKASGYTFTDYAMNWVRQAPGQGLEWMGWI |
| ID | NTQTGKPTYAQGFTGRFVFSLDTSVRTAYLQISSLKAEDTAVYFCTRLGTGNT |
| NO: | KGFAYWGQGTLVTVSSASTKGPSVFPLAPCSRSTSESTAALGCLVKDYFPEPV |
| 139 | TVSWNSGALTSGVHTFPAVLQSSGLYSLSSVVTVPSSSLGTKTYTCNVDHKPS |
| | NTKVDKRVESKYGPPCPPCPAPEFEGGPSVFLFPPKPKDTLYITREPEVTCVVV |
| | DVSQEDPEVQFNWYVDGVEVHNAKTKPREEQFNSTYRVVSVLTVLHQDWL |
| | NGKEYKCKVSNKGLPSSIEKTISKAKGQPREPQVYTLPPSQEEMTKNQVSLTC |
| | LVKGFYPSDIAVEWESNGQPENNYKTTPPVLDSDGSFFLYSRLTVDKSRWQE |
| | GNVFSCSVMHEALHNHYTQKSLSLSLG |
| SEQ | QIQLVQSGSELKKPGASVKISCKASGYTFTDYAMNWVRQAPGQGLEWMGWI |
| ID | NTQTGKPTYAQGFTGRFVFSLDTSVSTAYLQISSLKAEDTAVYFCTRLGTGNT |
| NO: | KGFAYWGQGTLVTVSSASTKGPSVFPLAPCSRSTSESTAALGCLVKDYFPEPV |
| 140 | TVSWNSGALTSGVHTFPAVLQSSGLYSLSSVVTVPSSSLGTKTYTCNVDHKPS |
| | NTKVDKRVESKYGPPCPPCPAPEFEGGPSVFLFPPKPKDTLYITREPEVTCVVV |
| | DVSQEDPEVQFNWYVDGVEVHNAKTKPREEQFNSTYRVVSVLTVLHQDWL |
| | NGKEYKCKVSNKGLPSSIEKTISKAKGQPREPQVYTLPPSQEEMTKNQVSLTC |
| | LVKGFYPSDIAVEWESNGQPENNYKTTPPVLDSDGSFFLYSRLTVDKSRWQE |
| | GNVFSCSVMHEALHNHYTQKSLSLSLG |
| SEQ | QIQLVQSGSELKKPGASVKISCKASGYTFTDYAMNWVRQAPGQGLEWMGWI |
| ID | NTQTGKPTYAQGFTGRFVFSLDTSVSTAYLQISSLKAEDTAVYYCTRLGTGNT |
| NO: | KGFAYWGQGTLVTVSSASTKGPSVFPLAPCSRSTSESTAALGCLVKDYFPEPV |
| 141 | TVSWNSGALTSGVHTFPAVLQSSGLYSLSSVVTVPSSSLGTKTYTCNVDHKPS |
| | NTKVDKRVESKYGPPCPPCPAPEFEGGPSVFLFPPKPKDTLYITREPEVTCVVV |
| | DVSQEDPEVQFNWYVDGVEVHNAKTKPREEQFNSTYRVVSVLTVLHQDWL |
| | NGKEYKCKVSNKGLPSSIEKTISKAKGQPREPQVYTLPPSQEEMTKNQVSLTC |
| | LVKGFYPSDIAVEWESNGQPENNYKTTPPVLDSDGSFFLYSRLTVDKSRWQE |
| | GNVFSCSVMHEALHNHYTQKSLSLSLG |
| SEQ | QIQLVQSGSELKKPGASVKVSCKASGYTFTDYAMNWVRQAPGQGLEWMGW |
| ID | INTQTGKPTYAQGFTGRFVFSLDTSVSTAYLQISSLKAEDTAVYFCTRLGTGN |
| NO: | TKGFAYWGQGTLVTVSSASTKGPSVFPLAPCSRSTSESTAALGCLVKDYFPEP |
| 142 | VTVSWNSGALTSGVHTFPAVLQSSGLYSLSSVVTVPSSSLGTKTYTCNVDHKP |
| | SNTKVDKRVESKYGPPCPPCPAPEFEGGPSVFLFPPKPKDTLYITREPEVTCVV |
| | VDVSQEDPEVQFNWYVDGVEVHNAKTKPREEQFNSTYRVVSVLTVLHQDW |
| | LNGKEYKCKVSNKGLPSSIEKTISKAKGQPREPQVYTLPPSQEEMTKNQVSLT |
| | CLVKGFYPSDIAVEWESNGQPENNYKTTPPVLDSDGSFFLYSRLTVDKSRWQ |
| | EGNVFSCSVMHEALHNHYTQKSLSLSLG |
| SEQ | QVQLVQSGAEVKKPGSSVKVSCKASGYTFTDYAMNWVRQAPGQGLEWMG |
| ID | WINTQTGKPTYAQKFQGRVTFTLDESTSTAYMELSSLRSEDTAVYYCTRLGT |
| NO: | GNTKGFAYWGQGTLVTVSSASTKGPSVFPLAPCSRSTSESTAALGCLVKDYFP |
| 143 | EPVTVSWNSGALTSGVHTFPAVLQSSGLYSLSSVVTVPSSSLGTKTYTCNVDH |
| | KPSNTKVDKRVESKYGPPCPPCPAPEFEGGPSVFLFPPKPKDTLYITREPEVTC |
| | VVVDVSQEDPEVQFNWYVDGVEVHNAKTKPREEQFNSTYRVVSVLTVLHQD |
| | WLNGKEYKCKVSNKGLPSSIEKTISKAKGQPREPQVYTLPPSQEEMTKNQVSL |
| | TCLVKGFYPSDIAVEWESNGQPENNYKTTPPVLDSDGSFFLYSRLTVDKSRW |
| | QEGNVFSCSVMHEALHNHYTQKSLSLSLG |
| SEQ | QVQLVQSGAEVKKPGASVKVSCKASGYTFTSYGISWVRQAPGQGLEWMGWI |
| ID | SAYNANTNYAQKLQGRVTMTTDTSTSTAYMELRSLRSDDTAAYYCARVTGI |
| NO: | TGTTIGPWGQGTMVTVSSASTKGPSVFPLAPSSKSTSGGTAALGCLVKDYFPE |
| 148 | PVTVSWNSGALTSGVHTFPAVLQSSGLYSLSSVVTVPSSSLGTQTYICNVNHK |
| | PSNTKVDKKVEPKSCDKTHTCPPCPAPEAAGGPSVFLFPPKPKDTLMISRTPEV |
| | TCVVVSVSHEDPEVKFNWYVDGVEVHNAKTKPREEQYNSTYRVVSVLTVLH |
| | QDWLNGKEYKCKVSNKALPAPIEKTISKAKGQPREPQVYTLPPSREEMTKNQ |
| | VSLTCLVKGFYPSDIAVEWESNGQPENNYKTTPPVLDSDGSFFLYSKLTVDKS |
| | RWQQGNVFSCSVMHEALHNHYTQKSLSLSPGK |
| SEQ | QVQLVQSGAEVKKPGASVKVSCKASGYTFTSYGISWVRQAPGQGLEWMGWI |
| ID | SAYNANVNYAQKLQGRVTMTTDTSTSTAYMELRSLRSDDTAAYYCARVTGI |
| NO: | TGTTIGPWGQGTMVTVSSASTKGPSVFPLAPSSKSTSGGTAALGCLVKDYFPE |
| 149 | PVTVSWNSGALTSGVHTFPAVLQSSGLYSLSSVVTVPSSSLGTQTYICNVNHK |
| | PSNTKVDKKVEPKSCDKTHTCPPCPAPEAAGGPSVFLFPPKPKDTLMISRTPEV |
| | TCVVVSVSHEDPEVKFNWYVDGVEVHNAKTKPREEQYNSTYRVVSVLTVLH |
| | QDWLNGKEYKCKVSNKALPAPIEKTISKAKGQPREPQVYTLPPSREEMTKNQ |
| | VSLTCLVKGFYPSDIAVEWESNGQPENNYKTTPPVLDSDGSFFLYSKLTVDKS |
| | RWQQGNVFSCSVMHEALHNHYTQKSLSLSPGK |
| SEQ | QVQLVQSGAEVKKPGASVKVSCKASGYTFTSYGISWVRQAPGQGLEWMGWI |
| ID | SAYNGNTNYAQKLQGRVTMTTDTSTSTAYMELRSLRSDDTAAYYCARVTGI |
| NO: | TGTTIDPWGQGTMVTVSSASTKGPSVFPLAPSSKSTSGGTAALGCLVKDYFPE |
| 150 | PVTVSWNSGALTSGVHTFPAVLQSSGLYSLSSVVTVPSSSLGTQTYICNVNHK |
| | PSNTKVDKKVEPKSCDKTHTCPPCPAPEAAGGPSVFLFPPKPKDTLMISRTPEV |
| | TCVVVSVSHEDPEVKFNWYVDGVEVHNAKTKPREEQYNSTYRVVSVLTVLH |
| | QDWLNGKEYKCKVSNKALPAPIEKTISKAKGQPREPQVYTLPPSREEMTKNQ |
| | VSLTCLVKGFYPSDIAVEWESNGQPENNYKTTPPVLDSDGSFFLYSKLTVDKS |
| | RWQQGNVFSCSVMHEALHNHYTQKSLSLSPGK |
| SEQ | QVQLVQSGAEVKKPGASVKVSCKASGYTFTSYGISWVRQAPGQGLEWMGWI |
| ID | SAYNSNTNYAQKLQGRVTMTTDTSTSTAYMELRSLRSDDTAAYYCARVTGIT |
| NO: | GTTIGPWGQGTMVTVSSASTKGPSVFPLAPSSKSTSGGTAALGCLVKDYFPEP |
| 151 | VTVSWNSGALTSGVHTFPAVLQSSGLYSLSSVVTVPSSSLGTQTYICNVNHKP |
| | SNTKVDKKVEPKSCDKTHTCPPCPAPEAAGGPSVFLFPPKPKDTLMISRTPEVT |
| | CVVVSVSHEDPEVKFNWYVDGVEVHNAKTKPREEQYNSTYRVVSVLTVLHQ |
| | DWLNGKEYKCKVSNKALPAPIEKTISKAKGQPREPQVYTLPPSREEMTKNQV |
| | SLTCLVKGFYPSDIAVEWESNGQPENNYKTTPPVLDSDGSFFLYSKLTVDKSR |
| | WQQGNVFSCSVMHEALHNHYTQKSLSLSPGK |
| SEQ | QVQLVQSGAEVKKPGASVKVSCKASGYTFTSYGISWVRQAPGQGLEWMGWI |
| ID | SAYNANTNYAQKLQGRVTMTTDTSTSTAYMELRSLRSDDTAAYYCARVTGI |
| NO: | TGTTIGPWGQGTMVTVSSASTKGPSVFPLAPCSRSTSESTAALGCLVKDYFPE |
| 152 | PVTVSWNSGALTSGVHTFPAVLQSSGLYSLSSVVTVPSSSLGTKTYTCNVDHK |
| | PSNTKVDKRVESKYGPPCPPCPAPEFEGGPSVFLFPPKPKDTLMISRTPEVTCV |
| | VVDVSQEDPEVQFNWYVDGVEVHNAKTKPREEQFNSTYRVVSVLIVLHQD |
| | WLNGKEYKCKVSNKGLPSSIEKTISKAKGQPREPQVYTLPPSQEEMTKNQVSL |
| | TCLVKGFYPSDIAVEWESNGQPENNYKTTPPVLDSDGSFFLYSRLTVDKSRW |
| | QEGNVFSCSVLHEALHSHYTQKSLSLSLGK |
| SEQ | QVQLVQSGAEVKKPGASVKVSCKASGYTFTSYGISWVRQAPGQGLEWMGWI |
| ID | SAYNANVNYAQKLQGRVTMTTDTSTSTAYMELRSLRSDDTAAYYCARVTGI |
| NO: | TGTTIGPWGQGTMVTVSSASTKGPSVFPLAPCSRSTSESTAALGCLVKDYFPE |
| 153 | PVTVSWNSGALTSGVHTFPAVLQSSGLYSLSSVVTVPSSSLGTKTYTCNVDHK |
| | PSNTKVDKRVESKYGPPCPPCPAPEFEGGPSVFLFPPKPKDTLMISRTPEVTCV |
| | VVDVSQEDPEVQFNWYVDGVEVHNAKTKPREEQFNSTYRVVSVLTVLHQD |
| | WLNGKEYKCKVSNKGLPSSIEKTISKAKGQPREPQVYTLPPSQEEMTKNQVSL |
| | TCLVKGFYPSDIAVEWESNGQPENNYKTTPPVLDSDGSFFLYSRLTVDKSRW |
| | QEGNVFSCSVLHEALHSHYTQKSLSLSLGK |
| SEQ | QVQLVQSGAEVKKPGASVKVSCKASGYTFTSYGISWVRQAPGQGLEWMGWI |
| ID | SAYNGNTNYAQKLQGRVTMTTDTSTSTAYMELRSLRSDDTAAYYCARVTGI |
| NO: | TGTTIDPWGQGTMVTVSSASTKGPSVFPLAPCSRSTSESTAALGCLVKDYFPE |
| 154 | PVTVSWNSGALTSGVHTFPAVLQSSGLYSLSSVVTVPSSSLGTKTYTCNVDHK |
| | PSNTKVDKRVESKYGPPCPPCPAPEFEGGPSVFLFPPKPKDTLMISRTPEVTCV |
| | VVDVSQEDPEVQFNWYVDGVEVHNAKTKPREEQFNSTYRVVSVLTVLHQD |
| | WLNGKEYKCKVSNKGLPSSIEKTISKAKGQPREPQVYTLPPSQEEMTKNQVSL |
| | TCLVKGFYPSDIAVEWESNGQPENNYKTTPPVLDSDGSFFLYSRLTVDKSRW |
| | QEGNVFSCSVLHEALHSHYTQKSLSLSLGK |
| SEQ | ASTKGPSVFPLAPCSRSTSESTAALGCLVKDYFPEPVTVSWNSGALTSGVHTF |
| ID | PAVLQSSGLYSLSSVVTVPSSSLGTKTYTCNVDHKPSNTKVDKRVESKYGPPC |
| NO: | PPCPAPEFEGGPSVFLFPPKPKDTLMISRTPEVTCVVVDVSQEDPEVQFNWYV |
| 155 | DGVEVHNAKTKPREEQFNSTYRVVSVLTVLHQDWLNGKEYKCKVSNKGLPS |
| | SIEKTISKAKGQPREPQVYTLPPSQEEMTKNQVSLTCLVKGFYPSDIAVEWESN |
| | GQPENNYKTTPPVLDSDGSFFLYSRLTVDKSRWQEGNVFSCSVLHEALHSHY |
| | TQKSLSLSLGKQVQLVQSGAEVKKPGASVKVSCKASGYTFTSYGISWVRQAP |
| | GQGLEWMGWISAYNSNTNYAQKLQGRVTMTTDTSTSTAYMELRSLRSDDTA |
| | AYYCARVTGITGTTIGPWGQGTMVTVSS |
| SEQ | QVQLVQSGAEVKKPGASVKVSCKASGYTFTSYGISWVRQAPGQGLEWMGWI |
| ID | SAYNANTNYAQKLQGRVTMTTDTSTSTAYMELRSLRSDDTAAYYCARVTGI |
| NO: | TGTTIGPWGQGTMVTVSSASTKGPSVFPLAPCSRSTSESTAALGCLVKDYFPE |
| 156 | PVTVSWNSGALTSGVHTFPAVLQSSGLYSLSSVVTVPSSSLGTKTYTCNVDHK |
| | PSNTKVDKRVESKYGPPCPPCPAPEFEGGPSVFLFPPKPKDTLYITREPEVTCV |
| | VVDVSQEDPEVQFNWYVDGVEVHNAKTKPREEQFNSTYRVVSVLTVLHQD |
| | WLNGKEYKCKVSNKGLPSSIEKTISKAKGQPREPQVYTLPPSQEEMTKNQVSL |
| | TCLVKGFYPSDIAVEWESNGQPENNYKTTPPVLDSDGSFFLYSRLTVDKSRW |
| | QEGNVFSCSVMHEALHNHYTQKSLSLSLGK |
| SEQ | QVQLVQSGAEVKKPGASVKVSCKASGYTFTSYGISWVRQAPGQGLEWMGWI |
| ID | SAYNANVNYAQKLQGRVTMTTDTSTSTAYMELRSLRSDDTAAYYCARVTGI |
| NO: | TGTTIGPWGQGTMVTVSSASTKGPSVFPLAPCSRSTSESTAALGCLVKDYFPE |
| 157 | PVTVSWNSGALTSGVHTFPAVLQSSGLYSLSSVVTVPSSSLGTKTYTCNVDHK |
| | PSNTKVDKRVESKYGPPCPPCPAPEFEGGPSVFLFPPKPKDTLYITREPEVTCV |
| | VVDVSQEDPEVQFNWYVDGVEVHNAKTKPREEQFNSTYRVVSVLTVLHQD |
| | WLNGKEYKCKVSNKGLPSSIEKTISKAKGQPREPQVYTLPPSQEEMTKNQVSL |
| | TCLVKGFYPSDIAVEWESNGQPENNYKTTPPVLDSDGSFFLYSRLTVDKSRW |
| | QEGNVFSCSVMHEALHNHYTQKSLSLSLGK |
| SEQ | QVQLVQSGAEVKKPGASVKVSCKASGYTFTSYGISWVRQAPGQGLEWMGWI |
| ID | SAYNGNTNYAQKLQGRVTMTTDTSTSTAYMELRSLRSDDTAAYYCARVTGI |
| NO: | TGTTIDPWGQGTMVTVSSASTKGPSVFPLAPCSRSTSESTAALGCLVKDYFPE |
| 158 | PVTVSWNSGALTSGVHTFPAVLQSSGLYSLSSVVTVPSSSLGTKTYTCNVDHK |
| | PSNTKVDKRVESKYGPPCPPCPAPEFEGGPSVFLFPPKPKDTLYITREPEVTCV |
| | VVDVSQEDPEVQFNWYVDGVEVHNAKTKPREEQFNSTYRVVSVLTVLHQD |
| | WLNGKEYKCKVSNKGLPSSIEKTISKAKGQPREPQVYTLPPSQEEMTKNQVSL |
| | TCLVKGFYPSDIAVEWESNGQPENNYKTTPPVLDSDGSFFLYSRLTVDKSRW |
| | QEGNVFSCSVMHEALHNHYTQKSLSLSLGK |
| SEQ | QVQLVQSGAEVKKPGASVKVSCKASGYTFTSYGISWVRQAPGQGLEWMGWI |
| ID | SAYNSNTNYAQKLQGRVTMTTDTSTSTAYMELRSLRSDDTAAYYCARVTGIT |
| NO: | GTTIGPWGQGTMVTVSSASTKGPSVFPLAPCSRSTSESTAALGCLVKDYFPEP |
| 159 | VTVSWNSGALTSGVHTFPAVLQSSGLYSLSSVVTVPSSSLGTKTYTCNVDHKP |
| | SNTKVDKRVESKYGPPCPPCPAPEFEGGPSVFLFPPKPKDTLYITREPEVTCVV |
| | VDVSQEDPEVQFNWYVDGVEVHNAKTKPREEQFNSTYRVVSVLTVLHQDW |
| | LNGKEYKCKVSNKGLPSSIEKTISKAKGQPREPQVYTLPPSQEEMTKNQVSLT |
| | CLVKGFYPSDIAVEWESNGQPENNYKTTPPVLDSDGSFFLYSRLTVDKSRWQ |
| | EGNVFSCSVMHEALHNHYTQKSLSLSLGK |
| SEQ | QVQLVQSGAEVKKPGASVKVSCKASGYTFTSYGISWVRQAPGQGLEWMGWI |
| ID | SAYNANTNYAQKLQGRVTMTTDTSTSTAYMELRSLRSDDTAAYYCARVTGI |
| NO: | TGTTIGPWGQGTMVTVSSASTKGPSVFPLAPSSKSTSGGTAALGCLVKDYFPE |
| 160 | PVTVSWNSGALTSGVHTFPAVLQSSGLYSLSSVVTVPSSSLGTQTYICNVNHK |
| | PSNTKVDKKVEPKSCDKTHTCPPCPAPEAAGGPSVFLFPPKPKDTLMISRTPEV |
| | TCVVVSVSHEDPEVKFNWYVDGVEVHNAKTKPREEQYNSTYRVVSVLTVLH |
| | QDWLNGKEYKCKVSNKALPAPIEKTISKAKGQPREPQVYTLPPSREEMTKNQ |
| | VSLTCLVKGFYPSDIAVEWESNGQPENNYKTTPPVLDSDGSFFLYSKLTVDKS |
| | RWQQGNVFSCSVMHEALHNHYTQKSLSLSPG |
| SEQ | QVQLVQSGAEVKKPGASVKVSCKASGYTFTSYGISWVRQAPGQGLEWMGWI |
| ID | SAYNANVNYAQKLQGRVTMTTDTSTSTAYMELRSLRSDDTAAYYCARVTGI |
| NO: | TGTTIGPWGQGTMVTVSSASTKGPSVFPLAPSSKSTSGGTAALGCLVKDYFPE |
| 161 | PVTVSWNSGALTSGVHTFPAVLQSSGLYSLSSVVTVPSSSLGTQTYICNVNHK |
| | PSNTKVDKKVEPKSCDKTHTCPPCPAPEAAGGPSVFLFPPKPKDTLMISRTPEV |
| | TCVVVSVSHEDPEVKFNWYVDGVEVHNAKTKPREEQYNSTYRVVSVLTVLH |
| | QDWLNGKEYKCKVSNKALPAPIEKTISKAKGQPREPQVYTLPPSREEMTKNQ |
| | VSLTCLVKGFYPSDIAVEWESNGQPENNYKTTPPVLDSDGSFFLYSKLTVDKS |
| | RWQQGNVFSCSVMHEALHNHYTQKSLSLSPG |
| SEQ | QVQLVQSGAEVKKPGASVKVSCKASGYTFTSYGISWVRQAPGQGLEWMGWI |
| ID | SAYNGNTNYAQKLQGRVTMTTDTSTSTAYMELRSLRSDDTAAYYCARVTGI |
| NO: | TGTTIDPWGQGTMVTVSSASTKGPSVFPLAPSSKSTSGGTAALGCLVKDYFPE |
| 162 | PVTVSWNSGALTSGVHTFPAVLQSSGLYSLSSVVTVPSSSLGTQTYICNVNHK |
| | PSNTKVDKKVEPKSCDKTHTCPPCPAPEAAGGPSVFLFPPKPKDTLMISRTPEV |
| | TCVVVSVSHEDPEVKFNWYVDGVEVHNAKTKPREEQYNSTYRVVSVLTVLH |
| | QDWLNGKEYKCKVSNKALPAPIEKTISKAKGQPREPQVYTLPPSREEMTKNQ |
| | VSLTCLVKGFYPSDIAVEWESNGQPENNYKTTPPVLDSDGSFFLYSKLTVDKS |
| | RWQQGNVFSCSVMHEALHNHYTQKSLSLSPG |
| SEQ | QVQLVQSGAEVKKPGASVKVSCKASGYTFTSYGISWVRQAPGQGLEWMGWI |
| ID | SAYNSNTNYAQKLQGRVTMTTDTSTSTAYMELRSLRSDDTAAYYCARVTGIT |
| NO: | GTTIGPWGQGTMVTVSSASTKGPSVFPLAPSSKSTSGGTAALGCLVKDYFPEP |
| 163 | VTVSWNSGALTSGVHTFPAVLQSSGLYSLSSVVTVPSSSLGTQTYICNVNHKP |
| | SNTKVDKKVEPKSCDKTHTCPPCPAPEAAGGPSVFLFPPKPKDTLMISRTPEVT |
| | CVVVSVSHEDPEVKFNWYVDGVEVHNAKTKPREEQYNSTYRVVSVLTVLHQ |
| | DWLNGKEYKCKVSNKALPAPIEKTISKAKGQPREPQVYTLPPSREEMTKNQV |
| | SLTCLVKGFYPSDIAVEWESNGQPENNYKTTPPVLDSDGSFFLYSKLTVDKSR |
| | WQQGNVFSCSVMHEALHNHYTQKSLSLSPG |
| SEQ | QVQLVQSGAEVKKPGASVKVSCKASGYTFTSYGISWVRQAPGQGLEWMGWI |
| ID | SAYNANTNYAQKLQGRVTMTTDTSTSTAYMELRSLRSDDTAAYYCARVTGI |
| NO: | TGTTIGPWGQGTMVTVSSASTKGPSVFPLAPCSRSTSESTAALGCLVKDYFPE |
| 164 | PVTVSWNSGALTSGVHTFPAVLQSSGLYSLSSVVTVPSSSLGTKTYTCNVDHK |
| | PSNTKVDKRVESKYGPPCPPCPAPEFEGGPSVFLFPPKPKDTLMISRTPEVTCV |
| | VVDVSQEDPEVQFNWYVDGVEVHNAKTKPREEQFNSTYRVVSVLTVLHQD |
| | WLNGKEYKCKVSNKGLPSSIEKTISKAKGQPREPQVYTLPPSQEEMTKNQVSL |
| | TCLVKGFYPSDIAVEWESNGQPENNYKTTPPVLDSDGSFFLYSRLTVDKSRW |
| | QEGNVFSCSVLHEALHSHYTQKSLSLSLG |
| SEQ | QVQLVQSGAEVKKPGASVKVSCKASGYTFTSYGISWVRQAPGQGLEWMGWI |
| ID | SAYNANVNYAQKLQGRVTMTTDTSTSTAYMELRSLRSDDTAAYYCARVTGI |
| NO: | TGTTIGPWGQGTMVTVSSASTKGPSVFPLAPCSRSTSESTAALGCLVKDYFPE |
| 165 | PVTVSWNSGALTSGVHTFPAVLQSSGLYSLSSVVTVPSSSLGTKTYTCNVDHK |
| | PSNTKVDKRVESKYGPPCPPCPAPEFEGGPSVFLFPPKPKDTLMISRTPEVTCV |
| | VVDVSQEDPEVQFNWYVDGVEVHNAKTKPREEQFNSTYRVVSVLTVLHQD |
| | WINGKEYKCKVSNKGLPSSIEKTISKAKGQPREPQVYTLPPSQEEMTKNQVSL |
| | TCLVKGFYPSDIAVEWESNGQPENNYKTTPPVLDSDGSFFLYSRLTVDKSRW |
| | QEGNVFSCSVLHEALHSHYTQKSLSLSLG |
| SEQ | QVQLVQSGAEVKKPGASVKVSCKASGYTFTSYGISWVRQAPGQGLEWMGWI |
| ID | SAYNGNTNYAQKLQGRVTMTTDTSTSTAYMELRSLRSDDTAAYYCARVTGI |
| NO: | TGTTIDPWGQGTMVTVSSASTKGPSVFPLAPCSRSTSESTAALGCLVKDYFPE |
| 166 | PVTVSWNSGALTSGVHTFPAVLQSSGLYSLSSVVTVPSSSLGTKTYTCNVDHK |
| | PSNTKVDKRVESKYGPPCPPCPAPEFEGGPSVFLFPPKPKDTLMISRTPEVTCV |
| | VVDVSQEDPEVQFNWYVDGVEVHNAKTKPREEQFNSTYRVVSVLTVLHQD |
| | WLNGKEYKCKVSNKGLPSSIEKTISKAKGQPREPQVYTLPPSQEEMTKNQVSL |
| | TCLVKGFYPSDIAVEWESNGQPENNYKTTPPVLDSDGSFFLYSRLTVDKSRW |
| | QEGNVFSCSVLHEALHSHYTQKSLSLSLG |
| SEQ | QVQLVQSGAEVKKPGASVKVSCKASGYTFTSYGISWVRQAPGQGLEWMGWI |
| ID | SAYNSNTNYAQKLQGRVTMTTDTSTSTAYMELRSLRSDDTAAYYCARVTGIT |
| NO: | GTTIGPWGQGTMVTVSSASTKGPSVFPLAPCSRSTSESTAALGCLVKDYFPEP |
| 167 | VTVSWNSGALTSGVHTFPAVLQSSGLYSLSSVVTVPSSSLGTKTYTCNVDHKP |
| | SNTKVDKRVESKYGPPCPPCPAPEFEGGPSVFLFPPKPKDTLMISRTPEVTCVV |
| | VDVSQEDPEVQFNWYVDGVEVHNAKTKPREEQFNSTYRVVSVLTVLHQDW |
| | LNGKEYKCKVSNKGLPSSIEKTISKAKGQPREPQVYTLPPSQEEMTKNQVSLT |
| | CLVKGFYPSDIAVEWESNGQPENNYKTTPPVLDSDGSFFLYSRLTVDKSRWQ |
| | EGNVFSCSVLHEALHSHYTQKSLSLSLG |
| SEQ | QVQLVQSGAEVKKPGASVKVSCKASGYTFTSYGISWVRQAPGQGLEWMGWI |
| ID | SAYNANTNYAQKLQGRVTMTTDTSTSTAYMELRSLRSDDTAAYYCARVTGI |
| NO: | TGTTIGPWGQGTMVTVSSASTKGPSVFPLAPCSRSTSESTAALGCLVKDYFPE |
| 168 | PVTVSWNSGALTSGVHTFPAVLQSSGLYSLSSVVTVPSSSLGTKTYTCNVDHK |
| | PSNTKVDKRVESKYGPPCPPCPAPEFEGGPSVFLFPPKPKDTLYITREPEVTCV |
| | VVDVSQEDPEVQFNWYVDGVEVHNAKTKPREEQFNSTYRVVSVLTVLHQD |
| | WLNGKEYKCKVSNKGLPSSIEKTISKAKGQPREPQVYTLPPSQEEMTKNQVSL |
| | TCLVKGFYPSDIAVEWESNGQPENNYKTTPPVLDSDGSFFLYSRLTVDKSRW |
| | QEGNVFSCSVMHEALHNHYTQKSLSLSLG |
| SEQ | QVQLVQSGAEVKKPGASVKVSCKASGYTFTSYGISWVRQAPGQGLEWMGWI |
| ID | SAYNANVNYAQKLQGRVTMTTDTSTSTAYMELRSLRSDDTAAYYCARVTGI |
| NO: | TGTTIGPWGQGTMVTVSSASTKGPSVFPLAPCSRSTSESTAALGCLVKDYFPE |
| 169 | PVTVSWNSGALTSGVHTFPAVLQSSGLYSLSSVVTVPSSSLGTKTYTCNVDHK |
| | PSNTKVDKRVESKYGPPCPPCPAPEFEGGPSVFLFPPKPKDTLYTTREPEVTCV |
| | VVDVSQEDPEVQFNWYVDGVEVHNAKTKPREEQFNSTYRVVSVLTVLHQD |
| | WLNGKEYKCKVSNKGLPSSIEKTISKAKGQPREPQVYTLPPSQEEMTKNQVSL |
| | TCLVKGFYPSDIAVEWESNGQPENNYKTTPPVLDSDGSFFLYSRLTVDKSRW |
| | QEGNVFSCSVMHEALHNHYTQKSLSLSLG |
| SEQ | QVQLVQSGAEVKKPGASVKVSCKASGYTFTSYGISWVRQAPGQGLEWMGWI |
| ID | SAYNGNTNYAQKLQGRVTMTTDTSTSTAYMELRSLRSDDTAAYYCARVTGI |
| NO: | TGTTIDPWGQGTMVTVSSASTKGPSVFPLAPCSRSTSESTAALGCLVKDYFPE |
| 170 | PVTVSWNSGALTSGVHTFPAVLQSSGLYSLSSVVTVPSSSLGTKTYTCNVDHK |
| | PSNTKVDKRVESKYGPPCPPCPAPEFEGGPSVFLFPPKPKDTLYITREPEVTCV |
| | VVDVSQEDPEVQFNWYVDGVEVHNAKTKPREEQFNSTYRVVSVLTVLHQD |
| | WLNGKEYKCKVSNKGLPSSIEKTISKAKGQPREPQVYTLPPSQEEMTKNQVSL |
| | TCLVKGFYPSDIAVEWESNGQPENNYKTTPPVLDSDGSFFLYSRLTVDKSRW |
| | QEGNVFSCSVMHEALHNHYTQKSLSLSLG |
| SEQ | QVQLVQSGAEVKKPGASVKVSCKASGYTFTSYGISWVRQAPGQGLEWMGWI |
| ID | SAYNSNTNYAQKLQGRVTMTTDTSTSTAYMELRSLRSDDTAAYYCARVTGIT |
| NO: | GTTIGPWGQGTMVTVSSASTKGPSVFPLAPCSRSTSESTAALGCLVKDYFPEP |
| 171 | VTVSWNSGALTSGVHTFPAVLQSSGLYSLSSVVTVPSSSLGTKTYTCNVDHKP |
| | SNTKVDKRVESKYGPPCPPCPAPEFEGGPSVFLFPPKPKDTLYITREPEVTCVV |
| | VDVSQEDPEVQFNWYVDGVEVHNAKTKPREEQFNSTYRVVSVLTVLHQDW |
| | LNGKEYKCKVSNKGLPSSIEKTISKAKGQPREPQVYTLPPSQEEMTKNQVSLT |
| | CLVKGFYPSDIAVEWESNGQPENNYKTTPPVLDSDGSFFLYSRLTVDKSRWQ |
| | EGNVFSCSVMHEALHNHYTQKSLSLSLG |
| SEQ | EVQLLESGGGLVQPGGSLRLSCAASGFSFSSSYYMCWVRQAPGKGLEWVSAI |
| ID | GTTRGSTYYADSVKGRFTISRDSSKNTVYLQMNSLRAEDTAVYYCARDATG |
| NO: | YRINTIGLYFNLWGQGTLVTVSSASTKGPSVFPLAPSSKSTSGGTAALGCLVK |
| 173 | DYFPEPVTVSWNSGALTSGVHTFPAVLQSSGLYSLSSVVTVPSSSLGTQTYICN |
| | VNHKPSNTKVDKKVEPKSCDKTHTCPPCPAPEAAGGPSVFLFPPKPKDTLMIS |
| | RTPEVTCVVVSVSHEDPEVKFNWYVDGVEVHNAKTKPREEQYNSTYRVVSV |
| | LTVLHQDWLNGKEYKCKVSNKALPAPIEKTISKAKGQPREPQVYTLPPSREE |
| | MTKNQVSLTCLVKGFYPSDIAVEWESNGQPENNYKTTPPVLDSDGSFFLYSK |
| | LTVDKSRWQQGNVFSCSVMHEALHNHYTQKSLSLSPGK |
| SEQ | EVQLLESGGGLVQPGGSLRLSCAASGFSFSSSYYMAWVRQAPGKGLEWVSAI |
| ID | GTTRGSTYYADSVKGRFTISRDNSKNTLYLQMNSLRAEDTAVYYCARDATG |
| NO: | YRINTIGLYFNLWGQGTLVTVSSASTKGPSVFPLAPSSKSTSGGTAALGCLVK |
| 174 | DYFPEPVTVSWNSGALTSGVHTFPAVLQSSGLYSLSSVVTVPSSSLGTQTYICN |
| | VNHKPSNTKVDKKVEPKSCDKTHTCPPCPAPEAAGGPSVFLFPPKPKDTLMIS |
| | RTPEVTCVVVSVSHEDPEVKFNWYVDGVEVHNAKTKPREEQYNSTYRVVSV |
| | LTVLHQDWLNGKEYKCKVSNKALPAPIEKTISKAKGQPREPQVYTLPPSREE |
| | MTKNQVSLTCLVKGFYPSDIAVEWESNGQPENNYKTTPPVLDSDGSFFLYSK |
| | LTVDKSRWQQGNVFSCSVMHEALHNHYTQKSLSLSPGK |
| SEQ | EVQLLESGGGLVQPGGSLRLSCAASGFSFSSSYYMAWVRQAPGKGLEWVSAI |
| ID | GTTRGSTYYADSVKGRFTISRDNSKNTVYLQMNSLRAEDTAVYYCARDATG |
| NO: | YRINTIGLYFNLWGQGTLVTVSSASTKGPSVFPLAPSSKSTSGGTAALGCLVK |
| 175 | DYFPEPVTVSWNSGALTSGVHTFPAVLQSSGLYSLSSVVTVPSSSLGTQTYICN |
| | VNHKPSNTKVDKKVEPKSCDKTHTCPPCPAPEAAGGPSVFLFPPKPKDTLMIS |
| | RTPEVTCVVVSVSHEDPEVKFNWYVDGVEVHNAKTKPREEQYNSTYRVVSV |
| | LTVLHQDWLNGKEYKCKVSNKALPAPIEKTISKAKGQPREPQVYTLPPSREE |
| | MTKNQVSLTCLVKGFYPSDIAVEWESNGQPENNYKTTPPVLDSDGSFFLYSK |
| | LTVDKSRWQQGNVFSCSVMHEALHNHYTQKSLSLSPGK |
| SEQ | EVQLLESGGGLVQPGGSLRLSCAASGFSFSSSYYMAWVRQAPGKGLEWVSAI |
| ID | GTTRGSTYYADSVKGRFTISRDSSKNTLYLQMNSLRAEDTAVYYCARDATGY |
| NO: | RINTIGLYFNLWGQGTLVTVSSASTKGPSVFPLAPSSKSTSGGTAALGCLVKD |
| 176 | YFPEPVTVSWNSGALTSGVHTFPAVLQSSGLYSLSSVVTVPSSSLGTQTYICNV |
| | NHKPSNTKVDKKVEPKSCDKTHTCPPCPAPEAAGGPSVFLFPPKPKDTLMISR |
| | TPEVTCVVVSVSHEDPEVKFNWYVDGVEVHNAKTKPREEQYNSTYRVVSVL |
| | TVLHQDWLNGKEYKCKVSNKALPAPIEKTISKAKGQPREPQVYTLPPSREEM |
| | TKNQVSLTCLVKGFYPSDIAVEWESNGQPENNYKTTPPVLDSDGSFFLYSKLT |
| | VDKSRWQQGNVFSCSVMHEALHNHYTQKSLSLSPGK |
| SEQ | EVQLLESGGGLVQPGGSLRLSCAASGFSFSSSYYMAWVRQAPGKGLEWVSAI |
| ID | GTTRGSTYYADSVKGRFTISRDSSKNTVYLQMNSLRAEDTAVYFCARDATGY |
| NO: | RINTIGLYFNLWGQGTLVTVSSASTKGPSVFPLAPSSKSTSGGTAALGCLVKD |
| 177 | YFPEPVTVSWNSGALTSGVHTFPAVLQSSGLYSLSSVVTVPSSSLGTQTYICNV |
| | NHKPSNTKVDKKVEPKSCDKTHTCPPCPAPEAAGGPSVFLFPPKPKDTLMISR |
| | TPEVTCVVVSVSHEDPEVKFNWYVDGVEVHNAKTKPREEQYNSTYRVVSVL |
| | TVLHQDWLNGKEYKCKVSNKALPAPIEKTISKAKGQPREPQVYTLPPSREEM |
| | TKNQVSLTCLVKGFYPSDIAVEWESNGQPENNYKTTPPVLDSDGSFFLYSKLT |
| | VDKSRWQQGNVFSCSVMHEALHNHYTQKSLSLSPGK |
| SEQ | EVQLLESGGGLVQPGGSLRLSCAASGFSFSSSYYMCWVRQAPGKGLEWVSCI |
| ID | GTTRGSTYYADSVKGRFTISRDNSKNTLYLQMNSLRAEDTAVYYCARDATG |
| NO: | YRINTIGLYFNLWGQGTLVTVSSASTKGPSVFPLAPSSKSTSGGTAALGCLVK |
| 178 | DYFPEPVTVSWNSGALTSGVHTFPAVLQSSGLYSLSSVVTVPSSSLGTQTYICN |
| | VNHKPSNTKVDKKVEPKSCDKTHTCPPCPAPEAAGGPSVFLFPPKPKDTLMIS |
| | RTPEVTCVVVSVSHEDPEVKFNWYVDGVEVHNAKTKPREEQYNSTYRVVSV |
| | LTVLHQDWLNGKEYKCKVSNKALPAPIEKTISKAKGQPREPQVYTLPPSREE |
| | MTKNQVSLTCLVKGFYPSDIAVEWESNGQPENNYKTTPPVLDSDGSFFLYSK |
| | LTVDKSRWQQGNVFSCSVMHEALHNHYTQKSLSLSPGK |
| SEQ | EVQLLESGGGLVQPGGSLRLSCAASGFSFSSSYYMCWVRQAPGKGLEWVSCI |
| ID | GTTRGSTYYADSVKGRFTISRDNSKNTVYLQMNSLRAEDTAVYYCARDATG |
| NO: | YRINTIGLYFNLWGQGTLVTVSSASTKGPSVFPLAPSSKSTSGGTAALGCLVK |
| 179 | DYFPEPVTVSWNSGALTSGVHTFPAVLQSSGLYSLSSVVTVPSSSLGTQTYICN |
| | VNHKPSNTKVDKKVEPKSCDKTHTCPPCPAPEAAGGPSVFLFPPKPKDTLMIS |
| | RTPEVTCVVVSVSHEDPEVKFNWYVDGVEVHNAKTKPREEQYNSTYRVVSV |
| | LTVLHQDWLNGKEYKCKVSNKALPAPIEKTISKAKGQPREPQVYTLPPSREE |
| | MTKNQVSLTCLVKGFYPSDIAVEWESNGQPENNYKTTPPVLDSDGSFFLYSK |
| | LTVDKSRWQQGNVFSCSVMHEALHNHYTQKSLSLSPGK |
| SEQ | EVQLLESGGGLVQPGGSLRLSCAASGFSFSSSYYMCWVRQAPGKGLEWVSCI |
| ID | GTTRGSTYYADSVKGRFTISRDSSKNTLYLQMNSLRAEDTAVYYCARDATGY |
| NO: | RINTIGLYFNLWGQGTLVTVSSASTKGPSVFPLAPSSKSTSGGTAALGCLVKD |
| 180 | YFPEPVTVSWNSGALTSGVHTFPAVLQSSGLYSLSSVVTVPSSSLGTQTYICNV |
| | NHKPSNTKVDKKVEPKSCDKTHTCPPCPAPEAAGGPSVFLFPPKPKDTLMISR |
| | TPEVTCVVVSVSHEDPEVKFNWYVDGVEVHNAKTKPREEQYNSTYRVVSVL |
| | TVLHQDWLNGKEYKCKVSNKALPAPIEKTISKAKGQPREPQVYTLPPSREEM |
| | TKNQVSLTCLVKGFYPSDIAVEWESNGQPENNYKTTPPVLDSDGSFFLYSKLT |
| | VDKSRWQQGNVFSCSVMHEALHNHYTQKSLSLSPGK |
| SEQ | EVQLLESGGGLVQPGGSLRLSCAASGFSFSSSYYMCWVRQAPGKGLEWVSCI |
| ID | GTTRGSTYYADSVKGRFTISRDSSKNTVYLQMNSLRAEDTAVYFCARDATGY |
| NO: | RINTIGLYFNLWGQGTLVTVSSASTKGPSVFPLAPSSKSTSGGTAALGCLVKD |
| 181 | YFPEPVTVSWNSGALTSGVHTFPAVLQSSGLYSLSSVVTVPSSSLGTQTYICNV |
| | NHKPSNTKVDKKVEPKSCDKTHTCPPCPAPEAAGGPSVFLFPPKPKDTLMISR |
| | TPEVTCVVVSVSHEDPEVKFNWYVDGVEVHNAKTKPREEQYNSTYRVVSVL |
| | TVLHQDWLNGKEYKCKVSNKALPAPIEKTISKAKGQPREPQVYTLPPSREEM |
| | TKNQVSLTCLVKGFYPSDIAVEWESNGQPENNYKTTPPVLDSDGSFFLYSKLT |
| | VDKSRWQQGNVFSCSVMHEALHNHYTQKSLSLSPGK |
| SEQ | EVQLLESGGGLVQPGGSLRLSCAASGFSFSSSYYMSWVRQAPGKGLEWVSAI |
| ID | GTTRGSTYYADSVKGRFTISRDNSKNTLYLQMNSLRAEDTAVYYCARDATG |
| NO: | YRINTIGLYFNLWGQGTLVTVSSASTKGPSVFPLAPSSKSTSGGTAALGCLVK |
| 182 | DYFPEPVTVSWNSGALTSGVHTFPAVLQSSGLYSLSSVVTVPSSSLGTQTYICN |
| | VNHKPSNTKVDKKVEPKSCDKTHTCPPCPAPEAAGGPSVFLFPPKPKDTLMIS |
| | RTPEVTCVVVSVSHEDPEVKFNWYVDGVEVHNAKTKPREEQYNSTYRVVSV |
| | LTVLHQDWLNGKEYKCKVSNKALPAPIEKTISKAKGQPREPQVYTLPPSREE |
| | MTKNQVSLTCLVKGFYPSDIAVEWESNGQPENNYKTTPPVLDSDGSFFLYSK |
| | LTVDKSRWQQGNVFSCSVMHEALHNHYTQKSLSLSPGK |
| SEQ | EVQLLESGGGLVQPGGSLRLSCAASGFSFSSSYYMSWVRQAPGKGLEWVSAI |
| ID | GTTRGSTYYADSVKGRFTISRDNSKNTVYLQMNSLRAEDTAVYYCARDATG |
| NO: | YRINTIGLYFNLWGQGTLVTVSSASTKGPSVFPLAPSSKSTSGGTAALGCLVK |
| 183 | DYFPEPVTVSWNSGALTSGVHTFPAVLQSSGLYSLSSVVTVPSSSLGTQTYICN |
| | VNHKPSNTKVDKKVEPKSCDKTHTCPPCPAPEAAGGPSVFLFPPKPKDTLMIS |
| | RTPEVTCVVVSVSHEDPEVKFNWYVDGVEVHNAKTKPREEQYNSTYRVVSV |
| | LTVLHQDWLNGKEYKCKVSNKALPAPIEKTISKAKGQPREPQVYTLPPSREE |
| | MTKNQVSLTCLVKGFYPSDIAVEWESNGQPENNYKTTPPVLDSDGSFFLYSK |
| | LTVDKSRWQQGNVFSCSVMHEALHNHYTQKSLSLSPGK |
| SEQ | EVQLLESGGGLVQPGGSLRLSCAASGFSFSSSYYMSWVRQAPGKGLEWVSAI |
| ID | GTTRGSTYYADSVKGRFTISRDSSKNTLYLQMNSLRAEDTAVYYCARDATGY |
| NO: | RINTIGLYFNLWGQGTLVTVSSASTKGPSVFPLAPSSKSTSGGTAALGCLVKD |
| 184 | YFPEPVTVSWNSGALTSGVHTFPAVLQSSGLYSLSSVVTVPSSSLGTQTYICNV |
| | NHKPSNTKVDKKVEPKSCDKTHTCPPCPAPEAAGGPSVFLFPPKPKDTLMISR |
| | TPEVTCVVVSVSHEDPEVKENWYVDGVEVHNAKTKPREEQYNSTYRVVSVL |
| | TVLHQDWLNGKEYKCKVSNKALPAPIEKTISKAKGQPREPQVYTLPPSREEM |
| | TKNQVSLTCLVKGFYPSDIAVEWESNGQPENNYKTTPPVLDSDGSFFLYSKLT |
| | VDKSRWQQGNVFSCSVMHEALHNHYTQKSLSLSPGK |
| SEQ | EVQLLESGGGLVQPGGSLRLSCAASGFSFSSSYYMSWVRQAPGKGLEWVSAI |
| ID | GTTRGSTYYADSVKGRFTISRDSSKNTVYLQMNSLRAEDTAVYFCARDATGY |
| NO: | RINTIGLYFNLWGQGTLVTVSSASTKGPSVFPLAPSSKSTSGGTAALGCLVKD |
| 185 | YFPEPVTVSWNSGALTSGVHTFPAVLQSSGLYSLSSVVTVPSSSLGTQTYICNV |
| | NHKPSNTKVDKKVEPKSCDKTHTCPPCPAPEAAGGPSVFLFPPKPKDTLMISR |
| | TPEVTCVVVSVSHEDPEVKFNWYVDGVEVHNAKTKPREEQYNSTYRVVSVL |
| | TVLHQDWLNGKEYKCKVSNKALPAPIEKTISKAKGQPREPQVYTLPPSREEM |
| | TKNQVSLTCLVKGFYPSDIAVEWESNGQPENNYKTTPPVLDSDGSFFLYSKLT |
| | VDKSRWQQGNVFSCSVMHEALHNHYTQKSLSLSPGK |
| SEQ | QSLEESGGDLVKPGASLTLTCRVSGFSFSSSYYMCWVRQAPGKGLEWIACIGT |
| ID | TRGSTYYATWAKGRFTISKISSTTVTLQMTSLTDADTATYFCARDATGYRINT |
| NO: | IGLYFNLWGPGTLVTVSSASTKGPSVFPLAPSSKSTSGGTAALGCLVKDYFPEP |
| 186 | VTVSWNSGALTSGVHTFPAVLQSSGLYSLSSVVTVPSSSLGTQTYICNVNHKP |
| | SNTKVDKKVEPKSCDKTHTCPPCPAPEAAGGPSVFLFPPKPKDTLMISRTPEVT |
| | CVVVSVSHEDPEVKFNWYVDGVEVHNAKTKPREEQYNSTYRVVSVLTVLHQ |
| | DWLNGKEYKCKVSNKALPAPIEKTISKAKGQPREPQVYTLPPSREEMTKNQV |
| | SLTCLVKGFYPSDIAVEWESNGQPENNYKTTPPVLDSDGSFFLYSKLTVDKSR |
| | WQQGNVFSCSVMHEALHNHYTQKSLSLSPGK |
| SEQ | EVQLLESGGGLVQPGGSLRLSCAASGFSFSSSYYMCWVRQAPGKGLEWVSAI |
| ID | GTTRGSTYYADSVKGRFTISRDSSKNTVYLQMNSLRAEDTAVYYCARDATG |
| NO: | YRINTIGLYFNLWGQGTLVTVSSASTKGPSVFPLAPCSRSTSESTAALGCLVKD |
| 187 | YFPEPVTVSWNSGALTSGVHTFPAVLQSSGLYSLSSVVTVPSSSLGTKTYTCN |
| | VDHKPSNTKVDKRVESKYGPPCPPCPAPEFEGGPSVFLFPPKPKDTLMISRTPE |
| | VTCVVVDVSQEDPEVQFNWYVDGVEVHNAKTKPREEQFNSTYRVVSVLTVL |
| | HQDWLNGKEYKCKVSNKGLPSSIEKTISKAKGQPREPQVYTLPPSQEEMTKN |
| | QVSLTCLVKGFYPSDIAVEWESNGQPENNYKTTPPVLDSDGSFFLYSRLTVDK |
| | SRWQEGNVFSCSVLHEALHSHYTQKSLSLSLGK |
| SEQ | EVQLLESGGGLVQPGGSLRLSCAASGFSFSSSYYMAWVRQAPGKGLEWVSAI |
| ID | GTTRGSTYYADSVKGRFTISRDNSKNTLYLQMNSLRAEDTAVYYCARDATG |
| NO: | YRINTIGLYFNLWGQGTLVTVSSASTKGPSVFPLAPCSRSTSESTAALGCLVKD |
| 188 | YFPEPVTVSWNSGALTSGVHTFPAVLQSSGLYSLSSVVTVPSSSLGTKTYTCN |
| | VDHKPSNTKVDKRVESKYGPPCPPCPAPEFEGGPSVFLFPPKPKDTLMISRTPE |
| | VTCVVVDVSQEDPEVQFNWYVDGVEVHNAKTKPREEQFNSTYRVVSVLTVL |
| | HQDWLNGKEYKCKVSNKGLPSSIEKTISKAKGQPREPQVYTLPPSQEEMTKN |
| | QVSLTCLVKGFYPSDIAVEWESNGQPENNYKTTPPVLDSDGSFFLYSRLTVDK |
| | SRWQEGNVFSCSVLHEALHSHYTQKSLSLSLGK |
| SEQ | EVQLLESGGGLVQPGGSLRLSCAASGFSFSSSYYMAWVRQAPGKGLEWVSAI |
| ID | GTTRGSTYYADSVKGRFTISRDNSKNTVYLQMNSLRAEDTAVYYCARDATG |
| NO: | YRINTIGLYFNLWGQGTLVTVSSASTKGPSVFPLAPCSRSTSESTAALGCLVKD |
| 189 | YFPEPVTVSWNSGALTSGVHTFPAVLQSSGLYSLSSVVTVPSSSLGTKTYTCN |
| | VDHKPSNTKVDKRVESKYGPPCPPCPAPEFEGGPSVFLFPPKPKDTLMISRTPE |
| | VTCVVVDVSQEDPEVQFNWYVDGVEVHNAKTKPREEQFNSTYRVVSVLTVL |
| | HQDWLNGKEYKCKVSNKGLPSSIEKTISKAKGQPREPQVYTLPPSQEEMTKN |
| | QVSLTCLVKGFYPSDIAVEWESNGQPENNYKTTPPVLDSDGSFFLYSRLTVDK |
| | SRWQEGNVFSCSVLHEALHSHYTQKSLSLSLGK |
| SEQ | ASTKGPSVFPLAPCSRSTSESTAALGCLVKDYFPEPVTVSWNSGALTSGVHTF |
| ID | PAVLQSSGLYSLSSVVTVPSSSLGTKTYTCNVDHKPSNTKVDKRVESKYGPPC |
| NO: | PPCPAPEFEGGPSVFLFPPKPKDTLMISRTPEVTCVVVDVSQEDPEVQFNWYV |
| 190 | DGVEVHNAKTKPREEQFNSTYRVVSVLIVLHQDWLNGKEYKCKVSNKGLPS |
| | SIEKTISKAKGQPREPQVYTLPPSQEEMTKNQVSLTCLVKGFYPSDIAVEWESN |
| | GQPENNYKTTPPVLDSDGSFFLYSRLTVDKSRWQEGNVFSCSVLHEALHSHY |
| | TQKSLSLSLGKEVQLLESGGGLVQPGGSLRLSCAASGFSFSSSYYMAWVRQA |
| | PGKGLEWVSAIGTTRGSTYYADSVKGRFTISRDSSKNTLYLQMNSLRAEDTA |
| | VYYCARDATGYRINTIGLYFNLWGQGTLVTVSS |
| SEQ | EVQLLESGGGLVQPGGSLRLSCAASGFSFSSSYYMAWVRQAPGKGLEWVSAI |
| ID | GTTRGSTYYADSVKGRFTISRDSSKNTVYLQMNSLRAEDTAVYFCARDATGY |
| NO: | RINTIGLYFNLWGQGTLVTVSSASTKGPSVFPLAPCSRSTSESTAALGCLVKDY |
| 191 | FPEPVTVSWNSGALTSGVHTFPAVLQSSGLYSLSSVVTVPSSSLGTKTYTCNV |
| | DHKPSNTKVDKRVESKYGPPCPPCPAPEFEGGPSVFLFPPKPKDTLMISRTPEV |
| | TCVVVDVSQEDPEVQFNWYVDGVEVHNAKTKPREEQFNSTYRVVSVLTVLH |
| | QDWLNGKEYKCKVSNKGLPSSIEKTISKAKGQPREPQVYTLPPSQEEMTKNQ |
| | VSLTCLVKGFYPSDIAVEWESNGQPENNYKTTPPVLDSDGSFFLYSRLTVDKS |
| | RWQEGNVFSCSVLHEALHSHYTQKSLSLSLGK |
| SEQ | EVQLLESGGGLVQPGGSLRLSCAASGFSFSSSYYMCWVRQAPGKGLEWVSCI |
| ID | GTTRGSTYYADSVKGRFTISRDNSKNTLYLQMNSLRAEDTAVYYCARDATG |
| NO: | YRINTIGLYFNLWGQGTLVTVSSASTKGPSVFPLAPCSRSTSESTAALGCLVKD |
| 192 | YFPEPVTVSWNSGALTSGVHTFPAVLQSSGLYSLSSVVTVPSSSLGTKTYTCN |
| | VDHKPSNTKVDKRVESKYGPPCPPCPAPEFEGGPSVFLFPPKPKDTLMISRTPE |
| | VTCVVVDVSQEDPEVQFNWYVDGVEVHNAKTKPREEQFNSTYRVVSVLTVL |
| | HQDWLNGKEYKCKVSNKGLPSSIEKTISKAKGQPREPQVYTLPPSQEEMTKN |
| | QVSLTCLVKGFYPSDIAVEWESNGQPENNYKTTPPVLDSDGSFFLYSRLTVDK |
| | SRWQEGNVFSCSVLHEALHSHYTQKSLSLSLGK |
| SEQ | EVQLLESGGGLVQPGGSLRLSCAASGFSFSSSYYMCWVRQAPGKGLEWVSCI |
| ID | GTTRGSTYYADSVKGRFTISRDNSKNTVYLQMNSLRAEDTAVYYCARDATG |
| NO: | YRINTIGLYFNLWGQGTLVTVSSASTKGPSVFPLAPCSRSTSESTAALGCLVKD |
| 193 | YFPEPVTVSWNSGALTSGVHTFPAVLQSSGLYSLSSVVTVPSSSLGTKTYTCN |
| | VDHKPSNTKVDKRVESKYGPPCPPCPAPEFEGGPSVFLFPPKPKDTLMISRTPE |
| | VTCVVVDVSQEDPEVQFNWYVDGVEVHNAKTKPREEQFNSTYRVVSVLTVL |
| | HQDWLNGKEYKCKVSNKGLPSSIEKTISKAKGQPREPQVYTLPPSQEEMTKN |
| | QVSLTCLVKGFYPSDIAVEWESNGQPENNYKTTPPVLDSDGSFFLYSRLTVDK |
| | SRWQEGNVFSCSVLHEALHSHYTQKSLSLSLGK |
| SEQ | EVQLLESGGGLVQPGGSLRLSCAASGFSFSSSYYMCWVRQAPGKGLEWVSCI |
| ID | GTTRGSTYYADSVKGRFTISRDSSKNTLYLQMNSLRAEDTAVYYCARDATGY |
| NO: | RINTIGLYFNLWGQGTLVTVSSASTKGPSVFPLAPCSRSTSESTAALGCLVKDY |
| 194 | FPEPVTVSWNSGALTSGVHTFPAVLQSSGLYSLSSVVTVPSSSLGTKTYTCNV |
| | DHKPSNTKVDKRVESKYGPPCPPCPAPEFEGGPSVFLFPPKPKDTLMISRTPEV |
| | TCVVVDVSQEDPEVQFNWYVDGVEVHNAKTKPREEQFNSTYRVVSVLTVLH |
| | QDWLNGKEYKCKVSNKGLPSSIEKTISKAKGQPREPQVYTLPPSQEEMTKNQ |
| | VSLTCLVKGFYPSDIAVEWESNGQPENNYKTTPPVLDSDGSFFLYSRLTVDKS |
| | RWQEGNVFSCSVLHEALHSHYTQKSLSLSLGK |
| SEQ | EVQLLESGGGLVQPGGSLRLSCAASGFSFSSSYYMCWVRQAPGKGLEWVSCI |
| ID | GTTRGSTYYADSVKGRFTISRDSSKNTVYLQMNSLRAEDTAVYFCARDATGY |
| NO: | RINTIGLYFNLWGQGTLVTVSSASTKGPSVFPLAPCSRSTSESTAALGCLVKDY |
| 195 | FPEPVTVSWNSGALTSGVHTFPAVLQSSGLYSLSSVVTVPSSSLGTKTYTCNV |
| | DHKPSNTKVDKRVESKYGPPCPPCPAPEFEGGPSVFLFPPKPKDTLMISRTPEV |
| | TCVVVDVSQEDPEVQFNWYVDGVEVHNAKTKPREEQFNSTYRVVSVLTVLH |
| | QDWLNGKEYKCKVSNKGLPSSIEKTISKAKGQPREPQVYTLPPSQEEMTKNQ |
| | VSLTCLVKGFYPSDIAVEWESNGQPENNYKTTPPVLDSDGSFFLYSRLTVDKS |
| | RWQEGNVFSCSVLHEALHSHYTQKSLSLSLGK |
| SEQ | EVQLLESGGGLVQPGGSLRLSCAASGFSFSSSYYMSWVRQAPGKGLEWVSAI |
| ID | GTTRGSTYYADSVKGRFTISRDNSKNTLYLQMNSLRAEDTAVYYCARDATG |
| NO: | YRINTIGLYFNLWGQGTLVTVSSASTKGPSVFPLAPCSRSTSESTAALGCLVKD |
| 196 | YFPEPVTVSWNSGALTSGVHTFPAVLQSSGLYSLSSVVTVPSSSLGTKTYTCN |
| | VDHKPSNTKVDKRVESKYGPPCPPCPAPEFEGGPSVFLFPPKPKDTLMISRTPE |
| | VTCVVVDVSQEDPEVQFNWYVDGVEVHNAKTKPREEQFNSTYRVVSVLTVL |
| | HQDWLNGKEYKCKVSNKGLPSSIEKTISKAKGQPREPQVYTLPPSQEEMTKN |
| | QVSLTCLVKGFYPSDIAVEWESNGQPENNYKTTPPVLDSDGSFFLYSRLTVDK |
| | SRWQEGNVFSCSVLHEALHSHYTQKSLSLSLGK |
| SEQ | EVQLLESGGGLVQPGGSLRLSCAASGFSFSSSYYMSWVRQAPGKGLEWVSAI |
| ID | GTTRGSTYYADSVKGRFTISRDNSKNTVYLQMNSLRAEDTAVYYCARDATG |
| NO: | YRINTIGLYFNLWGQGTLVTVSSASTKGPSVFPLAPCSRSTSESTAALGCLVKD |
| 197 | YFPEPVTVSWNSGALTSGVHTFPAVLQSSGLYSLSSVVTVPSSSLGTKTYTCN |
| | VDHKPSNTKVDKRVESKYGPPCPPCPAPEFEGGPSVFLFPPKPKDTLMISRTPE |
| | VTCVVVDVSQEDPEVQFNWYVDGVEVHNAKTKPREEQFNSTYRVVSVLTVL |
| | HQDWLNGKEYKCKVSNKGLPSSIEKTISKAKGQPREPQVYTLPPSQEEMTKN |
| | QVSLTCLVKGFYPSDIAVEWESNGQPENNYKTTPPVLDSDGSFFLYSRLTVDK |
| | SRWQEGNVFSCSVLHEALHSHYTQKSLSLSLGK |
| SEQ | EVQLLESGGGLVQPGGSLRLSCAASGFSFSSSYYMSWVRQAPGKGLEWVSAI |
| ID | GTTRGSTYYADSVKGRFTISRDSSKNTLYLQMNSLRAEDTAVYYCARDATGY |
| NO: | RINTIGLYFNLWGQGTLVTVSSASTKGPSVFPLAPCSRSTSESTAALGCLVKDY |
| 198 | FPEPVTVSWNSGALTSGVHTFPAVLQSSGLYSLSSVVTVPSSSLGTKTYTCNV |
| | DHKPSNTKVDKRVESKYGPPCPPCPAPEFEGGPSVFLFPPKPKDTLMISRTPEV |
| | TCVVVDVSQEDPEVQFNWYVDGVEVHNAKTKPREEQFNSTYRVVSVLTVLH |
| | QDWLNGKEYKCKVSNKGLPSSIEKTISKAKGQPREPQVYTLPPSQEEMTKNQ |
| | VSLTCLVKGFYPSDIAVEWESNGQPENNYKTTPPVLDSDGSFFLYSRLTVDKS |
| | RWQEGNVFSCSVLHEALHSHYTQKSLSLSLGK |
| SEQ | EVQLLESGGGLVQPGGSLRLSCAASGFSFSSSYYMSWVRQAPGKGLEWVSAI |
| ID | GTTRGSTYYADSVKGRFTISRDSSKNTVYLQMNSLRAEDTAVYFCARDATGY |
| NO: | RINTIGLYFNLWGQGTLVTVSSASTKGPSVFPLAPCSRSTSESTAALGCLVKDY |
| 199 | FPEPVTVSWNSGALTSGVHTFPAVLQSSGLYSLSSVVTVPSSSLGTKTYTCNV |
| | DHKPSNTKVDKRVESKYGPPCPPCPAPEFEGGPSVFLFPPKPKDTLMISRTPEV |
| | TCVVVDVSQEDPEVQFNWYVDGVEVHNAKTKPREEQFNSTYRVVSVLTVLH |
| | QDWLNGKEYKCKVSNKGLPSSIEKTISKAKGQPREPQVYTLPPSQEEMTKNQ |
| | VSLTCLVKGFYPSDIAVEWESNGQPENNYKTTPPVLDSDGSFFLYSRLTVDKS |
| | RWQEGNVFSCSVLHEALHSHYTQKSLSLSLGK |
| SEQ | QSLEESGGDLVKPGASLTLTCRVSGFSFSSSYYMCWVRQAPGKGLEWIACIGT |
| ID | TRGSTYYATWAKGRFTISKISSTTVTLQMTSLTDADTATYFCARDATGYRINT |
| NO: | IGLYFNLWGPGTLVTVSSASTKGPSVFPLAPCSRSTSESTAALGCLVKDYFPEP |
| 200 | VTVSWNSGALTSGVHTFPAVLQSSGLYSLSSVVTVPSSSLGTKTYTCNVDHKP |
| | SNTKVDKRVESKYGPPCPPCPAPEFEGGPSVFLFPPKPKDTLMISRTPEVTCVV |
| | VDVSQEDPEVQFNWYVDGVEVHNAKTKPREEQFNSTYRVVSVLTVLHQDW |
| | LNGKEYKCKVSNKGLPSSIEKTISKAKGQPREPQVYTLPPSQEEMTKNQVSLT |
| | CLVKGFYPSDIAVEWESNGQPENNYKTTPPVLDSDGSFFLYSRLTVDKSRWQ |
| | EGNVFSCSVLHEALHSHYTQKSLSLSLGK |
| SEQ | EVQLLESGGGLVQPGGSLRLSCAASGFSFSSSYYMCWVRQAPGKGLEWVSAI |
| ID | GTTRGSTYYADSVKGRFTISRDSSKNTVYLQMNSLRAEDTAVYYCARDATG |
| NO: | YRINTIGLYFNLWGQGTLVTVSSASTKGPSVFPLAPCSRSTSESTAALGCLVKD |
| 201 | YFPEPVTVSWNSGALTSGVHTFPAVLQSSGLYSLSSVVTVPSSSLGTKTYTCN |
| | VDHKPSNTKVDKRVESKYGPPCPPCPAPEFEGGPSVFLFPPKPKDTLYITREPE |
| | VTCVVVDVSQEDPEVQFNWYVDGVEVHNAKTKPREEQFNSTYRVVSVLTVL |
| | HQDWLNGKEYKCKVSNKGLPSSIEKTISKAKGQPREPQVYTLPPSQEEMTKN |
| | QVSLTCLVKGFYPSDIAVEWESNGQPENNYKTTPPVLDSDGSFFLYSRLTVDK |
| | SRWQEGNVFSCSVMHEALHNHYTQKSLSLSLGK |
| SEQ | EVQLLESGGGLVQPGGSLRLSCAASGFSFSSSYYMAWVRQAPGKGLEWVSAI |
| ID | GTTRGSTYYADSVKGRFTISRDNSKNTLYLQMNSLRAEDTAVYYCARDATG |
| NO: | YRINTIGLYFNLWGQGTLVTVSSASTKGPSVFPLAPCSRSTSESTAALGCLVKD |
| 202 | YFPEPVTVSWNSGALTSGVHTFPAVLQSSGLYSLSSVVTVPSSSLGTKTYTCN |
| | VDHKPSNTKVDKRVESKYGPPCPPCPAPEFEGGPSVFLFPPKPKDTLYITREPE |
| | VTCVVVDVSQEDPEVQFNWYVDGVEVHNAKTKPREEQFNSTYRVVSVLTVL |
| | HQDWLNGKEYKCKVSNKGLPSSIEKTISKAKGQPREPQVYTLPPSQEEMTKN |
| | QVSLTCLVKGFYPSDIAVEWESNGQPENNYKTTPPVLDSDGSFFLYSRLTVDK |
| | SRWQEGNVFSCSVMHEALHNHYTQKSLSLSLGK |
| SEO | EVQLLESGGGLVQPGGSLRLSCAASGFSFSSSYYMAWVRQAPGKGLEWVSAI |
| ID | GTTRGSTYYADSVKGRFTISRDNSKNTVYLQMNSLRAEDTAVYYCARDATG |
| NO: | YRINTIGLYFNLWGQGTLVTVSSASTKGPSVFPLAPCSRSTSESTAALGCLVKD |
| 203 | YFPEPVTVSWNSGALTSGVHTFPAVLQSSGLYSLSSVVTVPSSSLGTKTYTQN |
| | VDHKPSNTKVDKRVESKYGPPCPPCPAPEFEGGPSVFLFPPKPKDTLYITREPE |
| | VTCVVVDVSQEDPEVQFNWYVDGVEVHNAKTKPREEQFNSTYRVVSVLTVL |
| | HQDWLNGKEYKCKVSNKGLPSSIEKTISKAKGQPREPQVYTLPPSQEEMTKN |
| | QVSLTCLVKGFYPSDIAVEWESNGQPENNYKTTPPVLDSDGSFFLYSRLTVDK |
| | SRWQEGNVFSCSVMHEALHNHYTQKSLSLSLGK |
| SEQ | EVQLLESGGGLVQPGGSLRLSCAASGFSFSSSYYMAWVRQAPGKGLEWVSAI |
| ID | GTTRGSTYYADSVKGRFTISRDSSKNTLYLQMNSLRAEDTAVYYCARDATGY |
| NO: | RINTIGLYFNLWGQGTLVTVSSASTKGPSVFPLAPCSRSTSESTAALGCLVKDY |
| 204 | FPEPVTVSWNSGALTSGVHTFPAVLQSSGLYSLSSVVTVPSSSLGTKTYTCNV |
| | DHKPSNTKVDKRVESKYGPPCPPCPAPEFEGGPSVFLFPPKPKDTLYITREPEV |
| | TCVVVDVSQEDPEVQFNWYVDGVEVHNAKTKPREEQFNSTYRVVSVLTVLH |
| | QDWLNGKEYKCKVSNKGLPSSIEKTISKAKGQPREPQVYTLPPSQEEMTKNQ |
| | VSLTCLVKGFYPSDIAVEWESNGQPENNYKTTPPVLDSDGSFFLYSRLTVDKS |
| | RWQEGNVFSCSVMHEALHNHYTQKSLSLSLGK |
| SEQ | EVQLLESGGGLVQPGGSLRLSCAASGFSFSSSYYMAWVRQAPGKGLEWVSAI |
| ID | GTTRGSTYYADSVKGRFTISRDSSKNTVYLQMNSLRAEDTAVYFCARDATGY |
| NO: | RINTIGLYFNLWGQGTLVTVSSASTKGPSVFPLAPCSRSTSESTAALGCLVKDY |
| 205 | FPEPVTVSWNSGALTSGVHTFPAVLQSSGLYSLSSVVTVPSSSLGTKTYTCNV |
| | DHKPSNTKVDKRVESKYGPPCPPCPAPEFEGGPSVFLFPPKPKDTLYITREPEV |
| | TCVVVDVSQEDPEVQFNWYVDGVEVHNAKTKPREEQFNSTYRVVSVLTVLH |
| | QDWLNGKEYKCKVSNKGLPSSIEKTISKAKGQPREPQVYTLPPSQEEMTKNQ |
| | VSLTCLVKGFYPSDIAVEWESNGQPENNYKTTPPVLDSDGSFFLYSRLTVDKS |
| | RWQEGNVFSCSVMHEALHNHYTQKSLSLSLGK |
| SEQ | EVQLLESGGGLVQPGGSLRLSCAASGFSFSSSYYMCWVRQAPGKGLEWVSCI |
| ID | GTTRGSTYYADSVKGRFTISRDNSKNTLYLQMNSLRAEDTAVYYCARDATG |
| NO: | YRINTIGLYFNLWGQGTLVTVSSASTKGPSVFPLAPCSRSTSESTAALGCLVKD |
| 206 | YFPEPVTVSWNSGALTSGVHTFPAVLQSSGLYSLSSVVTVPSSSLGTKTYTCN |
| | VDHKPSNTKVDKRVESKYGPPCPPCPAPEFEGGPSVFLFPPKPKDTLYITREPE |
| | VTCVVVDVSQEDPEVQFNWYVDGVEVHNAKTKPREEQFNSTYRVVSVLTVL |
| | HQDWLNGKEYKCKVSNKGLPSSIEKTISKAKGQPREPQVYTLPPSQEEMTKN |
| | QVSLTCLVKGFYPSDIAVEWESNGQPENNYKTTPPVLDSDGSFFLYSRLTVDK |
| | SRWQEGNVFSCSVMHEALHNHYTQKSLSLSLGK |
| SEQ | EVQLLESGGGLVQPGGSLRLSCAASGFSFSSSYYMCWVRQAPGKGLEWVSCI |
| ID | GTTRGSTYYADSVKGRFTISRDNSKNTVYLQMNSLRAEDTAVYYCARDATG |
| NO: | YRINTIGLYFNLWGQGTLVTVSSASTKGPSVFPLAPCSRSTSESTAALGCLVKD |
| 207 | YFPEPVTVSWNSGALTSGVHTFPAVLQSSGLYSLSSVVTVPSSSLGTKTYTCN |
| | VDHKPSNTKVDKRVESKYGPPCPPCPAPEFEGGPSVFLFPPKPKDTLYITREPE |
| | VTCVVVDVSQEDPEVQFNWYVDGVEVHNAKTKPREEQFNSTYRVVSVLTVL |
| | HQDWLNGKEYKCKVSNKGLPSSIEKTISKAKGQPREPQVYTLPPSQEEMTKN |
| | QVSLTCLVKGFYPSDIAVEWESNGQPENNYKTTPPVLDSDGSFFLYSRLTVDK |
| | SRWQEGNVFSCSVMHEALHNHYTQKSLSLSLGK |
| SEQ | EVQLLESGGGLVQPGGSLRLSCAASGFSFSSSYYMCWVRQAPGKGLEWVSCI |
| ID | GTTRGSTYYADSVKGRFTISRDSSKNTLYLQMNSLRAEDTAVYYCARDATGY |
| NO: | RINTIGLYFNLWGQGTLVTVSSASTKGPSVFPLAPCSRSTSESTAALGCLVKDY |
| 208 | FPEPVTVSWNSGALTSGVHTFPAVLQSSGLYSLSSVVTVPSSSLGTKTYTCNV |
| | DHKPSNTKVDKRVESKYGPPCPPCPAPEFEGGPSVFLFPPKPKDTLYITREPEV |
| | TCVVVDVSQEDPEVQFNWYVDGVEVHNAKTKPREEQFNSTYRVVSVLTVLH |
| | QDWLNGKEYKCKVSNKGLPSSIEKTISKAKGQPREPQVYTLPPSQEEMTKNQ |
| | VSLTCLVKGFYPSDIAVEWESNGQPENNYKTTPPVLDSDGSFFLYSRLTVDKS |
| | RWQEGNVFSCSVMHEALHNHYTQKSLSLSLGK |
| SEQ | EVQLLESGGGLVQPGGSLRLSCAASGFSFSSSYYMCWVRQAPGKGLEWVSCI |
| ID | GTTRGSTYYADSVKGRFTISRDSSKNTVYLQMNSLRAEDTAVYFCARDATGY |
| NO: | RINTIGLYFNLWGQGTLVTVSSASTKGPSVFPLAPCSRSTSESTAALGCLVKDY |
| 209 | FPEPVTVSWNSGALTSGVHTFPAVLQSSGLYSLSSVVTVPSSSLGTKTYTCNV |
| | DHKPSNTKVDKRVESKYGPPCPPCPAPEFEGGPSVFLFPPKPKDTLYITREPEV |
| | TCVVVDVSQEDPEVQFNWYVDGVEVHNAKTKPREEQFNSTYRVVSVLTVLH |
| | QDWLNGKEYKCKVSNKGLPSSIEKTISKAKGQPREPQVYTLPPSQEEMTKNQ |
| | VSLTCLVKGFYPSDIAVEWESNGQPENNYKTTPPVLDSDGSFFLYSRLTVDKS |
| | RWQEGNVFSCSVMHEALHNHYTQKSLSLSLGK |
| SEQ | EVQLLESGGGLVQPGGSLRLSCAASGFSFSSSYYMSWVRQAPGKGLEWVSAI |
| ID | GTTRGSTYYADSVKGRFTISRDNSKNTLYLQMNSLRAEDTAVYYCARDATG |
| NO: | YRINTIGLYFNLWGQGTLVTVSSASTKGPSVFPLAPCSRSTSESTAALGCLVKD |
| 210 | YFPEPVTVSWNSGALTSGVHTFPAVLQSSGLYSLSSVVTVPSSSLGTKTYTCN |
| | VDHKPSNTKVDKRVESKYGPPCPPCPAPEFEGGPSVFLFPPKPKDTLYITREPE |
| | VTCVVVDVSQEDPEVQFNWYVDGVEVHNAKTKPREEQFNSTYRVVSVLTVL |
| | HQDWLNGKEYKCKVSNKGLPSSIEKTISKAKGQPREPQVYTLPPSQEEMTKN |
| | QVSLTCLVKGFYPSDIAVEWESNGQPENNYKTTPPVLDSDGSFFLYSRLTVDK |
| | SRWQEGNVFSCSVMHEALHNHYTQKSLSLSLGK |
| SEQ | EVQLLESGGGLVQPGGSLRLSCAASGFSFSSSYYMSWVRQAPGKGLEWVSAI |
| ID | GTTRGSTYYADSVKGRFTISRDNSKNTVYLQMNSLRAEDTAVYYCARDATG |
| NO: | YRINTIGLYFNLWGQGTLVTVSSASTKGPSVFPLAPCSRSTSESTAALGCLVKD |
| 211 | YFPEPVTVSWNSGALTSGVHTFPAVLQSSGLYSLSSVVTVPSSSLGTKTYTCN |
| | VDHKPSNTKVDKRVESKYGPPCPPCPAPEFEGGPSVFLFPPKPKDTLYITREPE |
| | VTCVVVDVSQEDPEVQFNWYVDGVEVHNAKTKPREEQFNSTYRVVSVLTVL |
| | HQDWLNGKEYKCKVSNKGLPSSIEKTISKAKGQPREPQVYTLPPSQEEMTKN |
| | QVSLTCLVKGFYPSDIAVEWESNGQPENNYKTTPPVLDSDGSFFLYSRLTVDK |
| | SRWQEGNVFSCSVMHEALHNHYTQKSLSLSLGK |
| SEQ | EVQLLESGGGLVQPGGSLRLSCAASGFSFSSSYYMSWVRQAPGKGLEWVSAI |
| ID | GTTRGSTYYADSVKGRFTISRDSSKNTLYLQMNSLRAEDTAVYYCARDATGY |
| NO: | RINTIGLYFNLWGQGTLVTVSSASTKGPSVFPLAPCSRSTSESTAALGCLVKDY |
| 212 | FPEPVTVSWNSGALTSGVHTFPAVLQSSGLYSLSSVVTVPSSSLGTKTYTCNV |
| | DHKPSNTKVDKRVESKYGPPCPPCPAPEFEGGPSVFLFPPKPKDTLYITREPEV |
| | TCVVVDVSQEDPEVQFNWYVDGVEVHNAKTKPREEQFNSTYRVVSVLTVLH |
| | QDWLNGKEYKCKVSNKGLPSSIEKTISKAKGQPREPQVYTLPPSQEEMTKNQ |
| | VSLTCLVKGFYPSDIAVEWESNGQPENNYKTTPPVLDSDGSFFLYSRLTVDKS |
| | RWQEGNVFSCSVMHEALHNHYTQKSLSLSLGK |
| SEQ | EVQLLESGGGLVQPGGSLRLSCAASGFSFSSSYYMSWVRQAPGKGLEWVSAI |
| ID | GTTRGSTYYADSVKGRFTISRDSSKNTVYLQMNSLRAEDTAVYFCARDATGY |
| NO: | RINTIGLYFNLWGQGTLVTVSSASTKGPSVFPLAPCSRSTSESTAALGCLVKDY |
| 213 | FPEPVTVSWNSGALTSGVHTFPAVLQSSGLYSLSSVVTVPSSSLGTKTYTCNV |
| | DHKPSNTKVDKRVESKYGPPCPPCPAPEFEGGPSVFLFPPKPKDTLYITREPEV |
| | TCVVVDVSQEDPEVQFNWYVDGVEVHNAKTKPREEQFNSTYRVVSVLTVLH |
| | QDWLNGKEYKCKVSNKGLPSSIEKTISKAKGQPREPQVYTLPPSQEEMTKNQ |
| | VSLTCLVKGFYPSDIAVEWESNGQPENNYKTTPPVLDSDGSFFLYSRLTVDKS |
| | RWQEGNVFSCSVMHEALHNHYTQKSLSLSLGK |
| SEQ | QSLEESGGDLVKPGASLTLTCRVSGFSFSSSYYMCWVRQAPGKGLEWIACIGT |
| ID | TRGSTYYATWAKGRFTISKISSTTVTLQMTSLTDADTATYFCARDATGYRINT |
| NO: | IGLYFNLWGPGTLVTVSSASTKGPSVFPLAPCSRSTSESTAALGCLVKDYFPEP |
| 214 | VTVSWNSGALTSGVHTFPAVLQSSGLYSLSSVVTVPSSSLGTKTYTCNVDHKP |
| | SNTKVDKRVESKYGPPCPPCPAPEFEGGPSVFLFPPKPKDTLYITREPEVTCVV |
| | VDVSQEDPEVQFNWYVDGVEVHNAKTKPREEQFNSTYRVVSVLTVLHQDW |
| | LNGKEYKCKVSNKGLPSSIEKTISKAKGQPREPQVYTLPPSQEEMTKNQVSLT |
| | CLVKGFYPSDIAVEWESNGQPENNYKTTPPVLDSDGSFFLYSRLTVDKSRWQ |
| | EGNVFSCSVMHEALHNHYTQKSLSLSLGK |
| SEQ | EVQLLESGGGLVQPGGSLRLSCAASGFSFSSSYYMCWVRQAPGKGLEWVSAI |
| ID | GTTRGSTYYADSVKGRFTISRDSSKNTVYLQMNSLRAEDTAVYYCARDATG |
| NO: | YRINTIGLYFNLWGQGTLVTVSSASTKGPSVFPLAPSSKSTSGGTAALGCLVK |
| 215 | DYFPEPVTVSWNSGALTSGVHTFPAVLQSSGLYSLSSVVTVPSSSLGTQTYICN |
| | VNHKPSNTKVDKKVEPKSCDKTHTCPPCPAPEAAGGPSVFLFPPKPKDTLMIS |
| | RTPEVTCVVVSVSHEDPEVKFNWYVDGVEVHNAKTKPREEQYNSTYRVVSV |
| | LTVLHQDWLNGKEYKCKVSNKALPAPIEKTISKAKGQPREPQVYTLPPSREE |
| | MTKNQVSLTCLVKGFYPSDIAVEWESNGQPENNYKTTPPVLDSDGSFFLYSK |
| | LTVDKSRWQQGNVFSCSVMHEALHNHYTQKSLSLSPG |
| SEQ | EVQLLESGGGLVQPGGSLRLSCAASGFSFSSSYYMAWVRQAPGKGLEWVSAI |
| ID | GTTRGSTYYADSVKGRFTISRDNSKNTLYLQMNSLRAEDTAVYYCARDATG |
| NO: | YRINTIGLYFNLWGQGTLVTVSSASTKGPSVFPLAPSSKSTSGGTAALGCLVK |
| 216 | DYFPEPVTVSWNSGALTSGVHTFPAVLQSSGLYSLSSVVTVPSSSLGTQTYICN |
| | VNHKPSNTKVDKKVEPKSCDKTHTCPPCPAPEAAGGPSVFLFPPKPKDTLMIS |
| | RTPEVTCVVVSVSHEDPEVKFNWYVDGVEVHNAKTKPREEQYNSTYRVVSV |
| | LTVLHQDWLNGKEYKCKVSNKALPAPIEKTISKAKGQPREPQVYTLPPSREE |
| | MTKNQVSLTCLVKGFYPSDIAVEWESNGQPENNYKTTPPVLDSDGSFFLYSK |
| | LTVDKSRWQQGNVFSCSVMHEALHNHYTQKSLSLSPG |
| SEQ | EVQLLESGGGLVQPGGSLRLSCAASGFSFSSSYYMAWVRQAPGKGLEWVSAI |
| ID | GTTRGSTYYADSVKGRFTISRDNSKNTVYLQMNSLRAEDTAVYYCARDATG |
| NO: | YRINTIGLYFNLWGQGTLVTVSSASTKGPSVFPLAPSSKSTSGGTAALGCLVK |
| 217 | DYFPEPVTVSWNSGALTSGVHTFPAVLQSSGLYSLSSVVTVPSSSLGTQTYICN |
| | VNHKPSNTKVDKKVEPKSCDKTHTCPPCPAPEAAGGPSVFLFPPKPKDTLMIS |
| | RTPEVTCVVVSVSHEDPEVKFNWYVDGVEVHNAKTKPREEQYNSTYRVVSV |
| | LTVLHQDWLNGKEYKCKVSNKALPAPIEKTISKAKGQPREPQVYTLPPSREE |
| | MTKNQVSLTCLVKGFYPSDIAVEWESNGQPENNYKTTPPVLDSDGSFFLYSK |
| | LTVDKSRWQQGNVFSCSVMHEALHNHYTQKSLSLSPG |
| SEQ | EVQLLESGGGLVQPGGSLRLSCAASGFSFSSSYYMAWVRQAPGKGLEWVSAI |
| ID | GTTRGSTYYADSVKGRFTISRDSSKNTLYLQMNSLRAEDTAVYYCARDATGY |
| NO: | RINTIGLYFNLWGQGTLVTVSSASTKGPSVFPLAPSSKSTSGGTAALGCLVKD |
| 218 | YFPEPVTVSWNSGALTSGVHTFPAVLQSSGLYSLSSVVTVPSSSLGTQTYICNV |
| | NHKPSNTKVDKKVEPKSCDKTHTCPPCPAPEAAGGPSVFLFPPKPKDTLMISR |
| | TPEVTCVVVSVSHEDPEVKFNWYVDGVEVHNAKTKPREEQYNSTYRVVSVL |
| | TVLHQDWLNGKEYKCKVSNKALPAPIEKTISKAKGQPREPQVYTLPPSREEM |
| | TKNQVSLTCLVKGFYPSDIAVEWESNGQPENNYKTTPPVLDSDGSFFLYSKLT |
| | VDKSRWQQGNVFSCSVMHEALHNHYTQKSLSLSPG |
| SEQ | EVQLLESGGGLVQPGGSLRLSCAASGFSFSSSYYMAWVRQAPGKGLEWVSAI |
| ID | GTTRGSTYYADSVKGRFTISRDSSKNTVYLQMNSLRAEDTAVYFCARDATGY |
| NO: | RINTIGLYFNLWGQGTLVTVSSASTKGPSVFPLAPSSKSTSGGTAALGCLVKD |
| 219 | YFPEPVTVSWNSGALTSGVHTFPAVLQSSGLYSLSSVVTVPSSSLGTQTYICNV |
| | NHKPSNTKVDKKVEPKSCDKTHTCPPCPAPEAAGGPSVFLFPPKPKDTLMISR |
| | TPEVTCVVVSVSHEDPEVKFNWYVDGVEVHNAKTKPREEQYNSTYRVVSVL |
| | TVLHQDWLNGKEYKCKVSNKALPAPIEKTISKAKGQPREPQVYTLPPSREEM |
| | TKNQVSLTCLVKGFYPSDIAVEWESNGQPENNYKTTPPVLDSDGSFFLYSKLT |
| | VDKSRWQQGNVFSCSVMHEALHNHYTQKSLSLSPG |
| SEQ | EVQLLESGGGLVQPGGSLRLSCAASGFSFSSSYYMCWVRQAPGKGLEWVSCI |
| ID | GTTRGSTYYADSVKGRFTISRDNSKNTLYLQMNSLRAEDTAVYYCARDATG |
| NO: | YRINTIGLYFNLWGQGTLVTVSSASTKGPSVFPLAPSSKSTSGGTAALGCLVK |
| 220 | DYFPEPVTVSWNSGALTSGVHTFPAVLQSSGLYSLSSVVTVPSSSLGTQTYICN |
| | VNHKPSNTKVDKKVEPKSCDKTHTCPPCPAPEAAGGPSVFLFPPKPKDTLMIS |
| | RTPEVTCVVVSVSHEDPEVKFNWYVDGVEVHNAKTKPREEQYNSTYRVVSV |
| | LTVLHQDWLNGKEYKCKVSNKALPAPIEKTISKAKGQPREPQVYTLPPSREE |
| | MTKNQVSLTCLVKGFYPSDIAVEWESNGQPENNYKTTPPVLDSDGSFFLYSK |
| | LTVDKSRWQQGNVFSCSVMHEALHNHYTQKSLSLSPG |
| SEQ | EVQLLESGGGLVQPGGSLRLSCAASGFSFSSSYYMCWVRQAPGKGLEWVSCI |
| ID | GTTRGSTYYADSVKGRFTISRDNSKNTVYLQMNSLRAEDTAVYYCARDATG |
| NO: | YRINTIGLYFNLWGQGTLVTVSSASTKGPSVFPLAPSSKSTSGGTAALGCLVK |
| 221 | DYFPEPVTVSWNSGALTSGVHTFPAVLQSSGLYSLSSVVTVPSSSLGTQTYICN |
| | VNHKPSNTKVDKKVEPKSCDKTHTCPPCPAPEAAGGPSVFLFPPKPKDTLMIS |
| | RTPEVTCVVVSVSHEDPEVKFNWYVDGVEVHNAKTKPREEQYNSTYRVVSV |
| | LTVLHQDWLNGKEYKCKVSNKALPAPIEKTISKAKGQPREPQVYTLPPSREE |
| | MTKNQVSLTCLVKGFYPSDIAVEWESNGQPENNYKTTPPVLDSDGSFFLYSK |
| | LTVDKSRWQQGNVFSCSVMHEALHNHYTQKSLSLSPG |
| SEQ | EVQLLESGGGLVQPGGSLRLSCAASGFSFSSSYYMCWVRQAPGKGLEWVSCI |
| ID | GTTRGSTYYADSVKGRFTISRDSSKNTLYLQMNSLRAEDTAVYYCARDATGY |
| NO: | RINTIGLYFNLWGQGTLVTVSSASTKGPSVFPLAPSSKSTSGGTAALGCLVKD |
| 222 | YFPEPVTVSWNSGALTSGVHTFPAVLQSSGLYSLSSVVTVPSSSLGTQTYICNV |
| | NHKPSNTKVDKKVEPKSCDKTHTCPPCPAPEAAGGPSVFLFPPKPKDTLMISR |
| | TPEVTCVVVSVSHEDPEVKENWYVDGVEVHNAKTKPREEQYNSTYRVVSVL |
| | TVLHQDWLNGKEYKCKVSNKALPAPIEKTISKAKGQPREPQVYTLPPSREEM |
| | TKNQVSLTCLVKGFYPSDIAVEWESNGQPENNYKTTPPVLDSDGSFFLYSKLT |
| | VDKSRWQQGNVFSCSVMHEALHNHYTQKSLSLSPG |
| SEQ | EVQLLESGGGLVQPGGSLRLSCAASGFSFSSSYYMCWVRQAPGKGLEWVSCI |
| ID | GTTRGSTYYADSVKGRFTISRDSSKNTVYLQMNSLRAEDTAVYFCARDATGY |
| NO: | RINTIGLYFNLWGQGTLVTVSSASTKGPSVFPLAPSSKSTSGGTAALGCLVKD |
| 223 | YFPEPVTVSWNSGALTSGVHTFPAVLQSSGLYSLSSVVTVPSSSLGTQTYICNV |
| | NHKPSNTKVDKKVEPKSCDKTHTCPPCPAPEAAGGPSVFLFPPKPKDTLMISR |
| | TPEVTCVVVSVSHEDPEVKFNWYVDGVEVHNAKTKPREEQYNSTYRVVSVL |
| | TVLHQDWLNGKEYKCKVSNKALPAPIEKTISKAKGQPREPQVYTLPPSREEM |
| | TKNQVSLTCLVKGFYPSDIAVEWESNGQPENNYKTTPPVLDSDGSFFLYSKLT |
| | VDKSRWQQGNVFSCSVMHEALHNHYTQKSLSLSPG |
| SEQ | EVQLLESGGGLVQPGGSLRLSCAASGFSFSSSYYMSWVRQAPGKGLEWVSAI |
| ID | GTTRGSTYYADSVKGRFTISRDNSKNTLYLQMNSLRAEDTAVYYCARDATG |
| NO: | YRINTIGLYFNLWGQGTLVTVSSASTKGPSVFPLAPSSKSTSGGTAALGCLVK |
| 224 | DYFPEPVTVSWNSGALTSGVHTFPAVLQSSGLYSLSSVVTVPSSSLGTQTYICN |
| | VNHKPSNTKVDKKVEPKSCDKTHTCPPCPAPEAAGGPSVFLFPPKPKDTLMIS |
| | RTPEVTCVVVSVSHEDPEVKFNWYVDGVEVHNAKTKPREEQYNSTYRVVSV |
| | LTVLHQDWLNGKEYKCKVSNKALPAPIEKTISKAKGQPREPQVYTLPPSREE |
| | MTKNQVSLTCLVKGFYPSDIAVEWESNGQPENNYKTTPPVLDSDGSFFLYSK |
| | LTVDKSRWQQGNVFSCSVMHEALHNHYTQKSLSLSPG |
| SEQ | EVQLLESGGGLVQPGGSLRLSCAASGFSFSSSYYMSWVRQAPGKGLEWVSAI |
| ID | GTTRGSTYYADSVKGRFTISRDNSKNTVYLQMNSLRAEDTAVYYCARDATG |
| NO: | YRINTIGLYFNLWGQGTLVTVSSASTKGPSVFPLAPSSKSTSGGTAALGCLVK |
| 225 | DYFPEPVTVSWNSGALTSGVHTFPAVLQSSGLYSLSSVVTVPSSSLGTQTYICN |
| | VNHKPSNTKVDKKVEPKSCDKTHTCPPCPAPEAAGGPSVFLFPPKPKDTLMIS |
| | RTPEVTCVVVSVSHEDPEVKFNWYVDGVEVHNAKTKPREEQYNSTYRVVSV |
| | LTVLHQDWLNGKEYKCKVSNKALPAPIEKTISKAKGQPREPQVYTLPPSREE |
| | MTKNQVSLTCLVKGFYPSDIAVEWESNGQPENNYKTTPPVLDSDGSFFLYSK |
| | LTVDKSRWQQGNVFSCSVMHEALHNHYTQKSLSLSPG |
| SEQ | EVQLLESGGGLVQPGGSLRLSCAASGFSFSSSYYMSWVRQAPGKGLEWVSAI |
| ID | GTTRGSTYYADSVKGRFTISRDSSKNTLYLQMNSLRAEDTAVYYCARDATGY |
| NO: | RINTIGLYFNLWGQGTLVTVSSASTKGPSVFPLAPSSKSTSGGTAALGCLVKD |
| 226 | YFPEPVTVSWNSGALTSGVHTFPAVLQSSGLYSLSSVVTVPSSSLGTQTYICNV |
| | NHKPSNTKVDKKVEPKSCDKTHTCPPCPAPEAAGGPSVFLFPPKPKDTLMISR |
| | TPEVTCVVVSVSHEDPEVKFNWYVDGVEVHNAKTKPREEQYNSTYRVVSVL |
| | TVLHQDWLNGKEYKCKVSNKALPAPIEKTISKAKGQPREPQVYTLPPSREEM |
| | TKNQVSLTCLVKGFYPSDIAVEWESNGQPENNYKTTPPVLDSDGSFFLYSKLT |
| | VDKSRWQQGNVFSCSVMHEALHNHYTQKSLSLSPG |
| SEQ | EVQLLESGGGLVQPGGSLRLSCAASGFSFSSSYYMSWVRQAPGKGLEWVSAI |
| ID | GTTRGSTYYADSVKGRFTISRDSSKNTVYLQMNSLRAEDTAVYFCARDATGY |
| NO: | RINTIGLYFNLWGQGTLVTVSSASTKGPSVFPLAPSSKSTSGGTAALGCLVKD |
| 227 | YFPEPVTVSWNSGALTSGVHTFPAVLQSSGLYSLSSVVTVPSSSLGTQTYICNV |
| | NHKPSNTKVDKKVEPKSCDKTHTCPPCPAPEAAGGPSVFLFPPKPKDTLMISR |
| | TPEVTCVVVSVSHEDPEVKFNWYVDGVEVHNAKTKPREEQYNSTYRVVSVL |
| | TVLHQDWLNGKEYKCKVSNKALPAPIEKTISKAKGQPREPQVYTLPPSREEM |
| | TKNQVSLTCLVKGFYPSDIAVEWESNGQPENNYKTTPPVLDSDGSFFLYSKLT |
| | VDKSRWQQGNVFSCSVMHEALHNHYTQKSLSLSPG |
| SEQ | QSLEESGGDLVKPGASLTLTCRVSGFSFSSSYYMCWVRQAPGKGLEWIACIGT |
| ID | TRGSTYYATWAKGRFTISKISSTTVTLQMTSLTDADTATYFCARDATGYRINT |
| NO: | IGLYFNLWGPGTLVTVSSASTKGPSVFPLAPSSKSTSGGTAALGCLVKDYFPEP |
| 228 | VTVSWNSGALTSGVHTFPAVLQSSGLYSLSSVVTVPSSSLGTQTYICNVNHKP |
| | SNTKVDKKVEPKSCDKTHTCPPCPAPEAAGGPSVFLFPPKPKDTLMISRTPEVT |
| | CVVVSVSHEDPEVKFNWYVDGVEVHNAKTKPREEQYNSTYRVVSVLTVLHQ |
| | DWLNGKEYKCKVSNKALPAPIEKTISKAKGQPREPQVYTLPPSREEMTKNQV |
| | SLTCLVKGFYPSDIAVEWESNGQPENNYKTTPPVLDSDGSFFLYSKLTVDKSR |
| | WQQGNVFSCSVMHEALHNHYTQKSLSLSPG |
| SEQ | EVQLLESGGGLVQPGGSLRLSCAASGFSFSSSYYMCWVRQAPGKGLEWVSAI |
| ID | GTTRGSTYYADSVKGRFTISRDSSKNTVYLQMNSLRAEDTAVYYCARDATG |
| NO: | YRINTIGLYFNLWGQGTLVTVSSASTKGPSVFPLAPCSRSTSESTAALGCLVKD |
| 229 | YFPEPVTVSWNSGALTSGVHTFPAVLQSSGLYSLSSVVTVPSSSLGTKTYTCN |
| | VDHKPSNTKVDKRVESKYGPPCPPCPAPEFEGGPSVFLFPPKPKDTLMISRTPE |
| | VTCVVVDVSQEDPEVQFNWYVDGVEVHNAKTKPREEQFNSTYRVVSVLTVL |
| | HQDWLNGKEYKCKVSNKGLPSSIEKTISKAKGQPREPQVYTLPPSQEEMTKN |
| | QVSLTCLVKGFYPSDIAVEWESNGQPENNYKTTPPVLDSDGSFFLYSRLTVDK |
| | SRWQEGNVFSCSVLHEALHSHYTQKSLSLSLG |
| SEQ | EVQLLESGGGLVQPGGSLRLSCAASGFSFSSSYYMAWVRQAPGKGLEWVSAI |
| ID | GTTRGSTYYADSVKGRFTISRDNSKNTLYLQMNSLRAEDTAVYYCARDATG |
| NO | YRINTIGLYFNLWGQGTLVTVSSASTKGPSVFPLAPCSRSTSESTAALGCLVKD |
| 230 | YFPEPVTVSWNSGALTSGVHTFPAVLQSSGLYSLSSVVTVPSSSLGTKTYTCN |
| | VDHKPSNTKVDKRVESKYGPPCPPCPAPEFEGGPSVFLFPPKPKDTLMISRTPE |
| | VTCVVVDVSQEDPEVQFNWYVDGVEVHNAKTKPREEQFNSTYRVVSVLTVL |
| | HQDWLNGKEYKCKVSNKGLPSSIEKTISKAKGQPREPQVYTLPPSQEEMTKN |
| | QVSLTCLVKGFYPSDIAVEWESNGQPENNYKTTPPVLDSDGSFFLYSRLTVDK |
| | SRWQEGNVFSCSVLHEALHSHYTQKSLSLSLG |
| SEQ | EVQLLESGGGLVQPGGSLRLSCAASGFSFSSSYYMAWVRQAPGKGLEWVSAI |
| ID | GTTRGSTYYADSVKGRFTISRDNSKNTVYLQMNSLRAEDTAVYYCARDATG |
| NO: | YRINTIGLYFNLWGQGTLVTVSSASTKGPSVFPLAPCSRSTSESTAALGCLVKD |
| 231 | YFPEPVTVSWNSGALTSGVHTFPAVLQSSGLYSLSSVVTVPSSSLGTKTYTCN |
| | VDHKPSNTKVDKRVESKYGPPCPPCPAPEFEGGPSVFLFPPKPKDTLMISRTPE |
| | VTCVVVDVSQEDPEVQFNWYVDGVEVHNAKTKPREEQFNSTYRVVSVLTVL |
| | HQDWLNGKEYKCKVSNKGLPSSIEKTISKAKGQPREPQVYTLPPSQEEMTKN |
| | QVSLTCLVKGFYPSDIAVEWESNGQPENNYKTTPPVLDSDGSFFLYSRLTVDK |
| | SRWQEGNVFSCSVLHEALHSHYTQKSLSLSLG |
| SEQ | EVQLLESGGGLVQPGGSLRLSCAASGFSFSSSYYMAWVRQAPGKGLEWVSAI |
| ID | GTTRGSTYYADSVKGRFTISRDSSKNTLYLQMNSLRAEDTAVYYCARDATGY |
| NO: | RINTIGLYFNLWGQGTLVTVSSASTKGPSVFPLAPCSRSTSESTAALGCLVKDY |
| 232 | FPEPVTVSWNSGALTSGVHTFPAVLQSSGLYSLSSVVTVPSSSLGTKTYTCNV |
| | DHKPSNTKVDKRVESKYGPPCPPCPAPEFEGGPSVFLFPPKPKDTLMISRTPEV |
| | TCVVVDVSQEDPEVQFNWYVDGVEVHNAKTKPREEQFNSTYRVVSVLTVLH |
| | QDWLNGKEYKCKVSNKGLPSSIEKTISKAKGQPREPQVYTLPPSQEEMTKNQ |
| | VSLTCLVKGFYPSDIAVEWESNGQPENNYKTTPPVLDSDGSFFLYSRLTVDKS |
| | RWQEGNVFSCSVLHEALHSHYTQKSLSLSLG |
| SEQ | EVQLLESGGGLVQPGGSLRLSCAASGFSFSSSYYMAWVRQAPGKGLEWVSAI |
| ID | GTTRGSTYYADSVKGRFTISRDSSKNTVYLQMNSLRAEDTAVYFCARDATGY |
| NO: | RINTIGLYFNLWGQGTLVTVSSASTKGPSVFPLAPCSRSTSESTAALGCLVKDY |
| 233 | FPEPVTVSWNSGALTSGVHTFPAVLQSSGLYSLSSVVTVPSSSLGTKTYTCNV |
| | DHKPSNTKVDKRVESKYGPPCPPCPAPEFEGGPSVFLFPPKPKDTLMISRTPEV |
| | TCVVVDVSQEDPEVQFNWYVDGVEVHNAKTKPREEQFNSTYRVVSVLTVLH |
| | QDWLNGKEYKCKVSNKGLPSSIEKTISKAKGQPREPQVYTLPPSQEEMTKNQ |
| | VSLTCLVKGFYPSDIAVEWESNGQPENNYKTTPPVLDSDGSFFLYSRLTVDKS |
| | RWQEGNVFSCSVLHEALHSHYTQKSLSLSLG |
| SEQ | EVQLLESGGGLVQPGGSLRLSCAASGFSFSSSYYMCWVRQAPGKGLEWVSCI |
| ID | GTTRGSTYYADSVKGRFTISRDNSKNTLYLQMNSLRAEDTAVYYCARDATG |
| NO: | YRINTIGLYFNLWGQGTLVTVSSASTKGPSVFPLAPCSRSTSESTAALGCLVKD |
| 234 | YFPEPVTVSWNSGALTSGVHTFPAVLQSSGLYSLSSVVTVPSSSLGTKTYTCN |
| | VDHKPSNTKVDKRVESKYGPPCPPCPAPEFEGGPSVFLFPPKPKDTLMISRTPE |
| | VTCVVVDVSQEDPEVQFNWYVDGVEVHNAKTKPREEQFNSTYRVVSVLTVL |
| | HQDWLNGKEYKCKVSNKGLPSSIEKTISKAKGQPREPQVYTLPPSQEEMTKN |
| | QVSLTCLVKGFYPSDIAVEWESNGQPENNYKTTPPVLDSDGSFFLYSRLTVDK |
| | SRWQEGNVFSCSVLHEALHSHYTQKSLSLSLG |
| SEQ | EVQLLESGGGLVQPGGSLRLSCAASGFSFSSSYYMCWVRQAPGKGLEWVSCI |
| ID | GTTRGSTYYADSVKGRFTISRDNSKNTVYLQMNSLRAEDTAVYYCARDATG |
| NO: | YRINTIGLYFNLWGQGTLVTVSSASTKGPSVFPLAPCSRSTSESTAALGCLVKD |
| 235 | YFPEPVTVSWNSGALTSGVHTFPAVLQSSGLYSLSSVVTVPSSSLGTKTYTCN |
| | VDHKPSNTKVDKRVESKYGPPCPPCPAPEFEGGPSVFLFPPKPKDTLMISRTPE |
| | VTCVVVDVSQEDPEVQFNWYVDGVEVHNAKTKPREEQFNSTYRVVSVLTVL |
| | HQDWLNGKEYKCKVSNKGLPSSIEKTISKAKGQPREPQVYTLPPSQEEMTKN |
| | QVSLTCLVKGFYPSDIAVEWESNGQPENNYKTTPPVLDSDGSFFLYSRLTVDK |
| | SRWQEGNVFSCSVLHEALHSHYTQKSLSLSLG |
| SEQ | EVQLLESGGGLVQPGGSLRLSCAASGFSFSSSYYMCWVRQAPGKGLEWVSCI |
| ID | GTTRGSTYYADSVKGRFTISRDSSKNTLYLQMNSLRAEDTAVYYCARDATGY |
| NO: | RINTIGLYFNLWGQGTLVTVSSASTKGPSVFPLAPCSRSTSESTAALGCLVKDY |
| 236 | FPEPVTVSWNSGALTSGVHTFPAVLQSSGLYSLSSVVTVPSSSLGTKTYTCNV |
| | DHKPSNTKVDKRVESKYGPPCPPCPAPEFEGGPSVFLFPPKPKDTLMISRTPEV |
| | TCVVVDVSQEDPEVQFNWYVDGVEVHNAKTKPREEQFNSTYRVVSVLTVLH |
| | QDWLNGKEYKCKVSNKGLPSSIEKTISKAKGQPREPQVYTLPPSQEEMTKNQ |
| | VSLTCLVKGFYPSDIAVEWESNGQPENNYKTTPPVLDSDGSFFLYSRLTVDKS |
| | RWQEGNVFSCSVLHEALHSHYTQKSLSLSLG |
| SEQ | EVQLLESGGGLVQPGGSLRLSCAASGFSFSSSYYMCWVRQAPGKGLEWVSCI |
| ID | GTTRGSTYYADSVKGRFTISRDSSKNTVYLQMNSLRAEDTAVYFCARDATGY |
| NO: | RINTIGLYFNLWGQGTLVTVSSASTKGPSVFPLAPCSRSTSESTAALGCLVKDY |
| 237 | FPEPVTVSWNSGALTSGVHTFPAVLQSSGLYSLSSVVTVPSSSLGTKTYTCNV |
| | DHKPSNTKVDKRVESKYGPPCPPCPAPEFEGGPSVFLFPPKPKDTLMISRTPEV |
| | TCVVVDVSQEDPEVQFNWYVDGVEVHNAKTKPREEQFNSTYRVVSVLTVLH |
| | QDWLNGKEYKCKVSNKGLPSSIEKTISKAKGQPREPQVYTLPPSQEEMTKNQ |
| | VSLTCLVKGFYPSDIAVEWESNGQPENNYKTTPPVLDSDGSFFLYSRLTVDKS |
| | RWQEGNVFSCSVLHEALHSHYTQKSLSLSLG |
| SEQ | EVQLLESGGGLVQPGGSLRLSCAASGFSFSSSYYMSWVRQAPGKGLEWVSAI |
| ID | GTTRGSTYYADSVKGRFTISRDNSKNTLYLQMNSLRAEDTAVYYCARDATG |
| NO: | YRINTIGLYFNLWGQGTLVTVSSASTKGPSVFPLAPCSRSTSESTAALGCLVKD |
| 238 | YFPEPVTVSWNSGALTSGVHTFPAVLQSSGLYSLSSVVTVPSSSLGTKTYTCN |
| | VDHKPSNTKVDKRVESKYGPPCPPCPAPEFEGGPSVFLFPPKPKDTLMISRTPE |
| | VTCVVVDVSQEDPEVQFNWYVDGVEVHNAKTKPREEQFNSTYRVVSVLTVL |
| | HQDWLNGKEYKCKVSNKGLPSSIEKTISKAKGQPREPQVYTLPPSQEEMTKN |
| | QVSLTCLVKGFYPSDIAVEWESNGQPENNYKTTPPVLDSDGSFFLYSRLTVDK |
| | SRWQEGNVFSCSVLHEALHSHYTQKSLSLSLG |
| SEQ | EVQLLESGGGLVQPGGSLRLSCAASGFSFSSSYYMSWVRQAPGKGLEWVSAI |
| ID | GTTRGSTYYADSVKGRFTISRDNSKNTVYLQMNSLRAEDTAVYYCARDATG |
| NO: | YRINTIGLYFNLWGQGTLVTVSSASTKGPSVFPLAPCSRSTSESTAALGCLVKD |
| 239 | YFPEPVTVSWNSGALTSGVHTFPAVLQSSGLYSLSSVVTVPSSSLGTKTYTCN |
| | VDHKPSNTKVDKRVESKYGPPCPPCPAPEFEGGPSVFLFPPKPKDTLMISRTPE |
| | VTCVVVDVSQEDPEVQFNWYVDGVEVHNAKTKPREEQFNSTYRVVSVLTVL |
| | HQDWLNGKEYKCKVSNKGLPSSIEKTISKAKGQPREPQVYTLPPSQEEMTKN |
| | QVSLTCLVKGFYPSDIAVEWESNGQPENNYKTTPPVLDSDGSFFLYSRLTVDK |
| | SRWQEGNVFSCSVLHEALHSHYTQKSLSLSLG |
| SEQ | EVQLLESGGGLVQPGGSLRLSCAASGFSFSSSYYMSWVRQAPGKGLEWVSAI |
| ID | GTTRGSTYYADSVKGRFTISRDSSKNTLYLQMNSLRAEDTAVYYCARDATGY |
| NO: | RINTIGLYFNLWGQGTLVTVSSASTKGPSVFPLAPCSRSTSESTAALGCLVKDY |
| 240 | FPEPVTVSWNSGALTSGVHTFPAVLQSSGLYSLSSVVTVPSSSLGTKTYTCNV |
| | DHKPSNTKVDKRVESKYGPPCPPCPAPEFEGGPSVFLFPPKPKDTLMISRTPEV |
| | TCVVVDVSQEDPEVQFNWYVDGVEVHNAKTKPREEQFNSTYRVVSVLTVLH |
| | QDWLNGKEYKCKVSNKGLPSSIEKTISKAKGQPREPQVYTLPPSQEEMTKNQ |
| | VSLTCLVKGFYPSDIAVEWESNGQPENNYKTTPPVLDSDGSFFLYSRLTVDKS |
| | RWQEGNVFSCSVLHEALHSHYTQKSLSLSLG |
| SEQ | EVQLLESGGGLVQPGGSLRLSCAASGFSFSSSYYMSWVRQAPGKGLEWVSAI |
| ID | GTTRGSTYYADSVKGRFTISRDSSKNTVYLQMNSLRAEDTAVYFCARDATGY |
| NO: | RINTIGLYFNLWGQGTLVTVSSASTKGPSVFPLAPCSRSTSESTAALGCLVKDY |
| 241 | FPEPVTVSWNSGALTSGVHTFPAVLQSSGLYSLSSVVTVPSSSLGTKTYTCNV |
| | DHKPSNTKVDKRVESKYGPPCPPCPAPEFEGGPSVFLFPPKPKDTLMISRTPEV |
| | TCVVVDVSQEDPEVQFNWYVDGVEVHNAKTKPREEQFNSTYRVVSVLTVLH |
| | QDWLNGKEYKCKVSNKGLPSSIEKTISKAKGQPREPQVYTLPPSQEEMTKNQ |
| | VSLTCLVKGFYPSDIAVEWESNGQPENNYKTTPPVLDSDGSFFLYSRLTVDKS |
| | RWQEGNVFSCSVLHEALHSHYTQKSLSLSLG |
| SEQ | QSLEESGGDLVKPGASLTLTCRVSGFSFSSSYYMCWVRQAPGKGLEWIACIGT |
| ID | TRGSTYYATWAKGRFTISKISSTTVTLQMTSLTDADTATYFCARDATGYRINT |
| NO: | IGLYFNLWGPGTLVTVSSASTKGPSVFPLAPCSRSTSESTAALGCLVKDYFPEP |
| 242 | VTVSWNSGALTSGVHTFPAVLQSSGLYSLSSVVTVPSSSLGTKTYTCNVDHKP |
| | SNTKVDKRVESKYGPPCPPCPAPEFEGGPSVFLFPPKPKDTLMISRTPEVTCVV |
| | VDVSQEDPEVQFNWYVDGVEVHNAKTKPREEQFNSTYRVVSVLTVLHQDW |
| | LNGKEYKCKVSNKGLPSSIEKTISKAKGQPREPQVYTLPPSQEEMTKNQVSLT |
| | CLVKGFYPSDIAVEWESNGQPENNYKTTPPVLDSDGSFFLYSRLTVDKSRWQ |
| | EGNVFSCSVLHEALHSHYTQKSLSLSLG |
| SEQ | ASTKGPSVFPLAPCSRSTSESTAALGCLVKDYFPEPVTVSWNSGALTSGVHTF |
| ID | PAVLQSSGLYSLSSVVTVPSSSLGTKTYTCNVDHKPSNTKVDKRVESKYGPPC |
| NO: | PPCPAPEFEGGPSVFLFPPKPKDTLYITREPEVTCVVVDVSQEDPEVQFNWYV |
| 243 | DGVEVHNAKTKPREEQFNSTYRVVSVLTVLHQDWLNGKEYKCKVSNKGLPS |
| | SIEKTISKAKGQPREPQVYTLPPSQEEMTKNQVSLTCLVKGFYPSDIAVEWESN |
| | GQPENNYKTTPPVLDSDGSFFLYSRLTVDKSRWQEGNVFSCSVMHEALHNHY |
| | TQKSLSLSLGEVQLLESGGGLVQPGGSLRLSCAASGFSFSSSYYMCWVRQAP |
| | GKGLEWVSAIGTTRGSTYYADSVKGRFTISRDSSKNTVYLQMNSLRAEDTAV |
| | YYCARDATGYRINTIGLYFNLWGQGTLVTVSS |
| SEQ | EVQLLESGGGLVQPGGSLRLSCAASGFSFSSSYYMAWVRQAPGKGLEWVSAI |
| ID | GTTRGSTYYADSVKGRFTISRDNSKNTLYLQMNSLRAEDTAVYYCARDATG |
| NO: | YRINTIGLYFNLWGQGTLVTVSSASTKGPSVFPLAPCSRSTSESTAALGCLVKD |
| 244 | YFPEPVTVSWNSGALTSGVHTFPAVLQSSGLYSLSSVVTVPSSSLGTKTYTCN |
| | VDHKPSNTKVDKRVESKYGPPCPPCPAPEFEGGPSVFLFPPKPKDTLYITREPE |
| | VTCVVVDVSQEDPEVQFNWYVDGVEVHNAKTKPREEQFNSTYRVVSVLTVL |
| | HQDWLNGKEYKCKVSNKGLPSSIEKTISKAKGQPREPQVYTLPPSQEEMTKN |
| | QVSLTCLVKGFYPSDIAVEWESNGQPENNYKTTPPVLDSDGSFFLYSRLTVDK |
| | SRWQEGNVFSCSVMHEALHNHYTQKSLSLSLG |
| SEQ | ASTKGPSVFPLAPCSRSTSESTAALGCLVKDYFPEPVTVSWNSGALTSGVHTF |
| ID | PAVLQSSGLYSLSSVVTVPSSSLGTKTYTCNVDHKPSNTKVDKRVESKYGPPC |
| NO: | PPCPAPEFEGGPSVFLFPPKPKDTLYITREPEVTCVVVDVSQEDPEVQFNWYV |
| 245 | DGVEVHNAKTKPREEQFNSTYRVVSVLTVLHQDWLNGKEYKCKVSNKGLPS |
| | SIEKTISKAKGQPREPQVYTLPPSQEEMTKNQVSLTCLVKGFYPSDIAVEWESN |
| | GQPENNYKTTPPVLDSDGSFFLYSRLTVDKSRWQEGNVFSCSVMHEALHNHY |
| | TQKSLSLSLGEVQLLESGGGLVQPGGSLRLSCAASGFSFSSSYYMAWVRQAP |
| | GKGLEWVSAIGTTRGSTYYADSVKGRFTISRDNSKNTVYLQMNSLRAEDTAV |
| | YYCARDATGYRINTIGLYFNLWGQGTLVTVSS |
| SEQ | EVQLLESGGGLVQPGGSLRLSCAASGFSFSSSYYMAWVRQAPGKGLEWVSAI |
| ID | GTTRGSTYYADSVKGRFTISRDSSKNTLYLQMNSLRAEDTAVYYCARDATGY |
| NO: | RINTIGLYFNLWGQGTLVTVSSASTKGPSVFPLAPCSRSTSESTAALGCLVKDY |
| 246 | FPEPVTVSWNSGALTSGVHTFPAVLQSSGLYSLSSVVTVPSSSLGTKTYTCNV |
| | DHKPSNTKVDKRVESKYGPPCPPCPAPEFEGGPSVFLFPPKPKDTLYITREPEV |
| | TCVVVDVSQEDPEVQFNWYVDGVEVHNAKTKPREEQFNSTYRVVSVLTVLH |
| | QDWLNGKEYKCKVSNKGLPSSIEKTISKAKGQPREPQVYTLPPSQEEMTKNQ |
| | VSLTCLVKGFYPSDIAVEWESNGQPENNYKTTPPVLDSDGSFFLYSRLTVDKS |
| | RWQEGNVFSCSVMHEALHNHYTQKSLSLSLG |
| SEQ | EVQLLESGGGLVQPGGSLRLSCAASGFSFSSSYYMAWVRQAPGKGLEWVSAI |
| ID | GTTRGSTYYADSVKGRFTISRDSSKNTVYLQMNSLRAEDTAVYFCARDATGY |
| NO: | RINTIGLYFNLWGQGTLVTVSSASTKGPSVFPLAPCSRSTSESTAALGCLVKDY |
| 247 | FPEPVTVSWNSGALTSGVHTFPAVLQSSGLYSLSSVVTVPSSSLGTKTYTCNV |
| | DHKPSNTKVDKRVESKYGPPCPPCPAPEFEGGPSVFLFPPKPKDTLYITREPEV |
| | TCVVVDVSQEDPEVQFNWYVDGVEVHNAKTKPREEQFNSTYRVVSVLTVLH |
| | QDWLNGKEYKCKVSNKGLPSSIEKTISKAKGQPREPQVYTLPPSQEEMTKNQ |
| | VSLTCLVKGFYPSDIAVEWESNGQPENNYKTTPPVLDSDGSFFLYSRLTVDKS |
| | RWQEGNVFSCSVMHEALHNHYTQKSLSLSLG |
| SEQ | EVQLLESGGGLVQPGGSLRLSCAASGFSFSSSYYMCWVRQAPGKGLEWVSCI |
| ID | GTTRGSTYYADSVKGRFTISRDNSKNTLYLQMNSLRAEDTAVYYCARDATG |
| NO: | YRINTIGLYFNLWGQGTLVTVSSASTKGPSVFPLAPCSRSTSESTAALGCLVKD |
| 248 | YFPEPVTVSWNSGALTSGVHTFPAVLQSSGLYSLSSVVTVPSSSLGTKTYTCN |
| | VDHKPSNTKVDKRVESKYGPPCPPCPAPEFEGGPSVFLFPPKPKDTLYITREPE |
| | VTCVVVDVSQEDPEVQFNWYVDGVEVHNAKTKPREEQFNSTYRVVSVLTVL |
| | HQDWLNGKEYKCKVSNKGLPSSIEKTISKAKGQPREPQVYTLPPSQEEMTKN |
| | QVSLTCLVKGFYPSDIAVEWESNGQPENNYKTTPPVLDSDGSFFLYSRLTVDK |
| | SRWQEGNVFSCSVMHEALHNHYTQKSLSLSLG |
| SEQ | EVQLLESGGGLVQPGGSLRLSCAASGFSFSSSYYMCWVRQAPGKGLEWVSCI |
| ID | GTTRGSTYYADSVKGRFTISRDNSKNTVYLQMNSLRAEDTAVYYCARDATG |
| NO: | YRINTIGLYFNLWGQGTLVTVSSASTKGPSVFPLAPCSRSTSESTAALGCLVKD |
| 249 | YFPEPVTVSWNSGALTSGVHTFPAVLQSSGLYSLSSVVTVPSSSLGTKTYTCN |
| | VDHKPSNTKVDKRVESKYGPPCPPCPAPEFEGGPSVFLFPPKPKDTLYITREPE |
| | VTCVVVDVSQEDPEVQFNWYVDGVEVHNAKTKPREEQFNSTYRVVSVLTVL |
| | HQDWLNGKEYKCKVSNKGLPSSIEKTISKAKGQPREPQVYTLPPSQEEMTKN |
| | QVSLTCLVKGFYPSDIAVEWESNGQPENNYKTTPPVLDSDGSFFLYSRLTVDK |
| | SRWQEGNVFSCSVMHEALHNHYTQKSLSLSLG |
| SEQ | EVQLLESGGGLVQPGGSLRLSCAASGFSFSSSYYMCWVRQAPGKGLEWVSCI |
| ID | GTTRGSTYYADSVKGRFTISRDSSKNTLYLQMNSLRAEDTAVYYCARDATGY |
| NO: | RINTIGLYFNLWGQGTLVTVSSASTKGPSVFPLAPCSRSTSESTAALGCLVKDY |
| 250 | FPEPVTVSWNSGALTSGVHTFPAVLQSSGLYSLSSVVTVPSSSLGTKTYTCNV |
| | DHKPSNTKVDKRVESKYGPPCPPCPAPEFEGGPSVFLFPPKPKDTLYITREPEV |
| | TCVVVDVSQEDPEVQFNWYVDGVEVHNAKTKPREEQFNSTYRVVSVLTVLH |
| | QDWLNGKEYKCKVSNKGLPSSIEKTISKAKGQPREPQVYTLPPSQEEMTKNQ |
| | VSLTCLVKGFYPSDIAVEWESNGQPENNYKTTPPVLDSDGSFFLYSRLTVDKS |
| | RWQEGNVFSCSVMHEALHNHYTQKSLSLSLG |
| SEQ | EVQLLESGGGLVQPGGSLRLSCAASGFSFSSSYYMCWVRQAPGKGLEWVSCI |
| ID | GTTRGSTYYADSVKGRFTISRDSSKNTVYLQMNSLRAEDTAVYFCARDATGY |
| NO: | RINTIGLYFNLWGQGTLVTVSSASTKGPSVFPLAPCSRSTSESTAALGCLVKDY |
| 251 | FPEPVTVSWNSGALTSGVHTFPAVLQSSGLYSLSSVVTVPSSSLGTKTYTCNV |
| | DHKPSNTKVDKRVESKYGPPCPPCPAPEFEGGPSVFLFPPKPKDTLYITREPEV |
| | TCVVVDVSQEDPEVQFNWYVDGVEVHNAKTKPREEQFNSTYRVVSVLTVLH |
| | QDWLNGKEYKCKVSNKGLPSSIEKTISKAKGQPREPQVYTLPPSQEEMTKNQ |
| | VSLTCLVKGFYPSDIAVEWESNGQPENNYKTTPPVLDSDGSFFLYSRLTVDKS |
| | RWQEGNVFSCSVMHEALHNHYTQKSLSLSLG |
| SEQ | EVQLLESGGGLVQPGGSLRLSCAASGFSFSSSYYMSWVRQAPGKGLEWVSAI |
| ID | GTTRGSTYYADSVKGRFTISRDNSKNTLYLQMNSLRAEDTAVYYCARDATG |
| NO: | YRINTIGLYFNLWGQGTLVTVSSASTKGPSVFPLAPCSRSTSESTAALGCLVKD |
| 252 | YFPEPVTVSWNSGALTSGVHTFPAVLQSSGLYSLSSVVTVPSSSLGTKTYTCN |
| | VDHKPSNTKVDKRVESKYGPPCPPCPAPEFEGGPSVFLFPPKPKDTLYITREPE |
| | VTCVVVDVSQEDPEVQFNWYVDGVEVHNAKTKPREEQFNSTYRVVSVLTVL |
| | HQDWLNGKEYKCKVSNKGLPSSIEKTISKAKGQPREPQVYTLPPSQEEMTKN |
| | QVSLTCLVKGFYPSDIAVEWESNGQPENNYKTTPPVLDSDGSFFLYSRLTVDK |
| | SRWQEGNVFSCSVMHEALHNHYTQKSLSLSLG |
| SEQ | EVQLLESGGGLVQPGGSLRLSCAASGFSFSSSYYMSWVRQAPGKGLEWVSAI |
| ID | GTTRGSTYYADSVKGRFTISRDNSKNTVYLQMNSLRAEDTAVYYCARDATG |
| NO: | YRINTIGLYFNLWGQGTLVTVSSASTKGPSVFPLAPCSRSTSESTAALGCLVKD |
| 253 | YFPEPVTVSWNSGALTSGVHTFPAVLQSSGLYSLSSVVTVPSSSLGTKTYTCN |
| | VDHKPSNTKVDKRVESKYGPPCPPCPAPEFEGGPSVFLFPPKPKDTLYITREPE |
| | VTCVVVDVSQEDPEVQFNWYVDGVEVHNAKTKPREEQFNSTYRVVSVLTVL |
| | HQDWLNGKEYKCKVSNKGLPSSIEKTISKAKGQPREPQVYTLPPSQEEMTKN |
| | QVSLTCLVKGFYPSDIAVEWESNGQPENNYKTTPPVLDSDGSFFLYSRLTVDK |
| | SRWQEGNVFSCSVMHEALHNHYTQKSLSLSLG |
| SEQ | EVQLLESGGGLVQPGGSLRLSCAASGFSFSSSYYMSWVRQAPGKGLEWVSAI |
| ID | GTTRGSTYYADSVKGRFTISRDSSKNTLYLQMNSLRAEDTAVYYCARDATGY |
| NO: | RINTIGLYFNLWGQGTLVTVSSASTKGPSVFPLAPCSRSTSESTAALGCLVKDY |
| 254 | FPEPVTVSWNSGALTSGVHTFPAVLQSSGLYSLSSVVTVPSSSLGTKTYTCNV |
| | DHKPSNTKVDKRVESKYGPPCPPCPAPEFEGGPSVFLFPPKPKDTLYITREPEV |
| | TCVVVDVSQEDPEVQFNWYVDGVEVHNAKTKPREEQFNSTYRVVSVLTVLH |
| | QDWLNGKEYKCKVSNKGLPSSIEKTISKAKGQPREPQVYTLPPSQEEMTKNQ |
| | VSLTCLVKGFYPSDIAVEWESNGQPENNYKTTPPVLDSDGSFFLYSRLTVDKS |
| | RWQEGNVFSCSVMHEALHNHYTQKSLSLSLG |
| SEQ | EVQLLESGGGLVQPGGSLRLSCAASGFSFSSSYYMSWVRQAPGKGLEWVSAI |
| ID | GTTRGSTYYADSVKGRFTISRDSSKNTVYLQMNSLRAEDTAVYFCARDATGY |
| NO: | RINTIGLYFNLWGQGTLVTVSSASTKGPSVFPLAPCSRSTSESTAALGCLVKDY |
| 255 | FPEPVTVSWNSGALTSGVHTFPAVLQSSGLYSLSSVVTVPSSSLGTKTYTCNV |
| | DHKPSNTKVDKRVESKYGPPCPPCPAPEFEGGPSVFLFPPKPKDTLYITREPEV |
| | TCVVVDVSQEDPEVQFNWYVDGVEVHNAKTKPREEQFNSTYRVVSVLTVLH |
| | QDWLNGKEYKCKVSNKGLPSSIEKTISKAKGQPREPQVYTLPPSQEEMTKNQ |
| | VSLTCLVKGFYPSDIAVEWESNGQPENNYKTTPPVLDSDGSFFLYSRLTVDKS |
| | RWQEGNVFSCSVMHEALHNHYTQKSLSLSLG |
| SEQ | QSLEESGGDLVKPGASLTLTCRVSGFSFSSSYYMCWVRQAPGKGLEWIACIGT |
| ID | TRGSTYYATWAKGRFTISKISSTTVTLQMTSLTDADTATYFCARDATGYRINT |
| NO: | IGLYFNLWGPGTLVTVSSASTKGPSVFPLAPCSRSTSESTAALGCLVKDYFPEP |
| 256 | VTVSWNSGALTSGVHTFPAVLQSSGLYSLSSVVTVPSSSLGTKTYTCNVDHKP |
| | SNTKVDKRVESKYGPPCPPCPAPEFEGGPSVFLFPPKPKDTLYITREPEVTCVV |
| | VDVSQEDPEVQFNWYVDGVEVHNAKTKPREEQFNSTYRVVSVLTVLHQDW |
| | LNGKEYKCKVSNKGLPSSIEKTISKAKGQPREPQVYTLPPSQEEMTKNQVSLT |
| | CLVKGFYPSDIAVEWESNGQPENNYKTTPPVLDSDGSFFLYSRLTVDKSRWQ |
| | EGNVFSCSVMHEALHNHYTQKSLSLSLG |

In some embodiments, the antibody, or any antibody fragment thereof, comprises a light chain selected from the Table 9 below.

TABLE 9
antibody light chains

| | |
|---|---|
| SEQ | DIQITQSPSSLSASVGDRVTITCKSSQSLLYSENNQDYLAWYQQKPGKVPKLLI |
| ID | YGASNRHTGVPSRFSGSGSGTDFTLTISSLQPEDVATYYCEQTYRYPFTFGQG |
| NO: | TKLEIKRTVAAPSVFIFPPSDEQLKSGTASVVCLLNNFYPREAKVQWKVDNAL |
| 144 | QSGNSQESVTEQDSKDSTYSLSSTLTLSKADYEKHKVYACEVTHQGLSSPVTK |
| | SFNRGEC |
| SEQ | DILINQSPASLTVSAGERVTMSCKSSQSLLYSENNQDYLAWYQQKPGQFPKLL |
| ID | IYGASNRHTGVPDRFTGSGSGTDFTLTISSVQAEDLADYYCEQTYRYPFTFGS |
| NO: | GTKLEIKRTVAAPSVFIFPPSDEQLKSGTASVVCLLNNFYPREAKVQWKVDNA |
| 145 | LQSGNSQESVTEQDSKDSTYSLSSTLTLSKADYEKHKVYACEVTHQGLSSPVT |
| | KSFNRGEC |
| SEQ | DIQITQSPSSLSASVGDRVTITCKSSQSLLYSENNQDYLAWYQQKPGKVPKLLI |
| ID | YGASNRHTGVPSRFSGSGSGTDFTLTISSLQPEDVATYYCEQTYRYPFTFGQG |
| NO: | TKLEIKRTVAAPSVFIFPPSDEQLKSGTASVVCLLNNFYPREAKVQWKVDNAL |
| 146 | QSGNSQESVTEQDSKDSTYSLSSTLTLSKADYEKHKVYACEVTHQGLSSPVTK |
| | SFNRGEC |
| SEQ | DIQMTQSPSSLSASVGDRVTITCKSSQSLLYSENNQDYLAWYQQKPGKVPKLL |
| ID | IYGASNRHTGVPSRFSGSGSGTDFTLTISSLQPEDVATYYCEQTYRYPFTFGQG |
| NO: | TKLEIKRTVAAPSVFIFPPSDEQLKSGTASVVCLLNNFYPREAKVQWKVDNAL |
| 147 | QSGNSQESVTEQDSKDSTYSLSSTLTLSKADYEKHKVYACEVTHQGLSSPVTK |
| | SFNRGEC |
| SEQ | DIQMTQSPSSLSASVGDRVTITCRASQSISSYLNWYQQKPGKAPKLLIYDASSL |
| ID | ESGVPSRFSGSGSGTEFTLTISSLQPDDFAVYYCQQYNNWPQTFGQGTKVEIK |
| NO: | RTVAAPSVFIFPPSDEQLKSGTASVVCLLNNFYPREAKVQWKVDNALQSGNS |
| 172 | QESVTEQDSKDSTYSLSSTLTLSKADYEKHKVYACEVTHQGLSSPVTKSFNRG |
| | EC |
| SEQ | DIVMTQSPDSLAVSLGERATINCQASQTIYSYLSWYQQKPGQPPKLLIYEASKL |
| ID | ASGVPDRFSGSGSGTDYTLTISSLQAEDVAVYYCQSYHGTASTEYNTFGGGT |
| NO: | KVEIKRTVAAPSVFIFPPSDEQLKSGTASVVCLLNNFYPREAKVQWKVDNAL |
| 257 | QSGNSQESVTEQDSKDSTYSLSSTLTLSKADYEKHKVYACEVTHQGLSSPVTK |
| | SFNRGEC |
| SEQ | ELTQTPSSVEAAVGGTPTIKCQASQTIYSYLSWYQQKPGQPPKLLIYEASKLAS |
| ID | GVPSRFSGSGSGTDYTLTISDLECADAATYYCQSYHGTASTEYNTFGGGTEVV |
| NO: | VKRTVAAPSVFIFPPSDEQLKSGTASVVCLLNNFYPREAKVQWKVDNALQSG |
| 258 | NSQESVTEQDSKDSTYSLSSTLTLSKADYEKHKVYACEVTHQGLSSPVTKSFN |
| | RGEC |

In some embodiments, antibodies described herein comprises a heavy chain selected from Table 8 and a light chain selected from Table 9.

In some embodiments, the antibody comprises a heavy chain selected from SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, or SEQ ID NO: 143; and a light chain selected from SEQ ID NO: 144, SEQ ID NO: 145, SEQ ID NO: 146, or SEQ ID NO: 147.

In some embodiments, the antibody comprises a heavy chain selected from SEQ ID NO: 148, SEQ ID NO: 149, SEQ ID NO: 150, SEQ ID NO: 151, SEQ ID NO: 152, SEQ ID NO: 153, SEQ ID NO: 154, SEQ ID NO: 155, SEQ ID NO: 156, SEQ ID NO: 157, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170, or SEQ ID NO: 171; and a light chain selected from SEQ ID NO: 172.

In some embodiments, the antibody comprises a heavy chain selected from SEQ ID NO: 173, SEQ ID NO: 174, SEQ ID NO: 175, SEQ ID NO: 176, SEQ ID NO: 177, SEQ ID NO: 178, SEQ ID NO: 179, SEQ ID NO: 180, SEQ ID NO: 181, SEQ ID NO: 182, SEQ ID NO: 183, SEQ ID NO: 184, SEQ ID NO: 185, SEQ ID NO: 186, SEQ ID NO: 187, SEQ ID NO: 188, SEQ ID NO: 189, SEQ ID NO: 190, SEQ ID NO: 191, SEQ ID NO: 192, SEQ ID NO: 193, SEQ ID NO: 194, SEQ ID NO: 195, SEQ ID NO: 196, SEQ ID NO: 197, SEQ ID NO: 198, SEQ ID NO: 199, SEQ ID NO: 200, SEQ ID NO: 201, SEQ ID NO: 202, SEQ ID NO: 203, SEQ ID NO: 204, SEQ ID NO: 205, SEQ ID NO: 206, SEQ ID NO: 207, SEQ ID NO: 208, SEQ ID NO: 209, SEQ ID NO: 210, SEQ ID NO: 211, SEQ ID NO: 212, SEQ ID NO: 213, SEQ ID NO: 214, SEQ ID NO: 215, SEQ ID NO: 216, SEQ ID NO: 217, SEQ ID NO: 218, SEQ ID NO: 219, SEQ ID NO: 220, SEQ ID NO: 221, SEQ ID NO: 222, SEQ ID NO: 223, SEQ ID NO: 224, SEQ ID NO: 225, SEQ ID NO: 226, SEQ ID NO: 227, SEQ ID NO: 228, SEQ ID NO: 229, SEQ ID NO: 230, SEQ ID NO: 231, SEQ ID NO: 232, SEQ ID NO: 233, SEQ ID NO: 234, SEQ ID NO: 235, SEQ ID NO: 236, SEQ ID NO: 237, SEQ ID NO: 238, SEQ ID NO: 239, SEQ ID NO: 240, SEQ ID NO: 241, SEQ ID NO: 242, SEQ ID NO: 243, SEQ ID NO: 244, SEQ ID NO: 245, SEQ ID NO: 246, SEQ ID NO: 247, SEQ ID NO: 248, SEQ ID NO: 249, SEQ ID NO: 250, SEQ ID NO: 251, SEQ ID NO: 252, SEQ ID NO: 253, SEQ ID NO: 254, SEQ ID NO: 255, or SEQ ID NO: 256; and a light chain selected from SEQ ID NO: 257, or SEQ ID NO: 258.

As provided for herein, the antibodies, or antigen-binding fragments thereof can be variants of the sequences. Accordingly, in some embodiments, a variant of an antibody or antigen-binding fragment thereof provided for herein is provided. In some embodiments, the variant comprises mutations selected from substitutions, deletions, insertions, or a combination thereof. In some embodiments, the variant comprises between 1 and 20 mutations. In some embodiments, the variant comprises between 1 and 10 mutations. In some embodiments, the variant comprises between 1 and 5 mutations. In some embodiments, the variant comprises 1, 2, 3, 4, 5, 6, 7, 8, 9, 10, 11, 12, 13, 14, 15, 16, 17, 18, 19, or 20 mutations. In some embodiments, the variant comprises at least 1 mutation. Accordingly, in some embodiments, the variant comprises up to or more than 20 mutations. In some embodiments, the variant comprises 1-10 mutations, wherein the mutations are selected from substitutions, deletions, insertions, or a combination thereof. In some embodiments, the variant comprises 1-10 mutations, wherein the mutations are conservative substitutions. Examples of conservative substitutions are as provided for herein (Table 1). Further, one of skill in the art will recognize and understand the substitutions that are encompassed by the term “conservative substitutions”. Such substitutions are within the scope of the present disclosure.

The antibody or antigen-binding fragment may comprise an antibody fragment as defined and provided for herein. In some embodiments, the antibody binding fragment is as provided for herein. In some embodiments, the antibody fragment is a scFv antibody, a Fab fragment, a Fab′ fragment, or a F(ab′)₂ fragment. In some embodiments, the antibody fragment is a scFv antibody. In some embodiments, the antibody fragment is a Fab fragment. In some embodiments, the antibody fragment is a Fab′ fragment. In some embodiments, the antibody fragment is a F(ab′)₂ fragment.

The sequences of the antibodies can be modified to yield human IgG antibodies. The conversion of the sequences provided herein can be modified to yield other types of antibodies. The CDRs can also be linked to other antibodies, proteins, or molecules to create antibody fragments that bind α11β1. This can be in the form of an antibody drug conjugate (“ADC”), or a multi-specific molecule. The sequences can also be made into chimeric antibodies as described herein.

In some embodiments, the antibody comprises an amino acid sequence comprising a sequence provided for herein or a fragment thereof. In some embodiments, the antibody is a monoclonal antibody. In some embodiments, the antibody is a polyclonal antibody. In some embodiments, the antibody is a humanized antibody. In some embodiments, the antibody comprises one or more amino acid sequences as provided herein, an antigen-binding fragments, thereof, or a human IgG variant thereof. “A human IgG variant thereof” refers to an antibody that has been modified to be a human IgG when the starting antibody is not a human IgG antibody.

As described herein the production of antibodies with a known sequence is routine and can be done by any method. The antibodies can also be modified to be chimeric antibodies or human antibodies. The antibodies can also be used in injectable pharmaceutical compositions. As also described herein, the antibodies can be isolated antibodies or engineered antibodies.

In some embodiments, “derivatives” of the antibodies, fragments, regions or derivatives thereof, which term includes those proteins encoded by truncated or modified genes to yield molecular species functionally resembling the immunoglobulin fragments are provided. The modifications include, but are not limited to, addition of genetic sequences coding for cytotoxic proteins such as plant and bacterial toxins. The modification can also include a reporter protein, such as a fluorescent or chemiluminescent tag. The fragments and derivatives can be produced in any manner.

The identification of these antibodies, or antigen-binding fragments thereof, provide the information necessary to generate additional monoclonal antibodies with similar binding characteristics and therapeutic or diagnostic utility that parallel the embodiments of this application.

The antibodies can be generated according the examples provided herein. Once the sequences are known, the antibodies can also be generated according to known methods. The antibodies can also be converted to different types, such as being converted to Human IgGs and the like. By converting the antibodies to a human antibody, a human subject should not identify the antibodies as foreign. The conversion of a non-human IgG antibody to a human IgG antibody is well known and can routinely be done once the native sequence is known. As discussed herein, the antibodies can be modified according to known methods. Such methods are described in, for example, Riechmann L, Clark M, Waldmann H, Winter G (1988). Reshaping human antibodies for therapy”. Nature 332 (6162): 332-323; Tsurushita N, Park M, Pakabunto K, Ong K, Avdalovic A, Fu H, Jia A, Vásquez M, Kumar S. (2004), which is incorporated by reference in its entirety.

In some embodiments, a host cell is provided. In some embodiments, the host cell comprises an antibody or antigen-binding fragment thereof as provided for herein. In some embodiments, the host cell comprises a nucleic acid molecule encoding for antibody or antigen-binding fragment thereof as provided for herein. In some embodiments, the host cell comprises a vector, said vector comprising comprises a nucleic acid molecule encoding for antibody or antigen-binding fragment thereof as provided for herein.

In some embodiments, the host cell produces an antibody or antigen-binding fragment thereof as provided for herein. In some embodiments, an antibody or antigen-binding fragment thereof as provided for herein is provided, wherein the antibody or antigen-binding fragment thereof as provided for herein is produced by the host cell.

In some embodiments, a method of producing a polypeptide is provided. In some embodiments, the polypeptide comprises a heavy chain variable region as provided for herein, a light chain variable region as provided for herein, or a combination thereof. In some embodiments, the method comprises growing a host cell under conditions so that the host cell expresses the polypeptide comprising the heavy chain variable region, light chain variable region, or combination thereof, and purifying the polypeptide comprising the heavy chain variable region, the light chain variable region, or a combination thereof, thereby producing the polypeptide. In some embodiments, the host cell is a host cell as provided for herein.

In some embodiments, a method of producing an antibody or antigen-binding fragment thereof is provided. In some embodiments, the antibody binds human α11β1. In some embodiments, the antigen-binding fragment binds human α11β1. In some embodiments, the method comprises growing a host cell under conditions so that the host cell expresses a polypeptide or polypeptides comprising the immunoglobulin heavy chain variable region and/or the immunoglobulin light chain variable region, thereby producing the antibody or the antigen-binding fragment of the antibody, and purifying the antibody or the antigen-binding fragment of the antibody. In some embodiments, the polypeptide or polypeptides comprise the immunoglobulin heavy chain variable region. In some embodiments, the polypeptide or polypeptides comprise the immunoglobulin light chain variable region. In some embodiments, the polypeptide or polypeptides comprise the immunoglobulin heavy chain variable region and the immunoglobulin light chain variable region. In some embodiments, the host cell is a host cell as provided for herein. In some embodiments, the antibody or antigen-binding fragment thereof is as provided for herein.

The polypeptide or antibody-producing cell contributing the nucleotide sequences encoding the antigen-binding region of the chimeric antibody can also be produced by transformation of a non-human, such as a primate, or a human cell. For example, a B lymphocyte which produces the antibody can be infected and transformed with a virus such as Epstein-Barr virus to yield an immortal antibody producing cell (Kozbor et al., Immunol. Today 4:72 79 (1983)). Alternatively, the B lymphocyte can be transformed by providing a transforming gene or transforming gene product, as is well-known in the art. See, e.g., Ausubel infra, Harlow infra, and Colligan infra, the contents of which references are incorporated entirely herein by reference. The cell fusions are accomplished by standard procedures well known to those skilled in the field of immunology, such as producing cells can also be a hybridoma which is generated by fusing a B-cell with an immortal myeloma cell. Fusion partner cell lines and methods for fusing and selecting hybridomas and screening for mAbs are well known in the art. See, e.g., Ausubel infra, Harlow infra, and Colligan infra, the contents of which references are incorporated entirely herein by reference.

The nucleic acid sequence encoding an antibody described herein can be genomic DNA or cDNA, or RNA (e.g. mRNA) which encodes at least one of the variable regions described herein. A convenient alternative to the use of chromosomal gene fragments as the source of DNA encoding the V region antigen-binding segment is the use of cDNA for the construction of chimeric immunoglobulin genes, e.g., as reported by Liu et al. (Proc. Natl. Acad. Sci., USA 84:3439 (1987) and J. Immunology 139:3521 (1987), which references are hereby entirely incorporated herein by reference. The use of cDNA requires that gene expression elements appropriate for the host cell be combined with the gene in order to achieve synthesis of the desired protein. The use of cDNA sequences is advantageous over genomic sequences (which contain introns), in that cDNA sequences can be expressed in bacteria or other hosts which lack appropriate RNA splicing systems.

For example, a cDNA encoding a V region antigen-binding segment able to detect, bind, to or neutralize A11B1s can be provided using known methods based on the use of the amino acid sequences provided herein. Because the genetic code is degenerate, more than one codon can be used to encode a particular amino acid (Watson, et al., infra). Using the genetic code, one or more different oligonucleotides can be identified, each of which would be capable of encoding the amino acid. The probability that a particular oligonucleotide will, in fact, constitute the actual encoding sequence can be estimated by considering abnormal base pairing relationships and the frequency with which a particular codon is actually used (to encode a particular amino acid) in eukaryotic or prokaryotic cells expressing an antibody or fragment. Such “codon usage rules” are disclosed by Lathe, et al., J. Molec. Biol. 183:1 12 (1985). Using the “codon usage rules” of Lathe, a single oligonucleotide, or a set of oligonucleotides, that contains a theoretical “most probable” nucleotide sequence capable of encoding an antibody variable or constant region sequences is identified.

The variable regions described herein can be combined with any type of constant region including a human constant region or murine constant region. Human genes which encode the constant (C) regions of the antibodies, fragments and regions can be derived from a human fetal liver library, by known methods. Human C region genes can be derived from any human cell including those which express and produce human immunoglobulins. The human C_{H} region can be derived from any of the known classes or isotypes of human H chains, including gamma, u, «, 8 or &, and subtypes thereof, such as G1, G2, G3 and G4. Since the H chain isotype is responsible for the various effector functions of an antibody, as well as antibody recycling by interaction with FcRn, the choice of C_{H} region will be guided by the desired effector functions, such as complement fixation, or activity in antibody-dependent cellular cytotoxicity (ADCC). Preferably, the C_{H} region is derived from gamma 1 (IgG1), gamma 3 (lgG3), gamma 4 (IgG4), or u (IgM). The human CL region can be derived from either human L chain isotype, kappa or lambda.

Methods of purifying polypeptides or proteins (including antibodies or antigen-binding fragments thereof) are known in the art, and any such method is within the scope of the present application. Further, the host cell is not limited by the examples recited above. Any suitable cell may be used to generate the polypeptides or proteins of the present disclosure.

### Pharmaceutical Compositions

In some embodiments, a pharmaceutical composition is provided. In some embodiments, the pharmaceutical composition comprises an antibody or antigen-binding fragment thereof as provided for herein.

In some embodiments, to prepare pharmaceutical or sterile compositions of the anti-α11β1 antibodies or other proteins provided herein, the antibody or antigen-binding fragment thereof or other proteins provided herein are admixed with a pharmaceutically acceptable carrier or excipient. See, e.g., Remington's Pharmaceutical Sciences and U.S. Pharmacopeia: National Formulary, Mack Publishing Company, Easton, PA (1984).

Formulations of therapeutic or the antibodies provided herein may be prepared by mixing with acceptable carriers, excipients, or stabilizers in the form of, e.g., lyophilized powders, slurries, aqueous solutions or suspensions (see, e.g., Hardman, et al. (2001) Goodman and Gilman's The Pharmacological Basis of Therapeutics, McGraw-Hill, New York, NY, Gennaro (2000) Remington: The Science and Practice of Pharmacy, Lippincott, Williams, and Wilkins, New York, NY; Avis, et al. (eds.) (1993) Pharmaceutical Dosage Forms: Parenteral Medications, Marcel Dekker, NY; Lieberman, et al. (eds.) (1990) Pharmaceutical Dosage Forms: Tablets, Marcel Dekker, NY; Lieberman, et al. (eds.) (1990) Pharmaceutical Dosage Forms: Disperse Systems, Marcel Dekker, NY; Weiner and Kotkoskie (2000) Excipient Toxicity and Safety, Marcel Dekker, Inc., New York, NY). In some embodiments, the antibodies are diluted to an appropriate concentration in a sodium acetate solution pH 5-6, and NaCl or sucrose is added for tonicity. Additional agents, such as polysorbate 20 or polysorbate 80, may be added to enhance stability.

Toxicity and therapeutic efficacy of the antibody compositions, administered alone or in combination with another agent, can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index (LD₅₀/ED₅₀). In particular aspects, antibodies exhibiting high therapeutic indices are desirable. The data obtained from these cell culture assays and animal studies can be used in formulating a range of dosage for use in human. The dosage of such compounds lies preferably within a range of circulating concentrations that include the ED₅₀ with little or no toxicity. The dosage may vary within this range depending upon the dosage form employed and the route of administration.

In some embodiments, a composition is administered to a subject in accordance with the Physicians' Desk Reference 2003 (Thomson Healthcare; 57th edition (Nov. 1, 2002)).

The mode of administration can vary. Suitable routes of administration include oral, rectal, transmucosal, intestinal, parenteral; intramuscular, subcutaneous, intradermal, intramedullary, intrathecal, direct intraventricular, intravenous, intraperitoneal, intranasal, intraocular, inhalation, insufflation, topical, cutaneous, transdermal, or intra-arterial. In some embodiments, the composition is an injectable pharmaceutical composition. In some embodiments, the composition is formulated for intravenous or subcutaneous injection. In some embodiments, the composition is formulated for intravenous injection. In some embodiments, the composition id formulated for subcutaneous injection.

In some embodiments, the antibody or antigen-binding fragment thereof can be administered by an invasive route such as by injection. In some embodiments, the antibodies or antigen-binding fragment thereof, or pharmaceutical composition thereof, is administered intravenously, subcutaneously, intramuscularly, intraarterially, intra-articularly (e.g. in arthritis joints), intratumorally, or by inhalation, aerosol delivery. Administration by non-invasive routes (e.g., orally; for example, in a pill, capsule or tablet) is also within the scope of the present embodiments.

In some embodiments, the anti-α11β1 antibody, or antigen-binding fragment thereof, is administered in combination with at least one additional therapeutic agent, such as, but not limited to any therapeutic used to treat the disorders provided for herein. In some embodiments, the antibody is administered in combination with another treatment for the disorders provided for herein.

Compositions can be administered with medical devices known in the art. For example, a pharmaceutical composition of the invention can be administered by injection with a hypodermic needle, including, e.g., a prefilled syringe or autoinjector.

The pharmaceutical compositions may also be administered with a needleless hypodermic injection device; such as the devices disclosed in U.S. Pat. Nos. 6,620,135; 6,096,002; 5,399,163; 5,383,851; 5,312,335; 5,064,413; 4,941,880; 4,790,824 or 4,596,556.

The pharmaceutical compositions may also be administered by infusion. Examples of well-known implants and modules form administering pharmaceutical compositions include: U.S. Pat. No. 4,487,603, which discloses an implantable micro-infusion pump for dispensing medication at a controlled rate; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments. Many other such implants, delivery systems, and modules are well known to those skilled in the art.

Alternately, one may administer the antibody in a local rather than systemic manner, for example, via injection of the antibody directly into an arthritic joint or pathogen-induced lesion characterized by immunopathology, often in a depot or sustained release formulation. Furthermore, one may administer the antibody in a targeted drug delivery system, for example, in a liposome coated with a tissue-specific antibody, targeting, for example, arthritic joint or pathogen-induced lesion characterized by immunopathology. The liposomes will be targeted to and taken up selectively by the afflicted tissue.

The administration regimen depends on several factors, including the serum or tissue turnover rate of the therapeutic antibody, the level of symptoms, the immunogenicity of the therapeutic antibody, and the accessibility of the target cells in the biological matrix. Preferably, the administration regimen delivers sufficient therapeutic antibody to effect improvement in the target disease state, while simultaneously minimizing undesired side effects. Accordingly, the amount of biologic delivered depends in part on the particular therapeutic antibody and the severity of the condition being treated. Guidance in selecting appropriate doses of therapeutic antibodies is available (see, e.g., Wawrzynczak (1996) Antibody Therapy, Bios Scientific Pub. Ltd, Oxfordshire, UK; Kresina (ed.) (1991) Monoclonal Antibodies, Cytokines and Arthritis, Marcel Dekker, New York, NY; Bach (ed.) (1993) Monoclonal Antibodies and Peptide Therapy in Autoimmune Diseases, Marcel Dekker, New York, NY; Baert, et al. (2003) New Engl. J. Med. 348:601-608; Milgrom et al. (1999) New Engl. J. Med. 341:1966-1973; Slamon et al. (2001) New Engl. J. Med. 344:783-792; Beniaminovitz et al. (2000) New Engl. J. Med. 342:613-619; Ghosh et al. (2003) New Engl. J. Med. 348:24-32; Lipsky et al. (2000) New Engl. J. Med. 343:1594-1602).

Determination of the appropriate dose can be made by the clinician, e.g., using parameters or factors known or suspected in the art to affect treatment. Generally, the dose begins with an amount somewhat less than the optimum dose and it is increased by small increments thereafter until the desired or optimum effect is achieved relative to any negative side effects. Important diagnostic measures include those of symptoms of, e.g., the inflammation or level of inflammatory cytokines produced. In general, it is desirable that a biologic that will be used is derived from the same species as the animal targeted for treatment, thereby minimizing any immune response to the reagent. In the case of human subjects, for example, chimeric, humanized and fully human antibodies are may be desirable.

Antibodies or antigen-binding fragments thereof can be provided by continuous infusion, or by doses administered, e.g., daily, 1-7 times per week, weekly, bi-weekly, monthly, bimonthly, or quarterly. In some embodiments, a total weekly dose is generally at least 0.05 μg/kg body weight, more generally at least 0.2 μg/kg, 0.5 μg/kg, 1 μg/kg, 10 μg/kg, 100 μg/kg, 0.25 mg/kg, 1.0 mg/kg, 2.0 mg/kg, 5.0 mg/ml, 10 mg/kg, 25 mg/kg, 50 mg/kg or more (see, e.g., Yang, et al. (2003) New Engl. J. Med. 349:427-434; Herold, et al. (2002) New Engl. J. Med. 346:1692-1698; Liu, et al. (1999) J. Neurol. Neurosurg. Psych. 67:451-456; Portielji, et al. (20003) Cancer Immunol. Immunother. 52:133-144). Doses may also be provided to achieve a pre-determined target concentration of the antibody in the subject's serum, such as 0.1, 0.3, 1, 3, 10, 30, 100, 300 μg/ml or more. In other embodiments, a fully human antibody is administered subcutaneously or intravenously, on a weekly, biweekly, “every 4 weeks,” monthly, bimonthly, or quarterly basis at 10, 20, 50, 80, 100, 200, 500, 1000 or 2500 mg/subject.

As used herein, “inhibit” or “treat” or “treatment” includes a postponement of development of the symptoms associated with a disorder and/or a reduction in the severity of the symptoms of such disorder. The terms further include ameliorating existing uncontrolled or unwanted symptoms, preventing additional symptoms, and ameliorating or preventing the underlying causes of such symptoms. Thus, the terms denote that a beneficial result has been conferred on a vertebrate subject with a disorder, disease or symptom, or with the potential to develop such a disorder, disease or symptom.

As used herein, the terms “therapeutically effective amount”, “therapeutically effective dose” and “effective amount” refer to an amount of the antibody, or antigen-binding fragment thereof, that, when administered alone or in combination with an additional therapeutic agent to a cell, tissue, or subject, is effective to cause a measurable improvement in one or more symptoms of a disease or condition or the progression of such disease or condition. A therapeutically effective dose further refers to that amount of the binding compound sufficient to result in at least partial amelioration of symptoms, e.g., treatment, healing, prevention or amelioration of the relevant medical condition, or an increase in rate of treatment, healing, prevention or amelioration of such conditions. When applied to an individual active ingredient administered alone, a therapeutically effective dose refers to that ingredient alone. When applied to a combination, a therapeutically effective dose refers to combined amounts of the active ingredients that result in the therapeutic effect, whether administered in combination, serially or simultaneously. An effective amount of a therapeutic will result in an improvement of a diagnostic measure or parameter by at least 10%; usually by at least 20%; preferably at least about 30%; more preferably at least 40%, and most preferably by at least 50%. An effective amount can also result in an improvement in a subjective measure in cases where subjective measures are used to assess disease severity. In some embodiments, an amount is a therapeutically effective amount if it is an amount that can be used to treat or ameliorate tumors or gastric tumors.

The term “subject” as used throughout includes any organism, such as an animal, including a mammal (e.g., rat, mouse, dog, cat, rabbit) and, for example, a human. In one embodiment, the subject is a human. A subject can also be referred to as a patient. In some embodiments, the subject is a subject in need thereof. A subject that is “in need thereof” refers to a subject that has been identified as requiring treatment for the condition that is to be treated and is treated with the specific intent of treating such condition. The conditions can be, for example, any of the conditions described herein.

### Methods

In some embodiments, the methods comprise administering a therapeutically or prophylactically effective amount of one or more antibodies or antigen-binding fragments of the antibodies described herein to a susceptible subject or to one exhibiting a condition in which α11β1 is known to have caused the pathology observed. Any active form of the antibody can be administered, including, but not limited to scFv, Fab and F(ab′)2 fragments and other forms of antibodies provided for herein.

The binding properties of an antibody described herein to α11β1 can be measured by methods known in the art, e.g., one of the following methods: BIACORE analysis, Enzyme Linked Immunosorbent Assay (ELISA), x-ray crystallography, sequence analysis and scanning mutagenesis. The binding interaction of an antibody and α11β1 can be analyzed using surface plasmon resonance (SPR). SPR or Biomolecular Interaction Analysis (BIA) detects bio-specific interactions in real time, without labeling any of the interactants. Changes in the mass at the binding surface (indicative of a binding event) of the BIA chip result in alterations of the refractive index of light near the surface. The changes in the refractivity generate a detectable signal, which are measured as an indication of real-time reactions between biological molecules. Methods for using SPR are described, for example, in U.S. Pat. No. 5,641,640; Raether (1988) Surface Plasmons Springer Verlag; Sjolander and Urbaniczky (1991) Anal. Chem. 63:2338-2345; Szabo et al. (1995) Curr. Opin. Struct. Biol. 5:699-705 and on-line resources provide by BIAcore International AB (Uppsala, Sweden). Additionally, a KinExA® (Kinetic Exclusion Assay) assay, available from Sapidyne Instruments (Boise, Id.) can also be used.

Information from SPR can be used to provide an accurate and quantitative measure of the equilibrium dissociation constant (K_{D}), and kinetic parameters, including Kₒₙ and K_{off}, for the binding of an antibody to α11β1. Such data can be used to compare different molecules. Information from SPR can also be used to develop structure-activity relationships (SAR). Variant amino acids at given positions can be identified that correlate with particular binding parameters, e.g., high affinity.

In certain embodiments, an antibody described herein exhibits high affinity for binding α11β1. In various embodiments, K_{D} of an antibody as described herein for α11β1 is less than about 10⁻⁴, 10⁻⁵, 10⁻⁶, 10⁻⁷, 10⁻⁸, 10⁻⁹, 10⁻¹⁰, 10⁻¹¹, 10⁻¹², 10⁻¹³, 10⁻¹⁴, or 10⁻¹⁵ M. In certain instances, K_{D} of an antibody as described herein for α11β1 is between 0.001 and 1 nM, e.g., 0.001 nM, 0.005 nM, 0.01 nM, 0.05 nM, 0.1 nM, 0.5 nM, or 1 nM.

In some embodiments, a use of an antibody or antigen-binding fragment as provided for herein or a pharmaceutical composition as provided for herein is provided. In some embodiments, the use is for the treatment of a α11β1 mediated disorder. In some embodiments, a use of an antibody or antigen-binding fragment as provided for herein is provided, the use for the treatment of a α11β1 mediated disorder. In some embodiments, a use of a pharmaceutical composition comprising an antibody or antigen binging fragment as provided for herein is provided, the use for the treatment of a A11B1a mediated disorder. In some embodiments, the pharmaceutical composition is as provided for herein. The antibody of any of the embodiments or pharmaceutical compositions thereof inhibit complement α11β1 activity in an individual having a complement-mediated disease or disorder.

The present disclosure provides use of an anti-α11β1 antibody of any of the embodiments or a pharmaceutical composition thereof in the manufacture of a medicament for inhibiting complement α11β1 activity. In some embodiments, the present disclosure provides use of an anti-α11β1 antibody of any of the embodiments or a pharmaceutical composition thereof in the manufacture of a medicament for inhibiting complement α11β1 activity in an individual having a α11β1-mediated disease or disorder.

The present disclosure provides an anti-α11β1 antibody of any of the embodiments or a pharmaceutical composition thereof for use in medical therapy.

Treatment of individuals may comprise the administration of a therapeutically effective amount of the antibodies described herein. The antibodies can be provided in a kit, such as those provided herein. The antibodies can be used or administered alone or in admixture with another therapeutic, analgesic, or diagnostic agent, such as provided for herein. In providing a patient with an antibody, or fragment thereof, capable of binding to α11β1, or an antibody capable of protecting against α11β1 in a recipient patient, the dosage of administered agent will vary depending upon such factors as the patient's age, weight, height, sex, general medical condition, previous medical history, etc.

An antibody, capable treating a condition associated with α11β1 activity or use to treat a α11β1 related pathology, is intended to be provided to subjects in an amount sufficient to affect a reduction, resolution, or amelioration in the α11β1 related symptom or pathology. Examples of such pathologies are provided for herein.

Accordingly, in some embodiments, methods of treating a subject with a α11β1 mediated disorder are provided. In some embodiments, the method comprises administering a pharmaceutical composition comprising an antibody, or antigen-binding fragment thereof, as provided herein. In some embodiments, the disorder is as provided for herein.

As provided for herein, the antibodies, or antigen-binding fragments thereof, can be administered with other therapeutics. These can be administered simultaneously or sequentially.

Kits are also provided which are useful for carrying out embodiments described herein. The present kits can comprise a first container containing or packaged in association with the above-described antibodies. The kit may also comprise another container containing or packaged in association solutions necessary or convenient for carrying out the embodiments. The containers can be made of glass, plastic or foil and can be a vial, bottle, pouch, tube, bag, etc. The kit may also contain written information, such as procedures for carrying out the embodiments or analytical information, such as the amount of reagent contained in the first container means. The container may be in another container apparatus, e.g. a box or a bag, along with the written information.

In some embodiments, antibodies that bind to an α11β1 protein are provided. In some embodiments, the antibodies are antibodies or antigen-binding fragments as provided for herein. In some embodiments, the antibodies or antigen-binding fragments comprise an amino acid sequence as provided for herein, or a variant thereof as provided for herein. In some embodiments, the antibody is isolated. In some embodiments, the antibody binds specifically to the active form of α11β1

In some embodiments, the antibody inhibits or neutralizes the function of an active form of α11β1 protein. As used herein, the term “neutralize” means that the activity or function of the protein is inhibited. The inhibition can be complete or partial. In some embodiments, the activity or function of the protein is inhibited at least 10, 20, 30, 40, 50, 60, 70, 80, 90, 95, or 99%. The percent inhibition can be based upon the function or activity of the protein in the absence of the antibody. In some embodiments, the antibody inhibits the function facilitated by α11β1.

In some embodiments, one or more anti-α11β1 antibodies described herein are used in a method of treating one or more disorders described herein. In some embodiments, the method comprises administering to a subject in need thereof a therapeutically effective amount of an antibody, or antigen-binding fragment thereof, described herein.

In some embodiments, one or more anti-α11β1 antibodies described herein are used in a method of treating a fibrotic disorder. Fibrosis is a process of scarring that manifests itself in many tissues in the body, typically as a result of inflammation or tissue damage. Increased production of extracellular matrix results in organ failure and, often, death. Diseases associated with fibrosis account for approximately 45% of all deaths in industrialized nations (Wynn, T. A., 2008, J Pathol. 214:199-210). The cells responsible for producing extracellular matrix (ECM) for tissue repair (and in fibrosis) are a specialized type of fibroblasts called myofibroblasts (MF). Although mechanisms of fibrosis have been extensively studied, this complex process is far from well understood. In order to focus on the most important drivers of fibrosis, published patient-derived datasets (SSc patient data and normal controls) were interrogated using an in-house derived novel data analysis methodology. This analysis lead to the identification of the type I collagen binding integrin α11β1 as one of the top targets for modulating fibrosis.

In some embodiments, a fibrotic disorder is or comprises idiopathic pulmonary fibrosis (IPF), chronic kidney disease, diabetic cardiomyopathy, primary sclerosing cholangitis (PSC), primary biliary cirrhosis (PBC), non-alcoholic fatty liver disease (NAFLD/NASH), Crohn's disease, ulcerative colitis, or systemic sclerosis (SSc). In some embodiments, a fibrotic disorder is or comprises atrial fibrosis, endomyocardial fibrosis, arthrofibrosis, mediastinal fibrosis, myelofibrosis, progressive massive fibrosis, retroperitoneal fibrosis or skeletal muscle fibrosis.

In some embodiments, a fibrotic disorder is or comprises idiopathic pulmonary fibrosis (IPF), chronic kidney disease, diabetic cardiomyopathy, primary sclerosing cholangitis (PSC), primary biliary cirrhosis (PBC), nonalcoholic fatty liver disease (NAFLD/NASH), Crohn's disease, ulcerative colitis, or systemic sclerosis. In some embodiments, a fibrotic disorder is or comprises atrial fibrosis, endomyocardial fibrosis, arthrofibrosis, mediastinal fibrosis, myelofibrosis, progressive massive fibrosis, retroperitoneal fibrosis or skeletal muscle fibrosis.

In some embodiments, an anti-α11β1 antibody described herein, upon administration to a subject, reduces one or more markers, signs and/or symptoms of a kidney-related disorder described herein. Markers, signs and/or symptoms of kidney-related disorders include, e.g., COL1A1, IL-6, TIMP-1, Hyaluronic acid, TGFβ, CTGF, PDGF, and MMP9. In some embodiments, upon administration to a subject, an anti-α11β1 antibody can reduce a measured marker, sign and/or symptom by at least about 10%, 15%, 20%, 25%, 30%, 35%, 40%, 50%, 55%, 60%, 65%, 70%, 75%, 80%, 85% or 90%, relative to a control (e.g., a level of measured marker, sign and/or symptom in the subject prior to administration of the antibody, a level of measured marker, sign and/or symptom in a subject suffering from the kidney-related disorder, and/or an average level of measured marker, sign and/or symptom in a population of subjects suffering from the kidney-related disorder).

In some embodiments, an anti-α11β1 antibody described herein reduces levels of COL1A1, IL-6, TIMP-1, Hyaluronic acid, TGFβ, CTGF, PDGF, MMP9, or a combination thereof by at least about 10%, 15%, 20%, 25%, 30%, 35%, 40%, 50%, 55%, 60%, 65%, 70%, 75%, 80%, 85% or 90%, relative to a control, as measured in a model of kidney-related disorder (e.g., human Precision-Cut Kidney Slices (PCKS), a ReninAAV Unx db/db mouse model, or a 5/6 Nephrectomy model). In some embodiments, markers, signs and/or symptoms of kidney-related disorders can be determined by measuring protein levels, RNA levels, DNA levels, or a combination thereof. In some embodiments, markers, signs and/or symptoms of kidney-related disorders can be determined using ELISA, PCR, RNAseq, a biochemical assay (e.g., an analytical procedure to detect and quantify cellular processes (e.g. apoptosis, cell signaling) or metabolic reactions), cytology, immunohistochemistry, or a combination thereof.

In some embodiments, markers, signs and/or symptoms of kidney-related disorders can be determined by testing a biological sample from a subject. Examples of suitable biological samples include, but are not limited to, serum, plasma, cerebrospinal fluid, urine, circulating blood cells (e.g., peripheral blood mononuclear cells), and biopsy specimens. In some embodiments, a sample comprises cells or tissues. In some embodiments, provided methods further comprises a step of lysing cells or performing a tissue biopsy and one or more markers include one or more intracellular markers. Biological samples suitable for the present disclosure may be fresh or frozen samples collected from a subject, or archival samples with known diagnosis, treatment and/or outcome history. Biological samples may be collected by any invasive or non-invasive means, such as, for example, by drawing CSF or blood from a subject, or using fine needle aspiration or needle biopsy, or by surgical biopsy. In some embodiments, biological samples may be used without or with limited processing of the sample.

In some embodiments, an anti-α11β1 antibody described herein, upon administration to a subject, are useful to promote catabolism and clearance of pathogenic antibodies, e.g., IgG and IgG autoantibodies in a subject, to reduce the immune response, e.g., to block immune complex-based activation of the immune response in a subject, and to treat immunological conditions or diseases in a subject. In particular, anti-α11β1 antibodies described herein are useful to reduce or treat an immune complex-based activation of an acute or chronic immune response. The acute immune response may be activated by a medical condition selected from the group consisting of pemphigus vulgaris, lupus nephritis, myasthenia gravis, Guillain-Barre syndrome, antibody-mediated rejection, catastrophic anti-phospholipid antibody syndrome, immune complex-mediated vasculitis, glomerulus, a channelopathy, neuromyelitis optica, autoimmune hearing loss, idiopathic thrombocytopenia purpura (ITP), autoimmune haemolytic anaemia (AIHA), immune neutropenia, dialated cardiomyopathy, and serum sickness. The chronic immune response may be activated by a medical condition selected from the group consisting of chronic inflammatory demyelinating polyneuropathy (CIDP), systemic lupus, a chronic form of a disorder indicated for acute treatment, reactive arthropathies, primary biliary cirrhosis, ulcerative colitis, and antineutrophil cytoplasmic antibody (ANCA)-associated vasculitis.

In some embodiments, an anti-α11β1 antibody described herein are useful to reduce or treat an immune response activated by an autoimmune disease. The autoimmune disease may be selected from the group consisting of alopecia areata, ankylosing spondylitis, antiphospholipid syndrome, Addison's disease, hemolytic anemia, autoimmune hepatitis, hepatitis, Behcets disease, bullous pemphigoid, cardiomyopathy, celiac sprue-dermatitis, chronic fatigue immune dysfunction syndrome, chronic inflammatory demyelinating polyneuropathy, Churg-Strauss syndrome, cicatricial pemphigoid, limited scleroderma (CREST syndrome), cold agglutinin disease, Crohn's disease, dermatomyositis, discoid lupus, essential mixed cryoglobulinemia, fibromyalgia, fibromyositis, Graves' disease, Hashimoto's thyroiditis, hypothyroidism, inflammatory bowel disease, autoimmune lymphoproliferative syndrome, idiopathic pulmonary fibrosis, IgA nephropathy, insulin dependent diabetes, juvenile arthritis, lichen planus, lupus, Meniere's Disease, mixed connective tissue disease, multiple sclerosis, pernicious anemia, polyarteritis nodosa, polychondritis, polyglandular syndromes, polymyalgia rheumatica, polymyositis, primary agammaglobulinemia, primary biliary cirrhosis, psoriasis, Raynaud's phenomenon, Reiter's syndrome, rheumatic fever, rheumatoid arthritis, sarcoidosis, scleroderma, Sjogren's syndrome, stiff-man syndrome, Takayasu arteritis, temporal arteritis, ulcerative colitis, uveitis, vitiligo, and Wegener's granulomatosis. In some embodiments, an anti-α11β1 antibody described herein are useful to reduce or treat an immune response activated by systemic lupus erythematosus, antiphospholipid syndrome, pemphigus vulgaris/bullous pemphigoid, antineutrophil cytoplasmic antibody (ANCA)-associated vasculitis, myasthenia gravis, or neuromyelitis optica.

In some embodiments, an anti-α11β1 antibody described herein are useful to reduce or treat inflammation diseases or disorders. In some embodiments, the inflammation disease or disorder is also an autoimmune disease or disorder as described herein. In some embodiments, inflammation diseases or disorders includes hidradenitis suppurativa, also known as acne inversa or Verneuil's disease.

One clinical feature of the tumor microenvironment is the interaction between tumor and stroma, which mainly relies on various integrins that interact with ECM components as well as growth factors. Such interaction can influence tumor survival, progression and eventually metastasis α11β1 has been reported to be overexpressed in cancer-associated fibroblasts (CAFs) of metastatic tumors, and its expression has been correlated with aggressive tumors in patients. For example, integrin al 1 was overexpressed in the stroma of most head and neck squamous cell carcinomas (HNSCC) and correlated positively with alpha smooth muscle actin expression (Parajuli et al., J. Oral Pathol. Med. 46:267-275 (2017)). Integrin al 1 was also overexpressed by CAFs in Pancreatic Ductal Adenocarcinoma (PD AC) stroma (Schnittert et al., FASEB J. 33:6609-6621 (2019)). In addition, integrin α11β1 overexpression in the tumor stroma has been associated with tumor growth and metastatic potential of non-small cell lung cancer (NSCLC), and high expression of ITGA11 (gene encoding integrin alpha-11 in humans) was associated with lower recurrence-free survival in all NSCLC patients; the same study showed that α11β1 overexpression in lung cancer cell lines resulted in increased migration and invasion (Ando et al., Cancer Sci. Ill: 200-208 (2020)).

Thus, in some embodiments, one or more anti-α11β1 antibodies described herein are used in a method of treating cancer, such as one or more of the following: head and neck squamous cell carcinomas, pancreatic ductal adenocarcinoma, non-small cell lung cancer, adrenocortical carcinoma, acute myeloid leukemia, bladder urothelial carcinoma, invasive breast carcinoma, cervical squamous cell carcinoma, cholangiocarcinoma, colorectal adenocarcinoma, diffuse large B-cell lymphoma, esophageal adenocarcinoma, glioblastoma multiforme, liver hepatocellular carcinoma, lung adenocarcinoma, lung squamous cell carcinoma, skin cutaneous melanoma, mesothelioma, ovarian serous cystadenocarcinoma, pheochromocytoma and paraganglioma, prostate adenocarcinoma, sarcoma, stomach adenocarcinoma, testicular germ cell tumors, thymoma, thyroid carcinoma, uterine corpus endometrial carcinoma, uterine carcinosarcoma, uveal melanoma, kidney renal clear cell carcinoma, kidney chromophobe, and kidney renal papillary cell carcinoma.

In some embodiments, an anti-α11β1 antibody described herein is administered in combination with one or more additional therapeutic agents, such as a chemotherapeutic agent or an oncolytic therapeutic agent. “Combination therapy”, as used herein, refers to those situations in which two or more different pharmaceutical agents are administered in overlapping regimens so that the subject is simultaneously exposed to both agents. When used in combination therapy, two or more different agents may be administered simultaneously or separately. Administration in combination can include simultaneous administration of the two or more agents in the same dosage form, simultaneous administration in separate dosage forms, and separate administration. That is, two or more agents can be formulated together in the same dosage form and administered simultaneously. Alternatively, two or more agents can be simultaneously administered, wherein the agents are present in separate formulations. In another alternative, a first agent can be administered just followed by one or more additional agents. In the separate administration protocol, two or more agents may be administered a few minutes apart, or a few hours apart, or a few days apart.

As used herein, the term “chemotherapeutic agent” or “oncolytic therapeutic agent” (e.g., anti-cancer drug, e.g., anti-cancer therapy, e.g., immune cell therapy) has its art understood meaning referring to one or more pro-apoptotic, cytostatic and/or cytotoxic agents, and/or hormonal agents, for example, specifically including agents utilized and/or recommended for use in treating one or more diseases, disorders or conditions associated with undesirable cell proliferation. In some embodiments, a chemotherapeutic agent and/or oncolytic therapeutic agent may be or comprise platinum compounds (e.g., cisplatin, carboplatin, and oxaliplatin), alkylating agents (e.g., cyclophosphamide, ifosfamide, chlorambucil, nitrogen mustard, thiotepa, melphalan, busulfan, procarbazine, streptozocin, temozolomide, dacarbazine, and bendamustine), anti-tumor antibiotics (e.g., daunorubicin, doxorubicin, idarubicin, epirubicin, mitoxantrone, bleomycin, mytomycin C, plicamycin, and dactinomycin), taxanes (e.g., paclitaxel and docetaxel), antimetabolites (e.g., 5-fluorouracil, cytarabine, premetrexed, thioguanine, floxuridine, capecitabine, and methotrexate), nucleoside analogues (e.g., fludarabine, clofarabine, cladribine, pentostatin, and nelarabine), topoisomerase inhibitors (e.g., topotecan and irinotecan), hypomethylating agents (e.g., azacitidine and decitabine), proteosome inhibitors (e.g., bortezomib), epipodophyllotoxins (e.g., etoposide and teniposide), DNA synthesis inhibitors (e.g., hydroxyurea), vinca alkaloids (e.g., vicristine, vindesine, vinorelbine, and vinblastine), tyrosine kinase inhibitors (e.g., imatinib, dasatinib, nilotinib, sorafenib, and sunitinib), nitrosoureas (e.g., carmustine, fotemustine, and lomustine), hexamethylmelamine, mitotane, angiogenesis inhibitors (e.g., thalidomide and lenalidomide), steroids (e.g., prednisone, dexamethasone, and prednisolone), hormonal agents (e.g., tamoxifen, raloxifene, leuprolide, bicaluatmide, granisetron, and flutamide), aromatase inhibitors (e.g., letrozole and anastrozole), arsenic trioxide, tretinoin, nonselective cyclooxygenase inhibitors (e.g., nonsteroidal anti-inflammatory agents, salicylates, aspirin, piroxicam, ibuprofen, indomethacin, naprosyn, diclofenac, tolmetin, ketoprofen, nabumetone, and oxaprozin), selective cyclooxygenase-2 (COX-2) inhibitors, or any combination thereof.

In certain embodiments, chemotherapeutic agents and/or oncolytic therapeutic agents for anti-cancer treatment comprise biological agents such as tumor-infiltrating lymphocytes, CAR T-cells, antibodies, antigens, therapeutic vaccines (e.g., made from a patient's own tumor cells or other substances such as antigens that are produced by certain tumors), immune-modulating agents (e.g., cytokines, e.g., immunomodulatory drugs or biological response modifiers), checkpoint inhibitors or other immunologic agents. In certain embodiments, immunologic agents include immunoglobins, immunostimulants (e.g., bacterial vaccines, colony stimulating factors, interferons, interleukins, therapeutic vaccines, vaccine combinations, viral vaccines) and/or immunosuppressive agents (e.g., calcineurin inhibitors, interleukin inhibitors, TNF alpha inhibitors). In certain embodiments, hormonal agents include agents for anti-androgen therapy (e.g., Ketoconazole, ABiraterone, TAK-700, TOK-001, Bicalutamide, Nilutamide, Flutamide, Enzalutamide, ARN-509).

Additional chemotherapeutic agents and/or oncolytic therapeutic agents include immune checkpoint therapeutics (e.g., pembrolizumab, nivolumab, ipilimumab, atezolizumab, avelumab, durvalumab, tremelimumab, or cemiplimab), other monoclonal antibodies (e.g., rituximab, cetuximab, panetumumab, tositumomab, trastuzumab, alemtuzumab, gemtuzumab ozogamicin, bevacizumab, catumaxomab, denosumab, obinutuzumab, ofatumumab, ramucirumab, pertuzumab, nimotuzumab, lambrolizumab, pidilizumab, siltuximab, BMS-936559, RG7446/MPDL3280A, MEDI4736), antibody-drug conjugates (e.g., brentuximab vedotin (ADCETRIS®, Seattle Genetics); ado-trastuzumab emtansine (KADCYLA®, Roche); Gemtuzumab ozogamicin (Wyeth); CMC-544; SAR3419; CDX-011; PSMA-ADC; BT-062; and IMGN901 (see, e.g., Sassoon et al., Methods Mol. Biol. 1045:1-27 (2013); Bouchard et al., Bioorganic Med. Chem. Lett. 24:5357-5363 (2014)), or any combination thereof.

In some embodiments, combined administration of an anti-α11β1 antibody and an additional therapeutic agent results in an improvement in cancer to an extent that is greater than one produced by either the anti-α11β1 antibody or the additional therapeutic agent alone. The difference between the combined effect and the effect of each agent alone can be a statistically significant difference. In some embodiments, the combined effect can be a synergistic effect. In some embodiments, combined administration of an anti-α11β1 antibody and an additional therapeutic agent allows administration of the additional therapeutic agent at a reduced dose, at a reduced number of doses, and/or at a reduced frequency of dosage compared to a standard dosing regimen, e.g., an approved dosing regimen for the additional therapeutic agent.

In some embodiments, treatment methods described herein are performed on subjects for whom other treatments of the medical condition have failed or have had less success in treatment through other means. Additionally, the treatment methods described herein can be performed in conjunction with one or more additional treatments of the medical condition. For instance, the method can comprise administering a cancer regimen, e.g., non-myeloablative chemotherapy, surgery, hormone therapy, and/or radiation, prior to, substantially simultaneously with, or after the administration of an anti-α11β1 antibody described herein, or composition thereof.

### Enumerated Embodiments

In some embodiments, the following embodiments are provided.

1. An antibody, or an antigen-binding fragment thereof, wherein the antibody or antigen-binding fragment thereof, comprises:
(i) a heavy chain variable region comprising heavy chain HCDR1, HCDR2, and HCDR3 sequences, wherein the heavy chain HCDR1 sequence has the amino acid sequence of SEQ ID NO: 1; the heavy chain HCDR2 has the amino acid sequence of SEQ ID NO: 2 and the heavy chain HCDR3 sequence has the amino acid sequence of SEQ ID NO: 3; or variants of any of the foregoing; and
(ii) a light chain variable region comprising light chain LCDR1, LCDR2, and LCDR3 sequences, wherein the light chain LCDR1 sequence has the amino acid sequence SEQ ID NO: 16; the light chain LCDR2 sequence has the amino acid sequence of SEQ ID NO: 17; and the light chain LCDR3 sequence has the amino acid sequence of SEQ ID NO: 18; or variants of any of the foregoing.

2. The antibody, or antigen-binding fragment thereof, of embodiment 1, wherein the heavy chain variable region comprises an amino acid sequence having at least 90%, 91%, 92%, 93%, 94%, 95%, 96%, 97%, 98%, or 99% sequence identity to SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, or SEQ ID NO: 15, provided that the heavy chain variable region comprises the sequences of HCDR1, HCDR2, and HCDR3 of embodiment 1.

3. The antibody, or antigen-binding fragment thereof, of embodiments 1 or 2, wherein the light chain variable region comprises an amino acid sequence having at least 90%, 91%, 92%, 93%, 94%, 95%, 96%, 97%, 98%, or 99% sequence identity to SEQ ID NO: 19, SEQ ID NO: 20, SEQ ID NO: 21, or SEQ ID NO: 22, provided that the light chain variable region comprises the sequences of LCDR1, LCDR2, and LCDR3 of embodiment 1.

4. The antibody, or antigen-binding fragment thereof, of any one of embodiments 1-3, comprising:
(i) a heavy chain variable region comprising an amino acid sequence having at least 90%, 91%, 92%, 93%, 94%, 95%, 96%, 97%, 98%, or 99% identity, or is identical, to SEQ ID NO: 11, provided that the heavy chain variable region comprises a HCDR1 of SEQ ID NO: 1, a HCDR2 of SEQ ID NO: 2, and a HCDR3 of SEQ ID NO: 3; and
(ii) a light chain variable region comprising an amino acid sequence having at least 90%, 91%, 92%, 93%, 94%, 95%, 96%, 97%, 98%, or 99% identity, or is identical, to SEQ ID NO: 21, provided that the light chain variable region comprises a LCDR1 of SEQ ID NO: 16, a LCDR2 of SEQ ID NO: 17, and a LCDR3 of SEQ ID NO: 18.

5. The antibody, or antigen-binding fragment thereof, of any one of embodiments 1-3, comprising:
(i) a heavy chain variable region comprising an amino acid sequence having at least 90%, 91%, 92%, 93%, 94%, 95%, 96%, 97%, 98%, or 99% identity, or is identical, to SEQ ID NO: 4, provided that the heavy chain variable region comprises a HCDR1 of SEQ ID NO: 1, a HCDR2 of SEQ ID NO: 2, and a HCDR3 of SEQ ID NO: 3; and
(ii) a light chain variable region comprising an amino acid sequence having at least 90%, 91%, 92%, 93%, 94%, 95%, 96%, 97%, 98%, or 99% identity, or is identical, to SEQ ID NO: 22, wherein the light chain variable region comprises a LCDR1 of SEQ ID NO: 16, a LCDR2 of SEQ ID NO: 17, and a LCDR3 of SEQ ID NO: 18.

6. The antibody, or antigen-binding fragment thereof, of any one of embodiments 1-3, comprising:
(i) a heavy chain variable region comprising an amino acid sequence having at least 90%, 91%, 92%, 93%, 94%, 95%, 96%, 97%, 98%, or 99% identity, or is identical, to SEQ ID NO: 4, provided that the heavy chain variable region comprises a HCDR1 of SEQ ID NO: 1, a HCDR2 of SEQ ID NO: 2, and a HCDR3 of SEQ ID NO: 3; and
(ii) a light chain variable region comprising an amino acid sequence having at least 90%, 91%, 92%, 93%, 94%, 95%, 96%, 97%, 98%, or 99% identity, or is identical, to SEQ ID NO: 21, provided that the light chain variable region comprises a LCDR1 of SEQ ID NO: 16, a LCDR2 of SEQ ID NO: 17, and a LCDR3 of SEQ ID NO: 18.

7. The antibody, or antigen-binding fragment thereof, of any one of embodiments 1-3, comprising:
(i) a heavy chain variable region comprising an amino acid sequence having at least 90%, 91%, 92%, 93%, 94%, 95%, 96%, 97%, 98%, or 99% identity, or is identical, to SEQ ID NO: 10, provided that the heavy chain variable region comprises a HCDR1 of SEQ ID NO: 1, a HCDR2 of SEQ ID NO: 2, and a HCDR3 of SEQ ID NO: 3; and
(ii) a light chain variable region comprising an amino acid sequence having at least 90%, 91%, 92%, 93%, 94%, 95%, 96%, 97%, 98%, or 99% identity, or is identical, to SEQ ID NO: 22, provided that the light chain variable region comprises a LCDR1 of SEQ ID NO: 16, a LCDR2 of SEQ ID NO: 17, and a LCDR3 of SEQ ID NO: 18.

8. The antibody, or antigen-binding fragment thereof, of any one of embodiments 1-3, comprising:
(i) a heavy chain variable region having at least 90%, 91%, 92%, 93%, 94%, 95%, 96%, 97%, 98%, or 99% identity, or is identical, to SEQ ID NO: 9, provided that the heavy chain variable region comprises a HCDR1 of SEQ ID NO: 1, a HCDR2 of SEQ ID NO: 2, and a HCDR3 of SEQ ID NO: 3; and
(ii) a light chain variable region having at least 90%, 91%, 92%, 93%, 94%, 95%, 96%, 97%, 98%, or 99% identity, or is identical, to SEQ ID NO: 21, provided that the light chain variable region comprises a LCDR1 of SEQ ID NO: 16, a LCDR2 of SEQ ID NO: 17, and a LCDR3 of SEQ ID NO: 18.

9. The antibody, or antigen-binding fragment thereof, of any one of embodiments 1-3, comprising:
(i) a heavy chain variable region comprising an amino acid sequence having at least 90%, 91%, 92%, 93%, 94%, 95%, 96%, 97%, 98%, or 99% identity, or is identical, to SEQ ID NO: 14, provided that the heavy chain variable region comprises a HCDR1 of SEQ ID NO: 1, a HCDR2 of SEQ ID NO: 2, and a HCDR3 of SEQ ID NO: 3; and
(ii) a light chain variable region comprising an amino acid sequence having at least 90%, 91%, 92%, 93%, 94%, 95%, 96%, 97%, 98%, or 99% identity, or is identical, to SEQ ID NO: 22, provided that the light chain variable region comprises a LCDR1 of SEQ ID NO: 16, a LCDR2 of SEQ ID NO: 17, and a LCDR3 of SEQ ID NO: 18.

10. The antibody, or antigen-binding fragment thereof, of any one of embodiments 1-3, comprising:
(i) a heavy chain variable region comprising an amino acid sequence having at least 90%, 91%, 92%, 93%, 94%, 95%, 96%, 97%, 98%, or 99% identity, or is identical, to SEQ ID NO: 13, provided that the heavy chain variable region comprises a HCDR1 of SEQ ID NO: 1, a HCDR2 of SEQ ID NO: 2, and a HCDR3 of SEQ ID NO: 3; and
(ii) a light chain variable region comprising an amino acid sequence having at least 90%, 91%, 92%, 93%, 94%, 95%, 96%, 97%, 98%, or 99% identity, or is identical, to SEQ ID NO: 21, provided that the light chain variable region comprises a LCDR1 of SEQ ID NO: 16, a LCDR2 of SEQ ID NO: 17, and a LCDR3 of SEQ ID NO: 18.

11. The antibody, or antigen-binding fragment thereof, of any one of embodiments 1-3, comprising:
(i) a heavy chain variable region comprising an amino acid sequence having at least 90%, 91%, 92%, 93%, 94%, 95%, 96%, 97%, 98%, or 99% identity, or is identical, to SEQ ID NO: 12, provided that the heavy chain variable region comprises a HCDR1 of SEQ ID NO: 1, a HCDR2 of SEQ ID NO: 2, and a HCDR3 of SEQ ID NO: 3; and
(ii) a light chain variable region comprising an amino acid sequence having at least 90%, 91%, 92%, 93%, 94%, 95%, 96%, 97%, 98%, or 99% identity, or is identical, to SEQ ID NO: 21, provided that the light chain variable region comprises a LCDR1 of SEQ ID NO: 16, a LCDR2 of SEQ ID NO: 17, and a LCDR3 of SEQ ID NO: 18.

12. An antibody, or an antigen-binding fragment thereof, wherein the antibody, or the antigen-binding fragment thereof, comprises:
(i) a heavy chain variable region comprising heavy chain HCDR1, HCDR2, and HCDR3 sequences, wherein the heavy chain HCDR1 sequence has the amino acid sequence of SEQ ID NO: 23; the heavy chain HCDR2 has the amino acid sequence of SEQ ID NO: 24, SEQ ID NO: 25, SEQ ID NO: 26, or SEQ ID NO: 27, and the heavy chain HCDR3 sequence has the amino acid sequence of SEQ ID NO: 28, or SEQ ID NO: 29, or variants of any of the foregoing; and
(ii) a light chain variable region comprising light chain LCDR1, LCDR2, and LCDR3 sequences, wherein the light chain LCDR1 sequence has the amino acid sequence SEQ ID NO: 34; the light chain LCDR2 sequence has the amino acid sequence of SEQ ID NO: 35; and the light chain LCDR3 sequence has the amino acid sequence of SEQ ID NO: 36; or variants of any of the foregoing.

13. The antibody, or antigen-binding fragment thereof, of embodiment 12, wherein the HCDR2 is SEQ ID NO: 24 and the HCDR3 is SEQ ID NO: 28.

14. The antibody, or antigen-binding fragment thereof, of embodiment 12, wherein the HCDR2 is SEQ ID NO: 24 and the HCDR3 is SEQ ID NO: 29.

15. The antibody, or antigen-binding fragment thereof, of embodiment 12, wherein the HCDR2 is SEQ ID NO: 25 and the HCDR3 is SEQ ID NO: 28.

16. The antibody, or antigen-binding fragment thereof, of embodiment 12, wherein the HCDR2 is SEQ ID NO: 25 and the HCDR3 is SEQ ID NO: 29.

17. The antibody, or antigen-binding fragment thereof, of embodiment 12, wherein the HCDR2 is SEQ ID NO: 26 and the HCDR3 is SEQ ID NO: 28.

18. The antibody, or antigen-binding fragment thereof, of embodiment 12, wherein the HCDR2 is SEQ ID NO: 26 and the HCDR3 is SEQ ID NO: 29.

19. The antibody, or antigen-binding fragment thereof, of embodiment 12, wherein the HCDR2 is SEQ ID NO: 27 and the HCDR3 is SEQ ID NO: 28.

20. The antibody, or antigen-binding fragment thereof, of embodiment 12, wherein the HCDR2 is SEQ ID NO: 27 and the HCDR3 is SEQ ID NO: 29.

21. The antibody, or antigen-binding fragment thereof, of any one of embodiments 12-20, wherein the heavy chain variable region comprises an amino acid sequence having at least 90% sequence identity to SEQ ID NO: 30, SEQ ID NO: 31, SEQ ID NO: 32, or SEQ ID NO: 33, provided that heavy chain variable region comprises the sequences of HCDR1, HCDR2, and HCDR3 of embodiment 12.

22. The antibody, or antigen-binding fragment thereof, any one of embodiments 12-21, wherein the light chain variable region comprises an amino acid sequence having at least 90% sequence identity to SEQ ID NO: 37, provided that the light chain variable region comprises the sequences of LCDR1, LCDR2, and LCDR3 of embodiment 12. 23. The antibody, or antigen-binding fragment thereof, of any one of embodiments 12-22, comprising:
(i) a heavy chain variable region comprising an amino acid sequence having at least 90%, 91%, 92%, 93%, 94%, 95%, 96%, 97%, 98%, or 99% identity, or is identical, to SEQ ID NO: 30, provided that the heavy chain variable region comprises a HCDR1 of SEQ ID NO: 23, a HCDR2 of SEQ ID NO: 24, and a HCDR3 of SEQ ID NO: 28; and
(ii) a light chain variable region comprising an amino acid sequence having at least 90%, 91%, 92%, 93%, 94%, 95%, 96%, 97%, 98%, or 99% identity, or is identical, to SEQ ID NO: 37, provided that the light chain variable region comprises a LCDR1 of SEQ ID NO: 34, a LCDR2 of SEQ ID NO: 35, and a LCDR3 of SEQ ID NO: 36.

24. The antibody, or antigen-binding fragment thereof, of any one of embodiments 12-22, comprising:
(i) a heavy chain variable region comprising an amino acid sequence having at least 90%, 91%, 92%, 93%, 94%, 95%, 96%, 97%, 98%, or 99% identity, or is identical, to SEQ ID NO: 31, provided that the heavy chain variable region comprises a HCDR1 of SEQ ID NO: 23, a HCDR2 of SEQ ID NO: 25, and a HCDR3 of SEQ ID NO: 28; and
(ii) a light chain variable region comprising an amino acid sequence having at least 90%, 91%, 92%, 93%, 94%, 95%, 96%, 97%, 98%, or 99% identity, or is identical, to SEQ ID NO: 37, provided that the light chain variable region comprises a LCDR1 of SEQ ID NO: 34, a LCDR2 of SEQ ID NO: 35, and a LCDR3 of SEQ ID NO: 36.

25. The antibody, or antigen-binding fragment thereof, of any one of embodiments 12-22, comprising:
(i) a heavy chain variable region comprising an amino acid sequence having at least 90%, 91%, 92%, 93%, 94%, 95%, 96%, 97%, 98%, or 99% identity, or is identical, to SEQ ID NO: 33, provided that the heavy chain variable region comprises a HCDR1 of SEQ ID NO: 23, a HCDR2 of SEQ ID NO: 27, and a HCDR3 of SEQ ID NO: 28; and
(ii) a light chain variable region comprising an amino acid sequence having at least 90%, 91%, 92%, 93%, 94%, 95%, 96%, 97%, 98%, or 99% identity, or is identical, to SEQ ID NO: 37, provided that the light chain variable region comprises a LCDR1 of SEQ ID NO: 34, a LCDR2 of SEQ ID NO: 35, and a LCDR3 of SEQ ID NO: 36.

26. An antibody, or an antigen-binding fragment thereof, wherein the antibody or antigen-binding fragment thereof, comprises:
(i) a heavy chain variable region comprising heavy chain HCDR1, HCDR2, and HCDR3 sequences, wherein the heavy chain HCDR1 sequence has the amino acid sequence of SEQ ID NO: 38, SEQ ID NO: 39, or SEQ ID NO: 40; the heavy chain HCDR2 has the amino acid sequence of SEQ ID NO: 41 or SEQ ID NO: 42; and the heavy chain HCDR3 sequence has the amino acid sequence of SEQ ID NO: 43; or variants of any of the foregoing; and
(ii) a light chain variable region comprising light chain LCDR1, LCDR2, and LCDR3 sequences, wherein the light chain LCDR1 sequence has the amino acid sequence SEQ ID NO: 58; the light chain LCDR2 sequence has the amino acid sequence of SEQ ID NO: 59; and the light chain LCDR3 sequence has the amino acid sequence of SEQ ID NO: 60; or variants of any of the foregoing.

27. The antibody, or antigen-binding fragment thereof, of embodiment 26, wherein the HCDR1 is SEQ ID NO: 38 and the HCDR2 is SEQ ID NO: 41.

28. The antibody, or antigen-binding fragment thereof, of embodiment 26, wherein the HCDR1 is SEQ ID NO: 38 and the HCDR2 is SEQ ID NO: 42.

29. The antibody, or antigen-binding fragment thereof, of embodiment 26, wherein the HCDR1 is SEQ ID NO: 39 and the HCDR2 is SEQ ID NO: 41.

30. The antibody, or antigen-binding fragment thereof, of embodiment 26, wherein the HCDR1 is SEQ ID NO: 39 and the HCDR2 is SEQ ID NO: 42.

31. The antibody, or antigen-binding fragment thereof, of embodiment 26, wherein the HCDR1 is SEQ ID NO: 40 and the HCDR2 is SEQ ID NO: 41.

32. The antibody, or antigen-binding fragment thereof, of embodiment 26, wherein the HCDR1 is SEQ ID NO: 40 and the HCDR2 is SEQ ID NO: 42.

33. The antibody, or antigen-binding fragment thereof, of any one of embodiments 26-32, wherein the heavy chain variable region comprising an amino acid sequence having at least 90% sequence identity to SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, or SEQ ID NO: 57, provided that heavy chain variable region comprises the sequences of HCDR1, HCDR2, and HCDR3 of embodiment 26.

34. The antibody, or antigen-binding fragment thereof, any one of embodiments 26-33, wherein the light chain variable region comprises an amino acid sequence having at least 90% sequence identity to SEQ ID NO: 61, or SEQ ID NO: 62, provided that the light chain variable region comprises the sequences of LCDR1, LCDR2, and LCDR3 of embodiment 26.

35. The antibody, or antigen-binding fragment thereof, of any one of embodiments 26-34, comprising:
(i) a heavy chain variable region comprising an amino acid sequence having at least 90%, 91%, 92%, 93%, 94%, 95%, 96%, 97%, 98%, or 99% identity, or is identical, to SEQ ID NO: 52, provided that the heavy chain variable region comprises a HCDR1 of SEQ ID NO: 39, a HCDR2 of SEQ ID NO: 42, and a HCDR3 of SEQ ID NO: 43; and
(ii) a light chain variable region comprising an amino acid sequence having at least 90%, 91%, 92%, 93%, 94%, 95%, 96%, 97%, 98%, or 99% identity, or is identical, to SEQ ID NO: 61, provided that the light chain variable region comprises a LCDR1 of SEQ ID NO: 58, a LCDR2 of SEQ ID NO: 59, and a LCDR3 of SEQ ID NO: 60.

36. The antibody, or antigen-binding fragment thereof, of any one of embodiments 26-34, comprising:
(i) a heavy chain variable region comprising an amino acid sequence having at least 90%, 91%, 92%, 93%, 94%, 95%, 96%, 97%, 98%, or 99% identity, or is identical, to SEQ ID NO: 49, provided that the heavy chain variable region comprises a HCDR1 of SEQ ID NO: 39, a HCDR2 of SEQ ID NO: 42, and a HCDR3 of SEQ ID NO: 43; and
(ii) a light chain variable region comprising an amino acid sequence having at least 90%, 91%, 92%, 93%, 94%, 95%, 96%, 97%, 98%, or 99% identity, or is identical, to SEQ ID NO: 61, provided that the light chain variable region comprises a LCDR1 of SEQ ID NO: 58, a LCDR2 of SEQ ID NO: 59, and a LCDR3 of SEQ ID NO: 60.

37. The antibody, or antigen-binding fragment thereof, of any one of embodiments 26-34, comprising:
(i) a heavy chain variable region comprising an amino acid sequence having at least 90%, 91%, 92%, 93%, 94%, 95%, 96%, 97%, 98%, or 99% identity, or is identical, to SEQ ID NO: 50, provided that the heavy chain variable region comprises a HCDR1 of SEQ ID NO: 39, a HCDR2 of SEQ ID NO: 42, and a HCDR3 of SEQ ID NO: 43; and
(ii) a light chain variable region comprising an amino acid sequence having at least 90%, 91%, 92%, 93%, 94%, 95%, 96%, 97%, 98%, or 99% identity, or is identical, to SEQ ID NO: 61, provided that the light chain variable region comprises a LCDR1 of SEQ ID NO: 58, a LCDR2 of SEQ ID NO: 59, and a LCDR3 of SEQ ID NO: 60.

38. The antibody, or antigen-binding fragment thereof, of any one of embodiments 26-34, comprising:
(i) a heavy chain variable region comprising an amino acid sequence having at least 90%, 91%, 92%, 93%, 94%, 95%, 96%, 97%, 98%, or 99% identity, or is identical, to SEQ ID NO: 56, provided that the heavy chain variable region comprises a HCDR1 of SEQ ID NO: 40, a HCDR2 of SEQ ID NO: 41, and a HCDR3 of SEQ ID NO: 43; and
(ii) a light chain variable region comprising an amino acid sequence having at least 90%, 91%, 92%, 93%, 94%, 95%, 96%, 97%, 98%, or 99% identity, or is identical, to SEQ ID NO: 61, provided that the light chain variable region comprises a LCDR1 of SEQ ID NO: 58, a LCDR2 of SEQ ID NO: 59, and a LCDR3 of SEQ ID NO: 60.

39. The antibody, or antigen-binding fragment thereof, of any one of embodiments 26-34, comprising:
(i) a heavy chain variable region comprising an amino acid sequence having at least 90%, 91%, 92%, 93%, 94%, 95%, 96%, 97%, 98%, or 99% identity, or is identical, to SEQ ID NO: 45, provided that the heavy chain variable region comprises a HCDR1 of SEQ ID NO: 38, a HCDR2 of SEQ ID NO: 41, and a HCDR3 of SEQ ID NO: 43; and
(ii) a light chain variable region comprising an amino acid sequence having at least 90%, 91%, 92%, 93%, 94%, 95%, 96%, 97%, 98%, or 99% identity, or is identical, to SEQ ID NO: 61, provided that the light chain variable region comprises a LCDR1 of SEQ ID NO: 58, a LCDR2 of SEQ ID NO: 59, and a LCDR3 of SEQ ID NO: 60.

40. The antibody, or antigen-binding fragment thereof, of any one of embodiments 26-34, comprising:
(i) a heavy chain variable region comprising an amino acid sequence having at least 90%, 91%, 92%, 93%, 94%, 95%, 96%, 97%, 98%, or 99% identity, or is identical, to SEQ ID NO: 53, provided that the heavy chain variable region comprises a HCDR1 of SEQ ID NO: 40, a HCDR2 of SEQ ID NO: 41, and a HCDR3 of SEQ ID NO: 43; and
(ii) a light chain variable region comprising an amino acid sequence having at least 90%, 91%, 92%, 93%, 94%, 95%, 96%, 97%, 98%, or 99% identity, or is identical, to SEQ ID NO: 61, provided that the light chain variable region comprises a LCDR1 of SEQ ID NO: 58, a LCDR2 of SEQ ID NO: 59, and a LCDR3 of SEQ ID NO: 60.

41. The antibody, or antigen-binding fragment thereof, of any one of embodiments 26-34, comprising:
(i) a heavy chain variable region comprising an amino acid sequence having at least 90%, 91%, 92%, 93%, 94%, 95%, 96%, 97%, 98%, or 99% identity, or is identical, to SEQ ID NO: 47, provided that the heavy chain variable region comprises a HCDR1 of SEQ ID NO: 38, a HCDR2 of SEQ ID NO: 41, and a HCDR3 of SEQ ID NO: 43; and
(ii) a light chain variable region comprising an amino acid sequence having at least 90%, 91%, 92%, 93%, 94%, 95%, 96%, 97%, 98%, or 99% identity, or is identical, to SEQ ID NO: 61, provided that the light chain variable region comprises a LCDR1 of SEQ ID NO: 58, a LCDR2 of SEQ ID NO: 59, and a LCDR3 of SEQ ID NO: 60.

42. The antibody, or antigen-binding fragment thereof, of any one of embodiments 26-34, comprising:
(i) a heavy chain variable region comprising an amino acid sequence having at least 90%, 91%, 92%, 93%, 94%, 95%, 96%, 97%, 98%, or 99% identity, or is identical, to SEQ ID NO: 55, provided that the heavy chain variable region comprises a HCDR1 of SEQ ID NO: 40, a HCDR2 of SEQ ID NO: 41, and a HCDR3 of SEQ ID NO: 43; and
(ii) a light chain variable region comprising an amino acid sequence having at least 90%, 91%, 92%, 93%, 94%, 95%, 96%, 97%, 98%, or 99% identity, or is identical, to SEQ ID NO: 61, provided that the light chain variable region comprises a LCDR1 of SEQ ID NO: 58, a LCDR2 of SEQ ID NO: 59, and a LCDR3 of SEQ ID NO: 60.

43. The antibody, or antigen-binding fragment thereof, of any one of embodiments 26-34, comprising:
(i) a heavy chain variable region comprising an amino acid sequence having at least 90%, 91%, 92%, 93%, 94%, 95%, 96%, 97%, 98%, or 99% identity, or is identical, to SEQ ID NO: 46, provided that the heavy chain variable region comprises a HCDR1 of SEQ ID NO: 38, a HCDR2 of SEQ ID NO: 41, and a HCDR3 of SEQ ID NO: 43; and
(ii) a light chain variable region comprising an amino acid sequence having at least 90%, 91%, 92%, 93%, 94%, 95%, 96%, 97%, 98%, or 99% identity, or is identical, to SEQ ID NO: 61, provided that the light chain variable region comprises a LCDR1 of SEQ ID NO: 58, a LCDR2 of SEQ ID NO: 59, and a LCDR3 of SEQ ID NO: 60.

44. The antibody, or antigen-binding fragment thereof, of any one of embodiments 26-34, comprising:
(i) a heavy chain variable region comprising an amino acid sequence having at least 90%, 91%, 92%, 93%, 94%, 95%, 96%, 97%, 98%, or 99% identity, or is identical, to SEQ ID NO: 54, provided that the heavy chain variable region comprises a HCDR1 of SEQ ID NO: 40, a HCDR2 of SEQ ID NO: 41, and a HCDR3 of SEQ ID NO: 43; and
(ii) a light chain variable region comprising an amino acid sequence having at least 90%, 91%, 92%, 93%, 94%, 95%, 96%, 97%, 98%, or 99% identity, or is identical, to SEQ ID NO: 61, provided that the light chain variable region comprises a LCDR1 of SEQ ID NO: 58, a LCDR2 of SEQ ID NO: 59, and a LCDR3 of SEQ ID NO: 60.

45. The antibody, or antigen-binding fragment thereof of any one of the preceding embodiments, wherein the antibody, or antigen-binding fragment thereof, is a scFv antibody, a Fab fragment, a Fab′ fragment, or an F(ab′)₂ fragment.

46. The antibody, or antigen-binding fragment thereof, of any one of the preceding embodiments, wherein the antibody is a monoclonal antibody.

47. The antibody, or antigen-binding fragment thereof, of any one of the preceding embodiments, wherein the antibody is a humanized antibody.

48. The antibody, or antigen-binding fragment thereof, of any one of the preceding embodiments, wherein the antibody, or antigen-binding fragment thereof, comprises a constant region, such as provided for herein.

49. The antibody, or antigen-binding fragment thereof, of any one of the preceding embodiments, wherein the antibody, or antigen-binding fragment thereof, comprises a constant region comprising an amino acid sequence of SEQ ID NO: 63, SEQ ID NO: 64, SEQ ID NO: 65, SEQ ID NO: 66, SEQ ID NO: 67, SEQ ID NO: 68, or SEQ ID NO: 69.

50. The antibody, or antigen-binding fragment thereof, of any one of the preceding embodiments, wherein the antibody, or antigen-binding fragment thereof, comprises a heavy chain constant region comprising an amino acid sequence of SEQ ID NO: 63, and a light chain constant region comprising an amino acid sequence of SEQ ID NO: 69.

51. The antibody, or antigen-binding fragment thereof, of any one of the preceding embodiments, wherein the heavy chain variable region and the light chain variable region are not linked by a linker.

52. The antibody, or antigen-binding fragment thereof, of any one of the preceding embodiments, wherein the heavy chain variable region and the light chain variable region are linked with a peptide linker.

53. The antibody, or antigen-binding fragment thereof, of embodiment 52, wherein the peptide linker comprises a sequence of (GGGGS)ₙ (SEQ ID NO: 70); (GGGGA)ₙ (SEQ ID NO: 71), or any combination thereof, wherein each n is independently 1-5.

54. A recombinant antibody, or an antigen-binding fragment thereof, that binds to human α11β1 integrin, wherein the antibody or antigen-binding fragment thereof comprises an amino acid sequence, or a variant thereof, as provided for herein or an amino acid sequence that is 90%, 91%, 92%, 93%, 94%, 95%, 96%, 97%, 98%, or 99% identical thereto.

55. An isolated nucleic acid molecule encoding an antibody, or an antigen-binding fragment thereof, a heavy chain variable region, a light chain variable region, heavy chain, or light chain, of any of the preceding embodiments.

56. An expression vector comprising the nucleic acid molecule of embodiment 55.

57. A host cell comprising the nucleic acid molecule of embodiment 55 or the vector of embodiment 56.

58. An antibody or antigen-binding fragment produced by the host cell of embodiment 57.

59. A pharmaceutical composition comprising an antibody, or antigen-binding fragment thereof, of any one of embodiments 1-54.

60. The pharmaceutical composition of embodiment 59, wherein the composition is formulated for intravenous or subcutaneous injection.

61. The pharmaceutical composition of embodiment 59, wherein the composition is an injectable pharmaceutical composition.

62. A method of producing a polypeptide comprising a heavy chain variable region and/or light chain variable region, the method comprising:
(a) growing or culturing the host cell of embodiment 57 under conditions so that the host cell expresses a polypeptide comprising the heavy chain variable region and/or the light chain variable region; and
(b) purifying the polypeptide comprising the heavy chain variable region and/or the light chain variable region.

63. A method of producing an antibody that binds human α11β1 integrin, or an antigen-binding fragment thereof, the method comprising:
(a) growing or culturing the host cell of embodiment 57 under conditions so that the host cell expresses a polypeptide or polypeptides comprising an immunoglobulin heavy chain variable region and/or an immunoglobulin light chain variable region, thereby producing the antibody or the antigen-binding fragment of the antibody; and
(b) purifying the antibody, or the antigen-binding fragment thereof.

64. A method of treating a subject with α11β1 integrin mediated disorder, the method comprising administering to the subject an antibody, or antigen-binding fragment thereof, of any one of embodiments 1-54 or a pharmaceutical composition of any one of embodiments 59-61.

65. The method of embodiment 64, wherein the disorder is a fibrotic disorder, an autoimmune disorder, an inflammation disorder, or cancer.

66. The method of embodiment 65, wherein the fibrotic disorder is or comprises idiopathic pulmonary fibrosis (IPF), chronic kidney disease, diabetic cardiomyopathy, primary sclerosing cholangitis (PSC), primary biliary cirrhosis (PBC), nonalcoholic fatty liver disease (NAFLD/NASH), Crohn's disease, ulcerative colitis, or systemic sclerosis. In some embodiments, a fibrotic disorder is or comprises atrial fibrosis, endomyocardial fibrosis, arthrofibrosis, mediastinal fibrosis, myelofibrosis, progressive massive fibrosis, retroperitoneal fibrosis or skeletal muscle fibrosis.

67. The method of embodiment 65, wherein the autoimmune disorder is or comprises alopecia areata, ankylosing spondylitis, antiphospholipid syndrome, Addison's disease, hemolytic anemia, autoimmune hepatitis, hepatitis, Behcets disease, bullous pemphigoid, cardiomyopathy, celiac sprue-dermatitis, chronic fatigue immune dysfunction syndrome, chronic inflammatory demyelinating polyneuropathy, Churg-Strauss syndrome, cicatricial pemphigoid, limited scleroderma (CREST syndrome), cold agglutinin disease, Crohn's disease, dermatomyositis, discoid lupus, essential mixed cryoglobulinemia, fibromyalgia, fibromyositis, Graves' disease, Hashimoto's thyroiditis, hypothyroidism, inflammatory bowel disease, autoimmune lymphoproliferative syndrome, idiopathic pulmonary fibrosis, IgA nephropathy, insulin dependent diabetes, juvenile arthritis, lichen planus, lupus, Meniere's Disease, mixed connective tissue disease, multiple sclerosis, pernicious anemia, polyarteritis nodosa, polychondritis, polyglandular syndromes, polymyalgia rheumatica, polymyositis, primary agammaglobulinemia, primary biliary cirrhosis, psoriasis, Raynaud's phenomenon, Reiter's syndrome, rheumatic fever, rheumatoid arthritis, sarcoidosis, scleroderma, Sjogren's syndrome, stiff-man syndrome, Takayasu arteritis, temporal arteritis, ulcerative colitis, uveitis, vitiligo, and Wegener's granulomatosis.

68. The method of embodiment 65, wherein the inflammation disorder is hidradenitis suppurativa.

69. The method of embodiment 65, wherein the cancer is or comprises head and neck squamous cell carcinomas, pancreatic ductal adenocarcinoma, non-small cell lung cancer, adrenocortical carcinoma, acute myeloid leukemia, bladder urothelial carcinoma, invasive breast carcinoma, cervical squamous cell carcinoma, cholangiocarcinoma, colorectal adenocarcinoma, diffuse large B-cell lymphoma, esophageal adenocarcinoma, glioblastoma multiforme, liver hepatocellular carcinoma, lung adenocarcinoma, lung squamous cell carcinoma, skin cutaneous melanoma, mesothelioma, ovarian serous cystadenocarcinoma, pheochromocytoma and paraganglioma, prostate adenocarcinoma, sarcoma, stomach adenocarcinoma, testicular germ cell tumors, thymoma, thyroid carcinoma, uterine corpus endometrial carcinoma, uterine carcinosarcoma, uveal melanoma, kidney renal clear cell carcinoma, kidney chromophobe, or kidney renal papillary cell carcinoma.

70. An antibody or antigen-binding fragment thereof of any one of embodiments 1-54 or a pharmaceutical composition of any one of embodiments 59-61, for the use in the treatment of a α11β1 integrin mediated disorder, such as those provided for herein.

71. An antibody or binding fragment thereof of any one of embodiments 1-54 or a pharmaceutical composition of any one of embodiments 59-61 for use as a medicament.

72. Use of the antibody or binding fragment thereof of any one of embodiments 1-54 or a pharmaceutical composition of any one of embodiments 59-61, for the treatment of a α11β1 integrin mediated disorder, such as those provided for herein.

### Examples

### General Methods

### A. Generation of Novel Antibodies Against α11β1

### Rat Immunization

Wistar rats were immunized with recombinant human α11β1 protein. An enzyme-linked immunosorbent assay (ELISA) was used to test an immune response against target human and mouse proteins. Subsequently, cell fusion (by electro fusion) was performed with animals that produced a good immune response. All fused cells were plated in a 96-well plate and supernatants were screened by ELISA against soluble human and mouse α11β1. Positive clones were counter-screened against human α1β1, α2β1, and α10β1. Clones that specifically bound to human and mouse α11β1 and did not bind to α1β1, α2β1, and α10β1 were selected, subcloned, expanded and cryopreserved. Purified antibodies were then generated from the selected clones and heavy chain and light chain variable domain sequences were obtained from each purified antibody.

### Rabbit Immunization

Rabbits were immunized employing a cell-based monoclonal antibody platform. Two rabbits were immunized with recombinant human α11β1 protein. Splenocytes from the immunized rabbits were sorted and selected against human β1, in order to reduce the number of β1-specific B cell clones. Sorted splenocytes were then cultured for approximately 1 week and culture supernatants were screened for binding to human α11β1. Top results were sequenced and subsequently rabbit antibodies were recombinantly produced using a HEK cell system.

### Mouse Immunization

10 mice from 5 different strains were immunized with an appropriate mixture of human α11β1, mouse α11β1 and tolerance breaking protein. Plasma titers were evaluated by ELISA against a mixture of human and mouse α11β1. Popliteal, inguinal, and iliac lymph nodes were collected. ELISA-positive anti-human/mouse α11β1 hybridomas were expanded and subjected to a secondary screen against human and mouse α11β1, control HIS protein, and a counter-screen was performed against human α1β1, α2β1, and α10β1. Supernatant IgG concentration was sufficient for functional screening. Selected hybridomas satisfying all the criteria were cloned and clonal hybridomas were confirmed by ELISA against human and mouse α11β1 and subsequently scaled-up and IgGs were purified. Heavy and light chain variable regions of selected hybridomas were then sequenced.

### Phage Library Display

Phage library display was employed for generation of fully human anti-α11β1 antibodies. Fully human anti-α11β1 antibodies were discovered using single chain fragment variable (scFv) antigen-binding fragments displayed on phages (phage display library). Three rounds of selection were performed on purified human and mouse α11β1 antigen as well as deselection against α10β1, to enrich for all subunit-specific antibodies. Subsequently, optimal populations were subcloned into a bacterial soluble expression vector, recombinant antibody expression was induced and supernatant was screened for binding via ELISA assays. Antibodies with appropriate binding profiles were sequenced and subsequently converted from scFv to IgG.

### B. ELISA

0.25 μg/mL of target antigen (recombinant human or mouse α11β1) was plated in a 96-well plate over night at 4° C. Plates were washed (PBS with 0.1% Tween-20), blocked (PBS with 2% BSA and 0.05% Tween-20) for 1 hour at room temperature and incubated with a range of antibody concentrations for 1 hour at room temperature. Subsequently, plates were washed and incubated with biotinylated anti-rabbit/mouse/human IgG in a 1:1000 dilution buffer and incubated for 1 hour at room temperature. After washing the plates, Streptavidin HRP was added at 1:200 in dilution buffer and incubated for 1 hour at room temperature. Ultra TMB ELISA substrate solution was added and the plates incubated for 5 minutes on a plate shaker. The reaction was stopped by adding stop solution to each well and plates were read at 450 nm.

### C. FACS

### Antibody Binding to CHO-K1

200,000 cells (wild-type CHO-K1 cells or CHO-K1 cells expressing human α11) were incubated with each antibody at desired concentrations in FACS buffer for 30 min at 4° C. Then, the cells were washed with FACS buffer and incubated with secondary antibody at 1:100 dilution for 30 minutes at 4° C. Cells were then washed and fixed with 1% PF A in PBS for 20 minutes at room temperature; washed again and read on a cytometer in FACS buffer.

### Antibody Binding to HPF/MF

Human Pulmonary Fibroblasts (ScienCell) were cultured in complete Fibroblast Growth Medium (ScienCell) until 80% confluent in T-150 flasks. Cells were washed and harvested using Accutase. Cells were seeded into T-150 flasks at 7,500 cells/cm² in complete FGF and cultured for 72 hours. Cells were then washed and starved in serum reduced medium for 24 hours. After starvation, cells were treated with TGFβ1 (R&D Systems) for 72 hours. Cells were harvested using Accutase and seeded in 96-well conical bottom plates. Cells were blocked with Heat Inactived Fetal Bovine Serum (Gibco) for 30 minutes at 4° C. Cells were then incubated with anti-al I antibodies at the doses described in each figure for 30 minutes at 4° C. A human anti-al I antibody (Creative BioLabs) was included as a positive control as well as the appropriate IgG isotype negative controls. Cells were washed twice and incubated with PE conjugated secondary antibodies specific to the IgG class of the anti-α11 antibodies being tested for 30 minutes at 4° C. Cells were washed twice and fixed in 1% PF A for 30 minutes. Cells were acquired on a FACS Verse (Benton Dickson) binding of each antibody. Data were analyzed by gating on single cells and determining the geometric Mean Fluorescence Intensity (gMFI) in the PE channel for each sample.

### D. Surface Plasmon Resonance (SPR)

The affinity of antibodies for human α11β1 was measured by surface plasm on resonance assay (SPR). Affinity was measured at pH 7.6 and 25° C. with a Biacore T200 instrument. Anti-HIS antibody was immobilized on the SPR sensor surface using EDC/NHS covalent attachment. HIS-tagged human α11β1 was captured on the sensor surface and a single cycle kinetics assay was used. Increasing concentrations of test antibody were injected in series over the sensor-bound α11β1. Dissociation was monitored for 1000 seconds. A sensor surface with only anti-HIS antibody and a series of blank injections were used to double-reference subtract the data. A 1:1 Langmuir model was fit to the data to estimate the kinetic association and dissociation constants. The affinity (equilibrium dissociation constant) of the interaction was calculated by dividing the kinetic dissociation constant by the kinetic association constant. Between injection cycles α11β1 and bound antibody were removed with an injection of 10 mM glycine at pH 1.5.

### E. Cell Adhesion Inhibition

0.6×10⁶ cells/mL were incubated with each antibody at a range of concentrations for 20 minutes at 37° C. E-Plate VIEW 96 PET plate coated with 100 ng/ml type I collagen or PBS overnight at room temperature was blocked in 3% BSA for 1 hour at room temperature. After washing the plate with PBS, cell and antibody mixture was added to the wells and the plate was places into an xCelligence machine. Cell adhesion was recorded over 6 hours. The time point of maximum cell adhesion was used for comparison relative to control.

### F. Fibroblast-to-Myofibroblasts Transition (FMT)

Human Pulmonary Fibroblasts (ScienCell) were cultured in complete Fibroblast Growth Medium (ScienCell) until 80% confluent. Cells were washed and harvested using Accutase. Cells were seeded onto tissue culture-treated 96 well plates at 20,000 cells/well in complete Fibroblast Growth Medium. After 24 hours, cells were washed and starved in serum reduced medium for an additional 24 hours. After starvation, cells were treated with TGFβ-1 (R&D Systems) with or without anti-al I antibodies. A polyclonal rabbit anti-human all antibody was used as a positive control. Appropriate IgG isotype controls were also included. After 48 hours, cells were harvested, fixed, permeabilized and stained with AlexaFluor488 Labeled Anti-aSMA (a-smooth muscle actin) (Invitrogen). Cells were acquired on a FACS Verse (Benton Dickson) to determine expression levels of aSMA. Data were analyzed by gating on single cells and determining the geometric Mean Fluorescence Intensity (gMFI) in the FITC channel for each sample. gMFI for each sample was normalized to the untreated control and presented as % inhibition.

### G. Collagen Gel Contraction Assay

24-well plates were blocked with 2% BSA in PBS overnight at 37° C. The following day, the plates were washed 3 times with PBS before being used in the assay. Human CHO cell lines expressing all were harvested and resuspended at 1.25×10⁶ in ExpiCHO Expression Medium (Gibco™ Cat #A2910002). The collagen gel solution was prepared by diluting 3 mg/mL stock collagen type I (Gibco™ Collagen I Rat Protein, Tail Cat #A1048301) to I mg/mL in the media containing the CHO cells. Sodium hydroxide was added to the solution to neutralize the pH and 400 μL of the collagen solution was added to each well of the 24-well plates. For the wells where the antibodies were included in the collagen gel solution, CHO cells were prepared at 2.5×10⁶ and the antibodies were prepared at 2× final concentration in ExpiCHO media. The cells and antibodies were then combined 1:1 before the addition of the stock collagen type I. The gels were allowed to polymerize for 60 minutes at 37° C. Antibodies were added to the ExpiCHO media, which was then layered on top of the polymerized gel (400 L/well). The gels were incubated for 6 days at 37° C. before gel contraction was quantified. Images of each well were analyzed using Image J and gel contraction was determined as a percentage of the initial gel area.

### H. Tumor Xenograft Model

Fifty-six female C.B-17 SCID mice were inoculated with A549 cells (5×10⁶ cells/mouse) subcutaneously in the flank. Once tumor volume reached ˜100 mm³, animals were randomized amongst 7 groups of 8 mice each. Mice were then treated intraperitonealy every 3 days for a total of 7 doses with isotype controls or novel mAbs 79E3E3, 16E10 and 9G04 (2 and 20 mg/kg) or with docetaxel at 10 mg/kg every 4 days for a total of 6 doses. Tumor volumes and body weights were recorded twice a week with a gap of 2-3 days in between two measurements until any of the following conditions defined were observed: loss of 20% or more body weight; tumors that inhibit normal physiological function such as eating, drinking, and mobility; ulcerated tumors; tumor size greater than 2000 mm³ and clinical observations of prostration, paralysis, seizures and hemorrhages.

### I. Precision-Cut Liver Slices (PCLS)

Precision-Cut Liver Slices (PCLS) were prepared from resected liver tissue and rested for 24 hours to allow the post-slicing stress period to elapse before experiments began. PCLS were cultured without exogenous challenge (Group 1), with 100 μg/mL control antibodies (Groups 2 and 3—either mouse IgG2a or rabbit IgG), or with a combination of TGF-β1 (3 ng/ml) and PDGF-ββ (50 ng/ml) (Groups 2-10). PCLS were cultured in the presence or absence of 10 μM Alk5i (Group 4) as a positive control or novel inhibitors (16E10, 79E3E3, and 9G05) at 2 escalating doses (10 and 100 μg/mL) in Groups 5-10. Each of the 10 groups included n=6 human PCLS prepared from a single human liver. PCLS culture media, including all stimuli and compounds, was refreshed and harvested at 24-hour intervals. Cell culture supernatant (n=⅔ paired wells) was collected every 24 hours and snap frozen for quantification of soluble outputs. All PCLS were harvested at 96 hours.

Tissue culture levels of markers of liver damage (lactate dehydrogenase (LDH) and aspartate transaminase (AST)) and hepatocyte function/viability (albumin) were quantified on all PCLS at all time points. Albumin secretion was quantified by ELISA as a marker of PCLS integrity and function. Levels of collagen la1, IL-6, hyaluronic acid and Timp-1 in the cell culture supernatants were quantified using R&D Duoset ELISA kits.

Total RNA extraction from PCLS was performed on all samples. RNeasy Mini kits (Qiagen) were used for RNA extraction. RNA was reverse-transcribed to cDNA and used in qPCR to measure transcript levels of Coll al, aSMA, TIMP-1, TGF-β1, IL-6 and ˜actin/GAPDH

### Example 1: Characterization of α11β1 Antibodies

Novel monoclonal antibodies against α11β1 were previously generated, as described in International Patent Publication WO 2021/127500, which is hereby incorporated by reference in its entirety. Further characterization of three antibodies, 1994-01-C7 (#9), 16E10, and 79E3E3, is described herein.

Biophysical profiles of 1994-01-C7, 16E10, and 79E3E3 are described in the following table (Table 10).

TABLE 10
Biophysical profiles of anti-α11β1 antibodies

| | 1994-01-C7 (#9) | | 16E10 | | 79E3E3 | |
|---|---|---|---|---|---|---|
| mAb source | Human Phage | | B cell sorting | | Rat hybridoma | |
| | Display | | | | | |
| Affinity for | 240 | pM | 48 | pM | 1.3 | nM |
| human α11β1 | | | | | | |
| by SPR (Kd) | | | | | | |
| CHO-α11β1 | 3.5 | nM | 0.3 | nM | 3.4 | nM |
| binding (EC50) | | | | | | |
| Pulmonary | 3.5 | nM | 0.35 | nM | 3.35 | nM |
| Myofibroblast | | | | | | |
| binding (EC50) | | | | | | |
| CHO-hu α11 | 3.3 | nM | 0.39 | nM | 9.4 | nM |
| adhesion | | | | | | |
| inhibition (IC50) | | | | | | |

| | | | |
|---|---|---|---|
| Inhibits CHO-α11 | Yes | Yes | Yes |
| mediated collagen | | | |
| gel contraction | | | |
| Cross reacts | No by SPR; binds | No | Yes, 210 pM |
| with mouse | cell-mu all | | |
| α11β1 | | | |

### Example 2: Anti-α11β1 Antibodies Reduce Pro-Fibrotic and Pro-Inflammatory Mediators in Human Fibrotic Precision Cut Kidney Slices

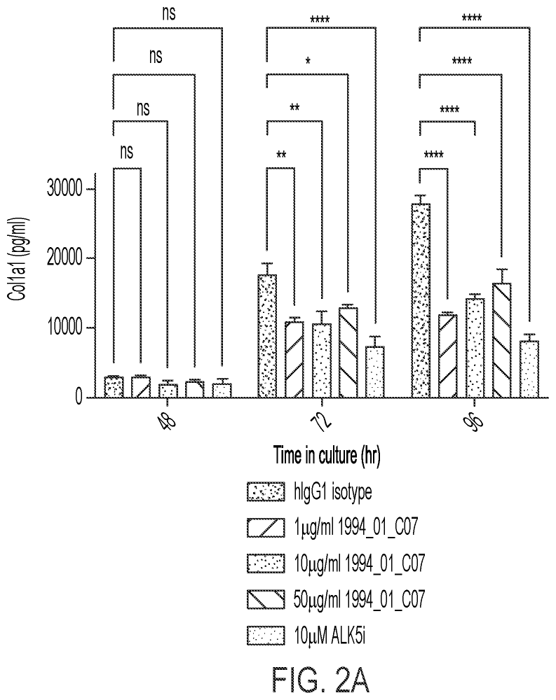

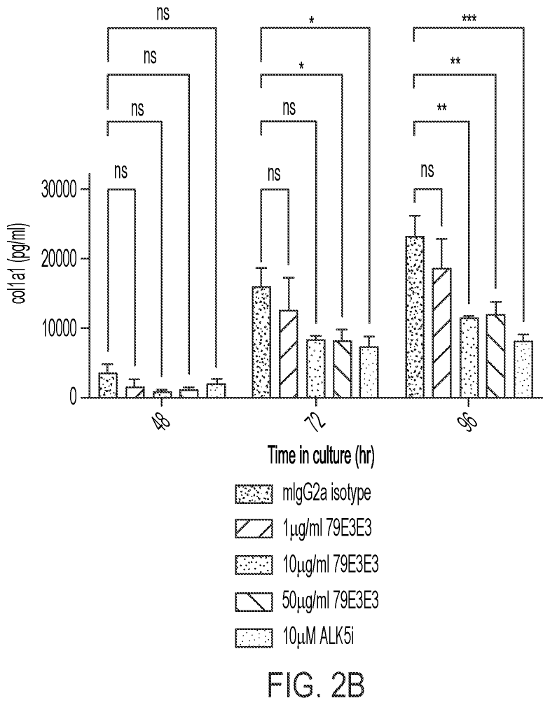

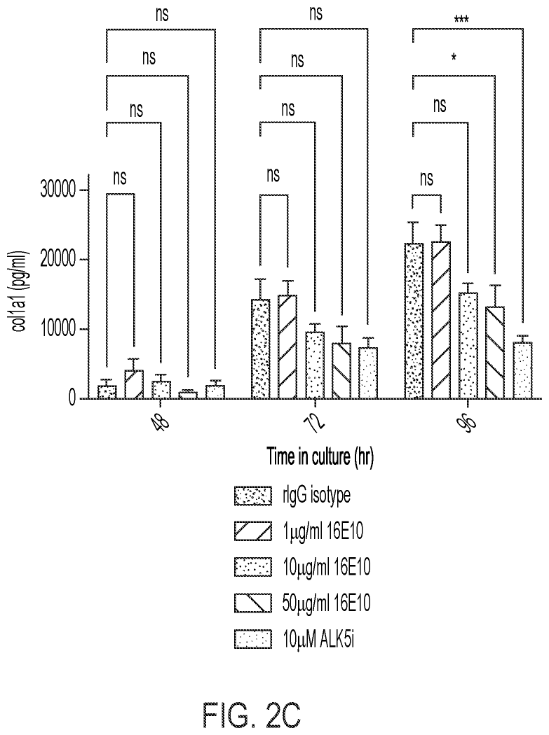

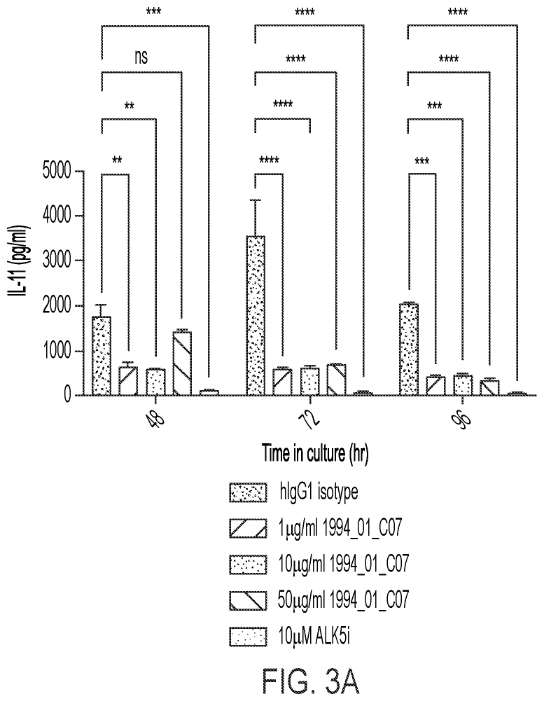

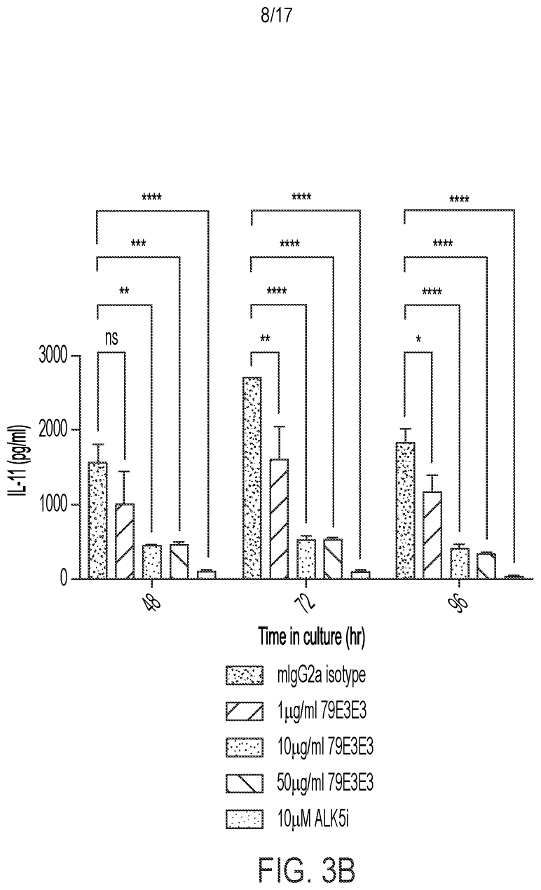

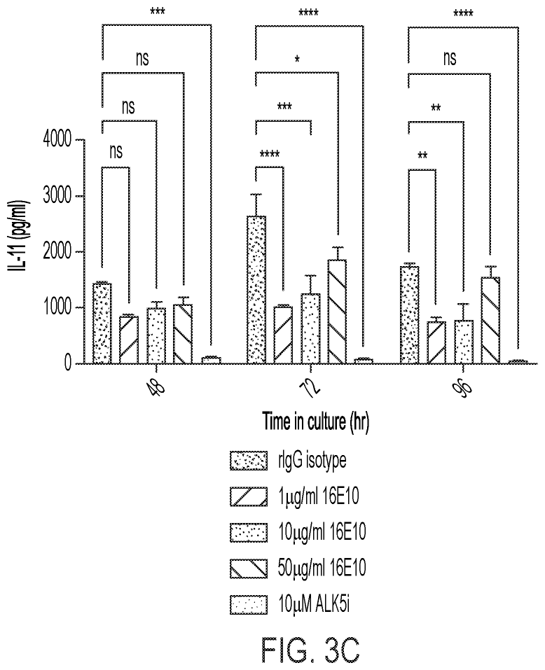

Precision cut kidney slices (PCKS) were treated with TGF-β1 and PDGFββ in the presence or absence of Alk5i and increasing doses of anti-α11β1 antibodies. The antibodies provided an inhibition of secreted pro-fibrotic and pro-inflammatory mediators Col1a1 (1994-01-C7 FIG. 2A, 79E3E3 FIG. 2B, and 16E10 FIG. 2C) and IL-11 (1994-01-C7 FIG. 3A, 79E3E3 FIG. 3B, and 16E10 FIG. 3C) in a dose dependent manner.

### Example 3: Additional Anti-α11β1 Antibody Variants

Additional variants, including humanized variants and alternative heavy and light chain CDR regions, were developed from 1994-01-C7, 16E10, and 79E3E3, as described herein.

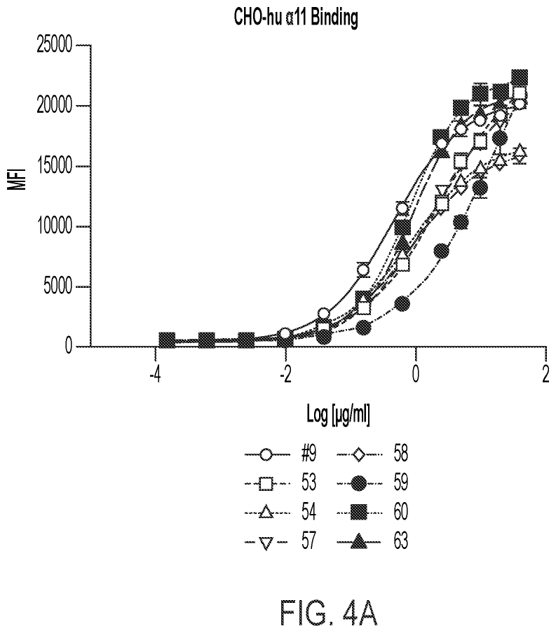

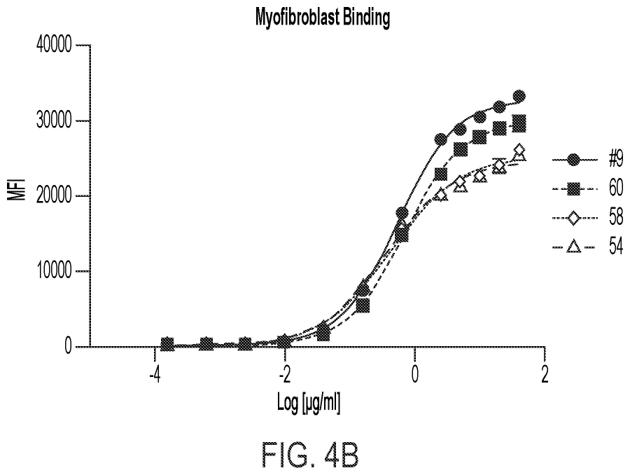

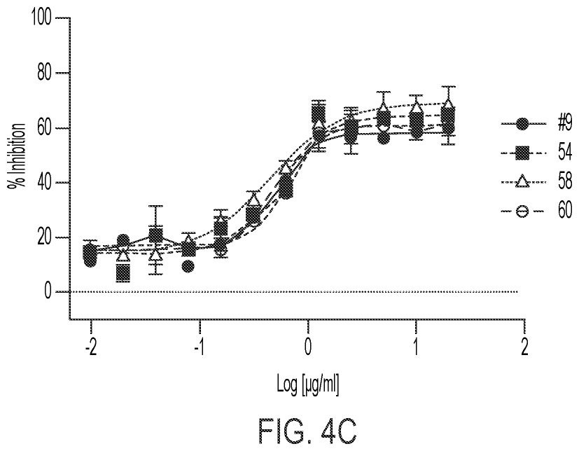

1994-01-C7 variants were compared in both CHO-hu α11 binding (FIG. 4A) and myofibroblast binding assays (FIG. 4B). Variants were also compared in an inhibition of cell adhesion assay (FIG. 4C). EC50 or IC50 results for each variant and assay is shown in Table 11 below.

TABLE 11
EC50/IC50 data for 1994-01-C7 variants

| | VH sequence | CHO-hu α11 | Myofibroblast | Inhibition of Cell |
|---|---|---|---|---|
| Antibody | VL sequence | binding EC50 | binding EC50 | Adhesion IC50 |
| 9 | SEQ ID NO: 32 | 3.0 | 3.6 | 3.4 |
| | SEQ ID NO: 37 | | | |
| 53 | — | 16.2 | — | — |
| 54 | SEQ ID NO: 30 | 5.5 | 2.4 | 3.5 |
| | SEQ ID NO: 37 | | | |
| 57 | — | 11.4 | — | — |
| 58 | SEQ ID NO: 31 | 5.94 | 2.87 | 3.0 |
| | SEQ ID NO: 37 | | | |
| 59 | | 1114.4 | — | — |
| 60 | SEQ ID NO: 33 | 5.3 | 4.6 | 4.2 |
| | SEQ ID NO: 37 | | | |
| 63 | — | 5.8 | — | — |

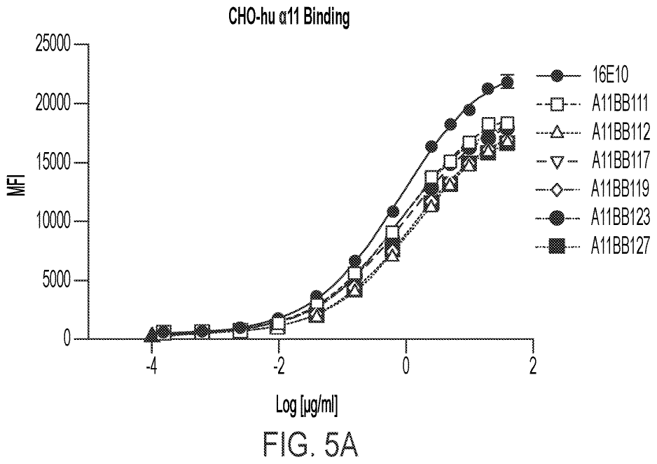

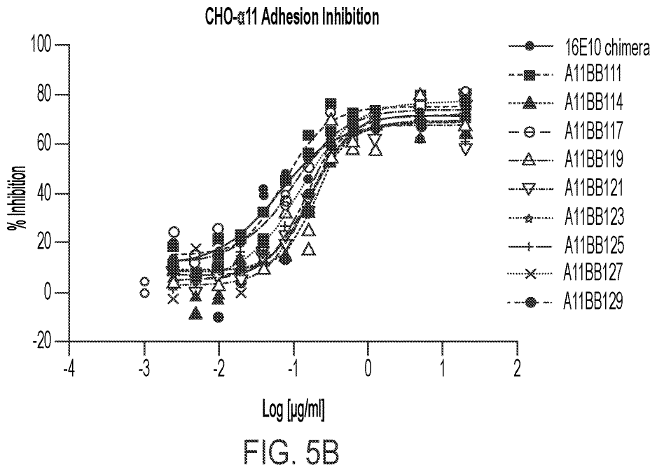

16E10 variants were compared in CHO-hu α11 binding (FIG. 5A) and CHO-α11 Adhesion Inhibition assays (FIG. 5B). EC50 or IC50 results for each variant and assay is shown in Table 12 below.

TABLE 12
EC50/IC50 data for 16E10 variants

| | | | CHO-α11 |
|---|---|---|---|
| | VH sequence | CHO-hu α11 | Adhesion |
| Antibody | VL sequence | binding EC50 | Inhibition IC50 |
| 16E10 | SEQ ID NO: 57 | 5.45 | 0.4 |
| | SEQ ID NO: 62 | | |
| A11BB111 | SEQ ID NO: 52 | 5.70 | — |
| | SEQ ID NO: 61 | | |
| A11BB112 | SEQ ID NO: 49 | 9.59 | 0.5 |
| | SEQ ID NO: 61 | | |
| A11BB114 | SEQ ID NO: 50 | — | 0.5 |
| | SEQ ID NO: 61 | | |
| A11BB117 | SEQ ID NO: 56 | 5.08 | 0.7 |
| | SEQ ID NO: 61 | | |
| A11BB119 | SEQ ID NO: 45 | 8.32 | 0.7 |
| | SEQ ID NO: 61 | | |
| A11BB121 | SEQ ID NO: 53 | — | 0.5 |
| | SEQ ID NO: 61 | | |
| A11BB123 | SEQ ID NO: 47 | 6.25 | 0.5 |
| | SEQ ID NO: 61 | | |
| A11BB125 | SEQ ID NO: 55 | — | 0.5 |
| | SEQ ID NO: 61 | | |
| A11BB127 | SEQ ID NO: 46 | 7.55 | 0.4 |
| | SEQ ID NO: 61 | | |
| A11BB129 | SEQ ID NO: 54 | — | 0.5 |
| | SEQ ID NO: 61 | | |

79E3E3 variants were compared in CHO-hu α11 binding, and the EC50 results are shown in Table 13 below.

TABLE 13
EC50 data for 79E3E3 variants

| | VH sequence | CHO-hu α11 |
|---|---|---|
| Antibody | VL sequence | binding EC50 |
| Wild Type | SEQ ID NO: 8 | 2.50 |
| | SEQ ID NO: 20 | |
| A11BB131 | SEQ ID NO: 11 | 2.49 |
| | SEQ ID NO: 21 | |
| A11BB133 | SEQ ID NO: 4 | 5.13 |
| | SEQ ID NO: 21 | |
| A11BB134 | SEQ ID NO: 10 | 3.49 |
| | SEQ ID NO: 22 | |
| A11BB136 | SEQ ID NO: 14 | 1.92 |
| | SEQ ID NO: 22 | |
| A11BB138 | SEQ ID NO: 12 | 4.63 |
| | SEQ ID NO: 21 | |
| A11BB139 | SEQ ID NO: 5 | 1.98 |
| | SEQ ID NO: 22 | |
| A11BB140 | SEQ ID NO: 7 | 2.50 |
| | SEQ ID NO: 22 | |
| A11BB141 | SEQ ID NO: 7 | 4.60 |
| | SEQ ID NO: 19 | |
| A11BB142 | SEQ ID NO: 6 | 2.07 |
| | SEQ ID NO: 22 | |
| A11BB143 | SEQ ID NO: 15 | 2.12 |
| | SEQ ID NO: 22 | |

### Example 4: α11β1 Signature Prominent in Rheumatoid Arthritis Tissue Samples

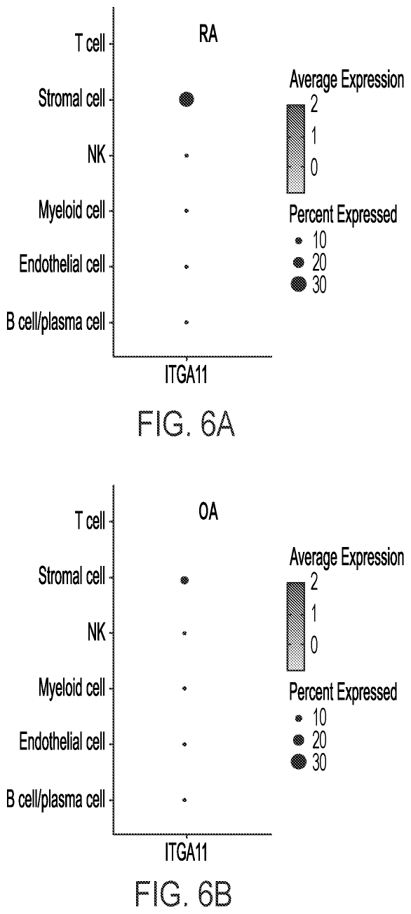

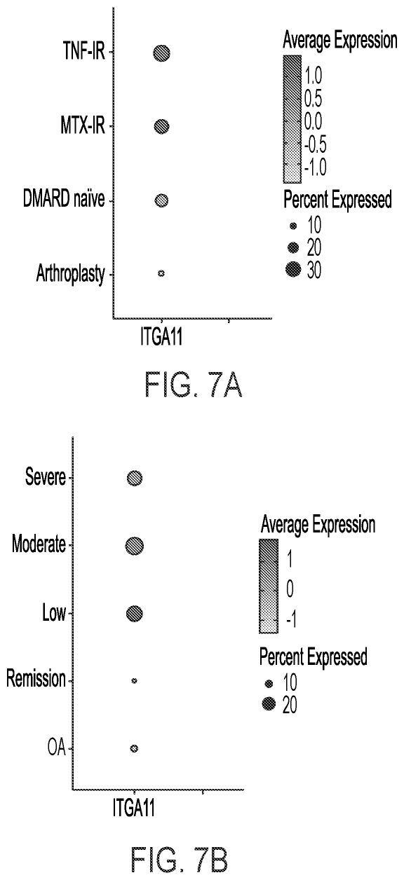

α11β1 is expressed in stromal cells from subjects with both rheumatoid arthritis (RA) and osteoarthritis (OA), but is expressed at a much higher rate in the RA stromal cells. FIG. 6A shows the average α11β1 expression and percent expressed in RA T-cells, stromal cells, natural killer (NK) cells, myeloid cells, endothelial cells, and B cells. α11β1 is only expressed in a substantial degree in the RA stromal cells, with around a 30% expression. FIG. 6B shows that α11β1 is also expressed in OA stromal cells compared to the other cell types, but with only around a 10% expression. Within the RA subjects, α11β1 is highly expressed in subjects deemed to be TNF inadequate responders, and is expressed in low to severe forms of RA. FIG. 7A shows the average α11β1 expression and percent expressed in TNF drug inadequate responders (TNF-IR), methotrexate inadequate responders (MTX-IR), disease modifying anti-rheumatic drug naïve (DMARD naïve), and subjects who have undergone arthroplasty. α11β1 is expressed the most in the TNF-IR group, although there is also expression in both the MTX-IR and DMARD naïve groups as well. FIG. 7B shows that α11β1 is expressed in low, moderate, and severe RA, however it is shows highest expression in low RA and slightly decreases expression as severity increases. α11β1 is not expressed in subjects in remission or that have OA.

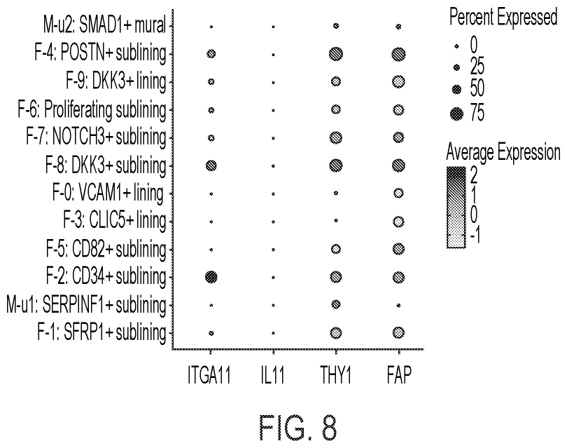

Lastly, α11β1 is selectively expressed in sublining fibroblast cells. FIG. 8 shows that α11β1 expression (ITFA11, first column) is very strong in fibroblast cells that are CD34+, which indicates they locate to the deep sublining area of the joint synovium.

Taken together, this data shows that α11β1 is highly expressed in RA stromal cells and in sublining fibroblast cells in subjects who are TNF-IR and MTX-IR. This information strongly indicates that α11β1 is strong target for therapeutic intervention for RA subjects, including subjects where traditional treatments have been inadequate.

### Example 5: Treatment of Rheumatoid Arthritis Using Anti-α11β1 Antibodies

A therapeutic composition comprising an anti-α11β1 antibody as provided for herein is administered to a patient with rheumatoid arthritis. The anti-α11β1 antibody targets fibroblast cells in the synovium of affected joints, and the rheumatoid arthritis is treated.

The embodiments and examples provided herein demonstrate anti-α11β1 antibodies provided for herein and their use in treating α11β1 mediated diseases, including inflammatory, fibrotic, and autoimmune disorders.

This specification contains citations and references, each of which is hereby incorporated by reference in their entirety for all purposes.

What is claimed is:

1. An antibody, or an antigen-binding fragment thereof, wherein the antibody or antigen-binding fragment thereof, comprises:
(i) a heavy chain variable region comprising heavy chain HCDR1, HCDR2, and HCDR3 sequences, wherein the heavy chain HCDR1 sequence has the amino acid sequence of SEQ ID NO: 1; the heavy chain HCDR2 has the amino acid sequence of SEQ ID NO: 2 and the heavy chain HCDR3 sequence has the amino acid sequence of SEQ ID NO: 3; or variants of any of the foregoing; and
(ii) a light chain variable region comprising light chain LCDR1, LCDR2, and LCDR3 sequences, wherein the light chain LCDR1 sequence has the amino acid sequence SEQ ID NO: 16; the light chain LCDR2 sequence has the amino acid sequence of SEQ ID NO: 17; and the light chain LCDR3 sequence has the amino acid sequence of SEQ ID NO: 18; or variants of any of the foregoing.

2. The antibody, or antigen-binding fragment thereof, of claim 1, wherein the heavy chain variable region comprises an amino acid sequence having at least 90%, 91%, 92%, 93%, 94%, 95%, 96%, 97%, 98%, or 99% sequence identity, or is identical to SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, or SEQ ID NO: 15, provided that the heavy chain variable region comprises the sequences of HCDR1, HCDR2, and HCDR3 of claim 1.

3. The antibody, or antigen-binding fragment thereof, of claim 1 or 2, wherein the light chain variable region comprises an amino acid sequence having at least 90%, 91%, 92%, 93%, 94%, 95%, 96%, 97%, 98%, or 99% sequence identity, or is identical to SEQ ID NO: 19, SEQ ID NO: 20, SEQ ID NO: 21, or SEQ ID NO: 22, provided that the light chain variable region comprises the sequences of LCDR1, LCDR2, and LCDR3 of claim 1.

4. The antibody, or antigen-binding fragment thereof, of any one of claims 1-3, comprising:
(i) a heavy chain variable region comprising an amino acid sequence having at least 90%, 91%, 92%, 93%, 94%, 95%, 96%, 97%, 98%, or 99% identity, or is identical, to SEQ ID NO: 11, provided that the heavy chain variable region comprises a HCDR1 of SEQ ID NO: 1, a HCDR2 of SEQ ID NO: 2, and a HCDR3 of SEQ ID NO: 3; and
(ii) a light chain variable region comprising an amino acid sequence having at least 90%, 91%, 92%, 93%, 94%, 95%, 96%, 97%, 98%, or 99% identity, or is identical, to SEQ ID NO: 21, provided that the light chain variable region comprises a LCDR1 of SEQ ID NO: 16, a LCDR2 of SEQ ID NO: 17, and a LCDR3 of SEQ ID NO: 18.

5. The antibody, or antigen-binding fragment thereof, of any one of claims 1-3, comprising:
(i) a heavy chain variable region comprising an amino acid sequence having at least 90%, 91%, 92%, 93%, 94%, 95%, 96%, 97%, 98%, or 99% identity, or is identical, to SEQ ID NO: 4, provided that the heavy chain variable region comprises a HCDR1 of SEQ ID NO: 1, a HCDR2 of SEQ ID NO: 2, and a HCDR3 of SEQ ID NO: 3; and
(ii) a light chain variable region comprising an amino acid sequence having at least 90%, 91%, 92%, 93%, 94%, 95%, 96%, 97%, 98%, or 99% identity, or is identical, to SEQ ID NO: 22, provided that the light chain variable region comprises a LCDR1 of SEQ ID NO: 16, a LCDR2 of SEQ ID NO: 17, and a LCDR3 of SEQ ID NO: 18.

6. The antibody, or antigen-binding fragment thereof, of any one of claims 1-3, comprising:
(i) a heavy chain variable region comprising an amino acid sequence having at least 90%, 91%, 92%, 93%, 94%, 95%, 96%, 97%, 98%, or 99% identity, or is identical, to SEQ ID NO: 4, provided that the heavy chain variable region comprises a HCDR1 of SEQ ID NO: 1, a HCDR2 of SEQ ID NO: 2, and a HCDR3 of SEQ ID NO: 3; and
(ii) a light chain variable region comprising an amino acid sequence having at least 90%, 91%, 92%, 93%, 94%, 95%, 96%, 97%, 98%, or 99% identity, or is identical, to SEQ ID NO: 21, provided that the light chain variable region comprises a LCDR1 of SEQ ID NO: 16, a LCDR2 of SEQ ID NO: 17, and a LCDR3 of SEQ ID NO: 18.

7. The antibody, or antigen-binding fragment thereof, of any one of claims 1-3, comprising:
(i) a heavy chain variable region comprising an amino acid sequence having at least 90%, 91%, 92%, 93%, 94%, 95%, 96%, 97%, 98%, or 99% identity, or is identical, to SEQ ID NO: 10, provided that the heavy chain variable region comprises a HCDR1 of SEQ ID NO: 1, a HCDR2 of SEQ ID NO: 2, and a HCDR3 of SEQ ID NO: 3; and
(ii) a light chain variable region comprising an amino acid sequence having at least 90%, 91%, 92%, 93%, 94%, 95%, 96%, 97%, 98%, or 99% identity, or is identical, to SEQ ID NO: 22, provided that the light chain variable region comprises a LCDR1 of SEQ ID NO: 16, a LCDR2 of SEQ ID NO: 17, and a LCDR3 of SEQ ID NO: 18.

8. The antibody, or antigen-binding fragment thereof, of any one of claims 1-3, comprising:
(i) a heavy chain variable region comprising an amino acid sequence having at least 90%, 91%, 92%, 93%, 94%, 95%, 96%, 97%, 98%, or 99% identity, or is identical, to SEQ ID NO: 9, provided that the heavy chain variable region comprises a HCDR1 of SEQ ID NO: 1, a HCDR2 of SEQ ID NO: 2, and a HCDR3 of SEQ ID NO: 3; and
(ii) a light chain variable region comprising an amino acid sequence having at least 90%, 91%, 92%, 93%, 94%, 95%, 96%, 97%, 98%, or 99% identity, or is identical, to SEQ ID NO: 21, provided that the light chain variable region comprises a LCDR1 of SEQ ID NO: 16, a LCDR2 of SEQ ID NO: 17, and a LCDR3 of SEQ ID NO: 18.

9. The antibody, or antigen-binding fragment thereof, of any one of claims 1-3, comprising:
(i) a heavy chain variable region comprising an amino acid sequence having at least 90%, 91%, 92%, 93%, 94%, 95%, 96%, 97%, 98%, or 99% identity, or is identical, to SEQ ID NO: 14, provided that the heavy chain variable region comprises a HCDR1 of SEQ ID NO: 1, a HCDR2 of SEQ ID NO: 2, and a HCDR3 of SEQ ID NO: 3; and
(ii) a light chain variable region comprising an amino acid sequence having at least 90%, 91%, 92%, 93%, 94%, 95%, 96%, 97%, 98%, or 99% identity, or is identical, to SEQ ID NO: 22, provided that the light chain variable region comprises a LCDR1 of SEQ ID NO: 16, a LCDR2 of SEQ ID NO: 17, and a LCDR3 of SEQ ID NO: 18.

10. The antibody, or antigen-binding fragment thereof, of any one of claims 1-3, comprising:
(i) a heavy chain variable region comprising an amino acid sequence having at least 90%, 91%, 92%, 93%, 94%, 95%, 96%, 97%, 98%, or 99% identity, or is identical, to SEQ ID NO: 13, provided that the heavy chain variable region comprises a HCDR1 of SEQ ID NO: 1, a HCDR2 of SEQ ID NO: 2, and a HCDR3 of SEQ ID NO: 3; and
(ii) a light chain variable region comprising an amino acid sequence having at least 90%, 91%, 92%, 93%, 94%, 95%, 96%, 97%, 98%, or 99% identity, or is identical, to SEQ ID NO: 21, provided that the light chain variable region comprises a LCDR1 of SEQ ID NO: 16, a LCDR2 of SEQ ID NO: 17, and a LCDR3 of SEQ ID NO: 18.

11. The antibody, or antigen-binding fragment thereof, of any one of claims 1-3, comprising:
(i) a heavy chain variable region comprising an amino acid sequence having at least 90%, 91%, 92%, 93%, 94%, 95%, 96%, 97%, 98%, or 99% identity, or is identical, to SEQ ID NO: 12, provided that the heavy chain variable region comprises a HCDR1 of SEQ ID NO: 1, a HCDR2 of SEQ ID NO: 2, and a HCDR3 of SEQ ID NO: 3; and
(ii) a light chain variable region comprising an amino acid sequence having at least 90%, 91%, 92%, 93%, 94%, 95%, 96%, 97%, 98%, or 99% identity, or is identical, to SEQ ID NO: 21, provided that the light chain variable region comprises a LCDR1 of SEQ ID NO: 16, a LCDR2 of SEQ ID NO: 17, and a LCDR3 of SEQ ID NO: 18.

12. An antibody, or an antigen-binding fragment thereof, wherein the antibody, or the antigen-binding fragment thereof, comprises:
(i) a heavy chain variable region comprising heavy chain HCDR1, HCDR2, and HCDR3 sequences, wherein the heavy chain HCDR1 sequence has the amino acid sequence of SEQ ID NO: 23; the heavy chain HCDR2 has the amino acid sequence of SEQ ID NO: 24, SEQ ID NO: 25, SEQ ID NO: 26, or SEQ ID NO: 27, and the heavy chain HCDR3 sequence has the amino acid sequence of SEQ ID NO: 28, or SEQ ID NO: 29, or variants of any of the foregoing; and
(ii) a light chain variable region comprising light chain LCDR1, LCDR2, and LCDR3 sequences, wherein the light chain LCDR1 sequence has the amino acid sequence SEQ ID NO: 34; the light chain LCDR2 sequence has the amino acid sequence of SEQ ID NO: 35; and the light chain LCDR3 sequence has the amino acid sequence of SEQ ID NO: 36; or variants of any of the foregoing.

13. The antibody, or antigen-binding fragment thereof, of claim 12, wherein the HCDR2 is SEQ ID NO: 24 and the HCDR3 is SEQ ID NO: 28.

14. The antibody, or antigen-binding fragment thereof, of claim 12, wherein the HCDR2 is SEQ ID NO: 24 and the HCDR3 is SEQ ID NO: 29.

15. The antibody, or antigen-binding fragment thereof, of claim 12, wherein the HCDR2 is SEQ ID NO: 25 and the HCDR3 is SEQ ID NO: 28.

16. The antibody, or antigen-binding fragment thereof, of claim 12, wherein the HCDR2 is SEQ ID NO: 25 and the HCDR3 is SEQ ID NO: 29.

17. The antibody, or antigen-binding fragment thereof, of claim 12, wherein the HCDR2 is SEQ ID NO: 26 and the HCDR3 is SEQ ID NO: 28.

18. The antibody, or antigen-binding fragment thereof, of claim 12, wherein the HCDR2 is SEQ ID NO: 26 and the HCDR3 is SEQ ID NO: 29.

19. The antibody, or antigen-binding fragment thereof, of claim 12, wherein the HCDR2 is SEQ ID NO: 27 and the HCDR3 is SEQ ID NO: 28.

20. The antibody, or antigen-binding fragment thereof, of claim 12, wherein the HCDR2 is SEQ ID NO: 27 and the HCDR3 is SEQ ID NO: 29.

21. The antibody, or antigen-binding fragment thereof, of any one of claims 12-20, wherein the heavy chain variable region comprises an amino acid sequence having at least 90% sequence identity to SEQ ID NO: 30, SEQ ID NO: 31, SEQ ID NO: 32, or SEQ ID NO: 33, provided that heavy chain variable region comprises the sequences of HCDR1, HCDR2, and HCDR3 of claim 12.

22. The antibody, or antigen-binding fragment thereof, any one of claims 12-21, wherein the light chain variable region comprises an amino acid sequence having at least 90% sequence identity to SEQ ID NO: 37, provided that the light chain variable region comprises the sequences of LCDR1, LCDR2, and LCDR3 of claim 12.

23. The antibody, or antigen-binding fragment thereof, of any one of claims 12-22, comprising:
(i) a heavy chain variable region comprising an amino acid sequence having at least 90%, 91%, 92%, 93%, 94%, 95%, 96%, 97%, 98%, or 99% identity, or is identical, to SEQ ID NO: 30, provided that the heavy chain variable region comprises a HCDR1 of SEQ ID NO: 23, a HCDR2 of SEQ ID NO: 24, and a HCDR3 of SEQ ID NO: 28; and
(ii) a light chain variable region comprising an amino acid sequence having at least 90%, 91%, 92%, 93%, 94%, 95%, 96%, 97%, 98%, or 99% identity, or is identical, to SEQ ID NO: 37, provided that the light chain variable region comprises a LCDR1 of SEQ ID NO: 34, a LCDR2 of SEQ ID NO: 35, and a LCDR3 of SEQ ID NO: 36.

24. The antibody, or antigen-binding fragment thereof, of any one of claims 12-22, comprising:
(i) a heavy chain variable region comprising an amino acid sequence having at least 90%, 91%, 92%, 93%, 94%, 95%, 96%, 97%, 98%, or 99% identity, or is identical, to SEQ ID NO: 31, provided that the heavy chain variable region comprises a HCDR1 of SEQ ID NO: 23, a HCDR2 of SEQ ID NO: 25, and a HCDR3 of SEQ ID NO: 28; and
(ii) a light chain variable region comprising an amino acid sequence having at least 90%, 91%, 92%, 93%, 94%, 95%, 96%, 97%, 98%, or 99% identity, or is identical, to SEQ ID NO: 37, provided that the light chain variable region comprises a LCDR1 of SEQ ID NO: 34, a LCDR2 of SEQ ID NO: 35, and a LCDR3 of SEQ ID NO: 36.

25. The antibody, or antigen-binding fragment thereof, of any one of claims 12-22, comprising:
(i) a heavy chain variable region comprising an amino acid sequence having at least 90%, 91%, 92%, 93%, 94%, 95%, 96%, 97%, 98%, or 99% identity, or is identical, to SEQ ID NO: 33, provided that the heavy chain variable region comprises a HCDR1 of SEQ ID NO: 23, a HCDR2 of SEQ ID NO: 27, and a HCDR3 of SEQ ID NO: 28; and
(ii) a light chain variable region comprising an amino acid sequence having at least 90%, 91%, 92%, 93%, 94%, 95%, 96%, 97%, 98%, or 99% identity, or is identical, to SEQ ID NO: 37, provided that the light chain variable region comprises a LCDR1 of SEQ ID NO: 34, a LCDR2 of SEQ ID NO: 35, and a LCDR3 of SEQ ID NO: 36.

26. An antibody, or an antigen-binding fragment thereof, wherein the antibody or antigen-binding fragment thereof, comprises:
(i) a heavy chain variable region comprising heavy chain HCDR1, HCDR2, and HCDR3 sequences, wherein the heavy chain HCDR1 sequence has the amino acid sequence of SEQ ID NO: 38, SEQ ID NO: 39, or SEQ ID NO: 40; the heavy chain HCDR2 has the amino acid sequence of SEQ ID NO: 41 or SEQ ID NO: 42; and the heavy chain HCDR3 sequence has the amino acid sequence of SEQ ID NO: 43; or variants of any of the foregoing; and
(ii) a light chain variable region comprising light chain LCDR1, LCDR2, and LCDR3 sequences, wherein the light chain LCDR1 sequence has the amino acid sequence SEQ ID NO: 58; the light chain LCDR2 sequence has the amino acid sequence of SEQ ID NO: 59; and the light chain LCDR3 sequence has the amino acid sequence of SEQ ID NO: 60; or variants of any of the foregoing.

27. The antibody, or antigen-binding fragment thereof, of claim 26, wherein the HCDR1 is SEQ ID NO: 38 and the HCDR2 is SEQ ID NO: 41.

28. The antibody, or antigen-binding fragment thereof, of claim 26, wherein the HCDR1 is SEQ ID NO: 38 and the HCDR2 is SEQ ID NO: 42.

29. The antibody, or antigen-binding fragment thereof, of claim 26, wherein the HCDR1 is SEQ ID NO: 39 and the HCDR2 is SEQ ID NO: 41.

30. The antibody, or antigen-binding fragment thereof, of claim 26, wherein the HCDR1 is SEQ ID NO: 39 and the HCDR2 is SEQ ID NO: 42.

31. The antibody, or antigen-binding fragment thereof, of claim 26, wherein the HCDR1 is SEQ ID NO: 40 and the HCDR2 is SEQ ID NO: 41.

32. The antibody, or antigen-binding fragment thereof, of claim 26, wherein the HCDR1 is SEQ ID NO: 40 and the HCDR2 is SEQ ID NO: 42.

33. The antibody, or antigen-binding fragment thereof, of any one of claims 26-32, wherein the heavy chain variable region comprising an amino acid sequence having at least 90% sequence identity to SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, or SEQ ID NO: 57, provided that heavy chain variable region comprises the sequences of HCDR1, HCDR2, and HCDR3 of claim 26.

34. The antibody, or antigen-binding fragment thereof, any one of claims 26-33, wherein the light chain variable region comprises an amino acid sequence having at least 90% sequence identity to SEQ ID NO: 61, or SEQ ID NO: 62, provided that the light chain variable region comprises the sequences of LCDR1, LCDR2, and LCDR3 of claim 26.

35. The antibody, or antigen-binding fragment thereof, of any one of claims 26-34, comprising:
(i) a heavy chain variable region comprising an amino acid sequence having at least 90%, 91%, 92%, 93%, 94%, 95%, 96%, 97%, 98%, or 99% identity, or is identical, to SEQ ID NO: 52, provided that the heavy chain variable region comprises a HCDR1 of SEQ ID NO: 39, a HCDR2 of SEQ ID NO: 42, and a HCDR3 of SEQ ID NO: 43; and
(ii) a light chain variable region comprising an amino acid sequence having at least 90%, 91%, 92%, 93%, 94%, 95%, 96%, 97%, 98%, or 99% identity, or is identical, to SEQ ID NO: 61, provided that the light chain variable region comprises a LCDR1 of SEQ ID NO: 58, a LCDR2 of SEQ ID NO: 59, and a LCDR3 of SEQ ID NO: 60.

36. The antibody, or antigen-binding fragment thereof, of any one of claims 26-34, comprising:
(i) a heavy chain variable region comprising an amino acid sequence having at least 90%, 91%, 92%, 93%, 94%, 95%, 96%, 97%, 98%, or 99% identity, or is identical, to SEQ ID NO: 49, provided that the heavy chain variable region comprises a HCDR1 of SEQ ID NO: 39, a HCDR2 of SEQ ID NO: 42, and a HCDR3 of SEQ ID NO: 43; and
(ii) a light chain variable region comprising an amino acid sequence having at least 90%, 91%, 92%, 93%, 94%, 95%, 96%, 97%, 98%, or 99% identity, or is identical, to SEQ ID NO: 61, provided that the light chain variable region comprises a LCDR1 of SEQ ID NO: 58, a LCDR2 of SEQ ID NO: 59, and a LCDR3 of SEQ ID NO: 60.

37. The antibody, or antigen-binding fragment thereof, of any one of claims 26-34, comprising:
(i) a heavy chain variable region comprising an amino acid sequence having at least 90%, 91%, 92%, 93%, 94%, 95%, 96%, 97%, 98%, or 99% identity, or is identical, to SEQ ID NO: 50, provided that the heavy chain variable region comprises a HCDR1 of SEQ ID NO: 39, a HCDR2 of SEQ ID NO: 42, and a HCDR3 of SEQ ID NO: 43; and
(ii) a light chain variable region comprising an amino acid sequence having at least 90%, 91%, 92%, 93%, 94%, 95%, 96%, 97%, 98%, or 99% identity, or is identical, to SEQ ID NO: 61, provided that the light chain variable region comprises a LCDR1 of SEQ ID NO: 58, a LCDR2 of SEQ ID NO: 59, and a LCDR3 of SEQ ID NO: 60.

38. The antibody, or antigen-binding fragment thereof, of any one of claims 26-34, comprising:
(i) a heavy chain variable region comprising an amino acid sequence having at least 90%, 91%, 92%, 93%, 94%, 95%, 96%, 97%, 98%, or 99% identity, or is identical, to SEQ ID NO: 56, provided that the heavy chain variable region comprises a HCDR1 of SEQ ID NO: 40, a HCDR2 of SEQ ID NO: 41, and a HCDR3 of SEQ ID NO: 43; and
(ii) a light chain variable region comprising an amino acid sequence having at least 90%, 91%, 92%, 93%, 94%, 95%, 96%, 97%, 98%, or 99% identity, or is identical, to SEQ ID NO: 61, provided that the light chain variable region comprises a LCDR1 of SEQ ID NO: 58, a LCDR2 of SEQ ID NO: 59, and a LCDR3 of SEQ ID NO: 60.

39. The antibody, or antigen-binding fragment thereof, of any one of claims 26-34, comprising:
(i) a heavy chain variable region comprising an amino acid sequence having at least 90%, 91%, 92%, 93%, 94%, 95%, 96%, 97%, 98%, or 99% identity, or is identical, to SEQ ID NO: 45, provided that the heavy chain variable region comprises a HCDR1 of SEQ ID NO: 38, a HCDR2 of SEQ ID NO: 41, and a HCDR3 of SEQ ID NO: 43; and
(ii) a light chain variable region comprising an amino acid sequence having at least 90%, 91%, 92%, 93%, 94%, 95%, 96%, 97%, 98%, or 99% identity, or is identical, to SEQ ID NO: 61, provided that the light chain variable region comprises a LCDR1 of SEQ ID NO: 58, a LCDR2 of SEQ ID NO: 59, and a LCDR3 of SEQ ID NO: 60.

40. The antibody, or antigen-binding fragment thereof, of any one of claims 26-34, comprising:
(i) a heavy chain variable region comprising an amino acid sequence having at least 90%, 91%, 92%, 93%, 94%, 95%, 96%, 97%, 98%, or 99% identity, or is identical, to SEQ ID NO: 53, provided that the heavy chain variable region comprises a HCDR1 of SEQ ID NO: 40, a HCDR2 of SEQ ID NO: 41, and a HCDR3 of SEQ ID NO: 43; and
(ii) a light chain variable region comprising an amino acid sequence having at least 90%, 91%, 92%, 93%, 94%, 95%, 96%, 97%, 98%, or 99% identity, or is identical, to SEQ ID NO: 61, provided that the light chain variable region comprises a LCDR1 of SEQ ID NO: 58, a LCDR2 of SEQ ID NO: 59, and a LCDR3 of SEQ ID NO: 60.

41. The antibody, or antigen-binding fragment thereof, of any one of claims 26-34, comprising:
(i) a heavy chain variable region comprising an amino acid sequence having at least 90%, 91%, 92%, 93%, 94%, 95%, 96%, 97%, 98%, or 99% identity, or is identical, to SEQ ID NO: 47, provided that the heavy chain variable region comprises a HCDR1 of SEQ ID NO: 38, a HCDR2 of SEQ ID NO: 41, and a HCDR3 of SEQ ID NO: 43; and
(ii) a light chain variable region comprising an amino acid sequence having at least 90%, 91%, 92%, 93%, 94%, 95%, 96%, 97%, 98%, or 99% identity, or is identical, to SEQ ID NO: 61, provided that the light chain variable region comprises a LCDR1 of SEQ ID NO: 58, a LCDR2 of SEQ ID NO: 59, and a LCDR3 of SEQ ID NO: 60.

42. The antibody, or antigen-binding fragment thereof, of any one of claims 26-34, comprising:
(i) a heavy chain variable region comprising an amino acid sequence having at least 90%, 91%, 92%, 93%, 94%, 95%, 96%, 97%, 98%, or 99% identity, or is identical, to SEQ ID NO: 55, provided that the heavy chain variable region comprises a HCDR1 of SEQ ID NO: 40, a HCDR2 of SEQ ID NO: 41, and a HCDR3 of SEQ ID NO: 43; and
(ii) a light chain variable region comprising an amino acid sequence having at least 90%, 91%, 92%, 93%, 94%, 95%, 96%, 97%, 98%, or 99% identity, or is identical, to SEQ ID NO: 61, provided that the light chain variable region comprises a LCDR1 of SEQ ID NO: 58, a LCDR2 of SEQ ID NO: 59, and a LCDR3 of SEQ ID NO: 60.

43. The antibody, or antigen-binding fragment thereof, of any one of claims 26-34, comprising:
(i) a heavy chain variable region comprising an amino acid sequence having at least 90%, 91%, 92%, 93%, 94%, 95%, 96%, 97%, 98%, or 99% identity, or is identical, to SEQ ID NO: 46, provided that the heavy chain variable region comprises a HCDR1 of SEQ ID NO: 38, a HCDR2 of SEQ ID NO: 41, and a HCDR3 of SEQ ID NO: 43; and
(ii) a light chain variable region comprising an amino acid sequence having at least 90%, 91%, 92%, 93%, 94%, 95%, 96%, 97%, 98%, or 99% identity, or is identical, to SEQ ID NO: 61, provided that the light chain variable region comprises a LCDR1 of SEQ ID NO: 58, a LCDR2 of SEQ ID NO: 59, and a LCDR3 of SEQ ID NO: 60.

44. The antibody, or antigen-binding fragment thereof, of any one of claims 26-34, comprising:
(i) a heavy chain variable region comprising an amino acid sequence having at least 90%, 91%, 92%, 93%, 94%, 95%, 96%, 97%, 98%, or 99% identity, or is identical, to SEQ ID NO: 54, provided that the heavy chain variable region comprises a HCDR1 of SEQ ID NO: 40, a HCDR2 of SEQ ID NO: 41, and a HCDR3 of SEQ ID NO: 43; and
(ii) a light chain variable region comprising an amino acid sequence having at least 90%, 91%, 92%, 93%, 94%, 95%, 96%, 97%, 98%, or 99% identity, or is identical, to SEQ ID NO: 61, provided that the light chain variable region comprises a LCDR1 of SEQ ID NO: 58, a LCDR2 of SEQ ID NO: 59, and a LCDR3 of SEQ ID NO: 60.

45. The antibody, or antigen-binding fragment thereof, of any one of the preceding claims, wherein the antibody, or antigen-binding fragment thereof, is a scFv antibody, a Fab fragment, a Fab′ fragment, or an F(ab′)₂ fragment.

46. The antibody, or antigen-binding fragment thereof, of any one of the preceding claims, wherein the antibody is a monoclonal antibody.

47. The antibody, or antigen-binding fragment thereof, of any one of the preceding claims, wherein the antibody is a humanized antibody.

48. The antibody, or antigen-binding fragment thereof, of any one of the preceding claims, wherein the antibody, or antigen-binding fragment thereof, comprises a constant region, such as provided for herein.

49. The antibody, or antigen-binding fragment thereof, of any one of the preceding claims, wherein the antibody, or antigen-binding fragment thereof, comprises a constant region comprising an amino acid sequence of SEQ ID NO: 63, SEQ ID NO: 64, SEQ ID NO: 65, SEQ ID NO: 66, SEQ ID NO: 67, SEQ ID NO: 68, or SEQ ID NO: 69.

50. The antibody, or antigen-binding fragment thereof, of any one of the preceding claims, wherein the antibody, or antigen-binding fragment thereof, comprises a heavy chain constant region comprising an amino acid sequence of SEQ ID NO: 63, and a light chain constant region comprising an amino acid sequence of SEQ ID NO: 69.

51. The antibody, or antigen-binding fragment thereof, of any one of the preceding claims, wherein the heavy chain variable region and the light chain variable region are not linked by a linker.

52. The antibody, or antigen-binding fragment thereof, of any one of the preceding claims, wherein the heavy chain variable region and the light chain variable region are linked with a peptide linker.

53. The antibody, or antigen-binding fragment thereof, of claim 52, wherein the peptide linker comprises a sequence of (GGGGS)ₙ (SEQ ID NO: 70); (GGGGA)ₙ (SEQ ID NO: 71), or any combination thereof, wherein each n is independently 1-5.

54. A recombinant antibody, or an antigen-binding fragment thereof, that binds to human α11β1 integrin, wherein the antibody or antigen-binding fragment thereof comprises an amino acid sequence, or a variant thereof, as provided for herein or an amino acid sequence that is 90%, 91%, 92%, 93%, 94%, 95%, 96%, 97%, 98%, or 99% identical thereto.

55. An isolated nucleic acid molecule encoding an antibody, or an antigen-binding fragment thereof, a heavy chain variable region, a light chain variable region, heavy chain, or light chain, of any of the preceding claims.

56. An expression vector comprising the nucleic acid molecule of claim 55.

57. A host cell comprising the nucleic acid molecule of claim 55 or the vector of claim 56.

58. An antibody or antigen-binding fragment produced by the host cell of claim 57.

59. A pharmaceutical composition comprising an antibody, or antigen-binding fragment thereof, of any one of claims 1-54.

60. The pharmaceutical composition of claim 59, wherein the composition is formulated for intravenous or subcutaneous injection.

61. The pharmaceutical composition of claim 59, wherein the composition is an injectable pharmaceutical composition.

62. A method of producing a polypeptide comprising a heavy chain variable region and/or light chain variable region, the method comprising:
(a) growing or culturing the host cell of claim 57 under conditions so that the host cell expresses a polypeptide comprising the heavy chain variable region and/or the light chain variable region; and
(b) purifying the polypeptide comprising the heavy chain variable region and/or the light chain variable region.

63. A method of producing an antibody that binds human «11β1 integrin, or an antigen-binding fragment thereof, the method comprising:
(a) growing or culturing the host cell of claim 57 under conditions so that the host cell expresses a polypeptide or polypeptides comprising an immunoglobulin heavy chain variable region and/or an immunoglobulin light chain variable region, thereby producing the antibody or the antigen-binding fragment of the antibody; and
(b) purifying the antibody, or the antigen-binding fragment thereof.

64. A method of treating a subject with α11β1 integrin mediated disorder, the method comprising administering to the subject an antibody, or antigen-binding fragment thereof, of any one of claims 1-54 or a pharmaceutical composition of any one of claims 59-61.

65. The method of claim 64, wherein the disorder is a fibrotic disorder, an autoimmune disorder, an inflammation disorder, or cancer.

66. The method of claim 65, wherein the fibrotic disorder is or comprises idiopathic pulmonary fibrosis (IPF), chronic kidney disease, diabetic cardiomyopathy, primary sclerosing cholangitis (PSC), primary biliary cirrhosis (PBC), nonalcoholic fatty liver disease (NAFLD/NASH), Crohn's disease, ulcerative colitis, or systemic sclerosis. In some embodiments, a fibrotic disorder is or comprises atrial fibrosis, endomyocardial fibrosis, arthrofibrosis, mediastinal fibrosis, myelofibrosis, progressive massive fibrosis, retroperitoneal fibrosis or skeletal muscle fibrosis.

67. The method of claim 65, wherein the autoimmune disorder is or comprises alopecia areata, ankylosing spondylitis, antiphospholipid syndrome, Addison's disease, hemolytic anemia, autoimmune hepatitis, hepatitis, Behcets disease, bullous pemphigoid, cardiomyopathy, celiac sprue-dermatitis, chronic fatigue immune dysfunction syndrome, chronic inflammatory demyelinating polyneuropathy, Churg-Strauss syndrome, cicatricial pemphigoid, limited scleroderma (CREST syndrome), cold agglutinin disease, Crohn's disease, dermatomyositis, discoid lupus, essential mixed cryoglobulinemia, fibromyalgia, fibromyositis, Graves' disease, Hashimoto's thyroiditis, hypothyroidism, inflammatory bowel disease, autoimmune lymphoproliferative syndrome, idiopathic pulmonary fibrosis, IgA nephropathy, insulin dependent diabetes, juvenile arthritis, lichen planus, lupus, Meniere's Disease, mixed connective tissue disease, multiple sclerosis, pernicious anemia, polyarteritis nodosa, polychondritis, polyglandular syndromes, polymyalgia rheumatica, polymyositis, primary agammaglobulinemia, primary biliary cirrhosis, psoriasis, Raynaud's phenomenon, Reiter's syndrome, rheumatic fever, rheumatoid arthritis, sarcoidosis, scleroderma, Sjogren's syndrome, stiff-man syndrome, Takayasu arteritis, temporal arteritis, ulcerative colitis, uveitis, vitiligo, and Wegener's granulomatosis.

68. The method of claim 65, wherein the inflammation disorder is hidradenitis suppurativa.

69. The method of claim 65, wherein the cancer is or comprises head and neck squamous cell carcinomas, pancreatic ductal adenocarcinoma, non-small cell lung cancer, adrenocortical carcinoma, acute myeloid leukemia, bladder urothelial carcinoma, invasive breast carcinoma, cervical squamous cell carcinoma, cholangiocarcinoma, colorectal adenocarcinoma, diffuse large B-cell lymphoma, esophageal adenocarcinoma, glioblastoma multiforme, liver hepatocellular carcinoma, lung adenocarcinoma, lung squamous cell carcinoma, skin cutaneous melanoma, mesothelioma, ovarian serous cystadenocarcinoma, pheochromocytoma and paraganglioma, prostate adenocarcinoma, sarcoma, stomach adenocarcinoma, testicular germ cell tumors, thymoma, thyroid carcinoma, uterine corpus endometrial carcinoma, uterine carcinosarcoma, uveal melanoma, kidney renal clear cell carcinoma, kidney chromophobe, or kidney renal papillary cell carcinoma

70. An antibody or antigen-binding fragment thereof of any one of claims 1-54 or a pharmaceutical composition of any one of claims 59-61, for the use in the treatment of a α11β1 integrin mediated disorder, such as those provided for herein.

71. An antibody or binding fragment thereof of any one of claims 1-54 or a pharmaceutical composition of any one of claims 59-61 for use as a medicament.

72. Use of the antibody or binding fragment thereof of any one of claims 1-54 or a pharmaceutical composition of any one of claims 59-61, for the treatment of a α11β1 integrin mediated disorder, such as those provided for herein.
